SEQUENCE LISTING (134 sequences)

SEQ ID NO: 1 - RNA, 21 nt, synthetic construct
  modified_base 1: mod_base = OTHER; note = 5 -cyclopropyl phosphonate-2 -O-methyluridine
  modified_base 1^2: mod_base = OTHER; note = phosphorothioate linkage
  modified_base 2: mod_base = OTHER; note = 2'-fluorouridine
  modified_base 2^3: mod_base = OTHER; note = phosphorothioate linkage
  modified_base 3: mod_base = um
  modified_base 3^4: mod_base = OTHER; note = phosphorothioate linkage
  modified_base 4: mod_base = OTHER; note = 2'-fluoroadenosine
  modified_base 5: mod_base = gm
  modified_base 6: mod_base = OTHER; note = 2'-O-methyladenosine
  modified_base 7: mod_base = gm
  modified_base 8: mod_base = um
  modified_base 9: mod_base = gm
  modified_base 10: mod_base = OTHER; note = 2'-O-methyladenosine
  modified_base 11: mod_base = gm
  modified_base 12: mod_base = OTHER; note = 2'-fluoroguanosine
  modified_base 13: mod_base = OTHER; note = 2'-O-methyladenosine
  modified_base 14: mod_base = OTHER; note = 2'-fluorouridine
  modified_base 15: mod_base = um
  modified_base 16: mod_base = OTHER; note = 2'-fluoroadenosine
  modified_base 17: mod_base = OTHER; note = 2'-O-methyladenosine
  modified_base 18: mod_base = OTHER; note = 2'-fluoroadenosine
  modified_base 19: mod_base = OTHER; note = 2'-O-methyladenosine
  modified_base 20: mod_base = OTHER; note = 2'-fluorouridine
  modified_base 20^21: mod_base = OTHER; note = phosphorothioate linkage
  modified_base 21: mod_base = gm
  source 1..21: mol_type = other RNA
tttagagtgaggattaaaatg

SEQ ID NO: 2 - RNA, 21 nt, synthetic construct
  modified_base 1: mod_base = OTHER; note = 5 -cyclopropyl phosphonate-2 -O-methyluridine
  modified_base 1^2: mod_base = OTHER; note = phosphorothioate linkage
  modified_base 2: mod_base = OTHER; note = 2'-fluorouridine
  modified_base 2^3: mod_base = OTHER; note = phosphorothioate linkage
  modified_base 3: mod_base = um
  modified_base 3^4: mod_base = OTHER; note = phosphorothioate linkage
  modified_base 4: mod_base = OTHER; note = 2'-fluoroadenosine
  modified_base 5: mod_base = gm
  modified_base 6: mod_base = OTHER; note = 2'-O-methyladenosine
  modified_base 7: mod_base = gm
  modified_base 8: mod_base = um
  modified_base 9: mod_base = gm
  modified_base 10: mod_base = OTHER; note = 2'-O-methyladenosine
  modified_base 11: mod_base = gm
  modified_base 12: mod_base = OTHER; note = 2'-fluoroguanosine
  modified_base 13: mod_base = OTHER; note = 2'-O-methyladenosine
  modified_base 14: mod_base = OTHER; note = 2'-fluorouridine
  modified_base 15: mod_base = um
  modified_base 16: mod_base = OTHER; note = 2'-fluoroadenosine
  modified_base 17: mod_base = OTHER; note = 2'-O-methyladenosine
  modified_base 18: mod_base = OTHER; note = 2'-fluoroadenosine
  modified_base 19: mod_base = OTHER; note = 2'-O-methyladenosine
  modified_base 20: mod_base = OTHER; note = 2'-fluorouridine
  modified_base 20^21: mod_base = OTHER; note = phosphorothioate linkage
  modified_base 21: mod_base = gm
  source 1..21: mol_type = other RNA
tttagagtgaggattaaaatg

SEQ ID NO: 3 - RNA, 21 nt, synthetic construct
  modified_base 1: mod_base = OTHER; note = 5 -cyclopropyl phosphonate-2 -O-methyluridine
  modified_base 1^2: mod_base = OTHER; note = phosphorothioate linkage
  modified_base 2: mod_base = OTHER; note = 2'-fluorouridine
  modified_base 2^3: mod_base = OTHER; note = phosphorothioate linkage
  modified_base 3: mod_base = um
  modified_base 3^4: mod_base = OTHER; note = phosphorothioate linkage
  modified_base 4: mod_base = OTHER; note = 2'-fluoroadenosine
  modified_base 5: mod_base = gm
  modified_base 6: mod_base = OTHER; note = 2'-O-methyladenosine
  modified_base 7: mod_base = gm
  modified_base 8: mod_base = um
  modified_base 9: mod_base = gm
  modified_base 10: mod_base = OTHER; note = 2'-O-methyladenosine
  modified_base 11: mod_base = gm
  modified_base 12: mod_base = OTHER; note = 2'-fluoroguanosine
  modified_base 13: mod_base = OTHER; note = 2'-O-methyladenosine
  modified_base 14: mod_base = OTHER; note = 2'-fluorouridine
  modified_base 15: mod_base = um
  modified_base 16: mod_base = OTHER; note = 2'-fluoroadenosine
  modified_base 17: mod_base = OTHER; note = 2'-O-methyladenosine
  modified_base 18: mod_base = OTHER; note = 2'-fluoroadenosine
  modified_base 19: mod_base = OTHER; note = 2'-O-methyladenosine
  modified_base 20: mod_base = OTHER; note = 2'-fluorouridine
  modified_base 20^21: mod_base = OTHER; note = phosphorothioate linkage
  modified_base 21: mod_base = gm
  source 1..21: mol_type = other RNA
tttagagtgaggattaaaatg

SEQ ID NO: 4 - RNA, 21 nt, synthetic construct
  modified_base 1: mod_base = OTHER; note = 5 -cyclopropyl phosphonate-2 -O-methyluridine
  modified_base 1^2: mod_base = OTHER; note = phosphorothioate linkage
  modified_base 2: mod_base = OTHER; note = 2'-fluorouridine
  modified_base 2^3: mod_base = OTHER; note = phosphorothioate linkage
  modified_base 3: mod_base = um
  modified_base 3^4: mod_base = OTHER; note = phosphorothioate linkage
  modified_base 4: mod_base = OTHER; note = 2'-fluoroadenosine
  modified_base 5: mod_base = gm
  modified_base 6: mod_base = OTHER; note = 2'-O-methyladenosine
  modified_base 7: mod_base = gm
  modified_base 8: mod_base = um
  modified_base 9: mod_base = gm
  modified_base 10: mod_base = OTHER; note = 2'-O-methyladenosine
  modified_base 11: mod_base = gm
  modified_base 12: mod_base = OTHER; note = 2'-fluoroguanosine
  modified_base 13: mod_base = OTHER; note = 2'-O-methyladenosine
  modified_base 14: mod_base = OTHER; note = 2'-fluorouridine
  modified_base 15: mod_base = um
  modified_base 16: mod_base = OTHER; note = 2'-fluoroadenosine
  modified_base 17: mod_base = OTHER; note = 2'-O-methyladenosine
  modified_base 18: mod_base = OTHER; note = 2'-fluoroadenosine
  modified_base 19: mod_base = OTHER; note = 2'-O-methyladenosine
  modified_base 20: mod_base = OTHER; note = 2'-fluorouridine
  modified_base 20^21: mod_base = OTHER; note = phosphorothioate linkage
  modified_base 21: mod_base = gm
  source 1..21: mol_type = other RNA
tttagagtgaggattaaaatg

SEQ ID NO: 5 - RNA, 21 nt, synthetic construct
  modified_base 1: mod_base = OTHER; note = 5 -cyclopropyl phosphonate-2 -O-methyluridine
  modified_base 1^2: mod_base = OTHER; note = phosphorothioate linkage
  modified_base 2: mod_base = OTHER; note = 2'-fluorouridine
  modified_base 2^3: mod_base = OTHER; note = phosphorothioate linkage
  modified_base 3: mod_base = um
  modified_base 3^4: mod_base = OTHER; note = phosphorothioate linkage
  modified_base 4: mod_base = OTHER; note = 2'-fluoroadenosine
  modified_base 5: mod_base = gm
  modified_base 6: mod_base = OTHER; note = 2'-O-methyladenosine
  modified_base 7: mod_base = gm
  modified_base 8: mod_base = um
  modified_base 9: mod_base = gm
  modified_base 10: mod_base = OTHER; note = 2'-O-methyladenosine
  modified_base 11: mod_base = gm
  modified_base 12: mod_base = OTHER; note = 2'-fluoroguanosine
  modified_base 13: mod_base = OTHER; note = 2'-O-methyladenosine
  modified_base 14: mod_base = OTHER; note = 2'-fluorouridine
  modified_base 15: mod_base = um
  modified_base 16: mod_base = OTHER; note = 2'-fluoroadenosine
  modified_base 17: mod_base = OTHER; note = 2'-O-methyladenosine
  modified_base 18: mod_base = OTHER; note = 2'-fluoroadenosine
  modified_base 19: mod_base = OTHER; note = 2'-O-methyladenosine
  modified_base 20: mod_base = OTHER; note = 2'-fluorouridine
  modified_base 20^21: mod_base = OTHER; note = phosphorothioate linkage
  modified_base 21: mod_base = gm
  source 1..21: mol_type = other RNA
tttagagtgaggattaaaatg

SEQ ID NO: 6 - RNA, 21 nt, synthetic construct
  modified_base 1: mod_base = OTHER; note = 5 -cyclopropyl phosphonate-2 -O-methyluridine
  modified_base 1^2: mod_base = OTHER; note = phosphorothioate linkage
  modified_base 2: mod_base = OTHER; note = 2'-fluorouridine
  modified_base 2^3: mod_base = OTHER; note = phosphorothioate linkage
  modified_base 3: mod_base = um
  modified_base 3^4: mod_base = OTHER; note = phosphorothioate linkage
  modified_base 4: mod_base = OTHER; note = 2'-fluoroadenosine
  modified_base 5: mod_base = gm
  modified_base 6: mod_base = OTHER; note = 2'-O-methyladenosine
  modified_base 7: mod_base = gm
  modified_base 8: mod_base = um
  modified_base 9: mod_base = gm
  modified_base 10: mod_base = OTHER; note = 2'-O-methyladenosine
  modified_base 11: mod_base = gm
  modified_base 12: mod_base = OTHER; note = 2'-fluoroguanosine
  modified_base 13: mod_base = OTHER; note = 2'-O-methyladenosine
  modified_base 14: mod_base = OTHER; note = 2'-fluorouridine
  modified_base 15: mod_base = um
  modified_base 16: mod_base = OTHER; note = 2'-fluoroadenosine
  modified_base 17: mod_base = OTHER; note = 2'-O-methyladenosine
  modified_base 18: mod_base = OTHER; note = 2'-fluoroadenosine
  modified_base 19: mod_base = OTHER; note = 2'-O-methyladenosine
  modified_base 20: mod_base = OTHER; note = 2'-fluorouridine
  modified_base 20^21: mod_base = OTHER; note = phosphorothioate linkage
  modified_base 21: mod_base = gm
  source 1..21: mol_type = other RNA
tttagagtgaggattaaaatg

SEQ ID NO: 7 - RNA, 21 nt, synthetic construct
  modified_base 1: mod_base = OTHER; note = 5 -cyclopropyl phosphonate-2 -O-methyluridine
  modified_base 1^2: mod_base = OTHER; note = phosphorothioate linkage
  modified_base 2: mod_base = OTHER; note = 2'-fluorouridine
  modified_base 2^3: mod_base = OTHER; note = phosphorothioate linkage
  modified_base 3: mod_base = um
  modified_base 3^4: mod_base = OTHER; note = phosphorothioate linkage
  modified_base 4: mod_base = OTHER; note = 2'-fluoroadenosine
  modified_base 5: mod_base = gm
  modified_base 6: mod_base = OTHER; note = 2'-O-methyladenosine
  modified_base 7: mod_base = gm
  modified_base 8: mod_base = um
  modified_base 9: mod_base = gm
  modified_base 10: mod_base = OTHER; note = 2'-O-methyladenosine
  modified_base 11: mod_base = gm
  modified_base 12: mod_base = OTHER; note = 2'-fluoroguanosine
  modified_base 13: mod_base = OTHER; note = 2'-O-methyladenosine
  modified_base 14: mod_base = OTHER; note = 2'-fluorouridine
  modified_base 15: mod_base = um
  modified_base 16: mod_base = OTHER; note = 2'-fluoroadenosine
  modified_base 17: mod_base = OTHER; note = 2'-O-methyladenosine
  modified_base 18: mod_base = OTHER; note = 2'-fluoroadenosine
  modified_base 19: mod_base = OTHER; note = 2'-O-methyladenosine
  modified_base 20: mod_base = OTHER; note = 2'-fluorouridine
  modified_base 20^21: mod_base = OTHER; note = phosphorothioate linkage
  modified_base 21: mod_base = gm
  source 1..21: mol_type = other RNA
tttagagtgaggattaaaatg

SEQ ID NO: 8 - RNA, 21 nt, synthetic construct
  modified_base 1: mod_base = OTHER; note = 5 -cyclopropyl phosphonate-2 -O-methyluridine
  modified_base 1^2: mod_base = OTHER; note = phosphorothioate linkage
  modified_base 2: mod_base = OTHER; note = 2'-fluorouridine
  modified_base 2^3: mod_base = OTHER; note = phosphorothioate linkage
  modified_base 3: mod_base = um
  modified_base 3^4: mod_base = OTHER; note = phosphorothioate linkage
  modified_base 4: mod_base = OTHER; note = 2'-fluoroadenosine
  modified_base 5: mod_base = gm
  modified_base 6: mod_base = OTHER; note = 2'-O-methyladenosine
  modified_base 7: mod_base = gm
  modified_base 8: mod_base = um
  modified_base 9: mod_base = gm
  modified_base 10: mod_base = OTHER; note = 2'-O-methyladenosine
  modified_base 11: mod_base = gm
  modified_base 12: mod_base = OTHER; note = 2'-fluoroguanosine
  modified_base 13: mod_base = OTHER; note = 2'-O-methyladenosine
  modified_base 14: mod_base = OTHER; note = 2'-fluorouridine
  modified_base 15: mod_base = um
  modified_base 16: mod_base = OTHER; note = 2'-fluoroadenosine
  modified_base 17: mod_base = OTHER; note = 2'-O-methyladenosine
  modified_base 18: mod_base = OTHER; note = 2'-fluoroadenosine
  modified_base 19: mod_base = OTHER; note = 2'-O-methyladenosine
  modified_base 20: mod_base = OTHER; note = 2'-fluorouridine
  modified_base 20^21: mod_base = OTHER; note = phosphorothioate linkage
  modified_base 21: mod_base = gm
  source 1..21: mol_type = other RNA
tttagagtgaggattaaaatg

SEQ ID NO: 9 - RNA, 21 nt, synthetic construct
  modified_base 1: mod_base = OTHER; note = 5 -cyclopropyl phosphonate-2 -O-methyluridine
  modified_base 1^2: mod_base = OTHER; note = phosphorothioate linkage
  modified_base 2: mod_base = OTHER; note = 2'-fluorouridine
  modified_base 2^3: mod_base = OTHER; note = phosphorothioate linkage
  modified_base 3: mod_base = um
  modified_base 3^4: mod_base = OTHER; note = phosphorothioate linkage
  modified_base 4: mod_base = OTHER; note = 2'-fluoroadenosine
  modified_base 5: mod_base = gm
  modified_base 6: mod_base = OTHER; note = 2'-O-methyladenosine
  modified_base 7: mod_base = gm
  modified_base 8: mod_base = um
  modified_base 9: mod_base = gm
  modified_base 10: mod_base = OTHER; note = 2'-O-methyladenosine
  modified_base 11: mod_base = gm
  modified_base 12: mod_base = OTHER; note = 2'-fluoroguanosine
  modified_base 13: mod_base = OTHER; note = 2'-O-methyladenosine
  modified_base 14: mod_base = OTHER; note = 2'-fluorouridine
  modified_base 15: mod_base = um
  modified_base 16: mod_base = OTHER; note = 2'-fluoroadenosine
  modified_base 17: mod_base = OTHER; note = 2'-O-methyladenosine
  modified_base 18: mod_base = OTHER; note = 2'-fluoroadenosine
  modified_base 19: mod_base = OTHER; note = 2'-O-methyladenosine
  modified_base 20: mod_base = OTHER; note = 2'-fluorouridine
  modified_base 20^21: mod_base = OTHER; note = phosphorothioate linkage
  modified_base 21: mod_base = gm
  source 1..21: mol_type = other RNA
tttagagtgaggattaaaatg

SEQ ID NO: 10 - RNA, 21 nt, synthetic construct
  modified_base 1: mod_base = OTHER; note = 5 -cyclopropyl phosphonate-2 -O-methyluridine
  modified_base 1^2: mod_base = OTHER; note = phosphorothioate linkage
  modified_base 2: mod_base = OTHER; note = 2'-fluorouridine
  modified_base 2^3: mod_base = OTHER; note = phosphorothioate linkage
  modified_base 3: mod_base = um
  modified_base 3^4: mod_base = OTHER; note = phosphorothioate linkage
  modified_base 4: mod_base = OTHER; note = 2'-fluoroadenosine
  modified_base 5: mod_base = gm
  modified_base 6: mod_base = OTHER; note = 2'-O-methyladenosine
  modified_base 7: mod_base = gm
  modified_base 8: mod_base = um
  modified_base 9: mod_base = gm
  modified_base 10: mod_base = OTHER; note = 2'-O-methyladenosine
  modified_base 11: mod_base = gm
  modified_base 12: mod_base = OTHER; note = 2'-fluoroguanosine
  modified_base 13: mod_base = OTHER; note = 2'-O-methyladenosine
  modified_base 14: mod_base = OTHER; note = 2'-fluorouridine
  modified_base 15: mod_base = um
  modified_base 16: mod_base = OTHER; note = 2'-fluoroadenosine
  modified_base 17: mod_base = OTHER; note = 2'-O-methyladenosine
  modified_base 18: mod_base = OTHER; note = 2'-fluoroadenosine
  modified_base 19: mod_base = OTHER; note = 2'-O-methyladenosine
  modified_base 20: mod_base = OTHER; note = 2'-fluorouridine
  modified_base 20^21: mod_base = OTHER; note = phosphorothioate linkage
  modified_base 21: mod_base = gm
  source 1..21: mol_type = other RNA
tttagagtgaggattaaaatg

SEQ ID NO: 11 - RNA, 21 nt, synthetic construct
  modified_base 1: mod_base = OTHER; note = 5 -cyclopropyl phosphonate-2 -O-methyluridine
  modified_base 1^2: mod_base = OTHER; note = phosphorothioate linkage
  modified_base 2: mod_base = OTHER; note = 2'-fluorouridine
  modified_base 2^3: mod_base = OTHER; note = phosphorothioate linkage
  modified_base 3: mod_base = um
  modified_base 3^4: mod_base = OTHER; note = phosphorothioate linkage
  modified_base 4: mod_base = OTHER; note = 2'-fluoroadenosine
  modified_base 5: mod_base = gm
  modified_base 6: mod_base = OTHER; note = 2'-O-methyladenosine
  modified_base 7: mod_base = gm
  modified_base 8: mod_base = um
  modified_base 9: mod_base = gm
  modified_base 10: mod_base = OTHER; note = 2'-O-methyladenosine
  modified_base 11: mod_base = gm
  modified_base 12: mod_base = OTHER; note = 2'-fluoroguanosine
  modified_base 13: mod_base = OTHER; note = 2'-O-methyladenosine
  modified_base 14: mod_base = OTHER; note = 2'-fluorouridine
  modified_base 15: mod_base = um
  modified_base 16: mod_base = OTHER; note = 2'-fluoroadenosine
  modified_base 17: mod_base = OTHER; note = 2'-O-methyladenosine
  modified_base 18: mod_base = OTHER; note = 2'-fluoroadenosine
  modified_base 19: mod_base = OTHER; note = 2'-O-methyladenosine
  modified_base 20: mod_base = OTHER; note = 2'-fluorouridine
  modified_base 20^21: mod_base = OTHER; note = phosphorothioate linkage
  modified_base 21: mod_base = gm
  source 1..21: mol_type = other RNA
tttagagtgaggattaaaatg

SEQ ID NO: 12 - RNA, 21 nt, synthetic construct
  modified_base 1: mod_base = OTHER; note = 5 -cyclopropyl phosphonate-2 -O-methyluridine
  modified_base 1^2: mod_base = OTHER; note = phosphorothioate linkage
  modified_base 2: mod_base = OTHER; note = 2'-fluorouridine
  modified_base 2^3: mod_base = OTHER; note = phosphorothioate linkage
  modified_base 3: mod_base = um
  modified_base 3^4: mod_base = OTHER; note = phosphorothioate linkage
  modified_base 4: mod_base = OTHER; note = 2'-fluoroadenosine
  modified_base 5: mod_base = gm
  modified_base 6: mod_base = OTHER; note = 2'-O-methyladenosine
  modified_base 7: mod_base = gm
  modified_base 8: mod_base = um
  modified_base 9: mod_base = gm
  modified_base 10: mod_base = OTHER; note = 2'-O-methyladenosine
  modified_base 11: mod_base = gm
  modified_base 12: mod_base = OTHER; note = 2'-fluoroguanosine
  modified_base 13: mod_base = OTHER; note = 2'-O-methyladenosine
  modified_base 14: mod_base = OTHER; note = 2'-fluorouridine
  modified_base 15: mod_base = um
  modified_base 16: mod_base = OTHER; note = 2'-fluoroadenosine
  modified_base 17: mod_base = OTHER; note = 2'-O-methyladenosine
  modified_base 18: mod_base = OTHER; note = 2'-fluoroadenosine
  modified_base 19: mod_base = OTHER; note = 2'-O-methyladenosine
  modified_base 20: mod_base = OTHER; note = 2'-fluorouridine
  modified_base 20^21: mod_base = OTHER; note = phosphorothioate linkage
  modified_base 21: mod_base = gm
  source 1..21: mol_type = other RNA
tttagagtgaggattaaaatg

SEQ ID NO: 13 - RNA, 21 nt, synthetic construct
  modified_base 1: mod_base = OTHER; note = 5 -cyclopropyl phosphonate-2 -O-methyluridine
  modified_base 1^2: mod_base = OTHER; note = phosphorothioate linkage
  modified_base 2: mod_base = OTHER; note = 2'-fluorouridine
  modified_base 2^3: mod_base = OTHER; note = phosphorothioate linkage
  modified_base 3: mod_base = um
  modified_base 3^4: mod_base = OTHER; note = phosphorothioate linkage
  modified_base 4: mod_base = OTHER; note = 2'-fluoroadenosine
  modified_base 5: mod_base = gm
  modified_base 6: mod_base = OTHER; note = 2'-O-methyladenosine
  modified_base 7: mod_base = gm
  modified_base 8: mod_base = um
  modified_base 9: mod_base = gm
  modified_base 10: mod_base = OTHER; note = 2'-O-methyladenosine
  modified_base 11: mod_base = gm
  modified_base 12: mod_base = OTHER; note = 2'-fluoroguanosine
  modified_base 13: mod_base = OTHER; note = 2'-O-methyladenosine
  modified_base 14: mod_base = OTHER; note = 2'-fluorouridine
  modified_base 15: mod_base = um
  modified_base 16: mod_base = OTHER; note = 2'-fluoroadenosine
  modified_base 17: mod_base = OTHER; note = 2'-O-methyladenosine
  modified_base 18: mod_base = OTHER; note = 2'-fluoroadenosine
  modified_base 19: mod_base = OTHER; note = 2'-O-methyladenosine
  modified_base 20: mod_base = OTHER; note = 2'-fluorouridine
  modified_base 20^21: mod_base = OTHER; note = phosphorothioate linkage
  modified_base 21: mod_base = gm
  source 1..21: mol_type = other RNA
tttagagtgaggattaaaatg

SEQ ID NO: 14 - RNA, 21 nt, synthetic construct
  modified_base 1: mod_base = OTHER; note = 5 -cyclopropyl phosphonate-2 -O-methyluridine
  modified_base 1^2: mod_base = OTHER; note = phosphorothioate linkage
  modified_base 2: mod_base = OTHER; note = 2'-fluorouridine
  modified_base 2^3: mod_base = OTHER; note = phosphorothioate linkage
  modified_base 3: mod_base = um
  modified_base 3^4: mod_base = OTHER; note = phosphorothioate linkage
  modified_base 4: mod_base = OTHER; note = 2'-fluoroadenosine
  modified_base 5: mod_base = gm
  modified_base 6: mod_base = OTHER; note = 2'-O-methyladenosine
  modified_base 7: mod_base = gm
  modified_base 8: mod_base = um
  modified_base 9: mod_base = gm
  modified_base 10: mod_base = OTHER; note = 2'-O-methyladenosine
  modified_base 11: mod_base = gm
  modified_base 12: mod_base = OTHER; note = 2'-fluoroguanosine
  modified_base 13: mod_base = OTHER; note = 2'-O-methyladenosine
  modified_base 14: mod_base = OTHER; note = 2'-fluorouridine
  modified_base 15: mod_base = um
  modified_base 16: mod_base = OTHER; note = 2'-fluoroadenosine
  modified_base 17: mod_base = OTHER; note = 2'-O-methyladenosine
  modified_base 18: mod_base = OTHER; note = 2'-fluoroadenosine
  modified_base 19: mod_base = OTHER; note = 2'-O-methyladenosine
  modified_base 20: mod_base = OTHER; note = 2'-fluorouridine
  modified_base 20^21: mod_base = OTHER; note = phosphorothioate linkage
  modified_base 21: mod_base = gm
  source 1..21: mol_type = other RNA
tttagagtgaggattaaaatg

SEQ ID NO: 15 - RNA, 21 nt, synthetic construct
  modified_base 1: mod_base = OTHER; note = 5 -cyclopropyl phosphonate-2 -O-methyluridine
  modified_base 1^2: mod_base = OTHER; note = phosphorothioate linkage
  modified_base 2: mod_base = OTHER; note = 2'-fluorouridine
  modified_base 2^3: mod_base = OTHER; note = phosphorothioate linkage
  modified_base 3: mod_base = um
  modified_base 3^4: mod_base = OTHER; note = phosphorothioate linkage
  modified_base 4: mod_base = OTHER; note = 2'-fluoroadenosine
  modified_base 5: mod_base = gm
  modified_base 6: mod_base = OTHER; note = 2'-O-methyladenosine
  modified_base 7: mod_base = gm
  modified_base 8: mod_base = um
  modified_base 9: mod_base = gm
  modified_base 10: mod_base = OTHER; note = 2'-O-methyladenosine
  modified_base 11: mod_base = gm
  modified_base 12: mod_base = OTHER; note = 2'-fluoroguanosine
  modified_base 13: mod_base = OTHER; note = 2'-O-methyladenosine
  modified_base 14: mod_base = OTHER; note = 2'-fluorouridine
  modified_base 15: mod_base = um
  modified_base 16: mod_base = OTHER; note = 2'-fluoroadenosine
  modified_base 17: mod_base = OTHER; note = 2'-O-methyladenosine
  modified_base 18: mod_base = OTHER; note = 2'-fluoroadenosine
  modified_base 19: mod_base = OTHER; note = 2'-O-methyladenosine
  modified_base 20: mod_base = OTHER; note = 2'-fluorouridine
  modified_base 20^21: mod_base = OTHER; note = phosphorothioate linkage
  modified_base 21: mod_base = gm
  source 1..21: mol_type = other RNA
tttagagtgaggattaaaatg

SEQ ID NO: 16 - RNA, 21 nt, synthetic construct
  modified_base 1: mod_base = OTHER; note = 5 -cyclopropyl phosphonate-2 -O-methyluridine
  modified_base 1^2: mod_base = OTHER; note = phosphorothioate linkage
  modified_base 2: mod_base = OTHER; note = 2'-fluorouridine
  modified_base 2^3: mod_base = OTHER; note = phosphorothioate linkage
  modified_base 3: mod_base = um
  modified_base 3^4: mod_base = OTHER; note = phosphorothioate linkage
  modified_base 4: mod_base = OTHER; note = 2'-fluoroadenosine
  modified_base 5: mod_base = gm
  modified_base 6: mod_base = OTHER; note = 2'-O-methyladenosine
  modified_base 7: mod_base = gm
  modified_base 8: mod_base = um
  modified_base 9: mod_base = gm
  modified_base 10: mod_base = OTHER; note = 2'-O-methyladenosine
  modified_base 11: mod_base = gm
  modified_base 12: mod_base = OTHER; note = 2'-fluoroguanosine
  modified_base 13: mod_base = OTHER; note = 2'-O-methyladenosine
  modified_base 14: mod_base = OTHER; note = 2'-fluorouridine
  modified_base 15: mod_base = um
  modified_base 16: mod_base = OTHER; note = 2'-fluoroadenosine
  modified_base 17: mod_base = OTHER; note = 2'-O-methyladenosine
  modified_base 18: mod_base = OTHER; note = 2'-fluoroadenosine
  modified_base 19: mod_base = OTHER; note = 2'-O-methyladenosine
  modified_base 20: mod_base = OTHER; note = 2'-fluorouridine
  modified_base 20^21: mod_base = OTHER; note = phosphorothioate linkage
  modified_base 21: mod_base = gm
  source 1..21: mol_type = other RNA
tttagagtgaggattaaaatg

SEQ ID NO: 17 - RNA, 21 nt, synthetic construct
  modified_base 1: mod_base = OTHER; note = 5 -cyclopropyl phosphonate-2 -O-methyluridine
  modified_base 1^2: mod_base = OTHER; note = phosphorothioate linkage
  modified_base 2: mod_base = OTHER; note = 2'-fluorouridine
  modified_base 2^3: mod_base = OTHER; note = phosphorothioate linkage
  modified_base 3: mod_base = um
  modified_base 3^4: mod_base = OTHER; note = phosphorothioate linkage
  modified_base 4: mod_base = OTHER; note = 2'-fluoroadenosine
  modified_base 5: mod_base = gm
  modified_base 6: mod_base = OTHER; note = 2'-O-methyladenosine
  modified_base 7: mod_base = gm
  modified_base 8: mod_base = um
  modified_base 9: mod_base = gm
  modified_base 10: mod_base = OTHER; note = 2'-O-methyladenosine
  modified_base 11: mod_base = gm
  modified_base 12: mod_base = OTHER; note = 2'-fluoroguanosine
  modified_base 13: mod_base = OTHER; note = 2'-O-methyladenosine
  modified_base 14: mod_base = OTHER; note = 2'-fluorouridine
  modified_base 15: mod_base = um
  modified_base 16: mod_base = OTHER; note = 2'-fluoroadenosine
  modified_base 17: mod_base = OTHER; note = 2'-O-methyladenosine
  modified_base 18: mod_base = OTHER; note = 2'-fluoroadenosine
  modified_base 19: mod_base = OTHER; note = 2'-O-methyladenosine
  modified_base 20: mod_base = OTHER; note = 2'-fluorouridine
  modified_base 20^21: mod_base = OTHER; note = phosphorothioate linkage
  modified_base 21: mod_base = gm
  source 1..21: mol_type = other RNA
tttagagtgaggattaaaatg

SEQ ID NO: 18 - RNA, 21 nt, synthetic construct
  modified_base 1: mod_base = OTHER; note = 5 -cyclopropyl phosphonate-2 -O-methyluridine
  modified_base 1^2: mod_base = OTHER; note = phosphorothioate linkage
  modified_base 2: mod_base = OTHER; note = 2'-fluorouridine
  modified_base 2^3: mod_base = OTHER; note = phosphorothioate linkage
  modified_base 3: mod_base = um
  modified_base 3^4: mod_base = OTHER; note = phosphorothioate linkage
  modified_base 4: mod_base = OTHER; note = 2'-fluoroadenosine
  modified_base 5: mod_base = gm
  modified_base 6: mod_base = OTHER; note = 2'-O-methyladenosine
  modified_base 7: mod_base = gm
  modified_base 8: mod_base = um
  modified_base 9: mod_base = gm
  modified_base 10: mod_base = OTHER; note = 2'-O-methyladenosine
  modified_base 11: mod_base = gm
  modified_base 12: mod_base = OTHER; note = 2'-fluoroguanosine
  modified_base 13: mod_base = OTHER; note = 2'-O-methyladenosine
  modified_base 14: mod_base = OTHER; note = 2'-fluorouridine
  modified_base 15: mod_base = um
  modified_base 16: mod_base = OTHER; note = 2'-fluoroadenosine
  modified_base 17: mod_base = OTHER; note = 2'-O-methyladenosine
  modified_base 18: mod_base = OTHER; note = 2'-fluoroadenosine
  modified_base 19: mod_base = OTHER; note = 2'-O-methyladenosine
  modified_base 20: mod_base = OTHER; note = 2'-fluorouridine
  modified_base 20^21: mod_base = OTHER; note = phosphorothioate linkage
  modified_base 21: mod_base = gm
  source 1..21: mol_type = other RNA
tttagagtgaggattaaaatg

SEQ ID NO: 19 - RNA, 21 nt, synthetic construct
  modified_base 1: mod_base = OTHER; note = 5 -cyclopropyl phosphonate-2 -O-methyluridine
  modified_base 1^2: mod_base = OTHER; note = phosphorothioate linkage
  modified_base 2: mod_base = OTHER; note = 2'-fluorouridine
  modified_base 2^3: mod_base = OTHER; note = phosphorothioate linkage
  modified_base 3: mod_base = um
  modified_base 3^4: mod_base = OTHER; note = phosphorothioate linkage
  modified_base 4: mod_base = OTHER; note = 2'-fluoroadenosine
  modified_base 5: mod_base = gm
  modified_base 6: mod_base = OTHER; note = 2'-O-methyladenosine
  modified_base 7: mod_base = gm
  modified_base 8: mod_base = um
  modified_base 9: mod_base = gm
  modified_base 10: mod_base = OTHER; note = 2'-O-methyladenosine
  modified_base 11: mod_base = gm
  modified_base 12: mod_base = OTHER; note = 2'-fluoroguanosine
  modified_base 13: mod_base = OTHER; note = 2'-O-methyladenosine
  modified_base 14: mod_base = OTHER; note = 2'-fluorouridine
  modified_base 15: mod_base = um
  modified_base 16: mod_base = OTHER; note = 2'-fluoroadenosine
  modified_base 17: mod_base = OTHER; note = 2'-O-methyladenosine
  modified_base 18: mod_base = OTHER; note = 2'-fluoroadenosine
  modified_base 19: mod_base = OTHER; note = 2'-O-methyladenosine
  modified_base 20: mod_base = OTHER; note = 2'-fluorouridine
  modified_base 20^21: mod_base = OTHER; note = phosphorothioate linkage
  modified_base 21: mod_base = gm
  source 1..21: mol_type = other RNA
tttagagtgaggattaaaatg

SEQ ID NO: 20 - RNA, 21 nt, synthetic construct
  modified_base 1: mod_base = OTHER; note = 5 -cyclopropyl phosphonate-2 -O-methyluridine
  modified_base 1^2: mod_base = OTHER; note = phosphorothioate linkage
  modified_base 2: mod_base = OTHER; note = 2'-fluorouridine
  modified_base 2^3: mod_base = OTHER; note = phosphorothioate linkage
  modified_base 3: mod_base = um
  modified_base 3^4: mod_base = OTHER; note = phosphorothioate linkage
  modified_base 4: mod_base = OTHER; note = 2'-fluoroadenosine
  modified_base 5: mod_base = gm
  modified_base 6: mod_base = OTHER; note = 2'-O-methyladenosine
  modified_base 7: mod_base = gm
  modified_base 8: mod_base = um
  modified_base 9: mod_base = gm
  modified_base 10: mod_base = OTHER; note = 2'-O-methyladenosine
  modified_base 11: mod_base = gm
  modified_base 12: mod_base = OTHER; note = 2'-fluoroguanosine
  modified_base 13: mod_base = OTHER; note = 2'-O-methyladenosine
  modified_base 14: mod_base = OTHER; note = 2'-fluorouridine
  modified_base 15: mod_base = um
  modified_base 16: mod_base = OTHER; note = 2'-fluoroadenosine
  modified_base 17: mod_base = OTHER; note = 2'-O-methyladenosine
  modified_base 18: mod_base = OTHER; note = 2'-fluoroadenosine
  modified_base 19: mod_base = OTHER; note = 2'-O-methyladenosine
  modified_base 20: mod_base = OTHER; note = 2'-fluorouridine
  modified_base 20^21: mod_base = OTHER; note = phosphorothioate linkage
  modified_base 21: mod_base = gm
  source 1..21: mol_type = other RNA
tttagagtgaggattaaaatg

SEQ ID NO: 21 - RNA, 21 nt, synthetic construct
  modified_base 1: mod_base = OTHER; note = 5 -cyclopropyl phosphonate-2 -O-methyluridine
  modified_base 1^2: mod_base = OTHER; note = phosphorothioate linkage
  modified_base 2: mod_base = OTHER; note = 2'-fluorouridine
  modified_base 2^3: mod_base = OTHER; note = phosphorothioate linkage
  modified_base 3: mod_base = um
  modified_base 3^4: mod_base = OTHER; note = phosphorothioate linkage
  modified_base 4: mod_base = OTHER; note = 2'-fluoroadenosine
  modified_base 5: mod_base = gm
  modified_base 6: mod_base = OTHER; note = 2'-O-methyladenosine
  modified_base 7: mod_base = gm
  modified_base 8: mod_base = um
  modified_base 9: mod_base = gm
  modified_base 10: mod_base = OTHER; note = 2'-O-methyladenosine
  modified_base 11: mod_base = gm
  modified_base 12: mod_base = OTHER; note = 2'-fluoroguanosine
  modified_base 13: mod_base = OTHER; note = 2'-O-methyladenosine
  modified_base 14: mod_base = OTHER; note = 2'-fluorouridine
  modified_base 15: mod_base = um
  modified_base 16: mod_base = OTHER; note = 2'-fluoroadenosine
  modified_base 17: mod_base = OTHER; note = 2'-O-methyladenosine
  modified_base 18: mod_base = OTHER; note = 2'-fluoroadenosine
  modified_base 19: mod_base = OTHER; note = 2'-O-methyladenosine
  modified_base 20: mod_base = OTHER; note = 2'-fluorouridine
  modified_base 20^21: mod_base = OTHER; note = phosphorothioate linkage
  modified_base 21: mod_base = gm
  source 1..21: mol_type = other RNA
tttagagtgaggattaaaatg

SEQ ID NO: 22 - RNA, 21 nt, synthetic construct
  modified_base 1: mod_base = OTHER; note = 5 -cyclopropyl phosphonate-2 -O-methyluridine
  modified_base 1^2: mod_base = OTHER; note = phosphorothioate linkage
  modified_base 2: mod_base = OTHER; note = 2'-fluorouridine
  modified_base 2^3: mod_base = OTHER; note = phosphorothioate linkage
  modified_base 3: mod_base = um
  modified_base 3^4: mod_base = OTHER; note = phosphorothioate linkage
  modified_base 4: mod_base = OTHER; note = 2'-fluoroadenosine
  modified_base 5: mod_base = gm
  modified_base 6: mod_base = OTHER; note = 2'-O-methyladenosine
  modified_base 7: mod_base = gm
  modified_base 8: mod_base = um
  modified_base 9: mod_base = gm
  modified_base 10: mod_base = OTHER; note = 2'-O-methyladenosine
  modified_base 11: mod_base = gm
  modified_base 12: mod_base = OTHER; note = 2'-fluoroguanosine
  modified_base 13: mod_base = OTHER; note = 2'-O-methyladenosine
  modified_base 14: mod_base = OTHER; note = 2'-fluorouridine
  modified_base 15: mod_base = um
  modified_base 16: mod_base = OTHER; note = 2'-fluoroadenosine
  modified_base 17: mod_base = OTHER; note = 2'-O-methyladenosine
  modified_base 18: mod_base = OTHER; note = 2'-fluoroadenosine
  modified_base 19: mod_base = OTHER; note = 2'-O-methyladenosine
  modified_base 20: mod_base = OTHER; note = 2'-fluorouridine
  modified_base 20^21: mod_base = OTHER; note = phosphorothioate linkage
  modified_base 21: mod_base = gm
  source 1..21: mol_type = other RNA
tttagagtgaggattaaaatg

SEQ ID NO: 23 - RNA, 21 nt, synthetic construct
  modified_base 1: mod_base = OTHER; note = 5 -cyclopropyl phosphonate-2 -O-methyluridine
  modified_base 1^2: mod_base = OTHER; note = phosphorothioate linkage
  modified_base 2: mod_base = OTHER; note = 2'-fluorouridine
  modified_base 2^3: mod_base = OTHER; note = phosphorothioate linkage
  modified_base 3: mod_base = um
  modified_base 3^4: mod_base = OTHER; note = phosphorothioate linkage
  modified_base 4: mod_base = OTHER; note = 2'-fluoroadenosine
  modified_base 5: mod_base = gm
  modified_base 6: mod_base = OTHER; note = 2'-O-methyladenosine
  modified_base 7: mod_base = gm
  modified_base 8: mod_base = um
  modified_base 9: mod_base = gm
  modified_base 10: mod_base = OTHER; note = 2'-O-methyladenosine
  modified_base 11: mod_base = gm
  modified_base 12: mod_base = OTHER; note = 2'-fluoroguanosine
  modified_base 13: mod_base = OTHER; note = 2'-O-methyladenosine
  modified_base 14: mod_base = OTHER; note = 2'-fluorouridine
  modified_base 15: mod_base = um
  modified_base 16: mod_base = OTHER; note = 2'-fluoroadenosine
  modified_base 17: mod_base = OTHER; note = 2'-O-methyladenosine
  modified_base 18: mod_base = OTHER; note = 2'-fluoroadenosine
  modified_base 19: mod_base = OTHER; note = 2'-O-methyladenosine
  modified_base 20: mod_base = OTHER; note = 2'-fluorouridine
  modified_base 20^21: mod_base = OTHER; note = phosphorothioate linkage
  modified_base 21: mod_base = gm
  source 1..21: mol_type = other RNA
tttagagtgaggattaaaatg

SEQ ID NO: 24 - RNA, 21 nt, synthetic construct
  modified_base 1: mod_base = OTHER; note = 5 -cyclopropyl phosphonate-2 -O-methyluridine
  modified_base 1^2: mod_base = OTHER; note = phosphorothioate linkage
  modified_base 2: mod_base = OTHER; note = 2'-fluorouridine
  modified_base 2^3: mod_base = OTHER; note = phosphorothioate linkage
  modified_base 3: mod_base = um
  modified_base 3^4: mod_base = OTHER; note = phosphorothioate linkage
  modified_base 4: mod_base = OTHER; note = 2'-fluoroadenosine
  modified_base 5: mod_base = gm
  modified_base 6: mod_base = OTHER; note = 2'-O-methyladenosine
  modified_base 7: mod_base = gm
  modified_base 8: mod_base = um
  modified_base 9: mod_base = gm
  modified_base 10: mod_base = OTHER; note = 2'-O-methyladenosine
  modified_base 11: mod_base = gm
  modified_base 12: mod_base = OTHER; note = 2'-fluoroguanosine
  modified_base 13: mod_base = OTHER; note = 2'-O-methyladenosine
  modified_base 14: mod_base = OTHER; note = 2'-fluorouridine
  modified_base 15: mod_base = um
  modified_base 16: mod_base = OTHER; note = 2'-fluoroadenosine
  modified_base 17: mod_base = OTHER; note = 2'-O-methyladenosine
  modified_base 18: mod_base = OTHER; note = 2'-fluoroadenosine
  modified_base 19: mod_base = OTHER; note = 2'-O-methyladenosine
  modified_base 20: mod_base = OTHER; note = 2'-fluorouridine
  modified_base 20^21: mod_base = OTHER; note = phosphorothioate linkage
  modified_base 21: mod_base = gm
  source 1..21: mol_type = other RNA
tttagagtgaggattaaaatg

SEQ ID NO: 25 - RNA, 21 nt, synthetic construct
  modified_base 1: mod_base = OTHER; note = 5 -cyclopropyl phosphonate-2 -O-methyluridine
  modified_base 1^2: mod_base = OTHER; note = phosphorothioate linkage
  modified_base 2: mod_base = OTHER; note = 2'-fluorouridine
  modified_base 2^3: mod_base = OTHER; note = phosphorothioate linkage
  modified_base 3: mod_base = um
  modified_base 3^4: mod_base = OTHER; note = phosphorothioate linkage
  modified_base 4: mod_base = OTHER; note = 2'-fluoroadenosine
  modified_base 5: mod_base = gm
  modified_base 6: mod_base = OTHER; note = 2'-O-methyladenosine
  modified_base 7: mod_base = gm
  modified_base 8: mod_base = um
  modified_base 9: mod_base = gm
  modified_base 10: mod_base = OTHER; note = 2'-O-methyladenosine
  modified_base 11: mod_base = gm
  modified_base 12: mod_base = OTHER; note = 2'-fluoroguanosine
  modified_base 13: mod_base = OTHER; note = 2'-O-methyladenosine
  modified_base 14: mod_base = OTHER; note = 2'-fluorouridine
  modified_base 15: mod_base = um
  modified_base 16: mod_base = OTHER; note = 2'-fluoroadenosine
  modified_base 17: mod_base = OTHER; note = 2'-O-methyladenosine
  modified_base 18: mod_base = OTHER; note = 2'-fluoroadenosine
  modified_base 19: mod_base = OTHER; note = 2'-O-methyladenosine
  modified_base 20: mod_base = OTHER; note = 2'-fluorouridine
  modified_base 20^21: mod_base = OTHER; note = phosphorothioate linkage
  modified_base 21: mod_base = gm
  source 1..21: mol_type = other RNA
tttagagtgaggattaaaatg

SEQ ID NO: 26 - RNA, 21 nt, synthetic construct
  modified_base 1: mod_base = OTHER; note = 5 -cyclopropyl phosphonate-2 -O-methyluridine
  modified_base 1^2: mod_base = OTHER; note = phosphorothioate linkage
  modified_base 2: mod_base = OTHER; note = 2'-fluorouridine
  modified_base 2^3: mod_base = OTHER; note = phosphorothioate linkage
  modified_base 3: mod_base = um
  modified_base 3^4: mod_base = OTHER; note = phosphorothioate linkage
  modified_base 4: mod_base = OTHER; note = 2'-fluoroadenosine
  modified_base 5: mod_base = gm
  modified_base 6: mod_base = OTHER; note = 2'-O-methyladenosine
  modified_base 7: mod_base = gm
  modified_base 8: mod_base = um
  modified_base 9: mod_base = gm
  modified_base 10: mod_base = OTHER; note = 2'-O-methyladenosine
  modified_base 11: mod_base = gm
  modified_base 12: mod_base = OTHER; note = 2'-fluoroguanosine
  modified_base 13: mod_base = OTHER; note = 2'-O-methyladenosine
  modified_base 14: mod_base = OTHER; note = 2'-fluorouridine
  modified_base 15: mod_base = um
  modified_base 16: mod_base = OTHER; note = 2'-fluoroadenosine
  modified_base 17: mod_base = OTHER; note = 2'-O-methyladenosine
  modified_base 18: mod_base = OTHER; note = 2'-fluoroadenosine
  modified_base 19: mod_base = OTHER; note = 2'-O-methyladenosine
  modified_base 20: mod_base = OTHER; note = 2'-fluorouridine
  modified_base 20^21: mod_base = OTHER; note = phosphorothioate linkage
  modified_base 21: mod_base = gm
  source 1..21: mol_type = other RNA
tttagagtgaggattaaaatg

SEQ ID NO: 27 - RNA, 21 nt, synthetic construct
  modified_base 1: mod_base = OTHER; note = 5 -cyclopropyl phosphonate-2 -O-methyluridine
  modified_base 1^2: mod_base = OTHER; note = phosphorothioate linkage
  modified_base 2: mod_base = OTHER; note = 2'-fluorouridine
  modified_base 2^3: mod_base = OTHER; note = phosphorothioate linkage
  modified_base 3: mod_base = um
  modified_base 3^4: mod_base = OTHER; note = phosphorothioate linkage
  modified_base 4: mod_base = OTHER; note = 2'-fluoroadenosine
  modified_base 5: mod_base = gm
  modified_base 6: mod_base = OTHER; note = 2'-O-methyladenosine
  modified_base 7: mod_base = gm
  modified_base 8: mod_base = um
  modified_base 9: mod_base = gm
  modified_base 10: mod_base = OTHER; note = 2'-O-methyladenosine
  modified_base 11: mod_base = gm
  modified_base 12: mod_base = OTHER; note = 2'-fluoroguanosine
  modified_base 13: mod_base = OTHER; note = 2'-O-methyladenosine
  modified_base 14: mod_base = OTHER; note = 2'-fluorouridine
  modified_base 15: mod_base = um
  modified_base 16: mod_base = OTHER; note = 2'-fluoroadenosine
  modified_base 17: mod_base = OTHER; note = 2'-O-methyladenosine
  modified_base 18: mod_base = OTHER; note = 2'-fluoroadenosine
  modified_base 19: mod_base = OTHER; note = 2'-O-methyladenosine
  modified_base 20: mod_base = OTHER; note = 2'-fluorouridine
  modified_base 20^21: mod_base = OTHER; note = phosphorothioate linkage
  modified_base 21: mod_base = gm
  source 1..21: mol_type = other RNA
tttagagtgaggattaaaatg

SEQ ID NO: 28 - RNA, 21 nt, synthetic construct
  modified_base 1: mod_base = OTHER; note = 5 -cyclopropyl phosphonate-2 -O-methyluridine
  modified_base 1^2: mod_base = OTHER; note = phosphorothioate linkage
  modified_base 2: mod_base = OTHER; note = 2'-fluoroguanosine
  modified_base 2^3: mod_base = OTHER; note = phosphorothioate linkage
  modified_base 3: mod_base = OTHER; note = 2'-O-methyladenosine
  modified_base 3^4: mod_base = OTHER; note = phosphorothioate linkage
  modified_base 4: mod_base = um
  modified_base 5: mod_base = OTHER; note = 2'-O-methyladenosine
  modified_base 6: mod_base = gm
  modified_base 7: mod_base = OTHER; note = 2 ,3 -seco-adenosine
  modified_base 8: mod_base = gm
  modified_base 9: mod_base = gm
  modified_base 10: mod_base = OTHER; note = 2'-fluoroadenosine
  modified_base 11: mod_base = um
  modified_base 12: mod_base = OTHER; note = 2'-fluorouridine
  modified_base 13: mod_base = OTHER; note = 2'-O-methyladenosine
  modified_base 14: mod_base = OTHER; note = 2'-fluoroadenosine
  modified_base 15: mod_base = OTHER; note = 2'-O-methyladenosine
  modified_base 16: mod_base = OTHER; note = 2'-fluoroguanosine
  modified_base 17: mod_base = um
  modified_base 18: mod_base = gm
  modified_base 19: mod_base = OTHER; note = 2'-O-methyladenosine
  modified_base 20: mod_base = gm
  modified_base 20^21: mod_base = OTHER; note = phosphorothioate linkage
  modified_base 21: mod_base = gm
  source 1..21: mol_type = other RNA
tgatagaggattaaagtgagg

SEQ ID NO: 29 - RNA, 21 nt, synthetic construct
  modified_base 1: mod_base = OTHER; note = 5 -cyclopropyl phosphonate-2 -O-methyluridine
  modified_base 1^2: mod_base = OTHER; note = phosphorothioate linkage
  modified_base 2: mod_base = OTHER; note = 2'-fluoroguanosine
  modified_base 2^3: mod_base = OTHER; note = phosphorothioate linkage
  modified_base 3: mod_base = OTHER; note = 2'-O-methyladenosine
  modified_base 3^4: mod_base = OTHER; note = phosphorothioate linkage
  modified_base 4: mod_base = um
  modified_base 5: mod_base = OTHER; note = 2'-O-methyladenosine
  modified_base 6: mod_base = gm
  modified_base 7: mod_base = OTHER; note = 2 ,3 -seco-adenosine
  modified_base 8: mod_base = gm
  modified_base 9: mod_base = gm
  modified_base 10: mod_base = OTHER; note = 2'-fluoroadenosine
  modified_base 11: mod_base = um
  modified_base 12: mod_base = OTHER; note = 2'-fluorouridine
  modified_base 13: mod_base = OTHER; note = 2'-O-methyladenosine
  modified_base 14: mod_base = OTHER; note = 2'-fluoroadenosine
  modified_base 15: mod_base = OTHER; note = 2'-O-methyladenosine
  modified_base 16: mod_base = OTHER; note = 2'-fluoroguanosine
  modified_base 17: mod_base = um
  modified_base 18: mod_base = gm
  modified_base 19: mod_base = OTHER; note = 2'-O-methyladenosine
  modified_base 20: mod_base = gm
  modified_base 20^21: mod_base = OTHER; note = phosphorothioate linkage
  modified_base 21: mod_base = gm
  source 1..21: mol_type = other RNA
tgatagaggattaaagtgagg

SEQ ID NO: 30 - RNA, 21 nt, synthetic construct
  modified_base 1: mod_base = OTHER; note = 5 -cyclopropyl phosphonate-2 -O-methyluridine
  modified_base 1^2: mod_base = OTHER; note = phosphorothioate linkage
  modified_base 2: mod_base = OTHER; note = 2'-fluoroguanosine
  modified_base 2^3: mod_base = OTHER; note = phosphorothioate linkage
  modified_base 3: mod_base = OTHER; note = 2'-O-methyladenosine
  modified_base 3^4: mod_base = OTHER; note = phosphorothioate linkage
  modified_base 4: mod_base = um
  modified_base 5: mod_base = OTHER; note = 2'-O-methyladenosine
  modified_base 6: mod_base = gm
  modified_base 7: mod_base = OTHER; note = 2 ,3 -seco-adenosine
  modified_base 8: mod_base = gm
  modified_base 9: mod_base = gm
  modified_base 10: mod_base = OTHER; note = 2'-fluoroadenosine
  modified_base 11: mod_base = um
  modified_base 12: mod_base = OTHER; note = 2'-fluorouridine
  modified_base 13: mod_base = OTHER; note = 2'-O-methyladenosine
  modified_base 14: mod_base = OTHER; note = 2'-fluoroadenosine
  modified_base 15: mod_base = OTHER; note = 2'-O-methyladenosine
  modified_base 16: mod_base = OTHER; note = 2'-fluoroguanosine
  modified_base 17: mod_base = um
  modified_base 18: mod_base = gm
  modified_base 19: mod_base = OTHER; note = 2'-O-methyladenosine
  modified_base 20: mod_base = gm
  modified_base 20^21: mod_base = OTHER; note = phosphorothioate linkage
  modified_base 21: mod_base = gm
  source 1..21: mol_type = other RNA
tgatagaggattaaagtgagg

SEQ ID NO: 31 - RNA, 21 nt, synthetic construct
  modified_base 1: mod_base = OTHER; note = 5 -cyclopropyl phosphonate-2 -O-methyluridine
  modified_base 1^2: mod_base = OTHER; note = phosphorothioate linkage
  modified_base 2: mod_base = OTHER; note = 2'-fluoroguanosine
  modified_base 2^3: mod_base = OTHER; note = phosphorothioate linkage
  modified_base 3: mod_base = OTHER; note = 2'-O-methyladenosine
  modified_base 3^4: mod_base = OTHER; note = phosphorothioate linkage
  modified_base 4: mod_base = um
  modified_base 5: mod_base = OTHER; note = 2'-O-methyladenosine
  modified_base 6: mod_base = gm
  modified_base 7: mod_base = OTHER; note = 2 ,3 -seco-adenosine
  modified_base 8: mod_base = gm
  modified_base 9: mod_base = gm
  modified_base 10: mod_base = OTHER; note = 2'-fluoroadenosine
  modified_base 11: mod_base = um
  modified_base 12: mod_base = OTHER; note = 2'-fluorouridine
  modified_base 13: mod_base = OTHER; note = 2'-O-methyladenosine
  modified_base 14: mod_base = OTHER; note = 2'-fluoroadenosine
  modified_base 15: mod_base = OTHER; note = 2'-O-methyladenosine
  modified_base 16: mod_base = OTHER; note = 2'-fluoroguanosine
  modified_base 17: mod_base = um
  modified_base 18: mod_base = gm
  modified_base 19: mod_base = OTHER; note = 2'-O-methyladenosine
  modified_base 20: mod_base = gm
  modified_base 20^21: mod_base = OTHER; note = phosphorothioate linkage
  modified_base 21: mod_base = gm
  source 1..21: mol_type = other RNA
tgatagaggattaaagtgagg

SEQ ID NO: 32 - RNA, 21 nt, synthetic construct
  modified_base 1: mod_base = OTHER; note = 5 -cyclopropyl phosphonate-2 -O-methyluridine
  modified_base 1^2: mod_base = OTHER; note = phosphorothioate linkage
  modified_base 2: mod_base = OTHER; note = 2'-fluoroguanosine
  modified_base 2^3: mod_base = OTHER; note = phosphorothioate linkage
  modified_base 3: mod_base = OTHER; note = 2'-O-methyladenosine
  modified_base 3^4: mod_base = OTHER; note = phosphorothioate linkage
  modified_base 4: mod_base = um
  modified_base 5: mod_base = OTHER; note = 2'-O-methyladenosine
  modified_base 6: mod_base = gm
  modified_base 7: mod_base = OTHER; note = 2 ,3 -seco-adenosine
  modified_base 8: mod_base = gm
  modified_base 9: mod_base = gm
  modified_base 10: mod_base = OTHER; note = 2'-fluoroadenosine
  modified_base 11: mod_base = um
  modified_base 12: mod_base = OTHER; note = 2'-fluorouridine
  modified_base 13: mod_base = OTHER; note = 2'-O-methyladenosine
  modified_base 14: mod_base = OTHER; note = 2'-fluoroadenosine
  modified_base 15: mod_base = OTHER; note = 2'-O-methyladenosine
  modified_base 16: mod_base = OTHER; note = 2'-fluoroguanosine
  modified_base 17: mod_base = um
  modified_base 18: mod_base = gm
  modified_base 19: mod_base = OTHER; note = 2'-O-methyladenosine
  modified_base 20: mod_base = gm
  modified_base 20^21: mod_base = OTHER; note = phosphorothioate linkage
  modified_base 21: mod_base = gm
  source 1..21: mol_type = other RNA
tgatagaggattaaagtgagg

SEQ ID NO: 33 - RNA, 21 nt, synthetic construct
  modified_base 1: mod_base = OTHER; note = 5 -cyclopropyl phosphonate-2 -O-methyluridine
  modified_base 1^2: mod_base = OTHER; note = phosphorothioate linkage
  modified_base 2: mod_base = OTHER; note = 2'-fluorouridine
  modified_base 2^3: mod_base = OTHER; note = phosphorothioate linkage
  modified_base 3: mod_base = um
  modified_base 3^4: mod_base = OTHER; note = phosphorothioate linkage
  modified_base 4: mod_base = OTHER; note = 2'-O-methyladenosine
  modified_base 5: mod_base = gm
  modified_base 6: mod_base = OTHER; note = 2'-fluoroadenosine
  modified_base 7: mod_base = gm
  modified_base 8: mod_base = OTHER; note = 2'-fluorouridine
  modified_base 9: mod_base = OTHER; note = 2'-fluoroguanosine
  modified_base 10: mod_base = OTHER; note = 2'-O-methyladenosine
  modified_base 11: mod_base = gm
  modified_base 12: mod_base = gm
  modified_base 13: mod_base = OTHER; note = 2'-O-methyladenosine
  modified_base 14: mod_base = OTHER; note = 2'-fluorouridine
  modified_base 15: mod_base = um
  modified_base 16: mod_base = OTHER; note = 2'-fluoroadenosine
  modified_base 17: mod_base = OTHER; note = 2'-O-methyladenosine
  modified_base 18: mod_base = OTHER; note = 2'-O-methyladenosine
  modified_base 19: mod_base = OTHER; note = 2'-O-methyladenosine
  modified_base 20: mod_base = um
  modified_base 20^21: mod_base = OTHER; note = phosphorothioate linkage
  modified_base 21: mod_base = gm
  source 1..21: mol_type = other RNA
tttagagtgaggattaaaatg

SEQ ID NO: 34 - RNA, 21 nt, synthetic construct
  modified_base 1: mod_base = OTHER; note = 5 -cyclopropyl phosphonate-2 -O-methyluridine
  modified_base 1^2: mod_base = OTHER; note = phosphorothioate linkage
  modified_base 2: mod_base = OTHER; note = 2'-fluorouridine
  modified_base 2^3: mod_base = OTHER; note = phosphorothioate linkage
  modified_base 3: mod_base = um
  modified_base 3^4: mod_base = OTHER; note = phosphorothioate linkage
  modified_base 4: mod_base = OTHER; note = 2'-O-methyladenosine
  modified_base 5: mod_base = gm
  modified_base 6: mod_base = OTHER; note = 2'-fluoroadenosine
  modified_base 7: mod_base = gm
  modified_base 8: mod_base = OTHER; note = 2'-fluorouridine
  modified_base 9: mod_base = OTHER; note = 2'-fluoroguanosine
  modified_base 10: mod_base = OTHER; note = 2'-O-methyladenosine
  modified_base 11: mod_base = gm
  modified_base 12: mod_base = gm
  modified_base 13: mod_base = OTHER; note = 2'-O-methyladenosine
  modified_base 14: mod_base = OTHER; note = 2'-fluorouridine
  modified_base 15: mod_base = um
  modified_base 16: mod_base = OTHER; note = 2'-fluoroadenosine
  modified_base 17: mod_base = OTHER; note = 2'-O-methyladenosine
  modified_base 18: mod_base = OTHER; note = 2'-O-methyladenosine
  modified_base 19: mod_base = OTHER; note = 2'-O-methyladenosine
  modified_base 20: mod_base = um
  modified_base 20^21: mod_base = OTHER; note = phosphorothioate linkage
  modified_base 21: mod_base = gm
  source 1..21: mol_type = other RNA
tttagagtgaggattaaaatg

SEQ ID NO: 35 - RNA, 21 nt, synthetic construct
  modified_base 1: mod_base = OTHER; note = 5 -cyclopropyl phosphonate-2 -O-methyluridine
  modified_base 1^2: mod_base = OTHER; note = phosphorothioate linkage
  modified_base 2: mod_base = OTHER; note = 2'-fluorouridine
  modified_base 2^3: mod_base = OTHER; note = phosphorothioate linkage
  modified_base 3: mod_base = um
  modified_base 3^4: mod_base = OTHER; note = phosphorothioate linkage
  modified_base 4: mod_base = OTHER; note = 2'-O-methyladenosine
  modified_base 5: mod_base = gm
  modified_base 6: mod_base = OTHER; note = 2'-fluoroadenosine
  modified_base 7: mod_base = gm
  modified_base 8: mod_base = OTHER; note = 2'-fluorouridine
  modified_base 9: mod_base = OTHER; note = 2'-fluoroguanosine
  modified_base 10: mod_base = OTHER; note = 2'-O-methyladenosine
  modified_base 11: mod_base = gm
  modified_base 12: mod_base = gm
  modified_base 13: mod_base = OTHER; note = 2'-O-methyladenosine
  modified_base 14: mod_base = OTHER; note = 2'-fluorouridine
  modified_base 15: mod_base = um
  modified_base 16: mod_base = OTHER; note = 2'-fluoroadenosine
  modified_base 17: mod_base = OTHER; note = 2'-O-methyladenosine
  modified_base 18: mod_base = OTHER; note = 2'-O-methyladenosine
  modified_base 19: mod_base = OTHER; note = 2'-O-methyladenosine
  modified_base 20: mod_base = um
  modified_base 20^21: mod_base = OTHER; note = phosphorothioate linkage
  modified_base 21: mod_base = gm
  source 1..21: mol_type = other RNA
tttagagtgaggattaaaatg

SEQ ID NO: 36 - RNA, 21 nt, synthetic construct
  modified_base 1: mod_base = OTHER; note = 5 -cyclopropyl phosphonate-2 -O-methyluridine
  modified_base 1^2: mod_base = OTHER; note = phosphorothioate linkage
  modified_base 2: mod_base = OTHER; note = 2'-fluorouridine
  modified_base 2^3: mod_base = OTHER; note = phosphorothioate linkage
  modified_base 3: mod_base = um
  modified_base 3^4: mod_base = OTHER; note = phosphorothioate linkage
  modified_base 4: mod_base = OTHER; note = 2'-fluoroadenosine
  modified_base 5: mod_base = gm
  modified_base 6: mod_base = OTHER; note = 2'-O-methyladenosine
  modified_base 7: mod_base = gm
  modified_base 8: mod_base = um
  modified_base 9: mod_base = gm
  modified_base 10: mod_base = OTHER; note = 2'-O-methyladenosine
  modified_base 11: mod_base = gm
  modified_base 12: mod_base = OTHER; note = 2'-fluoroguanosine
  modified_base 13: mod_base = OTHER; note = 2'-O-methyladenosine
  modified_base 14: mod_base = OTHER; note = 2'-fluorouridine
  modified_base 15: mod_base = um
  modified_base 16: mod_base = OTHER; note = 2'-fluoroadenosine
  modified_base 17: mod_base = OTHER; note = 2'-O-methyladenosine
  modified_base 18: mod_base = OTHER; note = 2'-fluoroadenosine
  modified_base 19: mod_base = OTHER; note = 2'-O-methyladenosine
  modified_base 20: mod_base = OTHER; note = 2'-fluorouridine
  modified_base 20^21: mod_base = OTHER; note = phosphorothioate linkage
  modified_base 21: mod_base = gm
  source 1..21: mol_type = other RNA
tttagagtgaggattaaaatg

SEQ ID NO: 37 - RNA, 21 nt, synthetic construct
  modified_base 1: mod_base = OTHER; note = 5 -cyclopropyl phosphonate-2 -O-methyluridine
  modified_base 1^2: mod_base = OTHER; note = phosphorothioate linkage
  modified_base 2: mod_base = OTHER; note = 2'-fluorouridine
  modified_base 2^3: mod_base = OTHER; note = phosphorothioate linkage
  modified_base 3: mod_base = um
  modified_base 3^4: mod_base = OTHER; note = phosphorothioate linkage
  modified_base 4: mod_base = OTHER; note = 2'-fluoroadenosine
  modified_base 5: mod_base = gm
  modified_base 6: mod_base = OTHER; note = 2'-O-methyladenosine
  modified_base 7: mod_base = gm
  modified_base 8: mod_base = um
  modified_base 9: mod_base = gm
  modified_base 10: mod_base = OTHER; note = 2'-O-methyladenosine
  modified_base 11: mod_base = gm
  modified_base 12: mod_base = OTHER; note = 2'-fluoroguanosine
  modified_base 13: mod_base = OTHER; note = 2'-O-methyladenosine
  modified_base 14: mod_base = OTHER; note = 2'-fluorouridine
  modified_base 15: mod_base = um
  modified_base 16: mod_base = OTHER; note = 2'-fluoroadenosine
  modified_base 17: mod_base = OTHER; note = 2'-O-methyladenosine
  modified_base 18: mod_base = OTHER; note = 2'-fluoroadenosine
  modified_base 19: mod_base = OTHER; note = 2'-O-methyladenosine
  modified_base 20: mod_base = OTHER; note = 2'-fluorouridine
  modified_base 20^21: mod_base = OTHER; note = phosphorothioate linkage
  modified_base 21: mod_base = gm
  source 1..21: mol_type = other RNA
tttagagtgaggattaaaatg

SEQ ID NO: 38 - RNA, 21 nt, synthetic construct
  modified_base 1: mod_base = OTHER; note = 5 -cyclopropyl phosphonate-2 -O-methyluridine
  modified_base 1^2: mod_base = OTHER; note = phosphorothioate linkage
  modified_base 2: mod_base = OTHER; note = 2'-fluoroguanosine
  modified_base 2^3: mod_base = OTHER; note = phosphorothioate linkage
  modified_base 3: mod_base = OTHER; note = 2'-O-methyladenosine
  modified_base 3^4: mod_base = OTHER; note = phosphorothioate linkage
  modified_base 4: mod_base = um
  modified_base 5: mod_base = OTHER; note = 2'-O-methyladenosine
  modified_base 6: mod_base = gm
  modified_base 7: mod_base = OTHER; note = 2'-fluoroadenosine
  modified_base 8: mod_base = gm
  modified_base 9: mod_base = gm
  modified_base 10: mod_base = OTHER; note = 2'-fluoroadenosine
  modified_base 11: mod_base = um
  modified_base 12: mod_base = OTHER; note = 2'-fluorouridine
  modified_base 13: mod_base = OTHER; note = 2'-O-methyladenosine
  modified_base 14: mod_base = OTHER; note = 2'-fluoroadenosine
  modified_base 15: mod_base = OTHER; note = 2'-O-methyladenosine
  modified_base 16: mod_base = OTHER; note = 2'-fluoroguanosine
  modified_base 17: mod_base = um
  modified_base 18: mod_base = gm
  modified_base 19: mod_base = OTHER; note = 2'-O-methyladenosine
  modified_base 20: mod_base = gm
  modified_base 20^21: mod_base = OTHER; note = phosphorothioate linkage
  modified_base 21: mod_base = gm
  source 1..21: mol_type = other RNA
tgatagaggattaaagtgagg

SEQ ID NO: 39 - RNA, 21 nt, synthetic construct
  modified_base 1: mod_base = OTHER; note = 5 -cyclopropyl phosphonate-2 -O-methyluridine
  modified_base 1^2: mod_base = OTHER; note = phosphorothioate linkage
  modified_base 2: mod_base = OTHER; note = 2'-fluoroguanosine
  modified_base 2^3: mod_base = OTHER; note = phosphorothioate linkage
  modified_base 3: mod_base = OTHER; note = 2'-O-methyladenosine
  modified_base 3^4: mod_base = OTHER; note = phosphorothioate linkage
  modified_base 4: mod_base = um
  modified_base 5: mod_base = OTHER; note = 2'-O-methyladenosine
  modified_base 6: mod_base = gm
  modified_base 7: mod_base = OTHER; note = 2'-fluoroadenosine
  modified_base 8: mod_base = gm
  modified_base 9: mod_base = gm
  modified_base 10: mod_base = OTHER; note = 2'-fluoroadenosine
  modified_base 11: mod_base = um
  modified_base 12: mod_base = OTHER; note = 2'-fluorouridine
  modified_base 13: mod_base = OTHER; note = 2'-O-methyladenosine
  modified_base 14: mod_base = OTHER; note = 2'-fluoroadenosine
  modified_base 15: mod_base = OTHER; note = 2'-O-methyladenosine
  modified_base 16: mod_base = OTHER; note = 2'-fluoroguanosine
  modified_base 17: mod_base = um
  modified_base 18: mod_base = gm
  modified_base 19: mod_base = OTHER; note = 2'-O-methyladenosine
  modified_base 20: mod_base = gm
  modified_base 20^21: mod_base = OTHER; note = phosphorothioate linkage
  modified_base 21: mod_base = gm
  source 1..21: mol_type = other RNA
tgatagaggattaaagtgagg

SEQ ID NO: 40 - RNA, 21 nt, synthetic construct
  modified_base 1: mod_base = OTHER; note = 5 -cyclopropyl phosphonate-2 -O-methyluridine
  modified_base 1^2: mod_base = OTHER; note = phosphorothioate linkage
  modified_base 2: mod_base = OTHER; note = 2'-fluoroguanosine
  modified_base 2^3: mod_base = OTHER; note = phosphorothioate linkage
  modified_base 3: mod_base = OTHER; note = 2'-O-methyladenosine
  modified_base 3^4: mod_base = OTHER; note = phosphorothioate linkage
  modified_base 4: mod_base = um
  modified_base 5: mod_base = OTHER; note = 2'-O-methyladenosine
  modified_base 6: mod_base = gm
  modified_base 7: mod_base = OTHER; note = 2 ,3 -seco-adenosine
  modified_base 8: mod_base = gm
  modified_base 9: mod_base = gm
  modified_base 10: mod_base = OTHER; note = 2'-fluoroadenosine
  modified_base 11: mod_base = um
  modified_base 12: mod_base = OTHER; note = 2'-fluorouridine
  modified_base 13: mod_base = OTHER; note = 2'-O-methyladenosine
  modified_base 14: mod_base = OTHER; note = 2'-fluoroadenosine
  modified_base 15: mod_base = OTHER; note = 2'-O-methyladenosine
  modified_base 16: mod_base = OTHER; note = 2'-fluoroguanosine
  modified_base 17: mod_base = um
  modified_base 18: mod_base = gm
  modified_base 19: mod_base = OTHER; note = 2'-O-methyladenosine
  modified_base 20: mod_base = gm
  modified_base 20^21: mod_base = OTHER; note = phosphorothioate linkage
  modified_base 21: mod_base = gm
  source 1..21: mol_type = other RNA
tgatagaggattaaagtgagg

SEQ ID NO: 41 - RNA, 21 nt, synthetic construct
  modified_base 1: mod_base = OTHER; note = 5 -cyclopropyl phosphonate-2 -O-methyluridine
  modified_base 2: mod_base = OTHER; note = 2'-fluoroadenosine
  modified_base 3: mod_base = gm
  modified_base 4: mod_base = OTHER; note = 2'-fluoroguanosine
  modified_base 5: mod_base = OTHER; note = 2'-O-methyladenosine
  modified_base 6: mod_base = um
  modified_base 7: mod_base = OTHER; note = 2'-O-methyladenosine
  modified_base 8: mod_base = OTHER; note = 2'-O-methyladenosine
  modified_base 9: mod_base = cm
  modified_base 10: mod_base = OTHER; note = 2'-O-methyladenosine
  modified_base 11: mod_base = gm
  modified_base 12: mod_base = OTHER; note = 2'-fluoroadenosine
  modified_base 13: mod_base = um
  modified_base 14: mod_base = OTHER; note = 2'-fluoroguanosine
  modified_base 15: mod_base = OTHER; note = 2'-O-methyladenosine
  modified_base 16: mod_base = OTHER; note = 2'-fluoroguanosine
  modified_base 17: mod_base = um
  modified_base 18: mod_base = um
  modified_base 19: mod_base = OTHER; note = 2'-O-methyladenosine
  modified_base 20: mod_base = OTHER; note = 2'-O-methyladenosine
  modified_base 20^21: mod_base = OTHER; note = phosphorothioate linkage
  modified_base 20^21: mod_base = OTHER; note = phosphorothioate linkage
  modified_base 21: mod_base = gm
  source 1..21: mol_type = other RNA
taggataacagatgagttaag

SEQ ID NO: 42 - RNA, 21 nt, synthetic construct
  modified_base 1: mod_base = OTHER; note = 5 -cyclopropyl phosphonate-2 -O-methyluridine
  modified_base 1^2: mod_base = OTHER; note = phosphorothioate linkage
  modified_base 2: mod_base = OTHER; note = 2'-fluoroguanosine
  modified_base 2^3: mod_base = OTHER; note = phosphorothioate linkage
  modified_base 3: mod_base = OTHER; note = 2'-O-methyladenosine
  modified_base 3^4: mod_base = OTHER; note = phosphorothioate linkage
  modified_base 4: mod_base = um
  modified_base 5: mod_base = OTHER; note = 2'-O-methyladenosine
  modified_base 6: mod_base = gm
  modified_base 7: mod_base = OTHER; note = 2 ,3 -seco-adenosine
  modified_base 8: mod_base = gm
  modified_base 9: mod_base = gm
  modified_base 10: mod_base = OTHER; note = 2'-fluoroadenosine
  modified_base 11: mod_base = um
  modified_base 12: mod_base = OTHER; note = 2'-fluorouridine
  modified_base 13: mod_base = OTHER; note = 2'-O-methyladenosine
  modified_base 14: mod_base = OTHER; note = 2'-fluoroadenosine
  modified_base 15: mod_base = OTHER; note = 2'-O-methyladenosine
  modified_base 16: mod_base = OTHER; note = 2'-fluoroguanosine
  modified_base 17: mod_base = um
  modified_base 18: mod_base = gm
  modified_base 19: mod_base = OTHER; note = 2'-O-methyladenosine
  modified_base 20: mod_base = gm
  modified_base 20^21: mod_base = OTHER; note = phosphorothioate linkage
  modified_base 21: mod_base = gm
  source 1..21: mol_type = other RNA
tgatagaggattaaagtgagg

SEQ ID NO: 43 - RNA, 21 nt, synthetic construct
  modified_base 1: mod_base = OTHER; note = 5 -cyclopropyl phosphonate-2 -O-methyluridine
  modified_base 1^2: mod_base = OTHER; note = phosphorothioate linkage
  modified_base 2: mod_base = OTHER; note = 2'-fluoroguanosine
  modified_base 2^3: mod_base = OTHER; note = phosphorothioate linkage
  modified_base 3: mod_base = OTHER; note = 2'-O-methyladenosine
  modified_base 3^4: mod_base = OTHER; note = phosphorothioate linkage
  modified_base 4: mod_base = um
  modified_base 5: mod_base = OTHER; note = 2'-O-methyladenosine
  modified_base 6: mod_base = gm
  modified_base 7: mod_base = OTHER; note = 2 ,3 -seco-adenosine
  modified_base 8: mod_base = gm
  modified_base 9: mod_base = gm
  modified_base 10: mod_base = OTHER; note = 2'-fluoroadenosine
  modified_base 11: mod_base = um
  modified_base 12: mod_base = OTHER; note = 2'-fluorouridine
  modified_base 13: mod_base = OTHER; note = 2'-O-methyladenosine
  modified_base 14: mod_base = OTHER; note = 2'-fluoroadenosine
  modified_base 15: mod_base = OTHER; note = 2'-O-methyladenosine
  modified_base 16: mod_base = OTHER; note = 2'-fluoroguanosine
  modified_base 17: mod_base = um
  modified_base 18: mod_base = gm
  modified_base 19: mod_base = OTHER; note = 2'-O-methyladenosine
  modified_base 20: mod_base = gm
  modified_base 20^21: mod_base = OTHER; note = phosphorothioate linkage
  modified_base 21: mod_base = gm
  source 1..21: mol_type = other RNA
tgatagaggattaaagtgagg

SEQ ID NO: 44 - RNA, 21 nt, synthetic construct
  modified_base 1: mod_base = OTHER; note = 5 -cyclopropyl phosphonate-2 -O-methyluridine
  modified_base 1^2: mod_base = OTHER; note = phosphorothioate linkage
  modified_base 2: mod_base = OTHER; note = 2'-fluoroguanosine
  modified_base 2^3: mod_base = OTHER; note = phosphorothioate linkage
  modified_base 3: mod_base = OTHER; note = 2'-O-methyladenosine
  modified_base 3^4: mod_base = OTHER; note = phosphorothioate linkage
  modified_base 4: mod_base = um
  modified_base 5: mod_base = OTHER; note = 2'-O-methyladenosine
  modified_base 6: mod_base = gm
  modified_base 7: mod_base = OTHER; note = 2 ,3 -seco-adenosine
  modified_base 8: mod_base = gm
  modified_base 9: mod_base = gm
  modified_base 10: mod_base = OTHER; note = 2'-fluoroadenosine
  modified_base 11: mod_base = um
  modified_base 12: mod_base = OTHER; note = 2'-fluorouridine
  modified_base 13: mod_base = OTHER; note = 2'-O-methyladenosine
  modified_base 14: mod_base = OTHER; note = 2'-fluoroadenosine
  modified_base 15: mod_base = OTHER; note = 2'-O-methyladenosine
  modified_base 16: mod_base = OTHER; note = 2'-fluoroguanosine
  modified_base 17: mod_base = um
  modified_base 18: mod_base = gm
  modified_base 19: mod_base = OTHER; note = 2'-O-methyladenosine
  modified_base 20: mod_base = gm
  modified_base 20^21: mod_base = OTHER; note = phosphorothioate linkage
  modified_base 21: mod_base = gm
  source 1..21: mol_type = other RNA
tgatagaggattaaagtgagg

SEQ ID NO: 45 - RNA, 21 nt, synthetic construct
  modified_base 1: mod_base = OTHER; note = 5 -cyclopropyl phosphonate-2 -O-methyluridine
  modified_base 1^2: mod_base = OTHER; note = phosphorothioate linkage
  modified_base 2: mod_base = OTHER; note = 2'-fluoroguanosine
  modified_base 2^3: mod_base = OTHER; note = phosphorothioate linkage
  modified_base 3: mod_base = OTHER; note = 2'-O-methyladenosine
  modified_base 3^4: mod_base = OTHER; note = phosphorothioate linkage
  modified_base 4: mod_base = um
  modified_base 5: mod_base = OTHER; note = 2'-O-methyladenosine
  modified_base 6: mod_base = gm
  modified_base 7: mod_base = OTHER; note = 2 ,3 -seco-adenosine
  modified_base 8: mod_base = gm
  modified_base 9: mod_base = gm
  modified_base 10: mod_base = OTHER; note = 2'-fluoroadenosine
  modified_base 11: mod_base = um
  modified_base 12: mod_base = OTHER; note = 2'-fluorouridine
  modified_base 13: mod_base = OTHER; note = 2'-O-methyladenosine
  modified_base 14: mod_base = OTHER; note = 2'-fluoroadenosine
  modified_base 15: mod_base = OTHER; note = 2'-O-methyladenosine
  modified_base 16: mod_base = OTHER; note = 2'-fluoroguanosine
  modified_base 17: mod_base = um
  modified_base 18: mod_base = gm
  modified_base 19: mod_base = OTHER; note = 2'-O-methyladenosine
  modified_base 20: mod_base = gm
  modified_base 20^21: mod_base = OTHER; note = phosphorothioate linkage
  modified_base 21: mod_base = gm
  source 1..21: mol_type = other RNA
tgatagaggattaaagtgagg

SEQ ID NO: 46 - RNA, 21 nt, synthetic construct
  modified_base 1: mod_base = OTHER; note = 5 -cyclopropyl phosphonate-2 -O-methyluridine
  modified_base 1^2: mod_base = OTHER; note = phosphorothioate linkage
  modified_base 2: mod_base = OTHER; note = 2'-fluoroguanosine
  modified_base 2^3: mod_base = OTHER; note = phosphorothioate linkage
  modified_base 3: mod_base = OTHER; note = 2'-O-methyladenosine
  modified_base 3^4: mod_base = OTHER; note = phosphorothioate linkage
  modified_base 4: mod_base = um
  modified_base 5: mod_base = OTHER; note = 2'-O-methyladenosine
  modified_base 6: mod_base = gm
  modified_base 7: mod_base = OTHER; note = 2 ,3 -seco-adenosine
  modified_base 8: mod_base = gm
  modified_base 9: mod_base = gm
  modified_base 10: mod_base = OTHER; note = 2'-fluoroadenosine
  modified_base 11: mod_base = um
  modified_base 12: mod_base = OTHER; note = 2'-fluorouridine
  modified_base 13: mod_base = OTHER; note = 2'-O-methyladenosine
  modified_base 14: mod_base = OTHER; note = 2'-fluoroadenosine
  modified_base 15: mod_base = OTHER; note = 2'-O-methyladenosine
  modified_base 16: mod_base = OTHER; note = 2'-fluoroguanosine
  modified_base 17: mod_base = um
  modified_base 18: mod_base = gm
  modified_base 19: mod_base = OTHER; note = 2'-O-methyladenosine
  modified_base 20: mod_base = gm
  modified_base 20^21: mod_base = OTHER; note = phosphorothioate linkage
  modified_base 21: mod_base = gm
  source 1..21: mol_type = other RNA
tgatagaggattaaagtgagg

SEQ ID NO: 47 - RNA, 21 nt, synthetic construct
  modified_base 1: mod_base = OTHER; note = 5 -cyclopropyl phosphonate-2 -O-methyluridine
  modified_base 1^2: mod_base = OTHER; note = phosphorothioate linkage
  modified_base 2: mod_base = OTHER; note = 2'-fluoroguanosine
  modified_base 2^3: mod_base = OTHER; note = phosphorothioate linkage
  modified_base 3: mod_base = OTHER; note = 2'-O-methyladenosine
  modified_base 3^4: mod_base = OTHER; note = phosphorothioate linkage
  modified_base 4: mod_base = um
  modified_base 5: mod_base = OTHER; note = 2'-O-methyladenosine
  modified_base 6: mod_base = gm
  modified_base 7: mod_base = OTHER; note = 2 ,3 -seco-adenosine
  modified_base 8: mod_base = gm
  modified_base 9: mod_base = gm
  modified_base 10: mod_base = OTHER; note = 2'-fluoroadenosine
  modified_base 11: mod_base = um
  modified_base 12: mod_base = OTHER; note = 2'-fluorouridine
  modified_base 13: mod_base = OTHER; note = 2'-O-methyladenosine
  modified_base 14: mod_base = OTHER; note = 2'-fluoroadenosine
  modified_base 15: mod_base = OTHER; note = 2'-O-methyladenosine
  modified_base 16: mod_base = OTHER; note = 2'-fluoroguanosine
  modified_base 17: mod_base = um
  modified_base 18: mod_base = gm
  modified_base 19: mod_base = OTHER; note = 2'-O-methyladenosine
  modified_base 20: mod_base = gm
  modified_base 20^21: mod_base = OTHER; note = phosphorothioate linkage
  modified_base 21: mod_base = gm
  source 1..21: mol_type = other RNA
tgatagaggattaaagtgagg

SEQ ID NO: 48 - RNA, 21 nt, synthetic construct
  modified_base 1: mod_base = OTHER; note = 5 -cyclopropyl phosphonate-2 -O-methyluridine
  modified_base 1^2: mod_base = OTHER; note = phosphorothioate linkage
  modified_base 2: mod_base = OTHER; note = 2'-fluoroguanosine
  modified_base 2^3: mod_base = OTHER; note = phosphorothioate linkage
  modified_base 3: mod_base = OTHER; note = 2'-O-methyladenosine
  modified_base 3^4: mod_base = OTHER; note = phosphorothioate linkage
  modified_base 4: mod_base = um
  modified_base 5: mod_base = OTHER; note = 2'-O-methyladenosine
  modified_base 6: mod_base = gm
  modified_base 7: mod_base = OTHER; note = 2 ,3 -seco-adenosine
  modified_base 8: mod_base = gm
  modified_base 9: mod_base = gm
  modified_base 10: mod_base = OTHER; note = 2'-fluoroadenosine
  modified_base 11: mod_base = um
  modified_base 12: mod_base = OTHER; note = 2'-fluorouridine
  modified_base 13: mod_base = OTHER; note = 2'-O-methyladenosine
  modified_base 14: mod_base = OTHER; note = 2'-fluoroadenosine
  modified_base 15: mod_base = OTHER; note = 2'-O-methyladenosine
  modified_base 16: mod_base = OTHER; note = 2'-fluoroguanosine
  modified_base 17: mod_base = um
  modified_base 18: mod_base = gm
  modified_base 19: mod_base = OTHER; note = 2'-O-methyladenosine
  modified_base 20: mod_base = gm
  modified_base 20^21: mod_base = OTHER; note = phosphorothioate linkage
  modified_base 21: mod_base = gm
  source 1..21: mol_type = other RNA
tgatagaggattaaagtgagg

SEQ ID NO: 49 - RNA, 21 nt, synthetic construct
  modified_base 1: mod_base = OTHER; note = 5 -cyclopropyl phosphonate-2 -O-methyluridine
  modified_base 1^2: mod_base = OTHER; note = phosphorothioate linkage
  modified_base 2: mod_base = OTHER; note = 2'-fluoroguanosine
  modified_base 2^3: mod_base = OTHER; note = phosphorothioate linkage
  modified_base 3: mod_base = OTHER; note = 2'-O-methyladenosine
  modified_base 3^4: mod_base = OTHER; note = phosphorothioate linkage
  modified_base 4: mod_base = um
  modified_base 5: mod_base = OTHER; note = 2'-O-methyladenosine
  modified_base 6: mod_base = gm
  modified_base 7: mod_base = OTHER; note = 2 ,3 -seco-adenosine
  modified_base 8: mod_base = gm
  modified_base 9: mod_base = gm
  modified_base 10: mod_base = OTHER; note = 2'-fluoroadenosine
  modified_base 11: mod_base = um
  modified_base 12: mod_base = OTHER; note = 2'-fluorouridine
  modified_base 13: mod_base = OTHER; note = 2'-O-methyladenosine
  modified_base 14: mod_base = OTHER; note = 2'-fluoroadenosine
  modified_base 15: mod_base = OTHER; note = 2'-O-methyladenosine
  modified_base 16: mod_base = OTHER; note = 2'-fluoroguanosine
  modified_base 17: mod_base = um
  modified_base 18: mod_base = gm
  modified_base 19: mod_base = OTHER; note = 2'-O-methyladenosine
  modified_base 20: mod_base = gm
  modified_base 20^21: mod_base = OTHER; note = phosphorothioate linkage
  modified_base 21: mod_base = gm
  source 1..21: mol_type = other RNA
tgatagaggattaaagtgagg

SEQ ID NO: 50 - RNA, 21 nt, synthetic construct
  modified_base 1: mod_base = OTHER; note = 5 -cyclopropyl phosphonate-2 -O-methyluridine
  modified_base 1^2: mod_base = OTHER; note = phosphorothioate linkage
  modified_base 2: mod_base = OTHER; note = 2'-fluoroguanosine
  modified_base 2^3: mod_base = OTHER; note = phosphorothioate linkage
  modified_base 3: mod_base = OTHER; note = 2'-O-methyladenosine
  modified_base 3^4: mod_base = OTHER; note = phosphorothioate linkage
  modified_base 4: mod_base = um
  modified_base 5: mod_base = OTHER; note = 2'-O-methyladenosine
  modified_base 6: mod_base = gm
  modified_base 7: mod_base = OTHER; note = 2 ,3 -seco-adenosine
  modified_base 8: mod_base = gm
  modified_base 9: mod_base = gm
  modified_base 10: mod_base = OTHER; note = 2'-fluoroadenosine
  modified_base 11: mod_base = um
  modified_base 12: mod_base = OTHER; note = 2'-fluorouridine
  modified_base 13: mod_base = OTHER; note = 2'-O-methyladenosine
  modified_base 14: mod_base = OTHER; note = 2'-fluoroadenosine
  modified_base 15: mod_base = OTHER; note = 2'-O-methyladenosine
  modified_base 16: mod_base = OTHER; note = 2'-fluoroguanosine
  modified_base 17: mod_base = um
  modified_base 18: mod_base = gm
  modified_base 19: mod_base = OTHER; note = 2'-O-methyladenosine
  modified_base 20: mod_base = gm
  modified_base 20^21: mod_base = OTHER; note = phosphorothioate linkage
  modified_base 21: mod_base = gm
  source 1..21: mol_type = other RNA
tgatagaggattaaagtgagg

SEQ ID NO: 51 - RNA, 21 nt, synthetic construct
  modified_base 1: mod_base = OTHER; note = 5 -cyclopropyl phosphonate-2 -O-methyluridine
  modified_base 1^2: mod_base = OTHER; note = phosphorothioate linkage
  modified_base 2: mod_base = OTHER; note = 2'-fluoroguanosine
  modified_base 2^3: mod_base = OTHER; note = phosphorothioate linkage
  modified_base 3: mod_base = OTHER; note = 2'-O-methyladenosine
  modified_base 3^4: mod_base = OTHER; note = phosphorothioate linkage
  modified_base 4: mod_base = um
  modified_base 5: mod_base = OTHER; note = 2'-O-methyladenosine
  modified_base 6: mod_base = gm
  modified_base 7: mod_base = OTHER; note = 2 ,3 -seco-adenosine
  modified_base 8: mod_base = gm
  modified_base 9: mod_base = gm
  modified_base 10: mod_base = OTHER; note = 2'-fluoroadenosine
  modified_base 11: mod_base = um
  modified_base 12: mod_base = OTHER; note = 2'-fluorouridine
  modified_base 13: mod_base = OTHER; note = 2'-O-methyladenosine
  modified_base 14: mod_base = OTHER; note = 2'-fluoroadenosine
  modified_base 15: mod_base = OTHER; note = 2'-O-methyladenosine
  modified_base 16: mod_base = OTHER; note = 2'-fluoroguanosine
  modified_base 17: mod_base = um
  modified_base 18: mod_base = gm
  modified_base 19: mod_base = OTHER; note = 2'-O-methyladenosine
  modified_base 20: mod_base = gm
  modified_base 20^21: mod_base = OTHER; note = phosphorothioate linkage
  modified_base 21: mod_base = gm
  source 1..21: mol_type = other RNA
tgatagaggattaaagtgagg

SEQ ID NO: 52 - RNA, 21 nt, synthetic construct
  modified_base 1: mod_base = OTHER; note = 5 -cyclopropyl phosphonate-2 -O-methyluridine
  modified_base 1^2: mod_base = OTHER; note = phosphorothioate linkage
  modified_base 2: mod_base = OTHER; note = 2'-fluoroguanosine
  modified_base 2^3: mod_base = OTHER; note = phosphorothioate linkage
  modified_base 3: mod_base = OTHER; note = 2'-O-methyladenosine
  modified_base 4: mod_base = OTHER; note = 2'-fluoroguanosine
  modified_base 5: mod_base = OTHER; note = 2'-O-methyladenosine
  modified_base 6: mod_base = um
  modified_base 7: mod_base = cm
  modified_base 8: mod_base = OTHER; note = 2'-O-methyladenosine
  modified_base 9: mod_base = cm
  modified_base 10: mod_base = OTHER; note = 2'-O-methyladenosine
  modified_base 11: mod_base = gm
  modified_base 12: mod_base = OTHER; note = 2'-fluoroadenosine
  modified_base 13: mod_base = OTHER; note = 2'-O-methyladenosine
  modified_base 14: mod_base = OTHER; note = 2'-fluorouridine
  modified_base 15: mod_base = cm
  modified_base 16: mod_base = OTHER; note = 2'-fluorouridine
  modified_base 17: mod_base = um
  modified_base 18: mod_base = cm
  modified_base 19: mod_base = OTHER; note = 2'-O-methyladenosine
  modified_base 19^20: mod_base = OTHER; note = phosphorothioate linkage
  modified_base 20: mod_base = OTHER; note = 2'-O-methyladenosine
  modified_base 20^21: mod_base = OTHER; note = phosphorothioate linkage
  modified_base 21: mod_base = cm
  source 1..21: mol_type = other RNA
tgagatcacagaatcttcaac

SEQ ID NO: 53 - RNA, 21 nt, synthetic construct
  modified_base 1: mod_base = OTHER; note = 5 -cyclopropyl phosphonate-2 -O-methyluridine
  modified_base 1^2: mod_base = OTHER; note = phosphorothioate linkage
  modified_base 2: mod_base = OTHER; note = 2'-fluorouridine
  modified_base 2^3: mod_base = OTHER; note = phosphorothioate linkage
  modified_base 3: mod_base = um
  modified_base 3^4: mod_base = OTHER; note = phosphorothioate linkage
  modified_base 4: mod_base = OTHER; note = 2'-fluoroadenosine
  modified_base 5: mod_base = gm
  modified_base 6: mod_base = OTHER; note = 2'-O-methyladenosine
  modified_base 7: mod_base = gm
  modified_base 8: mod_base = um
  modified_base 9: mod_base = gm
  modified_base 10: mod_base = OTHER; note = 2'-O-methyladenosine
  modified_base 11: mod_base = gm
  modified_base 12: mod_base = OTHER; note = 2'-fluoroguanosine
  modified_base 13: mod_base = OTHER; note = 2'-O-methyladenosine
  modified_base 14: mod_base = OTHER; note = 2'-fluorouridine
  modified_base 15: mod_base = um
  modified_base 16: mod_base = OTHER; note = 2'-fluoroadenosine
  modified_base 17: mod_base = OTHER; note = 2'-O-methyladenosine
  modified_base 18: mod_base = OTHER; note = 2'-fluoroadenosine
  modified_base 19: mod_base = OTHER; note = 2'-O-methyladenosine
  modified_base 20: mod_base = OTHER; note = 2'-fluorouridine
  modified_base 20^21: mod_base = OTHER; note = phosphorothioate linkage
  modified_base 21: mod_base = gm
  source 1..21: mol_type = other RNA
tttagagtgaggattaaaatg

SEQ ID NO: 54 - RNA, 21 nt, synthetic construct
  modified_base 1: mod_base = OTHER; note = 5 -cyclopropyl phosphonate-2 -O-methyluridine
  modified_base 1^2: mod_base = OTHER; note = phosphorothioate linkage
  modified_base 2: mod_base = OTHER; note = 2'-fluorouridine
  modified_base 2^3: mod_base = OTHER; note = phosphorothioate linkage
  modified_base 3: mod_base = um
  modified_base 3^4: mod_base = OTHER; note = phosphorothioate linkage
  modified_base 4: mod_base = OTHER; note = 2'-fluoroadenosine
  modified_base 5: mod_base = gm
  modified_base 6: mod_base = OTHER; note = 2'-O-methyladenosine
  modified_base 7: mod_base = gm
  modified_base 8: mod_base = um
  modified_base 9: mod_base = gm
  modified_base 10: mod_base = OTHER; note = 2'-O-methyladenosine
  modified_base 11: mod_base = gm
  modified_base 12: mod_base = OTHER; note = 2'-fluoroguanosine
  modified_base 13: mod_base = OTHER; note = 2'-O-methyladenosine
  modified_base 14: mod_base = OTHER; note = 2'-fluorouridine
  modified_base 15: mod_base = um
  modified_base 16: mod_base = OTHER; note = 2'-fluoroadenosine
  modified_base 17: mod_base = OTHER; note = 2'-O-methyladenosine
  modified_base 18: mod_base = OTHER; note = 2'-fluoroadenosine
  modified_base 19: mod_base = OTHER; note = 2'-O-methyladenosine
  modified_base 20: mod_base = OTHER; note = 2'-fluorouridine
  modified_base 20^21: mod_base = OTHER; note = phosphorothioate linkage
  modified_base 21: mod_base = gm
  source 1..21: mol_type = other RNA
tttagagtgaggattaaaatg

SEQ ID NO: 55 - RNA, 21 nt, synthetic construct
  modified_base 1: mod_base = OTHER; note = 5 -cyclopropyl phosphonate-2 -O-methyluridine
  modified_base 1^2: mod_base = OTHER; note = phosphorothioate linkage
  modified_base 2: mod_base = OTHER; note = 2'-fluorouridine
  modified_base 2^3: mod_base = OTHER; note = phosphorothioate linkage
  modified_base 3: mod_base = um
  modified_base 3^4: mod_base = OTHER; note = phosphorothioate linkage
  modified_base 4: mod_base = OTHER; note = 2'-fluoroadenosine
  modified_base 5: mod_base = gm
  modified_base 6: mod_base = OTHER; note = 2'-O-methyladenosine
  modified_base 7: mod_base = gm
  modified_base 8: mod_base = um
  modified_base 9: mod_base = gm
  modified_base 10: mod_base = OTHER; note = 2'-O-methyladenosine
  modified_base 11: mod_base = gm
  modified_base 12: mod_base = OTHER; note = 2'-fluoroguanosine
  modified_base 13: mod_base = OTHER; note = 2'-O-methyladenosine
  modified_base 14: mod_base = OTHER; note = 2'-fluorouridine
  modified_base 15: mod_base = um
  modified_base 16: mod_base = OTHER; note = 2'-fluoroadenosine
  modified_base 17: mod_base = OTHER; note = 2'-O-methyladenosine
  modified_base 18: mod_base = OTHER; note = 2'-fluoroadenosine
  modified_base 19: mod_base = OTHER; note = 2'-O-methyladenosine
  modified_base 20: mod_base = OTHER; note = 2'-fluorouridine
  modified_base 20^21: mod_base = OTHER; note = phosphorothioate linkage
  modified_base 21: mod_base = gm
  source 1..21: mol_type = other RNA
tttagagtgaggattaaaatg

SEQ ID NO: 56 - RNA, 21 nt, synthetic construct
  modified_base 1: mod_base = OTHER; note = 5 -cyclopropyl phosphonate-2 -O-methyluridine
  modified_base 1^2: mod_base = OTHER; note = phosphorothioate linkage
  modified_base 2: mod_base = OTHER; note = 2'-fluorouridine
  modified_base 2^3: mod_base = OTHER; note = phosphorothioate linkage
  modified_base 3: mod_base = um
  modified_base 3^4: mod_base = OTHER; note = phosphorothioate linkage
  modified_base 4: mod_base = OTHER; note = 2'-fluoroadenosine
  modified_base 5: mod_base = gm
  modified_base 6: mod_base = OTHER; note = 2'-O-methyladenosine
  modified_base 7: mod_base = gm
  modified_base 8: mod_base = um
  modified_base 9: mod_base = gm
  modified_base 10: mod_base = OTHER; note = 2'-O-methyladenosine
  modified_base 11: mod_base = gm
  modified_base 12: mod_base = OTHER; note = 2'-fluoroguanosine
  modified_base 13: mod_base = OTHER; note = 2'-O-methyladenosine
  modified_base 14: mod_base = OTHER; note = 2'-fluorouridine
  modified_base 15: mod_base = um
  modified_base 16: mod_base = OTHER; note = 2'-fluoroadenosine
  modified_base 17: mod_base = OTHER; note = 2'-O-methyladenosine
  modified_base 18: mod_base = OTHER; note = 2'-fluoroadenosine
  modified_base 19: mod_base = OTHER; note = 2'-O-methyladenosine
  modified_base 20: mod_base = OTHER; note = 2'-fluorouridine
  modified_base 20^21: mod_base = OTHER; note = phosphorothioate linkage
  modified_base 21: mod_base = gm
  source 1..21: mol_type = other RNA
tttagagtgaggattaaaatg

SEQ ID NO: 57 - RNA, 21 nt, synthetic construct
  modified_base 1: mod_base = OTHER; note = 5 -cyclopropyl phosphonate-2 -O-methyluridine
  modified_base 1^2: mod_base = OTHER; note = phosphorothioate linkage
  modified_base 2: mod_base = OTHER; note = 2'-fluorouridine
  modified_base 2^3: mod_base = OTHER; note = phosphorothioate linkage
  modified_base 3: mod_base = um
  modified_base 3^4: mod_base = OTHER; note = phosphorothioate linkage
  modified_base 4: mod_base = OTHER; note = 2'-fluoroadenosine
  modified_base 5: mod_base = gm
  modified_base 6: mod_base = OTHER; note = 2'-O-methyladenosine
  modified_base 7: mod_base = gm
  modified_base 8: mod_base = um
  modified_base 9: mod_base = gm
  modified_base 10: mod_base = OTHER; note = 2'-O-methyladenosine
  modified_base 11: mod_base = gm
  modified_base 12: mod_base = OTHER; note = 2'-fluoroguanosine
  modified_base 13: mod_base = OTHER; note = 2'-O-methyladenosine
  modified_base 14: mod_base = OTHER; note = 2'-fluorouridine
  modified_base 15: mod_base = um
  modified_base 16: mod_base = OTHER; note = 2'-fluoroadenosine
  modified_base 17: mod_base = OTHER; note = 2'-O-methyladenosine
  modified_base 18: mod_base = OTHER; note = 2'-fluoroadenosine
  modified_base 19: mod_base = OTHER; note = 2'-O-methyladenosine
  modified_base 20: mod_base = OTHER; note = 2'-fluorouridine
  modified_base 20^21: mod_base = OTHER; note = phosphorothioate linkage
  modified_base 21: mod_base = gm
  source 1..21: mol_type = other RNA
tttagagtgaggattaaaatg

SEQ ID NO: 58 - RNA, 21 nt, synthetic construct
  modified_base 1: mod_base = OTHER; note = 5 -cyclopropyl phosphonate-2 -O-methyluridine
  modified_base 1^2: mod_base = OTHER; note = phosphorothioate linkage
  modified_base 2: mod_base = OTHER; note = 2'-fluorouridine
  modified_base 2^3: mod_base = OTHER; note = phosphorothioate linkage
  modified_base 3: mod_base = um
  modified_base 3^4: mod_base = OTHER; note = phosphorothioate linkage
  modified_base 4: mod_base = OTHER; note = 2'-fluoroadenosine
  modified_base 5: mod_base = gm
  modified_base 6: mod_base = OTHER; note = 2'-O-methyladenosine
  modified_base 7: mod_base = gm
  modified_base 8: mod_base = um
  modified_base 9: mod_base = gm
  modified_base 10: mod_base = OTHER; note = 2'-O-methyladenosine
  modified_base 11: mod_base = gm
  modified_base 12: mod_base = OTHER; note = 2'-fluoroguanosine
  modified_base 13: mod_base = OTHER; note = 2'-O-methyladenosine
  modified_base 14: mod_base = OTHER; note = 2'-fluorouridine
  modified_base 15: mod_base = um
  modified_base 16: mod_base = OTHER; note = 2'-fluoroadenosine
  modified_base 17: mod_base = OTHER; note = 2'-O-methyladenosine
  modified_base 18: mod_base = OTHER; note = 2'-fluoroadenosine
  modified_base 19: mod_base = OTHER; note = 2'-O-methyladenosine
  modified_base 20: mod_base = OTHER; note = 2'-fluorouridine
  modified_base 20^21: mod_base = OTHER; note = phosphorothioate linkage
  modified_base 21: mod_base = gm
  source 1..21: mol_type = other RNA
tttagagtgaggattaaaatg

SEQ ID NO: 59 - RNA, 21 nt, synthetic construct
  modified_base 1: mod_base = OTHER; note = 5 -cyclopropyl phosphonate-2 -O-methyluridine
  modified_base 1^2: mod_base = OTHER; note = phosphorothioate linkage
  modified_base 2: mod_base = OTHER; note = 2'-fluorouridine
  modified_base 2^3: mod_base = OTHER; note = phosphorothioate linkage
  modified_base 3: mod_base = um
  modified_base 3^4: mod_base = OTHER; note = phosphorothioate linkage
  modified_base 4: mod_base = OTHER; note = 2'-fluoroadenosine
  modified_base 5: mod_base = gm
  modified_base 6: mod_base = OTHER; note = 2'-O-methyladenosine
  modified_base 7: mod_base = gm
  modified_base 8: mod_base = um
  modified_base 9: mod_base = gm
  modified_base 10: mod_base = OTHER; note = 2'-O-methyladenosine
  modified_base 11: mod_base = gm
  modified_base 12: mod_base = OTHER; note = 2'-fluoroguanosine
  modified_base 13: mod_base = OTHER; note = 2'-O-methyladenosine
  modified_base 14: mod_base = OTHER; note = 2'-fluorouridine
  modified_base 15: mod_base = um
  modified_base 16: mod_base = OTHER; note = 2'-fluoroadenosine
  modified_base 17: mod_base = OTHER; note = 2'-O-methyladenosine
  modified_base 18: mod_base = OTHER; note = 2'-fluoroadenosine
  modified_base 19: mod_base = OTHER; note = 2'-O-methyladenosine
  modified_base 20: mod_base = OTHER; note = 2'-fluorouridine
  modified_base 20^21: mod_base = OTHER; note = phosphorothioate linkage
  modified_base 21: mod_base = gm
  source 1..21: mol_type = other RNA
tttagagtgaggattaaaatg

SEQ ID NO: 60 - RNA, 21 nt, synthetic construct
  modified_base 1: mod_base = OTHER; note = 5 -cyclopropyl phosphonate-2 -O-methyluridine
  modified_base 1^2: mod_base = OTHER; note = phosphorothioate linkage
  modified_base 2: mod_base = OTHER; note = 2'-fluorouridine
  modified_base 2^3: mod_base = OTHER; note = phosphorothioate linkage
  modified_base 3: mod_base = um
  modified_base 3^4: mod_base = OTHER; note = phosphorothioate linkage
  modified_base 4: mod_base = OTHER; note = 2'-fluoroadenosine
  modified_base 5: mod_base = gm
  modified_base 6: mod_base = OTHER; note = 2'-O-methyladenosine
  modified_base 7: mod_base = gm
  modified_base 8: mod_base = um
  modified_base 9: mod_base = gm
  modified_base 10: mod_base = OTHER; note = 2'-O-methyladenosine
  modified_base 11: mod_base = gm
  modified_base 12: mod_base = OTHER; note = 2'-fluoroguanosine
  modified_base 13: mod_base = OTHER; note = 2'-O-methyladenosine
  modified_base 14: mod_base = OTHER; note = 2'-fluorouridine
  modified_base 15: mod_base = um
  modified_base 16: mod_base = OTHER; note = 2'-fluoroadenosine
  modified_base 17: mod_base = OTHER; note = 2'-O-methyladenosine
  modified_base 18: mod_base = OTHER; note = 2'-fluoroadenosine
  modified_base 19: mod_base = OTHER; note = 2'-O-methyladenosine
  modified_base 20: mod_base = OTHER; note = 2'-fluorouridine
  modified_base 20^21: mod_base = OTHER; note = phosphorothioate linkage
  modified_base 21: mod_base = gm
  source 1..21: mol_type = other RNA
tttagagtgaggattaaaatg

SEQ ID NO: 61 - RNA, 21 nt, synthetic construct
  modified_base 1: mod_base = OTHER; note = 5 -cyclopropyl phosphonate-2 -O-methyluridine
  modified_base 1^2: mod_base = OTHER; note = phosphorothioate linkage
  modified_base 2: mod_base = OTHER; note = 2'-fluorouridine
  modified_base 2^3: mod_base = OTHER; note = phosphorothioate linkage
  modified_base 3: mod_base = um
  modified_base 3^4: mod_base = OTHER; note = phosphorothioate linkage
  modified_base 4: mod_base = OTHER; note = 2'-fluoroadenosine
  modified_base 5: mod_base = gm
  modified_base 6: mod_base = OTHER; note = 2'-O-methyladenosine
  modified_base 7: mod_base = gm
  modified_base 8: mod_base = um
  modified_base 9: mod_base = gm
  modified_base 10: mod_base = OTHER; note = 2'-O-methyladenosine
  modified_base 11: mod_base = gm
  modified_base 12: mod_base = OTHER; note = 2'-fluoroguanosine
  modified_base 13: mod_base = OTHER; note = 2'-O-methyladenosine
  modified_base 14: mod_base = OTHER; note = 2'-fluorouridine
  modified_base 15: mod_base = um
  modified_base 16: mod_base = OTHER; note = 2'-fluoroadenosine
  modified_base 17: mod_base = OTHER; note = 2'-O-methyladenosine
  modified_base 18: mod_base = OTHER; note = 2'-fluoroadenosine
  modified_base 19: mod_base = OTHER; note = 2'-O-methyladenosine
  modified_base 20: mod_base = OTHER; note = 2'-fluorouridine
  modified_base 20^21: mod_base = OTHER; note = phosphorothioate linkage
  modified_base 21: mod_base = gm
  source 1..21: mol_type = other RNA
tttagagtgaggattaaaatg

SEQ ID NO: 62 - RNA, 21 nt, synthetic construct
  modified_base 1: mod_base = OTHER; note = 5 -cyclopropyl phosphonate-2 -O-methyluridine
  modified_base 1^2: mod_base = OTHER; note = phosphorothioate linkage
  modified_base 2: mod_base = OTHER; note = 2'-fluorouridine
  modified_base 2^3: mod_base = OTHER; note = phosphorothioate linkage
  modified_base 3: mod_base = um
  modified_base 3^4: mod_base = OTHER; note = phosphorothioate linkage
  modified_base 4: mod_base = OTHER; note = 2'-fluoroadenosine
  modified_base 5: mod_base = gm
  modified_base 6: mod_base = OTHER; note = 2'-O-methyladenosine
  modified_base 7: mod_base = gm
  modified_base 8: mod_base = um
  modified_base 9: mod_base = gm
  modified_base 10: mod_base = OTHER; note = 2'-O-methyladenosine
  modified_base 11: mod_base = gm
  modified_base 12: mod_base = OTHER; note = 2'-fluoroguanosine
  modified_base 13: mod_base = OTHER; note = 2'-O-methyladenosine
  modified_base 14: mod_base = OTHER; note = 2'-fluorouridine
  modified_base 15: mod_base = um
  modified_base 16: mod_base = OTHER; note = 2'-fluoroadenosine
  modified_base 17: mod_base = OTHER; note = 2'-O-methyladenosine
  modified_base 18: mod_base = OTHER; note = 2'-fluoroadenosine
  modified_base 19: mod_base = OTHER; note = 2'-O-methyladenosine
  modified_base 20: mod_base = OTHER; note = 2'-fluorouridine
  modified_base 20^21: mod_base = OTHER; note = phosphorothioate linkage
  modified_base 21: mod_base = gm
  source 1..21: mol_type = other RNA
tttagagtgaggattaaaatg

SEQ ID NO: 63 - RNA, 21 nt, synthetic construct
  modified_base 1: mod_base = OTHER; note = 5 -cyclopropyl phosphonate-2 -O-methyluridine
  modified_base 1^2: mod_base = OTHER; note = phosphorothioate linkage
  modified_base 2: mod_base = OTHER; note = 2'-fluorouridine
  modified_base 2^3: mod_base = OTHER; note = phosphorothioate linkage
  modified_base 3: mod_base = um
  modified_base 3^4: mod_base = OTHER; note = phosphorothioate linkage
  modified_base 4: mod_base = OTHER; note = 2'-fluoroadenosine
  modified_base 5: mod_base = gm
  modified_base 6: mod_base = OTHER; note = 2'-O-methyladenosine
  modified_base 7: mod_base = gm
  modified_base 8: mod_base = um
  modified_base 9: mod_base = gm
  modified_base 10: mod_base = OTHER; note = 2'-O-methyladenosine
  modified_base 11: mod_base = gm
  modified_base 12: mod_base = OTHER; note = 2'-fluoroguanosine
  modified_base 13: mod_base = OTHER; note = 2'-O-methyladenosine
  modified_base 14: mod_base = OTHER; note = 2'-fluorouridine
  modified_base 15: mod_base = um
  modified_base 16: mod_base = OTHER; note = 2'-fluoroadenosine
  modified_base 17: mod_base = OTHER; note = 2'-O-methyladenosine
  modified_base 18: mod_base = OTHER; note = 2'-fluoroadenosine
  modified_base 19: mod_base = OTHER; note = 2'-O-methyladenosine
  modified_base 20: mod_base = OTHER; note = 2'-fluorouridine
  modified_base 20^21: mod_base = OTHER; note = phosphorothioate linkage
  modified_base 21: mod_base = gm
  source 1..21: mol_type = other RNA
tttagagtgaggattaaaatg

SEQ ID NO: 64 - RNA, 21 nt, synthetic construct
  modified_base 1: mod_base = OTHER; note = 5 -cyclopropyl phosphonate-2 -O-methyluridine
  modified_base 1^2: mod_base = OTHER; note = phosphorothioate linkage
  modified_base 2: mod_base = OTHER; note = 2'-fluorouridine
  modified_base 2^3: mod_base = OTHER; note = phosphorothioate linkage
  modified_base 3: mod_base = um
  modified_base 3^4: mod_base = OTHER; note = phosphorothioate linkage
  modified_base 4: mod_base = OTHER; note = 2'-fluoroadenosine
  modified_base 5: mod_base = gm
  modified_base 6: mod_base = OTHER; note = 2'-O-methyladenosine
  modified_base 7: mod_base = gm
  modified_base 8: mod_base = um
  modified_base 9: mod_base = gm
  modified_base 10: mod_base = OTHER; note = 2'-O-methyladenosine
  modified_base 11: mod_base = gm
  modified_base 12: mod_base = OTHER; note = 2'-fluoroguanosine
  modified_base 13: mod_base = OTHER; note = 2'-O-methyladenosine
  modified_base 14: mod_base = OTHER; note = 2'-fluorouridine
  modified_base 15: mod_base = um
  modified_base 16: mod_base = OTHER; note = 2'-fluoroadenosine
  modified_base 17: mod_base = OTHER; note = 2'-O-methyladenosine
  modified_base 18: mod_base = OTHER; note = 2'-fluoroadenosine
  modified_base 19: mod_base = OTHER; note = 2'-O-methyladenosine
  modified_base 20: mod_base = OTHER; note = 2'-fluorouridine
  modified_base 20^21: mod_base = OTHER; note = phosphorothioate linkage
  modified_base 21: mod_base = gm
  source 1..21: mol_type = other RNA
tttagagtgaggattaaaatg

SEQ ID NO: 65 - RNA, 21 nt, synthetic construct
  modified_base 1: mod_base = OTHER; note = 5 -cyclopropyl phosphonate-2 -O-methyluridine
  modified_base 1^2: mod_base = OTHER; note = phosphorothioate linkage
  modified_base 2: mod_base = OTHER; note = 2'-fluorouridine
  modified_base 2^3: mod_base = OTHER; note = phosphorothioate linkage
  modified_base 3: mod_base = um
  modified_base 3^4: mod_base = OTHER; note = phosphorothioate linkage
  modified_base 4: mod_base = OTHER; note = 2'-fluoroadenosine
  modified_base 5: mod_base = gm
  modified_base 6: mod_base = OTHER; note = 2'-O-methyladenosine
  modified_base 7: mod_base = gm
  modified_base 8: mod_base = um
  modified_base 9: mod_base = gm
  modified_base 10: mod_base = OTHER; note = 2'-O-methyladenosine
  modified_base 11: mod_base = gm
  modified_base 12: mod_base = OTHER; note = 2'-fluoroguanosine
  modified_base 13: mod_base = OTHER; note = 2'-O-methyladenosine
  modified_base 14: mod_base = OTHER; note = 2'-fluorouridine
  modified_base 15: mod_base = um
  modified_base 16: mod_base = OTHER; note = 2'-fluoroadenosine
  modified_base 17: mod_base = OTHER; note = 2'-O-methyladenosine
  modified_base 18: mod_base = OTHER; note = 2'-fluoroadenosine
  modified_base 19: mod_base = OTHER; note = 2'-O-methyladenosine
  modified_base 20: mod_base = OTHER; note = 2'-fluorouridine
  modified_base 20^21: mod_base = OTHER; note = phosphorothioate linkage
  modified_base 21: mod_base = gm
  source 1..21: mol_type = other RNA
tttagagtgaggattaaaatg

SEQ ID NO: 66 - RNA, 21 nt, synthetic construct
  modified_base 1: mod_base = OTHER; note = 5 -cyclopropyl phosphonate-2 -O-methyluridine
  modified_base 1^2: mod_base = OTHER; note = phosphorothioate linkage
  modified_base 2: mod_base = OTHER; note = 2'-fluorouridine
  modified_base 2^3: mod_base = OTHER; note = phosphorothioate linkage
  modified_base 3: mod_base = um
  modified_base 3^4: mod_base = OTHER; note = phosphorothioate linkage
  modified_base 4: mod_base = OTHER; note = 2'-fluoroadenosine
  modified_base 5: mod_base = gm
  modified_base 6: mod_base = OTHER; note = 2'-O-methyladenosine
  modified_base 7: mod_base = gm
  modified_base 8: mod_base = um
  modified_base 9: mod_base = gm
  modified_base 10: mod_base = OTHER; note = 2'-O-methyladenosine
  modified_base 11: mod_base = gm
  modified_base 12: mod_base = OTHER; note = 2'-fluoroguanosine
  modified_base 13: mod_base = OTHER; note = 2'-O-methyladenosine
  modified_base 14: mod_base = OTHER; note = 2'-fluorouridine
  modified_base 15: mod_base = um
  modified_base 16: mod_base = OTHER; note = 2'-fluoroadenosine
  modified_base 17: mod_base = OTHER; note = 2'-O-methyladenosine
  modified_base 18: mod_base = OTHER; note = 2'-fluoroadenosine
  modified_base 19: mod_base = OTHER; note = 2'-O-methyladenosine
  modified_base 20: mod_base = OTHER; note = 2'-fluorouridine
  modified_base 20^21: mod_base = OTHER; note = phosphorothioate linkage
  modified_base 21: mod_base = gm
  source 1..21: mol_type = other RNA
tttagagtgaggattaaaatg

SEQ ID NO: 67 - RNA, 21 nt, synthetic construct
  modified_base 1: mod_base = OTHER; note = 5 -cyclopropyl phosphonate-2 -O-methyluridine
  modified_base 1^2: mod_base = OTHER; note = phosphorothioate linkage
  modified_base 2: mod_base = OTHER; note = 2'-fluorouridine
  modified_base 2^3: mod_base = OTHER; note = phosphorothioate linkage
  modified_base 3: mod_base = um
  modified_base 3^4: mod_base = OTHER; note = phosphorothioate linkage
  modified_base 4: mod_base = OTHER; note = 2'-fluoroadenosine
  modified_base 5: mod_base = gm
  modified_base 6: mod_base = OTHER; note = 2'-O-methyladenosine
  modified_base 7: mod_base = gm
  modified_base 8: mod_base = um
  modified_base 9: mod_base = gm
  modified_base 10: mod_base = OTHER; note = 2'-O-methyladenosine
  modified_base 11: mod_base = gm
  modified_base 12: mod_base = OTHER; note = 2'-fluoroguanosine
  modified_base 13: mod_base = OTHER; note = 2'-O-methyladenosine
  modified_base 14: mod_base = OTHER; note = 2'-fluorouridine
  modified_base 15: mod_base = um
  modified_base 16: mod_base = OTHER; note = 2'-fluoroadenosine
  modified_base 17: mod_base = OTHER; note = 2'-O-methyladenosine
  modified_base 18: mod_base = OTHER; note = 2'-fluoroadenosine
  modified_base 19: mod_base = OTHER; note = 2'-O-methyladenosine
  modified_base 20: mod_base = OTHER; note = 2'-fluorouridine
  modified_base 20^21: mod_base = OTHER; note = phosphorothioate linkage
  modified_base 21: mod_base = gm
  source 1..21: mol_type = other RNA
tttagagtgaggattaaaatg

SEQ ID NO: 68 - RNA, 21 nt, synthetic construct
  modified_base 1: mod_base = cm
  modified_base 2: mod_base = OTHER; note = 2'-O-methyladenosine
  modified_base 3: mod_base = um
  modified_base 4: mod_base = um
  modified_base 5: mod_base = um
  modified_base 6: mod_base = um
  modified_base 7: mod_base = OTHER; note = 2'-O-methyladenosine
  modified_base 8: mod_base = OTHER; note = 2'-O-methyladenosine
  modified_base 9: mod_base = OTHER; note = 2'-fluorouridine
  modified_base 10: mod_base = OTHER; note = 2'-fluorocytidine
  modified_base 11: mod_base = OTHER; note = 2'-fluorocytidine
  modified_base 12: mod_base = um
  modified_base 13: mod_base = cm
  modified_base 14: mod_base = OTHER; note = 2'-O-methyladenosine
  modified_base 15: mod_base = cm
  modified_base 16: mod_base = um
  modified_base 17: mod_base = cm
  modified_base 18: mod_base = um
  modified_base 19: mod_base = OTHER; note = 2'-O-methyladenosine
  modified_base 20: mod_base = OTHER; note = 2'-O-methyladenosine
  modified_base 21: mod_base = OTHER; note = 2'-O-methyladenosine
  source 1..21: mol_type = other RNA
cattttaatcctcactctaaa

SEQ ID NO: 69 - RNA, 21 nt, synthetic construct
  modified_base 1: mod_base = cm
  modified_base 2: mod_base = OTHER; note = 2'-O-methyladenosine
  modified_base 3: mod_base = um
  modified_base 4: mod_base = um
  modified_base 5: mod_base = um
  modified_base 6: mod_base = um
  modified_base 7: mod_base = OTHER; note = 2'-O-methyladenosine
  modified_base 8: mod_base = OTHER; note = 2'-O-methyladenosine
  modified_base 9: mod_base = OTHER; note = 2'-fluorouridine
  modified_base 10: mod_base = OTHER; note = 2'-fluorocytidine
  modified_base 11: mod_base = OTHER; note = 2'-fluorocytidine
  modified_base 12: mod_base = um
  modified_base 13: mod_base = cm
  modified_base 14: mod_base = OTHER; note = 2'-O-methyladenosine
  modified_base 15: mod_base = cm
  modified_base 16: mod_base = um
  modified_base 17: mod_base = cm
  modified_base 18: mod_base = um
  modified_base 19: mod_base = OTHER; note = 2'-O-methyladenosine
  modified_base 20: mod_base = OTHER; note = 2'-O-methyladenosine
  modified_base 21: mod_base = OTHER; note = 2'-O-methyladenosine
  source 1..21: mol_type = other RNA
cattttaatcctcactctaaa

SEQ ID NO: 70 - RNA, 21 nt, synthetic construct
  modified_base 1: mod_base = cm
  modified_base 2: mod_base = OTHER; note = 2'-O-methyladenosine
  modified_base 3: mod_base = um
  modified_base 4: mod_base = um
  modified_base 5: mod_base = um
  modified_base 6: mod_base = um
  modified_base 7: mod_base = OTHER; note = 2'-O-methyladenosine
  modified_base 8: mod_base = OTHER; note = 2'-O-methyladenosine
  modified_base 9: mod_base = OTHER; note = 2'-fluorouridine
  modified_base 10: mod_base = OTHER; note = 2'-fluorocytidine
  modified_base 11: mod_base = OTHER; note = 2'-fluorocytidine
  modified_base 12: mod_base = um
  modified_base 13: mod_base = cm
  modified_base 14: mod_base = OTHER; note = 2'-O-methyladenosine
  modified_base 15: mod_base = cm
  modified_base 16: mod_base = um
  modified_base 17: mod_base = cm
  modified_base 18: mod_base = um
  modified_base 19: mod_base = OTHER; note = 2'-O-methyladenosine
  modified_base 20: mod_base = OTHER; note = 2'-O-methyladenosine
  modified_base 21: mod_base = OTHER; note = 2'-O-methyladenosine
  source 1..21: mol_type = other RNA
cattttaatcctcactctaaa

SEQ ID NO: 71 - RNA, 21 nt, synthetic construct
  modified_base 1: mod_base = cm
  modified_base 2: mod_base = OTHER; note = 2'-O-methyladenosine
  modified_base 3: mod_base = um
  modified_base 4: mod_base = um
  modified_base 5: mod_base = um
  modified_base 6: mod_base = um
  modified_base 7: mod_base = OTHER; note = 2'-O-methyladenosine
  modified_base 8: mod_base = OTHER; note = 2'-O-methyladenosine
  modified_base 9: mod_base = OTHER; note = 2'-fluorouridine
  modified_base 10: mod_base = OTHER; note = 2'-fluorocytidine
  modified_base 11: mod_base = OTHER; note = 2'-fluorocytidine
  modified_base 12: mod_base = um
  modified_base 13: mod_base = cm
  modified_base 14: mod_base = OTHER; note = 2'-O-methyladenosine
  modified_base 15: mod_base = cm
  modified_base 16: mod_base = um
  modified_base 17: mod_base = cm
  modified_base 18: mod_base = um
  modified_base 19: mod_base = OTHER; note = 2'-O-methyladenosine
  modified_base 20: mod_base = OTHER; note = 2'-O-methyladenosine
  modified_base 21: mod_base = OTHER; note = 2'-O-methyladenosine
  source 1..21: mol_type = other RNA
cattttaatcctcactctaaa

SEQ ID NO: 72 - RNA, 21 nt, synthetic construct
  modified_base 1: mod_base = cm
  modified_base 2: mod_base = OTHER; note = 2'-O-methyladenosine
  modified_base 3: mod_base = um
  modified_base 4: mod_base = um
  modified_base 5: mod_base = um
  modified_base 6: mod_base = um
  modified_base 7: mod_base = OTHER; note = 2'-O-methyladenosine
  modified_base 8: mod_base = OTHER; note = 2'-O-methyladenosine
  modified_base 9: mod_base = OTHER; note = 2'-fluorouridine
  modified_base 10: mod_base = OTHER; note = 2'-fluorocytidine
  modified_base 11: mod_base = OTHER; note = 2'-fluorocytidine
  modified_base 12: mod_base = um
  modified_base 13: mod_base = cm
  modified_base 14: mod_base = OTHER; note = 2'-O-methyladenosine
  modified_base 15: mod_base = cm
  modified_base 16: mod_base = um
  modified_base 17: mod_base = cm
  modified_base 18: mod_base = um
  modified_base 19: mod_base = OTHER; note = 2'-O-methyladenosine
  modified_base 20: mod_base = OTHER; note = 2'-O-methyladenosine
  modified_base 21: mod_base = OTHER; note = 2'-O-methyladenosine
  source 1..21: mol_type = other RNA
cattttaatcctcactctaaa

SEQ ID NO: 73 - RNA, 21 nt, synthetic construct
  modified_base 1: mod_base = cm
  modified_base 2: mod_base = OTHER; note = 2'-O-methyladenosine
  modified_base 3: mod_base = um
  modified_base 4: mod_base = um
  modified_base 5: mod_base = um
  modified_base 6: mod_base = um
  modified_base 7: mod_base = OTHER; note = 2'-O-methyladenosine
  modified_base 8: mod_base = OTHER; note = 2'-O-methyladenosine
  modified_base 9: mod_base = OTHER; note = 2'-fluorouridine
  modified_base 10: mod_base = OTHER; note = 2'-fluorocytidine
  modified_base 11: mod_base = OTHER; note = 2'-fluorocytidine
  modified_base 12: mod_base = um
  modified_base 13: mod_base = cm
  modified_base 14: mod_base = OTHER; note = 2'-O-methyladenosine
  modified_base 15: mod_base = cm
  modified_base 16: mod_base = um
  modified_base 17: mod_base = cm
  modified_base 18: mod_base = um
  modified_base 19: mod_base = OTHER; note = 2'-O-methyladenosine
  modified_base 20: mod_base = OTHER; note = 2'-O-methyladenosine
  modified_base 21: mod_base = OTHER; note = 2'-O-methyladenosine
  source 1..21: mol_type = other RNA
cattttaatcctcactctaaa

SEQ ID NO: 74 - RNA, 21 nt, synthetic construct
  modified_base 1: mod_base = cm
  modified_base 2: mod_base = OTHER; note = 2'-O-methyladenosine
  modified_base 3: mod_base = um
  modified_base 4: mod_base = um
  modified_base 5: mod_base = um
  modified_base 6: mod_base = um
  modified_base 7: mod_base = OTHER; note = 2'-O-methyladenosine
  modified_base 8: mod_base = OTHER; note = 2'-O-methyladenosine
  modified_base 9: mod_base = OTHER; note = 2'-fluorouridine
  modified_base 10: mod_base = OTHER; note = 2'-fluorocytidine
  modified_base 11: mod_base = OTHER; note = 2'-fluorocytidine
  modified_base 12: mod_base = um
  modified_base 13: mod_base = cm
  modified_base 14: mod_base = OTHER; note = 2'-O-methyladenosine
  modified_base 15: mod_base = cm
  modified_base 16: mod_base = um
  modified_base 17: mod_base = cm
  modified_base 18: mod_base = um
  modified_base 19: mod_base = OTHER; note = 2'-O-methyladenosine
  modified_base 20: mod_base = OTHER; note = 2'-O-methyladenosine
  modified_base 21: mod_base = OTHER; note = 2'-O-methyladenosine
  source 1..21: mol_type = other RNA
cattttaatcctcactctaaa

SEQ ID NO: 75 - RNA, 21 nt, synthetic construct
  modified_base 1: mod_base = cm
  modified_base 2: mod_base = OTHER; note = 2'-O-methyladenosine
  modified_base 3: mod_base = um
  modified_base 4: mod_base = um
  modified_base 5: mod_base = um
  modified_base 6: mod_base = um
  modified_base 7: mod_base = OTHER; note = 2'-O-methyladenosine
  modified_base 8: mod_base = OTHER; note = 2'-O-methyladenosine
  modified_base 9: mod_base = OTHER; note = 2'-fluorouridine
  modified_base 10: mod_base = OTHER; note = 2'-fluorocytidine
  modified_base 11: mod_base = OTHER; note = 2'-fluorocytidine
  modified_base 12: mod_base = um
  modified_base 13: mod_base = cm
  modified_base 14: mod_base = OTHER; note = 2'-O-methyladenosine
  modified_base 15: mod_base = cm
  modified_base 16: mod_base = um
  modified_base 17: mod_base = cm
  modified_base 18: mod_base = um
  modified_base 19: mod_base = OTHER; note = 2'-O-methyladenosine
  modified_base 20: mod_base = OTHER; note = 2'-O-methyladenosine
  modified_base 21: mod_base = OTHER; note = 2'-O-methyladenosine
  source 1..21: mol_type = other RNA
cattttaatcctcactctaaa

SEQ ID NO: 76 - RNA, 21 nt, synthetic construct
  modified_base 1: mod_base = cm
  modified_base 2: mod_base = OTHER; note = 2'-O-methyladenosine
  modified_base 3: mod_base = um
  modified_base 4: mod_base = um
  modified_base 5: mod_base = um
  modified_base 6: mod_base = um
  modified_base 7: mod_base = OTHER; note = 2'-O-methyladenosine
  modified_base 8: mod_base = OTHER; note = 2'-O-methyladenosine
  modified_base 9: mod_base = OTHER; note = 2'-fluorouridine
  modified_base 10: mod_base = OTHER; note = 2'-fluorocytidine
  modified_base 11: mod_base = OTHER; note = 2'-fluorocytidine
  modified_base 12: mod_base = um
  modified_base 13: mod_base = cm
  modified_base 14: mod_base = OTHER; note = 2'-O-methyladenosine
  modified_base 15: mod_base = cm
  modified_base 16: mod_base = um
  modified_base 17: mod_base = cm
  modified_base 18: mod_base = um
  modified_base 19: mod_base = OTHER; note = 2'-O-methyladenosine
  modified_base 20: mod_base = OTHER; note = 2'-O-methyladenosine
  modified_base 21: mod_base = OTHER; note = 2'-O-methyladenosine
  source 1..21: mol_type = other RNA
cattttaatcctcactctaaa

SEQ ID NO: 77 - RNA, 21 nt, synthetic construct
  modified_base 1: mod_base = cm
  modified_base 2: mod_base = OTHER; note = 2'-O-methyladenosine
  modified_base 3: mod_base = um
  modified_base 4: mod_base = um
  modified_base 5: mod_base = um
  modified_base 6: mod_base = um
  modified_base 7: mod_base = OTHER; note = 2'-O-methyladenosine
  modified_base 8: mod_base = OTHER; note = 2'-O-methyladenosine
  modified_base 9: mod_base = OTHER; note = 2'-fluorouridine
  modified_base 10: mod_base = OTHER; note = 2'-fluorocytidine
  modified_base 11: mod_base = OTHER; note = 2'-fluorocytidine
  modified_base 12: mod_base = um
  modified_base 13: mod_base = cm
  modified_base 14: mod_base = OTHER; note = 2'-O-methyladenosine
  modified_base 15: mod_base = cm
  modified_base 16: mod_base = um
  modified_base 17: mod_base = cm
  modified_base 18: mod_base = um
  modified_base 19: mod_base = OTHER; note = 2'-O-methyladenosine
  modified_base 20: mod_base = OTHER; note = 2'-O-methyladenosine
  modified_base 21: mod_base = OTHER; note = 2'-O-methyladenosine
  source 1..21: mol_type = other RNA
cattttaatcctcactctaaa

SEQ ID NO: 78 - RNA, 21 nt, synthetic construct
  modified_base 1: mod_base = cm
  modified_base 2: mod_base = OTHER; note = 2'-O-methyladenosine
  modified_base 3: mod_base = um
  modified_base 4: mod_base = um
  modified_base 5: mod_base = um
  modified_base 6: mod_base = um
  modified_base 7: mod_base = OTHER; note = 2'-O-methyladenosine
  modified_base 8: mod_base = OTHER; note = 2'-O-methyladenosine
  modified_base 9: mod_base = OTHER; note = 2'-fluorouridine
  modified_base 10: mod_base = OTHER; note = 2'-fluorocytidine
  modified_base 11: mod_base = OTHER; note = 2'-fluorocytidine
  modified_base 12: mod_base = um
  modified_base 13: mod_base = cm
  modified_base 14: mod_base = OTHER; note = 2'-O-methyladenosine
  modified_base 15: mod_base = cm
  modified_base 16: mod_base = um
  modified_base 17: mod_base = cm
  modified_base 18: mod_base = um
  modified_base 19: mod_base = OTHER; note = 2'-O-methyladenosine
  modified_base 20: mod_base = OTHER; note = 2'-O-methyladenosine
  modified_base 21: mod_base = OTHER; note = 2'-O-methyladenosine
  source 1..21: mol_type = other RNA
cattttaatcctcactctaaa

SEQ ID NO: 79 - RNA, 21 nt, synthetic construct
  modified_base 1: mod_base = cm
  modified_base 2: mod_base = OTHER; note = 2'-O-methyladenosine
  modified_base 3: mod_base = um
  modified_base 4: mod_base = um
  modified_base 5: mod_base = um
  modified_base 6: mod_base = um
  modified_base 7: mod_base = OTHER; note = 2'-O-methyladenosine
  modified_base 8: mod_base = OTHER; note = 2'-O-methyladenosine
  modified_base 9: mod_base = OTHER; note = 2'-fluorouridine
  modified_base 10: mod_base = OTHER; note = 2'-fluorocytidine
  modified_base 11: mod_base = OTHER; note = 2'-fluorocytidine
  modified_base 12: mod_base = um
  modified_base 13: mod_base = cm
  modified_base 14: mod_base = OTHER; note = 2'-O-methyladenosine
  modified_base 15: mod_base = cm
  modified_base 16: mod_base = um
  modified_base 17: mod_base = cm
  modified_base 18: mod_base = um
  modified_base 19: mod_base = OTHER; note = 2'-O-methyladenosine
  modified_base 20: mod_base = OTHER; note = 2'-O-methyladenosine
  modified_base 21: mod_base = OTHER; note = 2'-O-methyladenosine
  source 1..21: mol_type = other RNA
cattttaatcctcactctaaa

SEQ ID NO: 80 - RNA, 21 nt, synthetic construct
  modified_base 1: mod_base = cm
  modified_base 2: mod_base = OTHER; note = 2'-O-methyladenosine
  modified_base 3: mod_base = um
  modified_base 4: mod_base = um
  modified_base 5: mod_base = um
  modified_base 6: mod_base = um
  modified_base 7: mod_base = OTHER; note = 2'-O-methyladenosine
  modified_base 8: mod_base = OTHER; note = 2'-O-methyladenosine
  modified_base 9: mod_base = OTHER; note = 2'-fluorouridine
  modified_base 10: mod_base = OTHER; note = 2'-fluorocytidine
  modified_base 11: mod_base = OTHER; note = 2'-fluorocytidine
  modified_base 12: mod_base = um
  modified_base 13: mod_base = cm
  modified_base 14: mod_base = OTHER; note = 2'-O-methyladenosine
  modified_base 15: mod_base = cm
  modified_base 16: mod_base = um
  modified_base 17: mod_base = cm
  modified_base 18: mod_base = um
  modified_base 19: mod_base = OTHER; note = 2'-O-methyladenosine
  modified_base 20: mod_base = OTHER; note = 2'-O-methyladenosine
  modified_base 21: mod_base = OTHER; note = 2'-O-methyladenosine
  source 1..21: mol_type = other RNA
cattttaatcctcactctaaa

SEQ ID NO: 81 - RNA, 21 nt, synthetic construct
  modified_base 1: mod_base = cm
  modified_base 2: mod_base = OTHER; note = 2'-O-methyladenosine
  modified_base 3: mod_base = um
  modified_base 4: mod_base = um
  modified_base 5: mod_base = um
  modified_base 6: mod_base = um
  modified_base 7: mod_base = OTHER; note = 2'-O-methyladenosine
  modified_base 8: mod_base = OTHER; note = 2'-O-methyladenosine
  modified_base 9: mod_base = OTHER; note = 2'-fluorouridine
  modified_base 10: mod_base = OTHER; note = 2'-fluorocytidine
  modified_base 11: mod_base = OTHER; note = 2'-fluorocytidine
  modified_base 12: mod_base = um
  modified_base 13: mod_base = cm
  modified_base 14: mod_base = OTHER; note = 2'-O-methyladenosine
  modified_base 15: mod_base = cm
  modified_base 16: mod_base = um
  modified_base 17: mod_base = cm
  modified_base 18: mod_base = um
  modified_base 19: mod_base = OTHER; note = 2'-O-methyladenosine
  modified_base 20: mod_base = OTHER; note = 2'-O-methyladenosine
  modified_base 21: mod_base = OTHER; note = 2'-O-methyladenosine
  source 1..21: mol_type = other RNA
cattttaatcctcactctaaa

SEQ ID NO: 82 - RNA, 21 nt, synthetic construct
  modified_base 1: mod_base = cm
  modified_base 2: mod_base = OTHER; note = 2'-O-methyladenosine
  modified_base 3: mod_base = um
  modified_base 4: mod_base = um
  modified_base 5: mod_base = um
  modified_base 6: mod_base = um
  modified_base 7: mod_base = OTHER; note = 2'-O-methyladenosine
  modified_base 8: mod_base = OTHER; note = 2'-O-methyladenosine
  modified_base 9: mod_base = OTHER; note = 2'-fluorouridine
  modified_base 10: mod_base = OTHER; note = 2'-fluorocytidine
  modified_base 11: mod_base = OTHER; note = 2'-fluorocytidine
  modified_base 12: mod_base = um
  modified_base 13: mod_base = cm
  modified_base 14: mod_base = OTHER; note = 2'-O-methyladenosine
  modified_base 15: mod_base = cm
  modified_base 16: mod_base = um
  modified_base 17: mod_base = cm
  modified_base 18: mod_base = um
  modified_base 19: mod_base = OTHER; note = 2'-O-methyladenosine
  modified_base 20: mod_base = OTHER; note = 2'-O-methyladenosine
  modified_base 21: mod_base = OTHER; note = 2'-O-methyladenosine
  source 1..21: mol_type = other RNA
cattttaatcctcactctaaa

SEQ ID NO: 83 - RNA, 21 nt, synthetic construct
  modified_base 1: mod_base = cm
  modified_base 2: mod_base = OTHER; note = 2'-O-methyladenosine
  modified_base 3: mod_base = um
  modified_base 4: mod_base = um
  modified_base 5: mod_base = um
  modified_base 6: mod_base = um
  modified_base 7: mod_base = OTHER; note = 2'-O-methyladenosine
  modified_base 8: mod_base = OTHER; note = 2'-O-methyladenosine
  modified_base 9: mod_base = OTHER; note = 2'-fluorouridine
  modified_base 10: mod_base = OTHER; note = 2'-fluorocytidine
  modified_base 11: mod_base = OTHER; note = 2'-fluorocytidine
  modified_base 12: mod_base = um
  modified_base 13: mod_base = cm
  modified_base 14: mod_base = OTHER; note = 2'-O-methyladenosine
  modified_base 15: mod_base = cm
  modified_base 16: mod_base = um
  modified_base 17: mod_base = cm
  modified_base 18: mod_base = um
  modified_base 19: mod_base = OTHER; note = 2'-O-methyladenosine
  modified_base 20: mod_base = OTHER; note = 2'-O-methyladenosine
  modified_base 21: mod_base = OTHER; note = 2'-O-methyladenosine
  source 1..21: mol_type = other RNA
cattttaatcctcactctaaa

SEQ ID NO: 84 - RNA, 21 nt, synthetic construct
  modified_base 1: mod_base = cm
  modified_base 2: mod_base = OTHER; note = 2'-O-methyladenosine
  modified_base 3: mod_base = um
  modified_base 4: mod_base = um
  modified_base 5: mod_base = um
  modified_base 6: mod_base = um
  modified_base 7: mod_base = OTHER; note = 2'-O-methyladenosine
  modified_base 8: mod_base = OTHER; note = 2'-O-methyladenosine
  modified_base 9: mod_base = OTHER; note = 2'-fluorouridine
  modified_base 10: mod_base = OTHER; note = 2'-fluorocytidine
  modified_base 11: mod_base = OTHER; note = 2'-fluorocytidine
  modified_base 12: mod_base = um
  modified_base 13: mod_base = cm
  modified_base 14: mod_base = OTHER; note = 2'-O-methyladenosine
  modified_base 15: mod_base = cm
  modified_base 16: mod_base = um
  modified_base 17: mod_base = cm
  modified_base 18: mod_base = um
  modified_base 19: mod_base = OTHER; note = 2'-O-methyladenosine
  modified_base 20: mod_base = OTHER; note = 2'-O-methyladenosine
  modified_base 21: mod_base = OTHER; note = 2'-O-methyladenosine
  source 1..21: mol_type = other RNA
cattttaatcctcactctaaa

SEQ ID NO: 85 - RNA, 21 nt, synthetic construct
  modified_base 1: mod_base = cm
  modified_base 2: mod_base = OTHER; note = 2'-O-methyladenosine
  modified_base 3: mod_base = um
  modified_base 4: mod_base = um
  modified_base 5: mod_base = um
  modified_base 6: mod_base = um
  modified_base 7: mod_base = OTHER; note = 2'-O-methyladenosine
  modified_base 8: mod_base = OTHER; note = 2'-O-methyladenosine
  modified_base 9: mod_base = OTHER; note = 2'-fluorouridine
  modified_base 10: mod_base = OTHER; note = 2'-fluorocytidine
  modified_base 11: mod_base = OTHER; note = 2'-fluorocytidine
  modified_base 12: mod_base = um
  modified_base 13: mod_base = cm
  modified_base 14: mod_base = OTHER; note = 2'-O-methyladenosine
  modified_base 15: mod_base = cm
  modified_base 16: mod_base = um
  modified_base 17: mod_base = cm
  modified_base 18: mod_base = um
  modified_base 19: mod_base = OTHER; note = 2'-O-methyladenosine
  modified_base 20: mod_base = OTHER; note = 2'-O-methyladenosine
  modified_base 21: mod_base = OTHER; note = 2'-O-methyladenosine
  source 1..21: mol_type = other RNA
cattttaatcctcactctaaa

SEQ ID NO: 86 - RNA, 21 nt, synthetic construct
  modified_base 1: mod_base = cm
  modified_base 2: mod_base = OTHER; note = 2'-O-methyladenosine
  modified_base 3: mod_base = um
  modified_base 4: mod_base = um
  modified_base 5: mod_base = um
  modified_base 6: mod_base = um
  modified_base 7: mod_base = OTHER; note = 2'-O-methyladenosine
  modified_base 8: mod_base = OTHER; note = 2'-O-methyladenosine
  modified_base 9: mod_base = OTHER; note = 2'-fluorouridine
  modified_base 10: mod_base = OTHER; note = 2'-fluorocytidine
  modified_base 11: mod_base = OTHER; note = 2'-fluorocytidine
  modified_base 12: mod_base = um
  modified_base 13: mod_base = cm
  modified_base 14: mod_base = OTHER; note = 2'-O-methyladenosine
  modified_base 15: mod_base = cm
  modified_base 16: mod_base = um
  modified_base 17: mod_base = cm
  modified_base 18: mod_base = um
  modified_base 19: mod_base = OTHER; note = 2'-O-methyladenosine
  modified_base 20: mod_base = OTHER; note = 2'-O-methyladenosine
  modified_base 21: mod_base = OTHER; note = 2'-O-methyladenosine
  source 1..21: mol_type = other RNA
cattttaatcctcactctaaa

SEQ ID NO: 87 - RNA, 21 nt, synthetic construct
  modified_base 1: mod_base = cm
  modified_base 2: mod_base = OTHER; note = 2'-O-methyladenosine
  modified_base 3: mod_base = um
  modified_base 4: mod_base = um
  modified_base 5: mod_base = um
  modified_base 6: mod_base = um
  modified_base 7: mod_base = OTHER; note = 2'-O-methyladenosine
  modified_base 8: mod_base = OTHER; note = 2'-O-methyladenosine
  modified_base 9: mod_base = OTHER; note = 2'-fluorouridine
  modified_base 10: mod_base = OTHER; note = 2'-fluorocytidine
  modified_base 11: mod_base = OTHER; note = 2'-fluorocytidine
  modified_base 12: mod_base = um
  modified_base 13: mod_base = cm
  modified_base 14: mod_base = OTHER; note = 2'-O-methyladenosine
  modified_base 15: mod_base = cm
  modified_base 16: mod_base = um
  modified_base 17: mod_base = cm
  modified_base 18: mod_base = um
  modified_base 19: mod_base = OTHER; note = 2'-O-methyladenosine
  modified_base 20: mod_base = OTHER; note = 2'-O-methyladenosine
  modified_base 21: mod_base = OTHER; note = 2'-O-methyladenosine
  source 1..21: mol_type = other RNA
cattttaatcctcactctaaa

SEQ ID NO: 88 - RNA, 21 nt, synthetic construct
  modified_base 1: mod_base = cm
  modified_base 2: mod_base = OTHER; note = 2'-O-methyladenosine
  modified_base 3: mod_base = um
  modified_base 4: mod_base = um
  modified_base 5: mod_base = um
  modified_base 6: mod_base = um
  modified_base 7: mod_base = OTHER; note = 2'-O-methyladenosine
  modified_base 8: mod_base = OTHER; note = 2'-O-methyladenosine
  modified_base 9: mod_base = OTHER; note = 2'-fluorouridine
  modified_base 10: mod_base = OTHER; note = 2'-fluorocytidine
  modified_base 11: mod_base = OTHER; note = 2'-fluorocytidine
  modified_base 12: mod_base = um
  modified_base 13: mod_base = cm
  modified_base 14: mod_base = OTHER; note = 2'-O-methyladenosine
  modified_base 15: mod_base = cm
  modified_base 16: mod_base = um
  modified_base 17: mod_base = cm
  modified_base 18: mod_base = um
  modified_base 19: mod_base = OTHER; note = 2'-O-methyladenosine
  modified_base 20: mod_base = OTHER; note = 2'-O-methyladenosine
  modified_base 21: mod_base = OTHER; note = 2'-O-methyladenosine
  source 1..21: mol_type = other RNA
cattttaatcctcactctaaa

SEQ ID NO: 89 - RNA, 21 nt, synthetic construct
  modified_base 1: mod_base = cm
  modified_base 2: mod_base = OTHER; note = 2'-O-methyladenosine
  modified_base 3: mod_base = um
  modified_base 4: mod_base = um
  modified_base 5: mod_base = um
  modified_base 6: mod_base = um
  modified_base 7: mod_base = OTHER; note = 2'-O-methyladenosine
  modified_base 8: mod_base = OTHER; note = 2'-O-methyladenosine
  modified_base 9: mod_base = OTHER; note = 2'-fluorouridine
  modified_base 10: mod_base = OTHER; note = 2'-fluorocytidine
  modified_base 11: mod_base = OTHER; note = 2'-fluorocytidine
  modified_base 12: mod_base = um
  modified_base 13: mod_base = cm
  modified_base 14: mod_base = OTHER; note = 2'-O-methyladenosine
  modified_base 15: mod_base = cm
  modified_base 16: mod_base = um
  modified_base 17: mod_base = cm
  modified_base 18: mod_base = um
  modified_base 19: mod_base = OTHER; note = 2'-O-methyladenosine
  modified_base 20: mod_base = OTHER; note = 2'-O-methyladenosine
  modified_base 21: mod_base = OTHER; note = 2'-O-methyladenosine
  source 1..21: mol_type = other RNA
cattttaatcctcactctaaa

SEQ ID NO: 90 - RNA, 21 nt, synthetic construct
  modified_base 1: mod_base = cm
  modified_base 2: mod_base = OTHER; note = 2'-O-methyladenosine
  modified_base 3: mod_base = um
  modified_base 4: mod_base = um
  modified_base 5: mod_base = um
  modified_base 6: mod_base = um
  modified_base 7: mod_base = OTHER; note = 2'-O-methyladenosine
  modified_base 8: mod_base = OTHER; note = 2'-O-methyladenosine
  modified_base 9: mod_base = OTHER; note = 2'-fluorouridine
  modified_base 10: mod_base = OTHER; note = 2'-fluorocytidine
  modified_base 11: mod_base = OTHER; note = 2'-fluorocytidine
  modified_base 12: mod_base = um
  modified_base 13: mod_base = cm
  modified_base 14: mod_base = OTHER; note = 2'-O-methyladenosine
  modified_base 15: mod_base = cm
  modified_base 16: mod_base = um
  modified_base 17: mod_base = cm
  modified_base 18: mod_base = um
  modified_base 19: mod_base = OTHER; note = 2'-O-methyladenosine
  modified_base 20: mod_base = OTHER; note = 2'-O-methyladenosine
  modified_base 21: mod_base = OTHER; note = 2'-O-methyladenosine
  source 1..21: mol_type = other RNA
cattttaatcctcactctaaa

SEQ ID NO: 91 - RNA, 21 nt, synthetic construct
  modified_base 1: mod_base = cm
  modified_base 2: mod_base = OTHER; note = 2'-O-methyladenosine
  modified_base 3: mod_base = um
  modified_base 4: mod_base = um
  modified_base 5: mod_base = um
  modified_base 6: mod_base = um
  modified_base 7: mod_base = OTHER; note = 2'-O-methyladenosine
  modified_base 8: mod_base = OTHER; note = 2'-O-methyladenosine
  modified_base 9: mod_base = OTHER; note = 2'-fluorouridine
  modified_base 10: mod_base = OTHER; note = 2'-fluorocytidine
  modified_base 11: mod_base = OTHER; note = 2'-fluorocytidine
  modified_base 12: mod_base = um
  modified_base 13: mod_base = cm
  modified_base 14: mod_base = OTHER; note = 2'-O-methyladenosine
  modified_base 15: mod_base = cm
  modified_base 16: mod_base = um
  modified_base 17: mod_base = cm
  modified_base 18: mod_base = um
  modified_base 19: mod_base = OTHER; note = 2'-O-methyladenosine
  modified_base 20: mod_base = OTHER; note = 2'-O-methyladenosine
  modified_base 21: mod_base = OTHER; note = 2'-O-methyladenosine
  source 1..21: mol_type = other RNA
cattttaatcctcactctaaa

SEQ ID NO: 92 - RNA, 21 nt, synthetic construct
  modified_base 1: mod_base = cm
  modified_base 2: mod_base = OTHER; note = 2'-O-methyladenosine
  modified_base 3: mod_base = um
  modified_base 4: mod_base = um
  modified_base 5: mod_base = um
  modified_base 6: mod_base = um
  modified_base 7: mod_base = OTHER; note = 2'-O-methyladenosine
  modified_base 8: mod_base = OTHER; note = 2'-O-methyladenosine
  modified_base 9: mod_base = OTHER; note = 2'-fluorouridine
  modified_base 10: mod_base = OTHER; note = 2'-fluorocytidine
  modified_base 11: mod_base = OTHER; note = 2'-fluorocytidine
  modified_base 12: mod_base = um
  modified_base 13: mod_base = cm
  modified_base 14: mod_base = OTHER; note = 2'-O-methyladenosine
  modified_base 15: mod_base = cm
  modified_base 16: mod_base = um
  modified_base 17: mod_base = cm
  modified_base 18: mod_base = um
  modified_base 19: mod_base = OTHER; note = 2'-O-methyladenosine
  modified_base 20: mod_base = OTHER; note = 2'-O-methyladenosine
  modified_base 21: mod_base = OTHER; note = 2'-O-methyladenosine
  source 1..21: mol_type = other RNA
cattttaatcctcactctaaa

SEQ ID NO: 93 - RNA, 21 nt, synthetic construct
  modified_base 1: mod_base = cm
  modified_base 2: mod_base = OTHER; note = 2'-O-methyladenosine
  modified_base 3: mod_base = um
  modified_base 4: mod_base = um
  modified_base 5: mod_base = um
  modified_base 6: mod_base = um
  modified_base 7: mod_base = OTHER; note = 2'-O-methyladenosine
  modified_base 8: mod_base = OTHER; note = 2'-O-methyladenosine
  modified_base 9: mod_base = OTHER; note = 2'-fluorouridine
  modified_base 10: mod_base = OTHER; note = 2'-fluorocytidine
  modified_base 11: mod_base = OTHER; note = 2'-fluorocytidine
  modified_base 12: mod_base = um
  modified_base 13: mod_base = cm
  modified_base 14: mod_base = OTHER; note = 2'-O-methyladenosine
  modified_base 15: mod_base = cm
  modified_base 16: mod_base = um
  modified_base 17: mod_base = cm
  modified_base 18: mod_base = um
  modified_base 19: mod_base = OTHER; note = 2'-O-methyladenosine
  modified_base 20: mod_base = OTHER; note = 2'-O-methyladenosine
  modified_base 21: mod_base = OTHER; note = 2'-O-methyladenosine
  source 1..21: mol_type = other RNA
cattttaatcctcactctaaa

SEQ ID NO: 94 - RNA, 21 nt, synthetic construct
  modified_base 1: mod_base = cm
  modified_base 2: mod_base = OTHER; note = 2'-O-methyladenosine
  modified_base 3: mod_base = um
  modified_base 4: mod_base = um
  modified_base 5: mod_base = um
  modified_base 6: mod_base = um
  modified_base 7: mod_base = OTHER; note = 2'-O-methyladenosine
  modified_base 8: mod_base = OTHER; note = 2'-O-methyladenosine
  modified_base 9: mod_base = OTHER; note = 2'-fluorouridine
  modified_base 10: mod_base = OTHER; note = 2'-fluorocytidine
  modified_base 11: mod_base = OTHER; note = 2'-fluorocytidine
  modified_base 12: mod_base = um
  modified_base 13: mod_base = cm
  modified_base 14: mod_base = OTHER; note = 2'-O-methyladenosine
  modified_base 15: mod_base = cm
  modified_base 16: mod_base = um
  modified_base 17: mod_base = cm
  modified_base 18: mod_base = um
  modified_base 19: mod_base = OTHER; note = 2'-O-methyladenosine
  modified_base 20: mod_base = OTHER; note = 2'-O-methyladenosine
  modified_base 21: mod_base = OTHER; note = 2'-O-methyladenosine
  source 1..21: mol_type = other RNA
cattttaatcctcactctaaa

SEQ ID NO: 95 - RNA, 21 nt, synthetic construct
  modified_base 1: mod_base = cm
  modified_base 2: mod_base = cm
  modified_base 3: mod_base = um
  modified_base 4: mod_base = cm
  modified_base 5: mod_base = OTHER; note = 2'-O-methyladenosine
  modified_base 6: mod_base = cm
  modified_base 7: mod_base = um
  modified_base 8: mod_base = um
  modified_base 9: mod_base = OTHER; note = 2'-fluorouridine
  modified_base 10: mod_base = OTHER; note = 2'-fluoroadenosine
  modified_base 11: mod_base = OTHER; note = 2'-fluoroadenosine
  modified_base 12: mod_base = um
  modified_base 13: mod_base = cm
  modified_base 14: mod_base = cm
  modified_base 15: mod_base = um
  modified_base 16: mod_base = cm
  modified_base 17: mod_base = um
  modified_base 18: mod_base = OTHER; note = 2'-O-methyladenosine
  modified_base 19: mod_base = um
  modified_base 20: mod_base = cm
  modified_base 21: mod_base = OTHER; note = 2'-O-methyladenosine
  source 1..21: mol_type = other RNA
cctcactttaatcctctatca

SEQ ID NO: 96 - RNA, 21 nt, synthetic construct
  modified_base 1: mod_base = cm
  modified_base 2: mod_base = cm
  modified_base 3: mod_base = um
  modified_base 4: mod_base = cm
  modified_base 5: mod_base = OTHER; note = 2'-O-methyladenosine
  modified_base 6: mod_base = cm
  modified_base 7: mod_base = um
  modified_base 8: mod_base = um
  modified_base 9: mod_base = OTHER; note = 2'-fluorouridine
  modified_base 10: mod_base = OTHER; note = 2'-fluoroadenosine
  modified_base 11: mod_base = OTHER; note = 2'-fluoroadenosine
  modified_base 12: mod_base = um
  modified_base 13: mod_base = cm
  modified_base 14: mod_base = cm
  modified_base 15: mod_base = um
  modified_base 16: mod_base = cm
  modified_base 17: mod_base = um
  modified_base 18: mod_base = OTHER; note = 2'-O-methyladenosine
  modified_base 19: mod_base = um
  modified_base 20: mod_base = cm
  modified_base 21: mod_base = OTHER; note = 2'-O-methyladenosine
  source 1..21: mol_type = other RNA
cctcactttaatcctctatca

SEQ ID NO: 97 - RNA, 21 nt, synthetic construct
  modified_base 1: mod_base = cm
  modified_base 2: mod_base = cm
  modified_base 3: mod_base = um
  modified_base 4: mod_base = cm
  modified_base 5: mod_base = OTHER; note = 2'-O-methyladenosine
  modified_base 6: mod_base = cm
  modified_base 7: mod_base = um
  modified_base 8: mod_base = um
  modified_base 9: mod_base = OTHER; note = 2'-fluorouridine
  modified_base 10: mod_base = OTHER; note = 2'-fluoroadenosine
  modified_base 11: mod_base = OTHER; note = 2'-fluoroadenosine
  modified_base 12: mod_base = um
  modified_base 13: mod_base = cm
  modified_base 14: mod_base = cm
  modified_base 15: mod_base = um
  modified_base 16: mod_base = cm
  modified_base 17: mod_base = um
  modified_base 18: mod_base = OTHER; note = 2'-O-methyladenosine
  modified_base 19: mod_base = um
  modified_base 20: mod_base = cm
  modified_base 21: mod_base = OTHER; note = 2'-O-methyladenosine
  source 1..21: mol_type = other RNA
cctcactttaatcctctatca

SEQ ID NO: 98 - RNA, 21 nt, synthetic construct
  modified_base 1: mod_base = cm
  modified_base 2: mod_base = cm
  modified_base 3: mod_base = um
  modified_base 4: mod_base = cm
  modified_base 5: mod_base = OTHER; note = 2'-O-methyladenosine
  modified_base 6: mod_base = cm
  modified_base 7: mod_base = um
  modified_base 8: mod_base = um
  modified_base 9: mod_base = OTHER; note = 2'-fluorouridine
  modified_base 10: mod_base = OTHER; note = 2'-fluoroadenosine
  modified_base 11: mod_base = OTHER; note = 2'-fluoroadenosine
  modified_base 12: mod_base = um
  modified_base 13: mod_base = cm
  modified_base 14: mod_base = cm
  modified_base 15: mod_base = um
  modified_base 16: mod_base = cm
  modified_base 17: mod_base = um
  modified_base 18: mod_base = OTHER; note = 2'-O-methyladenosine
  modified_base 19: mod_base = um
  modified_base 20: mod_base = cm
  modified_base 21: mod_base = OTHER; note = 2'-O-methyladenosine
  source 1..21: mol_type = other RNA
cctcactttaatcctctatca

SEQ ID NO: 99 - RNA, 21 nt, synthetic construct
  modified_base 1: mod_base = cm
  modified_base 2: mod_base = cm
  modified_base 3: mod_base = um
  modified_base 4: mod_base = cm
  modified_base 5: mod_base = OTHER; note = 2'-O-methyladenosine
  modified_base 6: mod_base = cm
  modified_base 7: mod_base = um
  modified_base 8: mod_base = um
  modified_base 9: mod_base = OTHER; note = 2'-fluorouridine
  modified_base 10: mod_base = OTHER; note = 2'-fluoroadenosine
  modified_base 11: mod_base = OTHER; note = 2'-fluoroadenosine
  modified_base 12: mod_base = um
  modified_base 13: mod_base = cm
  modified_base 14: mod_base = cm
  modified_base 15: mod_base = um
  modified_base 16: mod_base = cm
  modified_base 17: mod_base = um
  modified_base 18: mod_base = OTHER; note = 2'-O-methyladenosine
  modified_base 19: mod_base = um
  modified_base 20: mod_base = cm
  modified_base 21: mod_base = OTHER; note = 2'-O-methyladenosine
  source 1..21: mol_type = other RNA
cctcactttaatcctctatca

SEQ ID NO: 100 - RNA, 21 nt, synthetic construct
  modified_base 1: mod_base = cm
  modified_base 2: mod_base = OTHER; note = 2'-O-methyladenosine
  modified_base 3: mod_base = um
  modified_base 4: mod_base = um
  modified_base 5: mod_base = um
  modified_base 6: mod_base = OTHER; note = 2'C16 alkoxy uridine
  modified_base 7: mod_base = OTHER; note = 2'-fluoroadenosine
  modified_base 8: mod_base = OTHER; note = 2'-O-methyladenosine
  modified_base 9: mod_base = OTHER; note = 2'-fluorouridine
  modified_base 10: mod_base = OTHER; note = 2'-fluorocytidine
  modified_base 11: mod_base = OTHER; note = 2'-fluorocytidine
  modified_base 12: mod_base = um
  modified_base 13: mod_base = cm
  modified_base 14: mod_base = OTHER; note = 2'-O-methyladenosine
  modified_base 15: mod_base = cm
  modified_base 16: mod_base = um
  modified_base 17: mod_base = cm
  modified_base 18: mod_base = um
  modified_base 19: mod_base = OTHER; note = 2'-O-methyladenosine
  modified_base 20: mod_base = OTHER; note = 2'-O-methyladenosine
  modified_base 21: mod_base = OTHER; note = 2'-O-methyladenosine
  source 1..21: mol_type = other RNA
cattttaatcctcactctaaa

SEQ ID NO: 101 - RNA, 21 nt, synthetic construct
  modified_base 1: mod_base = cm
  modified_base 2: mod_base = OTHER; note = 2'-O-methyladenosine
  modified_base 3: mod_base = um
  modified_base 4: mod_base = um
  modified_base 5: mod_base = um
  modified_base 6: mod_base = um
  modified_base 7: mod_base = OTHER; note = 2'-fluoroadenosine
  modified_base 8: mod_base = OTHER; note = 2'-O-methyladenosine
  modified_base 9: mod_base = OTHER; note = 2'-fluorouridine
  modified_base 10: mod_base = OTHER; note = 2'-fluorocytidine
  modified_base 11: mod_base = OTHER; note = 2'-fluorocytidine
  modified_base 12: mod_base = um
  modified_base 13: mod_base = cm
  modified_base 14: mod_base = OTHER; note = 2'-O-methyladenosine
  modified_base 15: mod_base = cm
  modified_base 16: mod_base = um
  modified_base 17: mod_base = cm
  modified_base 18: mod_base = um
  modified_base 19: mod_base = OTHER; note = 2'-O-methyladenosine
  modified_base 20: mod_base = OTHER; note = 2'-O-methyladenosine
  modified_base 21: mod_base = OTHER; note = 2'-O-methyladenosine
  source 1..21: mol_type = other RNA
cattttaatcctcactctaaa

SEQ ID NO: 102 - RNA, 21 nt, synthetic construct
  modified_base 1: mod_base = cm
  modified_base 2: mod_base = OTHER; note = 2'-O-methyladenosine
  modified_base 3: mod_base = um
  modified_base 4: mod_base = um
  modified_base 5: mod_base = um
  modified_base 6: mod_base = OTHER; note = 2'-16-hydroxy-C16 alkoxy uridine
  modified_base 7: mod_base = OTHER; note = 2'-fluoroadenosine
  modified_base 8: mod_base = OTHER; note = 2'-O-methyladenosine
  modified_base 9: mod_base = OTHER; note = 2'-fluorouridine
  modified_base 10: mod_base = OTHER; note = 2'-fluorocytidine
  modified_base 11: mod_base = OTHER; note = 2'-fluorocytidine
  modified_base 12: mod_base = um
  modified_base 13: mod_base = cm
  modified_base 14: mod_base = OTHER; note = 2'-O-methyladenosine
  modified_base 15: mod_base = cm
  modified_base 16: mod_base = um
  modified_base 17: mod_base = cm
  modified_base 18: mod_base = um
  modified_base 19: mod_base = OTHER; note = 2'-O-methyladenosine
  modified_base 20: mod_base = OTHER; note = 2'-O-methyladenosine
  modified_base 21: mod_base = OTHER; note = 2'-O-methyladenosine
  source 1..21: mol_type = other RNA
cattttaatcctcactctaaa

SEQ ID NO: 103 - RNA, 21 nt, synthetic construct
  modified_base 1: mod_base = cm
  modified_base 2: mod_base = OTHER; note = 2'-O-methyladenosine
  modified_base 3: mod_base = um
  modified_base 4: mod_base = um
  modified_base 5: mod_base = um
  modified_base 6: mod_base = um
  modified_base 7: mod_base = OTHER; note = 2'-O-methyladenosine
  modified_base 8: mod_base = OTHER; note = 2'-O-methyladenosine
  modified_base 9: mod_base = OTHER; note = 2'-fluorouridine
  modified_base 10: mod_base = OTHER; note = 2'-fluorocytidine
  modified_base 11: mod_base = OTHER; note = 2'-fluorocytidine
  modified_base 12: mod_base = um
  modified_base 13: mod_base = cm
  modified_base 14: mod_base = OTHER; note = 2'-O-methyladenosine
  modified_base 15: mod_base = cm
  modified_base 16: mod_base = um
  modified_base 17: mod_base = cm
  modified_base 18: mod_base = um
  modified_base 19: mod_base = OTHER; note = 2'-O-methyladenosine
  modified_base 20: mod_base = OTHER; note = 2'-O-methyladenosine
  modified_base 21: mod_base = OTHER; note = 2'-O-methyladenosine
  source 1..21: mol_type = other RNA
cattttaatcctcactctaaa

SEQ ID NO: 104 - RNA, 21 nt, synthetic construct
  modified_base 1: mod_base = cm
  modified_base 2: mod_base = OTHER; note = 2'-O-methyladenosine
  modified_base 3: mod_base = um
  modified_base 4: mod_base = um
  modified_base 5: mod_base = um
  modified_base 6: mod_base = um
  modified_base 7: mod_base = OTHER; note = 2'-O-methyladenosine
  modified_base 8: mod_base = OTHER; note = 2'-O-methyladenosine
  modified_base 9: mod_base = OTHER; note = 2'-fluorouridine
  modified_base 10: mod_base = OTHER; note = 2'-fluorocytidine
  modified_base 11: mod_base = OTHER; note = 2'-fluorocytidine
  modified_base 12: mod_base = um
  modified_base 13: mod_base = cm
  modified_base 14: mod_base = OTHER; note = 2'-O-methyladenosine
  modified_base 15: mod_base = cm
  modified_base 16: mod_base = um
  modified_base 17: mod_base = cm
  modified_base 18: mod_base = um
  modified_base 19: mod_base = OTHER; note = 2'-O-methyladenosine
  modified_base 20: mod_base = OTHER; note = 2'-O-methyladenosine
  modified_base 21: mod_base = OTHER; note = 2'-O-methyladenosine
  source 1..21: mol_type = other RNA
cattttaatcctcactctaaa

SEQ ID NO: 105 - RNA, 21 nt, synthetic construct
  modified_base 1: mod_base = cm
  modified_base 2: mod_base = cm
  modified_base 3: mod_base = um
  modified_base 4: mod_base = cm
  modified_base 5: mod_base = OTHER; note = 2'-O-methyladenosine
  modified_base 6: mod_base = cm
  modified_base 7: mod_base = OTHER; note = 2'C16 alkoxy uridine
  modified_base 8: mod_base = um
  modified_base 9: mod_base = OTHER; note = 2'-fluorouridine
  modified_base 10: mod_base = OTHER; note = 2'-fluoroadenosine
  modified_base 11: mod_base = OTHER; note = 2'-fluoroadenosine
  modified_base 12: mod_base = um
  modified_base 13: mod_base = cm
  modified_base 14: mod_base = cm
  modified_base 15: mod_base = um
  modified_base 16: mod_base = cm
  modified_base 17: mod_base = um
  modified_base 18: mod_base = OTHER; note = 2'-O-methyladenosine
  modified_base 19: mod_base = um
  modified_base 20: mod_base = cm
  modified_base 21: mod_base = OTHER; note = 2'-O-methyladenosine
  source 1..21: mol_type = other RNA
cctcactttaatcctctatca

SEQ ID NO: 106 - RNA, 21 nt, synthetic construct
  modified_base 1: mod_base = cm
  modified_base 2: mod_base = cm
  modified_base 3: mod_base = um
  modified_base 4: mod_base = cm
  modified_base 5: mod_base = OTHER; note = 2'-O-methyladenosine
  modified_base 6: mod_base = cm
  modified_base 7: mod_base = um
  modified_base 8: mod_base = um
  modified_base 9: mod_base = OTHER; note = 2'-fluorouridine
  modified_base 10: mod_base = OTHER; note = 2'-fluoroadenosine
  modified_base 11: mod_base = OTHER; note = 2'-fluoroadenosine
  modified_base 12: mod_base = um
  modified_base 13: mod_base = cm
  modified_base 14: mod_base = cm
  modified_base 15: mod_base = OTHER; note = 2'C16 alkoxy uridine
  modified_base 16: mod_base = cm
  modified_base 17: mod_base = um
  modified_base 18: mod_base = OTHER; note = 2'-O-methyladenosine
  modified_base 19: mod_base = um
  modified_base 20: mod_base = cm
  modified_base 21: mod_base = OTHER; note = 2'-O-methyladenosine
  source 1..21: mol_type = other RNA
cctcactttaatcctctatca

SEQ ID NO: 107 - RNA, 21 nt, synthetic construct
  modified_base 1: mod_base = cm
  modified_base 2: mod_base = cm
  modified_base 3: mod_base = um
  modified_base 4: mod_base = cm
  modified_base 5: mod_base = OTHER; note = 2'-O-methyladenosine
  modified_base 6: mod_base = cm
  modified_base 7: mod_base = um
  modified_base 8: mod_base = um
  modified_base 9: mod_base = OTHER; note = 2'-fluorouridine
  modified_base 10: mod_base = OTHER; note = 2'-fluoroadenosine
  modified_base 11: mod_base = OTHER; note = 2'-fluoroadenosine
  modified_base 12: mod_base = um
  modified_base 13: mod_base = cm
  modified_base 14: mod_base = cm
  modified_base 15: mod_base = um
  modified_base 16: mod_base = cm
  modified_base 17: mod_base = um
  modified_base 18: mod_base = OTHER; note = 2'-O-methyladenosine
  modified_base 19: mod_base = um
  modified_base 20: mod_base = cm
  modified_base 21: mod_base = OTHER; note = 2'-O-methyladenosine
  source 1..21: mol_type = other RNA
cctcactttaatcctctatca

SEQ ID NO: 108 - RNA, 21 nt, synthetic construct
  modified_base 1: mod_base = cm
  modified_base 2: mod_base = um
  modified_base 3: mod_base = um
  modified_base 4: mod_base = OTHER; note = 2'-O-methyladenosine
  modified_base 5: mod_base = OTHER; note = 2'-O-methyladenosine
  modified_base 6: mod_base = cm
  modified_base 7: mod_base = um
  modified_base 8: mod_base = cm
  modified_base 9: mod_base = OTHER; note = 2'-fluoroadenosine
  modified_base 10: mod_base = OTHER; note = 2'-fluorouridine
  modified_base 11: mod_base = OTHER; note = 2'-fluorocytidine
  modified_base 12: mod_base = um
  modified_base 13: mod_base = gm
  modified_base 14: mod_base = um
  modified_base 15: mod_base = um
  modified_base 16: mod_base = OTHER; note = 2'-O-methyladenosine
  modified_base 17: mod_base = um
  modified_base 18: mod_base = cm
  modified_base 19: mod_base = cm
  modified_base 20: mod_base = um
  modified_base 21: mod_base = OTHER; note = 2'-O-methyladenosine
  source 1..21: mol_type = other RNA
cttaactcatctgttatccta

SEQ ID NO: 109 - RNA, 21 nt, synthetic construct
  modified_base 1: mod_base = cm
  modified_base 2: mod_base = cm
  modified_base 3: mod_base = um
  modified_base 4: mod_base = cm
  modified_base 5: mod_base = OTHER; note = 2'-O-methyladenosine
  modified_base 6: mod_base = cm
  modified_base 7: mod_base = um
  modified_base 8: mod_base = um
  modified_base 9: mod_base = OTHER; note = 2'-fluorouridine
  modified_base 10: mod_base = OTHER; note = 2'-fluoroadenosine
  modified_base 11: mod_base = OTHER; note = 2'-fluoroadenosine
  modified_base 12: mod_base = um
  modified_base 13: mod_base = cm
  modified_base 14: mod_base = cm
  modified_base 15: mod_base = um
  modified_base 16: mod_base = cm
  modified_base 17: mod_base = um
  modified_base 18: mod_base = OTHER; note = 2'-O-methyladenosine
  modified_base 19: mod_base = um
  modified_base 20: mod_base = cm
  modified_base 21: mod_base = OTHER; note = 2'-O-methyladenosine
  source 1..21: mol_type = other RNA
cctcactttaatcctctatca

SEQ ID NO: 110 - RNA, 21 nt, synthetic construct
  modified_base 1: mod_base = cm
  modified_base 2: mod_base = cm
  modified_base 3: mod_base = um
  modified_base 4: mod_base = cm
  modified_base 5: mod_base = OTHER; note = 2'-O-methyladenosine
  modified_base 6: mod_base = cm
  modified_base 7: mod_base = um
  modified_base 8: mod_base = um
  modified_base 9: mod_base = OTHER; note = 2'-fluorouridine
  modified_base 10: mod_base = OTHER; note = 2'-fluoroadenosine
  modified_base 11: mod_base = OTHER; note = 2'-fluoroadenosine
  modified_base 12: mod_base = um
  modified_base 13: mod_base = cm
  modified_base 14: mod_base = cm
  modified_base 15: mod_base = um
  modified_base 16: mod_base = cm
  modified_base 17: mod_base = um
  modified_base 18: mod_base = OTHER; note = 2'-O-methyladenosine
  modified_base 19: mod_base = um
  modified_base 20: mod_base = cm
  modified_base 21: mod_base = OTHER; note = 2'-O-methyladenosine
  source 1..21: mol_type = other RNA
cctcactttaatcctctatca

SEQ ID NO: 111 - RNA, 21 nt, synthetic construct
  modified_base 1: mod_base = cm
  modified_base 2: mod_base = cm
  modified_base 3: mod_base = um
  modified_base 4: mod_base = cm
  modified_base 5: mod_base = OTHER; note = 2'-O-methyladenosine
  modified_base 6: mod_base = cm
  modified_base 7: mod_base = OTHER; note = 2'C16 alkoxy uridine
  modified_base 8: mod_base = um
  modified_base 9: mod_base = OTHER; note = 2'-fluorouridine
  modified_base 10: mod_base = OTHER; note = 2'-fluoroadenosine
  modified_base 11: mod_base = OTHER; note = 2'-fluoroadenosine
  modified_base 12: mod_base = um
  modified_base 13: mod_base = cm
  modified_base 14: mod_base = cm
  modified_base 15: mod_base = um
  modified_base 16: mod_base = cm
  modified_base 17: mod_base = um
  modified_base 18: mod_base = OTHER; note = 2'-O-methyladenosine
  modified_base 19: mod_base = um
  modified_base 20: mod_base = cm
  modified_base 21: mod_base = OTHER; note = 2'-O-methyladenosine
  source 1..21: mol_type = other RNA
cctcactttaatcctctatca

SEQ ID NO: 112 - RNA, 21 nt, synthetic construct
  modified_base 1: mod_base = cm
  modified_base 2: mod_base = cm
  modified_base 3: mod_base = um
  modified_base 4: mod_base = cm
  modified_base 5: mod_base = OTHER; note = 2'-O-methyladenosine
  modified_base 6: mod_base = cm
  modified_base 7: mod_base = um
  modified_base 8: mod_base = um
  modified_base 9: mod_base = OTHER; note = 2'-fluorouridine
  modified_base 10: mod_base = OTHER; note = 2'-fluoroadenosine
  modified_base 11: mod_base = OTHER; note = 2'-fluoroadenosine
  modified_base 12: mod_base = um
  modified_base 13: mod_base = cm
  modified_base 14: mod_base = cm
  modified_base 15: mod_base = OTHER; note = 2'C16 alkoxy uridine
  modified_base 16: mod_base = cm
  modified_base 17: mod_base = um
  modified_base 18: mod_base = OTHER; note = 2'-O-methyladenosine
  modified_base 19: mod_base = um
  modified_base 20: mod_base = cm
  modified_base 21: mod_base = OTHER; note = 2'-O-methyladenosine
  source 1..21: mol_type = other RNA
cctcactttaatcctctatca

SEQ ID NO: 113 - RNA, 21 nt, synthetic construct
  modified_base 1: mod_base = cm
  modified_base 2: mod_base = cm
  modified_base 3: mod_base = um
  modified_base 4: mod_base = cm
  modified_base 5: mod_base = OTHER; note = 2'-O-methyladenosine
  modified_base 6: mod_base = cm
  modified_base 7: mod_base = um
  modified_base 8: mod_base = um
  modified_base 9: mod_base = OTHER; note = 2'-fluorouridine
  modified_base 10: mod_base = OTHER; note = 2'-fluoroadenosine
  modified_base 11: mod_base = OTHER; note = 2'-fluoroadenosine
  modified_base 12: mod_base = um
  modified_base 13: mod_base = cm
  modified_base 14: mod_base = cm
  modified_base 15: mod_base = um
  modified_base 16: mod_base = cm
  modified_base 17: mod_base = um
  modified_base 18: mod_base = OTHER; note = 2'-O-methyladenosine
  modified_base 19: mod_base = um
  modified_base 20: mod_base = cm
  modified_base 21: mod_base = OTHER; note = 2'-O-methyladenosine
  source 1..21: mol_type = other RNA
cctcactttaatcctctatca

SEQ ID NO: 114 - RNA, 21 nt, synthetic construct
  modified_base 1: mod_base = cm
  modified_base 2: mod_base = cm
  modified_base 3: mod_base = um
  modified_base 4: mod_base = cm
  modified_base 5: mod_base = OTHER; note = 2'-O-methyladenosine
  modified_base 6: mod_base = cm
  modified_base 7: mod_base = um
  modified_base 8: mod_base = um
  modified_base 9: mod_base = OTHER; note = 2'-fluorouridine
  modified_base 10: mod_base = OTHER; note = 2'-fluoroadenosine
  modified_base 11: mod_base = OTHER; note = 2'-fluoroadenosine
  modified_base 12: mod_base = um
  modified_base 13: mod_base = cm
  modified_base 14: mod_base = cm
  modified_base 15: mod_base = um
  modified_base 16: mod_base = cm
  modified_base 17: mod_base = um
  modified_base 18: mod_base = OTHER; note = 2'-O-methyladenosine
  modified_base 19: mod_base = um
  modified_base 20: mod_base = cm
  modified_base 21: mod_base = OTHER; note = 2'-O-methyladenosine
  source 1..21: mol_type = other RNA
cctcactttaatcctctatca

SEQ ID NO: 115 - RNA, 21 nt, synthetic construct
  modified_base 1: mod_base = cm
  modified_base 2: mod_base = cm
  modified_base 3: mod_base = um
  modified_base 4: mod_base = cm
  modified_base 5: mod_base = OTHER; note = 2'-O-methyladenosine
  modified_base 6: mod_base = cm
  modified_base 7: mod_base = um
  modified_base 8: mod_base = um
  modified_base 9: mod_base = OTHER; note = 2'-fluorouridine
  modified_base 10: mod_base = OTHER; note = 2'-fluoroadenosine
  modified_base 11: mod_base = OTHER; note = 2'-fluoroadenosine
  modified_base 12: mod_base = um
  modified_base 13: mod_base = cm
  modified_base 14: mod_base = cm
  modified_base 15: mod_base = um
  modified_base 16: mod_base = cm
  modified_base 17: mod_base = um
  modified_base 18: mod_base = OTHER; note = 2'-O-methyladenosine
  modified_base 19: mod_base = um
  modified_base 20: mod_base = cm
  modified_base 21: mod_base = OTHER; note = 2'-O-methyladenosine
  source 1..21: mol_type = other RNA
cctcactttaatcctctatca

SEQ ID NO: 116 - RNA, 21 nt, synthetic construct
  modified_base 1: mod_base = cm
  modified_base 2: mod_base = cm
  modified_base 3: mod_base = um
  modified_base 4: mod_base = cm
  modified_base 5: mod_base = OTHER; note = 2'-O-methyladenosine
  modified_base 6: mod_base = cm
  modified_base 7: mod_base = um
  modified_base 8: mod_base = um
  modified_base 9: mod_base = OTHER; note = 2'-fluorouridine
  modified_base 10: mod_base = OTHER; note = 2'-fluoroadenosine
  modified_base 11: mod_base = OTHER; note = 2'-fluoroadenosine
  modified_base 12: mod_base = um
  modified_base 13: mod_base = cm
  modified_base 14: mod_base = cm
  modified_base 15: mod_base = um
  modified_base 16: mod_base = cm
  modified_base 17: mod_base = um
  modified_base 18: mod_base = OTHER; note = 2'-O-methyladenosine
  modified_base 19: mod_base = um
  modified_base 20: mod_base = cm
  modified_base 21: mod_base = OTHER; note = 2'-O-methyladenosine
  source 1..21: mol_type = other RNA
cctcactttaatcctctatca

SEQ ID NO: 117 - RNA, 21 nt, synthetic construct
  modified_base 1: mod_base = cm
  modified_base 2: mod_base = cm
  modified_base 3: mod_base = um
  modified_base 4: mod_base = cm
  modified_base 5: mod_base = OTHER; note = 2'-O-methyladenosine
  modified_base 6: mod_base = cm
  modified_base 7: mod_base = um
  modified_base 8: mod_base = um
  modified_base 9: mod_base = OTHER; note = 2'-fluorouridine
  modified_base 10: mod_base = OTHER; note = 2'-fluoroadenosine
  modified_base 11: mod_base = OTHER; note = 2'-fluoroadenosine
  modified_base 12: mod_base = um
  modified_base 13: mod_base = cm
  modified_base 14: mod_base = cm
  modified_base 15: mod_base = um
  modified_base 16: mod_base = cm
  modified_base 17: mod_base = um
  modified_base 18: mod_base = OTHER; note = 2'-O-methyladenosine
  modified_base 19: mod_base = um
  modified_base 20: mod_base = cm
  modified_base 21: mod_base = OTHER; note = 2'-O-methyladenosine
  source 1..21: mol_type = other RNA
cctcactttaatcctctatca

SEQ ID NO: 118 - RNA, 21 nt, synthetic construct
  modified_base 1: mod_base = cm
  modified_base 2: mod_base = cm
  modified_base 3: mod_base = um
  modified_base 4: mod_base = cm
  modified_base 5: mod_base = OTHER; note = 2'-O-methyladenosine
  modified_base 6: mod_base = cm
  modified_base 7: mod_base = um
  modified_base 8: mod_base = um
  modified_base 9: mod_base = OTHER; note = 2'-fluorouridine
  modified_base 10: mod_base = OTHER; note = 2'-fluoroadenosine
  modified_base 11: mod_base = OTHER; note = 2'-fluoroadenosine
  modified_base 12: mod_base = um
  modified_base 13: mod_base = cm
  modified_base 14: mod_base = cm
  modified_base 15: mod_base = um
  modified_base 16: mod_base = cm
  modified_base 17: mod_base = um
  modified_base 18: mod_base = OTHER; note = 2'-O-methyladenosine
  modified_base 19: mod_base = um
  modified_base 20: mod_base = cm
  modified_base 21: mod_base = OTHER; note = 2'-O-methyladenosine
  source 1..21: mol_type = other RNA
cctcactttaatcctctatca

SEQ ID NO: 119 - RNA, 21 nt, synthetic construct
  modified_base 1: mod_base = gm
  modified_base 2: mod_base = um
  modified_base 3: mod_base = um
  modified_base 4: mod_base = gm
  modified_base 5: mod_base = OTHER; note = 2'-O-methyladenosine
  modified_base 6: mod_base = OTHER; note = 2'-O-methyladenosine
  modified_base 7: mod_base = gm
  modified_base 8: mod_base = OTHER; note = 2'-O-methyladenosine
  modified_base 9: mod_base = OTHER; note = 2'-fluorouridine
  modified_base 10: mod_base = um
  modified_base 11: mod_base = OTHER; note = 2'-fluorocytidine
  modified_base 12: mod_base = um
  modified_base 13: mod_base = OTHER; note = 2'-fluoroguanosine
  modified_base 14: mod_base = um
  modified_base 15: mod_base = gm
  modified_base 16: mod_base = OTHER; note = 2'-O-methyladenosine
  modified_base 17: mod_base = um
  modified_base 18: mod_base = cm
  modified_base 19: mod_base = um
  modified_base 20: mod_base = cm
  modified_base 21: mod_base = OTHER; note = 2'-O-methyladenosine
  source 1..21: mol_type = other RNA
gttgaagattctgtgatctca

SEQ ID NO: 120 - RNA, 21 nt, synthetic construct
  modified_base 1: mod_base = cm
  modified_base 2: mod_base = OTHER; note = 2'-O-methyladenosine
  modified_base 3: mod_base = um
  modified_base 4: mod_base = um
  modified_base 5: mod_base = um
  modified_base 6: mod_base = um
  modified_base 7: mod_base = OTHER; note = 2'-O-methyladenosine
  modified_base 8: mod_base = OTHER; note = 2'-O-methyladenosine
  modified_base 9: mod_base = OTHER; note = 2'-fluorouridine
  modified_base 10: mod_base = OTHER; note = 2'-fluorocytidine
  modified_base 11: mod_base = OTHER; note = 2'-fluorocytidine
  modified_base 12: mod_base = um
  modified_base 13: mod_base = cm
  modified_base 14: mod_base = OTHER; note = 2'-O-methyladenosine
  modified_base 15: mod_base = cm
  modified_base 16: mod_base = um
  modified_base 17: mod_base = cm
  modified_base 18: mod_base = um
  modified_base 19: mod_base = OTHER; note = 2'-O-methyladenosine
  modified_base 20: mod_base = OTHER; note = 2'-O-methyladenosine
  modified_base 21: mod_base = OTHER; note = 2'-O-methyladenosine
  source 1..21: mol_type = other RNA
cattttaatcctcactctaaa

SEQ ID NO: 121 - RNA, 21 nt, synthetic construct
  modified_base 1: mod_base = cm
  modified_base 2: mod_base = OTHER; note = 2'-O-methyladenosine
  modified_base 3: mod_base = um
  modified_base 4: mod_base = um
  modified_base 5: mod_base = um
  modified_base 6: mod_base = um
  modified_base 7: mod_base = OTHER; note = 2'-O-methyladenosine
  modified_base 8: mod_base = OTHER; note = 2'-O-methyladenosine
  modified_base 9: mod_base = OTHER; note = 2'-fluorouridine
  modified_base 10: mod_base = OTHER; note = 2'-fluorocytidine
  modified_base 11: mod_base = OTHER; note = 2'-fluorocytidine
  modified_base 12: mod_base = um
  modified_base 13: mod_base = cm
  modified_base 14: mod_base = OTHER; note = 2'-O-methyladenosine
  modified_base 15: mod_base = cm
  modified_base 16: mod_base = um
  modified_base 17: mod_base = cm
  modified_base 18: mod_base = um
  modified_base 19: mod_base = OTHER; note = 2'-O-methyladenosine
  modified_base 20: mod_base = OTHER; note = 2'-O-methyladenosine
  modified_base 21: mod_base = OTHER; note = 2'-O-methyladenosine
  source 1..21: mol_type = other RNA
cattttaatcctcactctaaa

SEQ ID NO: 122 - RNA, 21 nt, synthetic construct
  modified_base 1: mod_base = cm
  modified_base 2: mod_base = OTHER; note = 2'-O-methyladenosine
  modified_base 3: mod_base = um
  modified_base 4: mod_base = um
  modified_base 5: mod_base = um
  modified_base 6: mod_base = um
  modified_base 7: mod_base = OTHER; note = 2'-O-methyladenosine
  modified_base 8: mod_base = OTHER; note = 2'-O-methyladenosine
  modified_base 9: mod_base = OTHER; note = 2'-fluorouridine
  modified_base 10: mod_base = OTHER; note = 2'-fluorocytidine
  modified_base 11: mod_base = OTHER; note = 2'-fluorocytidine
  modified_base 12: mod_base = um
  modified_base 13: mod_base = cm
  modified_base 14: mod_base = OTHER; note = 2'-O-methyladenosine
  modified_base 15: mod_base = cm
  modified_base 16: mod_base = um
  modified_base 17: mod_base = cm
  modified_base 18: mod_base = um
  modified_base 19: mod_base = OTHER; note = 2'-O-methyladenosine
  modified_base 20: mod_base = OTHER; note = 2'-O-methyladenosine
  modified_base 21: mod_base = OTHER; note = 2'-O-methyladenosine
  source 1..21: mol_type = other RNA
cattttaatcctcactctaaa

SEQ ID NO: 123 - RNA, 21 nt, synthetic construct
  modified_base 1: mod_base = cm
  modified_base 2: mod_base = OTHER; note = 2'-O-methyladenosine
  modified_base 3: mod_base = um
  modified_base 4: mod_base = um
  modified_base 5: mod_base = um
  modified_base 6: mod_base = um
  modified_base 7: mod_base = OTHER; note = 2'-O-methyladenosine
  modified_base 8: mod_base = OTHER; note = 2'-O-methyladenosine
  modified_base 9: mod_base = OTHER; note = 2'-fluorouridine
  modified_base 10: mod_base = OTHER; note = 2'-fluorocytidine
  modified_base 11: mod_base = OTHER; note = 2'-fluorocytidine
  modified_base 12: mod_base = um
  modified_base 13: mod_base = cm
  modified_base 14: mod_base = OTHER; note = 2'-O-methyladenosine
  modified_base 15: mod_base = cm
  modified_base 16: mod_base = um
  modified_base 17: mod_base = cm
  modified_base 18: mod_base = um
  modified_base 19: mod_base = OTHER; note = 2'-O-methyladenosine
  modified_base 20: mod_base = OTHER; note = 2'-O-methyladenosine
  modified_base 21: mod_base = OTHER; note = 2'-O-methyladenosine
  source 1..21: mol_type = other RNA
cattttaatcctcactctaaa

SEQ ID NO: 124 - RNA, 21 nt, synthetic construct
  modified_base 1: mod_base = cm
  modified_base 2: mod_base = OTHER; note = 2'-O-methyladenosine
  modified_base 3: mod_base = um
  modified_base 4: mod_base = um
  modified_base 5: mod_base = um
  modified_base 6: mod_base = um
  modified_base 7: mod_base = OTHER; note = 2'-O-methyladenosine
  modified_base 8: mod_base = OTHER; note = 2'-O-methyladenosine
  modified_base 9: mod_base = OTHER; note = 2'-fluorouridine
  modified_base 10: mod_base = OTHER; note = 2'-fluorocytidine
  modified_base 11: mod_base = OTHER; note = 2'-fluorocytidine
  modified_base 12: mod_base = um
  modified_base 13: mod_base = cm
  modified_base 14: mod_base = OTHER; note = 2'-O-methyladenosine
  modified_base 15: mod_base = cm
  modified_base 16: mod_base = um
  modified_base 17: mod_base = cm
  modified_base 18: mod_base = um
  modified_base 19: mod_base = OTHER; note = 2'-O-methyladenosine
  modified_base 20: mod_base = OTHER; note = 2'-O-methyladenosine
  modified_base 21: mod_base = OTHER; note = 2'-O-methyladenosine
  source 1..21: mol_type = other RNA
cattttaatcctcactctaaa

SEQ ID NO: 125 - RNA, 21 nt, synthetic construct
  modified_base 1: mod_base = cm
  modified_base 2: mod_base = OTHER; note = 2'-O-methyladenosine
  modified_base 3: mod_base = um
  modified_base 4: mod_base = um
  modified_base 5: mod_base = um
  modified_base 6: mod_base = um
  modified_base 7: mod_base = OTHER; note = 2'-O-methyladenosine
  modified_base 8: mod_base = OTHER; note = 2'-O-methyladenosine
  modified_base 9: mod_base = OTHER; note = 2'-fluorouridine
  modified_base 10: mod_base = OTHER; note = 2'-fluorocytidine
  modified_base 11: mod_base = OTHER; note = 2'-fluorocytidine
  modified_base 12: mod_base = um
  modified_base 13: mod_base = cm
  modified_base 14: mod_base = OTHER; note = 2'-O-methyladenosine
  modified_base 15: mod_base = cm
  modified_base 16: mod_base = um
  modified_base 17: mod_base = cm
  modified_base 18: mod_base = um
  modified_base 19: mod_base = OTHER; note = 2'-O-methyladenosine
  modified_base 20: mod_base = OTHER; note = 2'-O-methyladenosine
  modified_base 21: mod_base = OTHER; note = 2'-O-methyladenosine
  source 1..21: mol_type = other RNA
cattttaatcctcactctaaa

SEQ ID NO: 126 - RNA, 21 nt, synthetic construct
  modified_base 1: mod_base = cm
  modified_base 2: mod_base = OTHER; note = 2'-O-methyladenosine
  modified_base 3: mod_base = um
  modified_base 4: mod_base = um
  modified_base 5: mod_base = um
  modified_base 6: mod_base = um
  modified_base 7: mod_base = OTHER; note = 2'-O-methyladenosine
  modified_base 8: mod_base = OTHER; note = 2'-O-methyladenosine
  modified_base 9: mod_base = OTHER; note = 2'-fluorouridine
  modified_base 10: mod_base = OTHER; note = 2'-fluorocytidine
  modified_base 11: mod_base = OTHER; note = 2'-fluorocytidine
  modified_base 12: mod_base = um
  modified_base 13: mod_base = cm
  modified_base 14: mod_base = OTHER; note = 2'-O-methyladenosine
  modified_base 15: mod_base = cm
  modified_base 16: mod_base = um
  modified_base 17: mod_base = cm
  modified_base 18: mod_base = um
  modified_base 19: mod_base = OTHER; note = 2'-O-methyladenosine
  modified_base 20: mod_base = OTHER; note = 2'-O-methyladenosine
  modified_base 21: mod_base = OTHER; note = 2'-O-methyladenosine
  source 1..21: mol_type = other RNA
cattttaatcctcactctaaa

SEQ ID NO: 127 - RNA, 21 nt, synthetic construct
  modified_base 1: mod_base = cm
  modified_base 2: mod_base = OTHER; note = 2'-O-methyladenosine
  modified_base 3: mod_base = um
  modified_base 4: mod_base = um
  modified_base 5: mod_base = um
  modified_base 6: mod_base = um
  modified_base 7: mod_base = OTHER; note = 2'-O-methyladenosine
  modified_base 8: mod_base = OTHER; note = 2'-O-methyladenosine
  modified_base 9: mod_base = OTHER; note = 2'-fluorouridine
  modified_base 10: mod_base = OTHER; note = 2'-fluorocytidine
  modified_base 11: mod_base = OTHER; note = 2'-fluorocytidine
  modified_base 12: mod_base = um
  modified_base 13: mod_base = cm
  modified_base 14: mod_base = OTHER; note = 2'-O-methyladenosine
  modified_base 15: mod_base = cm
  modified_base 16: mod_base = um
  modified_base 17: mod_base = cm
  modified_base 18: mod_base = um
  modified_base 19: mod_base = OTHER; note = 2'-O-methyladenosine
  modified_base 20: mod_base = OTHER; note = 2'-O-methyladenosine
  modified_base 21: mod_base = OTHER; note = 2'-O-methyladenosine
  source 1..21: mol_type = other RNA
cattttaatcctcactctaaa

SEQ ID NO: 128 - RNA, 21 nt, synthetic construct
  modified_base 1: mod_base = cm
  modified_base 2: mod_base = OTHER; note = 2'-O-methyladenosine
  modified_base 3: mod_base = um
  modified_base 4: mod_base = um
  modified_base 5: mod_base = um
  modified_base 6: mod_base = um
  modified_base 7: mod_base = OTHER; note = 2'-O-methyladenosine
  modified_base 8: mod_base = OTHER; note = 2'-O-methyladenosine
  modified_base 9: mod_base = OTHER; note = 2'-fluorouridine
  modified_base 10: mod_base = OTHER; note = 2'-fluorocytidine
  modified_base 11: mod_base = OTHER; note = 2'-fluorocytidine
  modified_base 12: mod_base = um
  modified_base 13: mod_base = cm
  modified_base 14: mod_base = OTHER; note = 2'-O-methyladenosine
  modified_base 15: mod_base = cm
  modified_base 16: mod_base = um
  modified_base 17: mod_base = cm
  modified_base 18: mod_base = um
  modified_base 19: mod_base = OTHER; note = 2'-O-methyladenosine
  modified_base 20: mod_base = OTHER; note = 2'-O-methyladenosine
  modified_base 21: mod_base = OTHER; note = 2'-O-methyladenosine
  source 1..21: mol_type = other RNA
cattttaatcctcactctaaa

SEQ ID NO: 129 - RNA, 21 nt, synthetic construct
  modified_base 1: mod_base = cm
  modified_base 2: mod_base = OTHER; note = 2'-O-methyladenosine
  modified_base 3: mod_base = um
  modified_base 4: mod_base = um
  modified_base 5: mod_base = um
  modified_base 6: mod_base = um
  modified_base 7: mod_base = OTHER; note = 2'-O-methyladenosine
  modified_base 8: mod_base = OTHER; note = 2'-O-methyladenosine
  modified_base 9: mod_base = OTHER; note = 2'-fluorouridine
  modified_base 10: mod_base = OTHER; note = 2'-fluorocytidine
  modified_base 11: mod_base = OTHER; note = 2'-fluorocytidine
  modified_base 12: mod_base = um
  modified_base 13: mod_base = cm
  modified_base 14: mod_base = OTHER; note = 2'-O-methyladenosine
  modified_base 15: mod_base = cm
  modified_base 16: mod_base = um
  modified_base 17: mod_base = cm
  modified_base 18: mod_base = um
  modified_base 19: mod_base = OTHER; note = 2'-O-methyladenosine
  modified_base 20: mod_base = OTHER; note = 2'-O-methyladenosine
  modified_base 21: mod_base = OTHER; note = 2'-O-methyladenosine
  source 1..21: mol_type = other RNA
cattttaatcctcactctaaa

SEQ ID NO: 130 - RNA, 21 nt, synthetic construct
  modified_base 1: mod_base = cm
  modified_base 2: mod_base = OTHER; note = 2'-O-methyladenosine
  modified_base 3: mod_base = um
  modified_base 4: mod_base = um
  modified_base 5: mod_base = um
  modified_base 6: mod_base = um
  modified_base 7: mod_base = OTHER; note = 2'-O-methyladenosine
  modified_base 8: mod_base = OTHER; note = 2'-O-methyladenosine
  modified_base 9: mod_base = OTHER; note = 2'-fluorouridine
  modified_base 10: mod_base = OTHER; note = 2'-fluorocytidine
  modified_base 11: mod_base = OTHER; note = 2'-fluorocytidine
  modified_base 12: mod_base = um
  modified_base 13: mod_base = cm
  modified_base 14: mod_base = OTHER; note = 2'-O-methyladenosine
  modified_base 15: mod_base = cm
  modified_base 16: mod_base = um
  modified_base 17: mod_base = cm
  modified_base 18: mod_base = um
  modified_base 19: mod_base = OTHER; note = 2'-O-methyladenosine
  modified_base 20: mod_base = OTHER; note = 2'-O-methyladenosine
  modified_base 21: mod_base = OTHER; note = 2'-O-methyladenosine
  source 1..21: mol_type = other RNA
cattttaatcctcactctaaa

SEQ ID NO: 131 - RNA, 21 nt, synthetic construct
  modified_base 1: mod_base = cm
  modified_base 2: mod_base = OTHER; note = 2'-O-methyladenosine
  modified_base 3: mod_base = um
  modified_base 4: mod_base = um
  modified_base 5: mod_base = um
  modified_base 6: mod_base = um
  modified_base 7: mod_base = OTHER; note = 2'-O-methyladenosine
  modified_base 8: mod_base = OTHER; note = 2'-O-methyladenosine
  modified_base 9: mod_base = OTHER; note = 2'-fluorouridine
  modified_base 10: mod_base = OTHER; note = 2'-fluorocytidine
  modified_base 11: mod_base = OTHER; note = 2'-fluorocytidine
  modified_base 12: mod_base = um
  modified_base 13: mod_base = cm
  modified_base 14: mod_base = OTHER; note = 2'-O-methyladenosine
  modified_base 15: mod_base = cm
  modified_base 16: mod_base = um
  modified_base 17: mod_base = cm
  modified_base 18: mod_base = um
  modified_base 19: mod_base = OTHER; note = 2'-O-methyladenosine
  modified_base 20: mod_base = OTHER; note = 2'-O-methyladenosine
  modified_base 21: mod_base = OTHER; note = 2'-O-methyladenosine
  source 1..21: mol_type = other RNA
cattttaatcctcactctaaa

SEQ ID NO: 132 - RNA, 21 nt, synthetic construct
  modified_base 1: mod_base = cm
  modified_base 2: mod_base = OTHER; note = 2'-O-methyladenosine
  modified_base 3: mod_base = um
  modified_base 4: mod_base = um
  modified_base 5: mod_base = um
  modified_base 6: mod_base = um
  modified_base 7: mod_base = OTHER; note = 2'-O-methyladenosine
  modified_base 8: mod_base = OTHER; note = 2'-O-methyladenosine
  modified_base 9: mod_base = OTHER; note = 2'-fluorouridine
  modified_base 10: mod_base = OTHER; note = 2'-fluorocytidine
  modified_base 11: mod_base = OTHER; note = 2'-fluorocytidine
  modified_base 12: mod_base = um
  modified_base 13: mod_base = cm
  modified_base 14: mod_base = OTHER; note = 2'-O-methyladenosine
  modified_base 15: mod_base = cm
  modified_base 16: mod_base = um
  modified_base 17: mod_base = cm
  modified_base 18: mod_base = um
  modified_base 19: mod_base = OTHER; note = 2'-O-methyladenosine
  modified_base 20: mod_base = OTHER; note = 2'-O-methyladenosine
  modified_base 21: mod_base = OTHER; note = 2'-O-methyladenosine
  source 1..21: mol_type = other RNA
cattttaatcctcactctaaa

SEQ ID NO: 133 - RNA, 21 nt, synthetic construct
  modified_base 1: mod_base = cm
  modified_base 2: mod_base = OTHER; note = 2'-O-methyladenosine
  modified_base 3: mod_base = um
  modified_base 4: mod_base = um
  modified_base 5: mod_base = um
  modified_base 6: mod_base = um
  modified_base 7: mod_base = OTHER; note = 2'-O-methyladenosine
  modified_base 8: mod_base = OTHER; note = 2'-O-methyladenosine
  modified_base 9: mod_base = OTHER; note = 2'-fluorouridine
  modified_base 10: mod_base = OTHER; note = 2'-fluorocytidine
  modified_base 11: mod_base = OTHER; note = 2'-fluorocytidine
  modified_base 12: mod_base = um
  modified_base 13: mod_base = cm
  modified_base 14: mod_base = OTHER; note = 2'-O-methyladenosine
  modified_base 15: mod_base = cm
  modified_base 16: mod_base = um
  modified_base 17: mod_base = cm
  modified_base 18: mod_base = um
  modified_base 19: mod_base = OTHER; note = 2'-O-methyladenosine
  modified_base 20: mod_base = OTHER; note = 2'-O-methyladenosine
  modified_base 21: mod_base = OTHER; note = 2'-O-methyladenosine
  source 1..21: mol_type = other RNA
cattttaatcctcactctaaa

SEQ ID NO: 134 - RNA, 21 nt, synthetic construct
  modified_base 1: mod_base = cm
  modified_base 2: mod_base = OTHER; note = 2'-O-methyladenosine
  modified_base 3: mod_base = um
  modified_base 4: mod_base = um
  modified_base 5: mod_base = um
  modified_base 6: mod_base = um
  modified_base 7: mod_base = OTHER; note = 2'-O-methyladenosine
  modified_base 8: mod_base = OTHER; note = 2'-O-methyladenosine
  modified_base 9: mod_base = OTHER; note = 2'-fluorouridine
  modified_base 10: mod_base = OTHER; note = 2'-fluorocytidine
  modified_base 11: mod_base = OTHER; note = 2'-fluorocytidine
  modified_base 12: mod_base = um
  modified_base 13: mod_base = cm
  modified_base 14: mod_base = OTHER; note = 2'-O-methyladenosine
  modified_base 15: mod_base = cm
  modified_base 16: mod_base = um
  modified_base 17: mod_base = cm
  modified_base 18: mod_base = um
  modified_base 19: mod_base = OTHER; note = 2'-O-methyladenosine
  modified_base 20: mod_base = OTHER; note = 2'-O-methyladenosine
  modified_base 21: mod_base = OTHER; note = 2'-O-methyladenosine
  source 1..21: mol_type = other RNA
cattttaatcctcactctaaa

(19) United States
(12) Patent Application Publication
(10) Pub. No.: US 2025/0223597 A1
(43) Pub. Date: Jul. 10, 2025

(54) LIPID CONJUGATES FOR THE DELIVERY OF THERAPEUTIC AGENTS TO CNS TISSUE

(71) Applicant: Arrowhead Pharmaceuticals, Inc., Pasadena, CA (US)

(72) Inventors: Xiaokai Li, San Diego, CA (US); Tao Pei, Middleton, WI (US); Phillip Lazzara, Madison, WI (US); Susan Phan, Fitchburg, WI (US); Teng Ai, San Diego, CA (US); Feng Liu, Valley Center, CA (US); George Naclerio, III, Madison, WI (US); Andrei V. Blokhin, Fitchburg, WI (US); Susan Ramos-Hunter, Jamaica Plain, MA (US); Jan Maciejewski, Madison, WI (US)

(21) Appl. No.: 18/980,153

(22) Filed: Dec. 13, 2024

Related U.S. Application Data
(63) Continuation of application No. PCT/US23/68439, filed on Jun. 14, 2023.
(60) Provisional application No. 63/495,505, filed on Apr. 11, 2023.
(60) Provisional application No. 63/352,485, filed on Jun. 15, 2022.

(51) Int. Cl. C12N 15/113

(52) U.S. Cl. CPC C12N 15/1137; C12N 15/113; C12N 15/1138; C12N 2310/14; C12N 2310/317; C12N 2310/3515; C12N 2320/32

(57) ABSTRACT

Disclosed herein are compounds comprising lipid PK/PD modulators for delivery of oligonucleotide-based agents, e.g., double-stranded RNAi agents, to certain cell types, such for example, CNS cells, in vivo. The PK/PD modulators disclosed herein, when conjugated to an oligonucleotide-based therapeutic or diagnostic agent, such as an RNAi agent, can enhance the delivery of the composition to the specified cells being targeted to facilitate the inhibition of gene expression in those cells.

### CROSS REFERENCE TO RELATED APPLICATIONS

This application is a continuation application of International Application No. PCT/US2023/068439, filed on Jun. 14, 2023, which claims the benefit of priority of U.S. Provisional Patent Application Ser. No. 63/495,505, filed on Apr. 11, 2023, and U.S. Provisional Patent Application Ser. No. 63/352,485, filed on Jun. 15, 2022, the contents of each of which are incorporated herein by reference in their entirety.

### SEQUENCE LISTING

This application contains a Sequence Listing which has been submitted in XML format and is hereby incorporated by reference in its entirety. The XML copy is named 30707-WO_ST26_SeqListing.xml, was created on Jun. 13, 2023, and is 1519 kb in size.

### FIELD OF THE INVENTION

The present disclosure relates to lipid conjugates for the delivery of oligonucleotide-based agents, e.g., double-stranded RNAi agents, to certain central nervous system (CNS) cell types in vivo, for inhibition of genes that are expressed in those cells.

### BACKGROUND

Oligonucleotide-based agents, such as antisense agents and double-stranded RNA interference (RNAi) agents, have shown great promise and have the potential to revolutionize the field of medicine and the availability to patients of potent therapeutic treatment options. However, the effective delivery of oligonucleotide-based agents, and double-stranded therapeutic RNAi agents in particular, has long been a challenge in developing viable therapeutic pharmaceutical agents. This is particularly the case when trying to achieve specific and selective delivery of oligonucleotide-based agents to extra-hepatic (i.e., non-hepatocyte) cells.

While various attempts over the past several years have been made to direct oligonucleotide-based agents to certain extra-hepatic cell types, including central nervous system cells, adipocytes, cardiac myocytes, and the like, using, for example, cholesterol conjugates (which is non-specific and has the known disadvantage of distributing to various undesired tissues and organs) and lipid-nanoparticles (LNPs) (which have been frequently reported to have toxicity concerns), to date none have achieved suitable delivery. As a result, there remains a need for a delivery vehicle to direct oligonucleotide-based agents, and RNAi agents in particular, to non-hepatocyte cell types.

### SUMMARY OF THE INVENTION

Disclosed herein are compounds comprising a lipid conjugated (or connected) to an oligonucleotide-based agent for delivery to CNS tissue. Lipid PK/PD modulator precursors are also disclosed herein.

One aspect of the invention provides for double-stranded oligonucleotides wherein a lipid is conjugated to one of the terminal nucleotides of one of the strands. In some embodiments, the lipid is conguated to the 5′ terminal nucleotide of one of the strands. In some embodiments, the lipid is conguated to the 3′ terminal nucleotide of one of the strands. In some embodiments the lipid conjugated to a terminal nucleotide of one of the strands is saturated. In some embodiments, the lipid is unsaturated. In some embodiments, the lipid is a sterol. In some embodiments, the lipid is a saturated lipid having between 12 and 30 carbon atoms. In some embodiments, the lipid is a straight chain lipid having 16 carbon atoms. In some embodiments, the lipid contains a hydroxyl moiety. In some embodiments, the lipid is cholesteryl.

In another aspect, the invention provides compounds comprising an oligonucleotide wherein a hydroxy lipid is conjugated to an internal nucleotide. In some embodiments, a hydroxy lipid comprises an aliphatic chain comprising one or more hydroxyl (—OH) functional groups. In some embodiments, the hydroxy group is conjugated to the distal carbon of the aliphatic chain relative to the internal nucleotide (i.e. the carbon atom farthest from the internal nucleotide.) In some embodiments, the hydroxy lipid is conjugated to the 2′ carbon of the internal nucleotide. In some embodiments, the hydroxy lipid consists of 12-24 carbon atoms. In some embodiments, the hydroxy lipid consists of 16 carbon atoms.

Unless otherwise defined, all technical and scientific terms used herein have the same meaning as commonly understood by one of ordinary skill in the art. Although methods and materials similar or equivalent to those described herein can be used in the practice or testing of the present invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

Other objects, features, aspects, and advantages of the invention will be apparent from the following detailed description, accompanying figures, and from the claims.

### DETAILED DESCRIPTION

### Lipid PK/PD Modulators

Described herein are compounds comprising PK/PD modulators conjugated to oligonucleotide-based agent(s) to provide delivery of payloads, such as RNA interference (RNAi) agents, to cells in vivo. Without being bound to any particular theory, it is believed that the compounds described herein modulate the pharmacokinetic and or pharmacodynamic properties of corresponding delivery vehicles, thereby increasing the RNAi-induced knockdown of the target gene in a cell. The compounds described herein may facilitate delivery to certain cell types, including but not limited to CNS cell types, including but not limited to neurons, astrocytes, oligodentrocytes, microglia and endothelial cells.

The present invention provides a lipid delivery platform for oligonucleotides, methods of using the lipid delivery platform, and methods of making the lipid delivery platform.

As used herein and as would be understood by one skilled in the art, a polyethylene glycol (PEG) unit refers to repeating units of the formula —(CH₂CH₂O)—. It will be appreciated that, in the chemical structures disclosed herein, PEG units may be depicted as —(CH₂CH₂O)—, —(OCH₂CH₂)—, or —(CH₂OCH₂)—. It will also be appreciated that a numeral indicating the number of repeating PEG units may be placed on either side of the parentheses depicting the PEG units.

Another aspect of the invention provides a process for making compounds comprising an RNAi agent and a lipid moiety.

In some embodiments, the method comprises conjugating an oligonucleotide-based agent comprising a first reactive moiety with a compound comprising a lipid and a second reactive moiety to form a compound comprising an RNAi agent and a lipid moiety.

In some embodiments, the first reactive moiety is selected from the group consisting of a hydroxy and an amine reactive group. In some embodiments, the first reactive moiety is an amine. In some embodiments, the first reactive moiety is a hydroxy group.

In some embodiments, the second reactive moiety is selected from the group consisting of ester (including but not limited to activated esters such as tetrafluorophenoxy esters and para-nitrophenoxy esters), sulfone (including but not limited to sufonyl halides) and phosphoramidite. In some embodiments, the second reactive moiety is an ester. In some embodiments, the second reactive moiety is a sulfone. In some embodiments, the second reactive moiety is a phosphoramidite.

Compounds of formula LP-128p, LP-132p, LP-183p, LP-183 phosphoramidite, LP-183r-p, LP-200p, LP-232p, LP-233p, LP-242p, LP-243p, LP-245p, LP-249p, LP-257p, LP-259p, LP-260p, LP-262p, LP-269p, LP-273p, LP-274p, LP-276p, LP-283p, LP-286p, LP-287p, LP-289p, LP-290p, LP-293p, LP-296p, LP-300p, LP-303p, LP-304p, LP-310p, LP-383p, LP-395p, LP-396p, LP-409p, LP-429p, LP-430p, LP-431p, LP-435p, LP-439p, LP-440p, LP-441p, LP-456p, LP-462p, LP-463p, LP-464p, LP-465p, LP-466p, LP-493p (2′ internal), (2C8C12) phosphoramidite, (2C6C10) phosphoramidite, HO-C16 phosphoramidite, C16 phosphoramidite, and C22 phosphoramidite shown in Table 1, below and described herein may be referred to as “pharmacokinetic and/or pharmacodynamic modulator precursors” (hereinafter, “PK/PD modulator precursors”). It will also be appreciated that portions of said compounds may be referred to as “pharmacokinetic and/or pharmacodynamic modulators” (hereinafter, “PK/PD modulators”). When used to refer to a portion of a compound of formula LP-128b, LP-132b, LP-183b, LP-183r-b LP-200b, LP-232b, LP-233b, LP-242b, LP-243b, LP-245b, LP-249b, LP-257b, LP-259b, LP-260b, LP-262b, LP-269b, LP-273b, LP-274b, LP-276b, LP-283b, LP-286b, LP-287b, LP-289b, LP-290b, LP-293b, LP-296b, LP-300b, LP-303b, LP-304b, LP-310b, LP-383b, LP-395b, LP-396b, LP-409b, LP-429b, LP-430b, LP-431b, LP-435b, LP-439b, LP-440b, LP-441b, LP-456b, LP-462b, LP-463b, LP-464b, LP-465b, LP-466b, LP-493b (2′ internal), (2C8C12)b, (2C6C10)b, HO-C16b, C16b, C22b, LP-128c, LP-132c, LP-183c, LP-183r-c, LP-200c, LP-232c, LP-233c, LP-242c, LP-243c, LP-245c, LP-249c, LP-257c, LP-259c, LP-260c, LP-262c, LP-269c, LP-273c, LP-274c, LP-276c, LP-283c, LP-286c, LP-287c, LP-289c, LP-290c, LP-293c, LP-296c, LP-300c, LP-303c, LP-304c, LP-310c, LP-383c, LP-395c, LP-396c, LP-409c, LP-429c, LP-430c, LP-431c, LP-435c, LP-440c, LP-441c, LP-456c, LP-462c, LP-463c, LP-464c, LP-465c, LP-466c, and LP-493c (2′ internal), shown in Tables 3 and 3a below the term “PK/PD modulator” refers to the portion of the compound excluding R (i.e., the oligonucleotide-based agent).

A PK/PD modulator is linked to an oligonucleotide-based agent, such as an RNAi agent, to facilitate delivery of the RNAi agent to the desired cells or tissues. PK/PD modulator precursors can be synthetized having reactive moieties, including but not limited to activated ester groups and phosphoramidites, that readily facilitate linkage to one or more linking groups on an RNAi agent. Chemical reaction syntheses to link such PK/PD modulator precursors to RNAi agents are generally known in the art. The terms “PK/PD modulator” and “lipid PK/PD modulator” may be used interchangeably herein.

PK/PD modulator precursors selected from the group consisting of LP-128p, LP-132p, LP-183p, LP-183 phosphoramidite, LP-183r-p, LP-200p, LP-232p, LP-233p, LP-242p, LP-243p, LP-245p, LP-249p, LP-257p, LP-259p, LP-260p, LP-262p, LP-269p, LP-273p, LP-274p, LP-276p, LP-283p, LP-286p, LP-287p, LP-289p, LP-290p, LP-293p, LP-296p, LP-300p, LP-303p, LP-304p, LP-310p, LP-383p, LP-395p, LP-396p, LP-409p, LP-429p, LP-430p, LP-431p, LP-435p, LP-439p, LP-440p, LP-441p, LP-456p, LP-462p, LP-463p, LP-464p, LP-465p, LP-466p, LP-493p (2′ internal), (2C8C12) phosphoramidite, (2C6C10) phosphoramidite, HO-C16 phosphoramidite, C16 phosphoramidite, and C22 phosphoramidite as shown in Table 1 can be used as starting materials to link to RNAi agents. The PK/PD modulator precursors may be covalently attached to an RNAi agent using any known method in the art. For example, in some embodiments, activated ester PK/PD modulator precursors may be reacted with an amine-containing moiety on the 5′ end of the sense strand.

TABLE 1
Lipid PK/PD modulator precursors

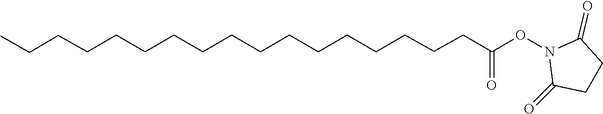 [SMILES: CCCCCCCCCCCCCCCCCC(=O)ON1C(=O)CCC1=O]

LP-128p
Purchased from Santa Cruz #sc-219228

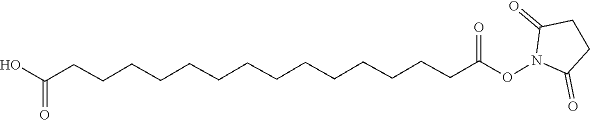 [SMILES: O=C(O)CCCCCCCCCCCCCCC(=O)ON1C(=O)CCC1=O]

LP-132p
Commercially available

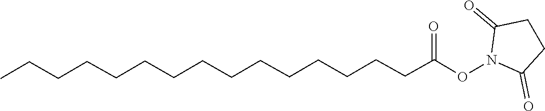 [SMILES: CCCCCCCCCCCCCCCC(=O)ON1C(=O)CCC1=O]

LP-183p
Purchased from Sigma

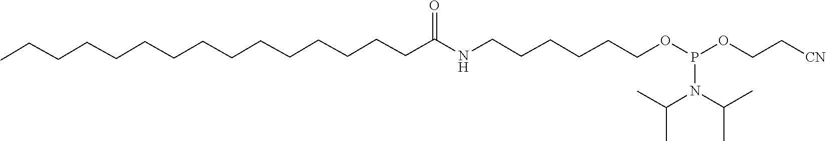 [SMILES: CCCCCCCCCCCCCCCC(=O)NCCCCCCOP(OCCC#N)N(C(C)C)C(C)C]

LP-183 phosphoramidite

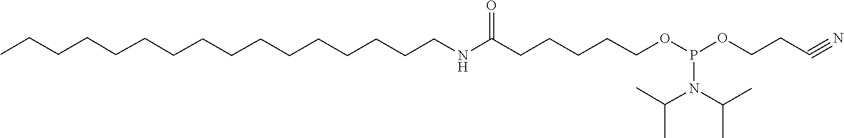 [SMILES: CCCCCCCCCCCCCCCCNC(=O)CCCCCOP(OCCC#N)N(C(C)C)C(C)C]

LP-183r-p

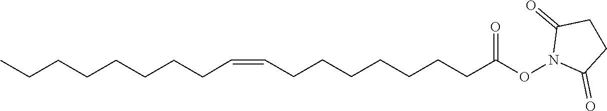 [SMILES: CCCCCCCC/C=C\CCCCCCCC(=O)ON1C(=O)CCC1=O]

LP-200p
Purchased from BroadPharm

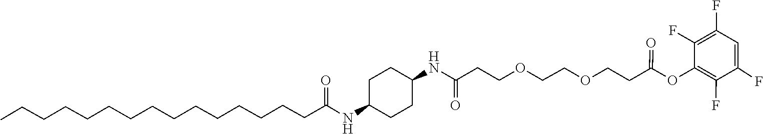 [SMILES: CCCCCCCCCCCCCCCC(=O)N[C@H]1CC[C@@H](NC(=O)CCOCCOCCC(=O)Oc2c(F)c(F)cc(F)c2F)CC1]

LP-232p

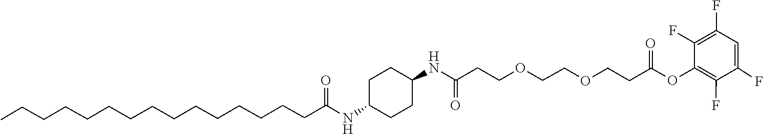 [SMILES: CCCCCCCCCCCCCCCC(=O)N[C@H]1CC[C@H](NC(=O)CCOCCOCCC(=O)Oc2c(F)c(F)cc(F)c2F)CC1]

LP-233p

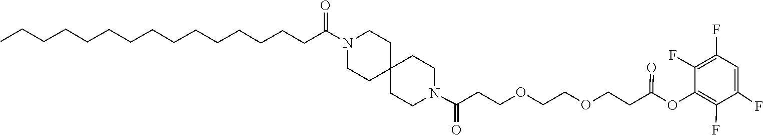 [SMILES: CCCCCCCCCCCCCCCC(=O)N1CCC2(CC1)CCN(C(=O)CCOCCOCCC(=O)Oc1c(F)c(F)cc(F)c1F)CC2]

LP-242p

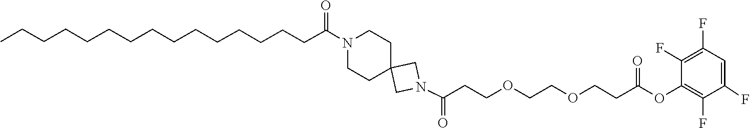 [SMILES: CCCCCCCCCCCCCCCC(=O)N1CCC2(CC1)CN(C(=O)CCOCCOCCC(=O)Oc1c(F)c(F)cc(F)c1F)C2]

LP-243p

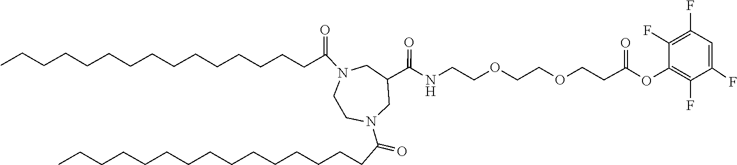 [SMILES: CCCCCCCCCCCCCCCC(=O)N1CCN(C(=O)CCCCCCCCCCCCCCC)CC(C(=O)NCCOCCOCCC(=O)Oc2c(F)c(F)cc(F)c2F)C1]

LP-245p

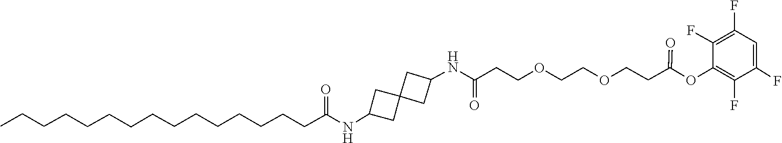 [SMILES: CCCCCCCCCCCCCCCC(=O)NC1CC2(C1)CC(NC(=O)CCOCCOCCC(=O)Oc1c(F)c(F)cc(F)c1F)C2]

LP-249p

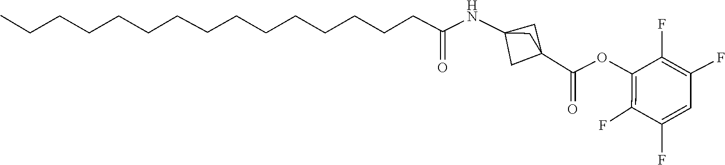 [SMILES: CCCCCCCCCCCCCCCC(=O)NC12CC(C(=O)Oc3c(F)c(F)cc(F)c3F)(C1)C2]

LP-257p

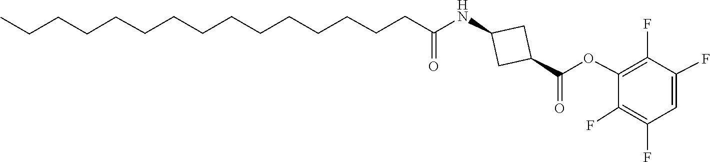 [SMILES: CCCCCCCCCCCCCCCC(=O)N[C@H]1C[C@@H](C(=O)Oc2c(F)c(F)cc(F)c2F)C1]

LP-259p

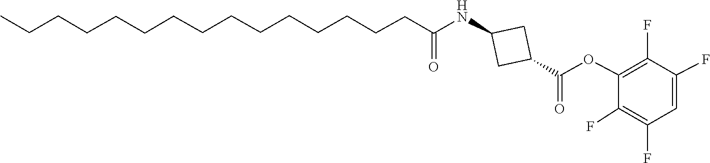 [SMILES: CCCCCCCCCCCCCCCC(=O)N[C@H]1C[C@H](C(=O)Oc2c(F)c(F)cc(F)c2F)C1]

LP-260p

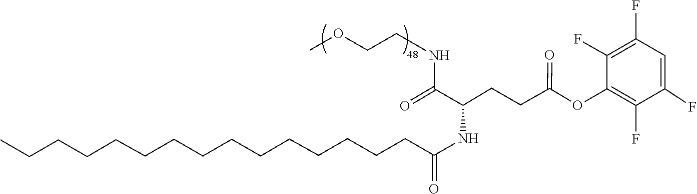

LP-262p

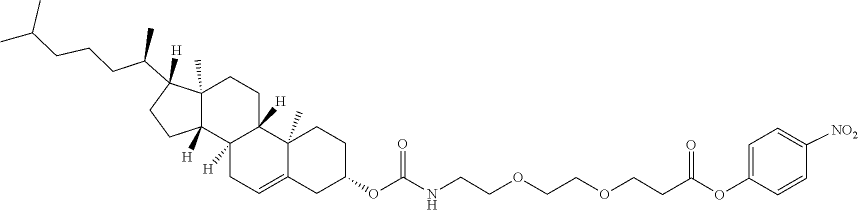 [SMILES: CC(C)CCC[C@@H](C)[C@H]1CC[C@H]2[C@@H]3CC=C4C[C@@H](OC(=O)NCCOCCOCCC(=O)Oc5ccc([N+](=O)[O-])cc5)CC[C@]4(C)[C@H]3CC[C@]12C]

LP-269p

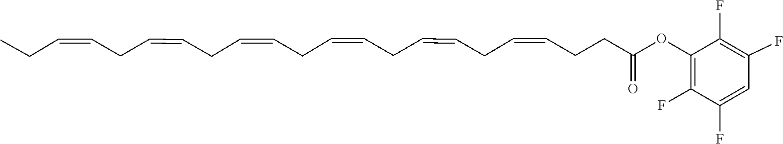 [SMILES: CC/C=C\C/C=C\C/C=C\C/C=C\C/C=C\C/C=C\CCC(=O)Oc1c(F)c(F)cc(F)c1F]

LP-273p

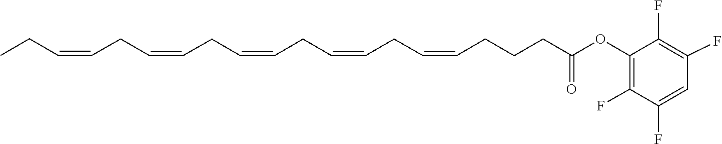 [SMILES: CC/C=C\C/C=C\C/C=C\C/C=C\C/C=C\CCCC(=O)Oc1c(F)c(F)cc(F)c1F]

LP-274p

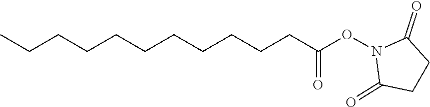 [SMILES: CCCCCCCCCCCC(=O)ON1C(=O)CCC1=O]

LP-276p
Purchased from eMolecule

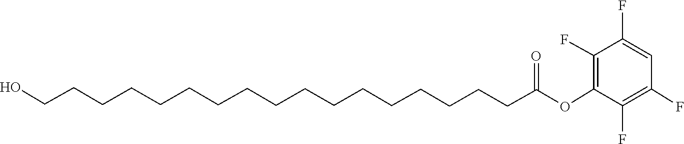 [SMILES: O=C(CCCCCCCCCCCCCCCCCO)Oc1c(F)c(F)cc(F)c1F]

LP-283p

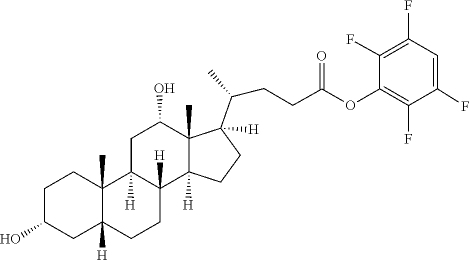

LP-286p

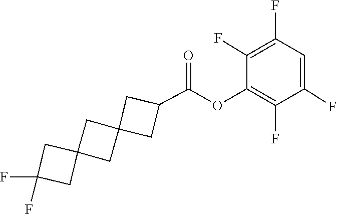 [SMILES: O=C(Oc1c(F)c(F)cc(F)c1F)C1CC2(C1)CC1(CC(F)(F)C1)C2]

LP-287p

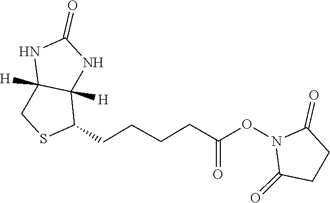 [SMILES: O=C1N[C@H]2[C@H](CS[C@H]2CCCCC(=O)ON2C(=O)CCC2=O)N1]

LP-289p
Purchased from Sigma

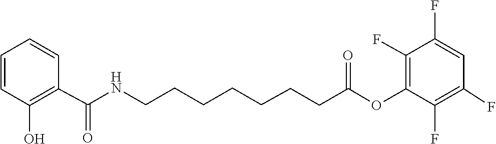 [SMILES: O=C(CCCCCCCNC(=O)c1ccccc1O)Oc1c(F)c(F)cc(F)c1F]

LP-290p

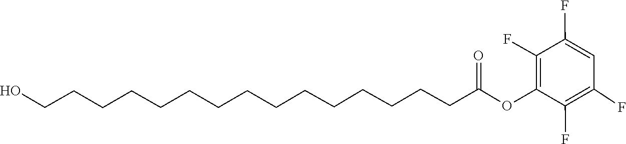 [SMILES: O=C(CCCCCCCCCCCCCCCO)Oc1c(F)c(F)cc(F)c1F]

LP-293p

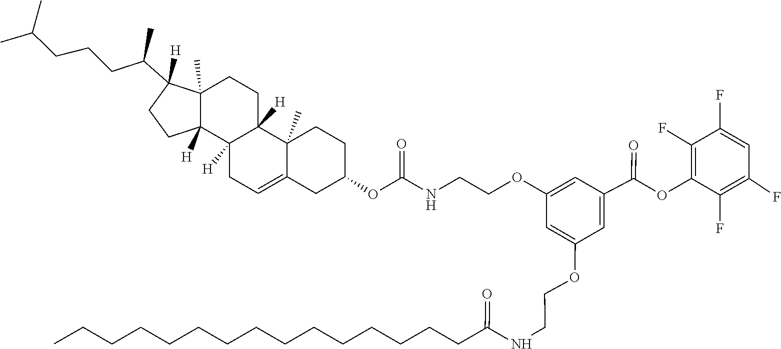 [SMILES: CCCCCCCCCCCCCCCC(=O)NCCOc1cc(OCCNC(=O)O[C@H]2CC[C@@]3(C)C(=CC[C@H]4[C@@H]5CC[C@H]([C@H](C)CCCC(C)C)[C@@]5(C)CC[C@@H]43)C2)cc(C(=O)Oc2c(F)c(F)cc(F)c2F)c1]

LP-296p

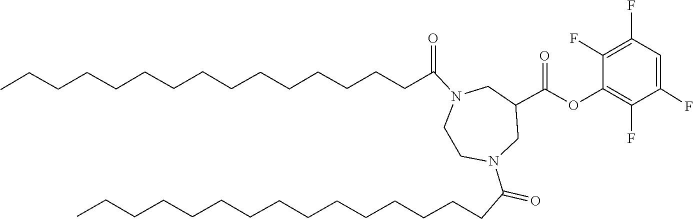 [SMILES: CCCCCCCCCCCCCCCC(=O)N1CCN(C(=O)CCCCCCCCCCCCCCC)CC(C(=O)Oc2c(F)c(F)cc(F)c2F)C1]

LP-300p

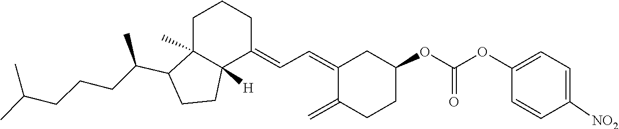 [SMILES: C=C1CC[C@H](OC(=O)Oc2ccc([N+](=O)[O-])cc2)C/C1=C/C=C1\CCC[C@]2(C)C([C@H](C)CCCC(C)C)CC[C@@H]12]

LP-303p

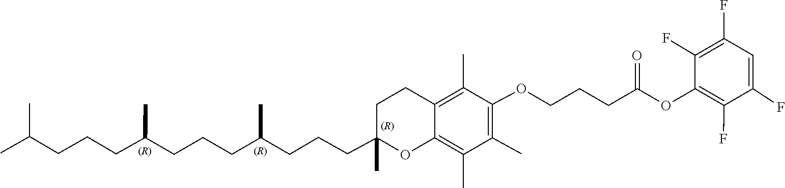

LP-304p

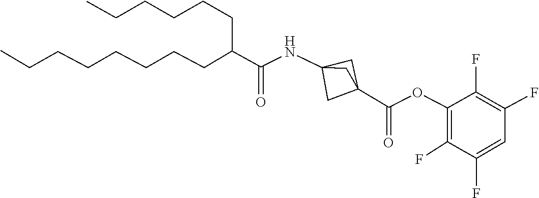 [SMILES: CCCCCCCCC(CCCCCC)C(=O)NC12CC(C(=O)Oc3c(F)c(F)cc(F)c3F)(C1)C2]

LP-310p

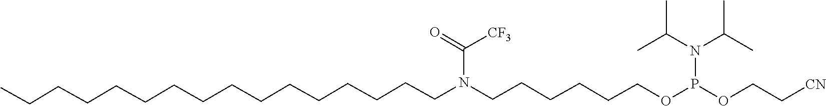 [SMILES: CCCCCCCCCCCCCCCCN(CCCCCCOP(OCCC#N)N(C(C)C)C(C)C)C(=O)C(F)(F)F]

LP-383p

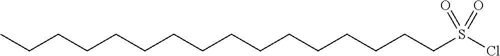 [SMILES: CCCCCCCCCCCCCCCCS(=O)(=O)Cl]

LP-395p
(commercially available)

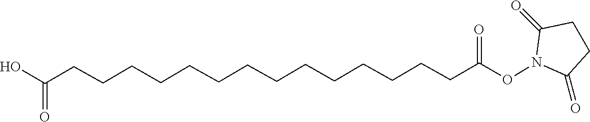 [SMILES: O=C(O)CCCCCCCCCCCCCCC(=O)ON1C(=O)CCC1=O]

LP-396p
(commercially available)

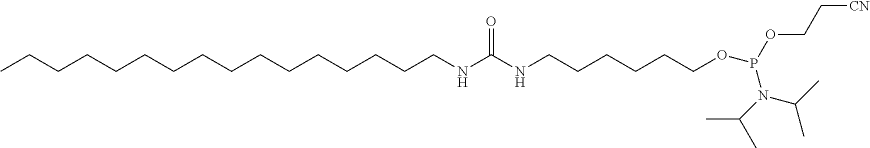 [SMILES: CCCCCCCCCCCCCCCCNC(=O)NCCCCCCOP(OCCC#N)N(C(C)C)C(C)C]

LP-409p

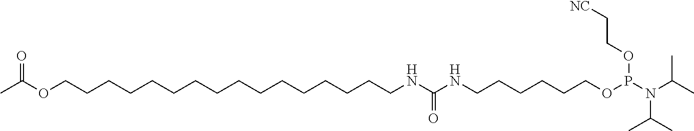 [SMILES: CC(=O)OCCCCCCCCCCCCCCCCNC(=O)NCCCCCCOP(OCCC#N)N(C(C)C)C(C)C]

LP-429p

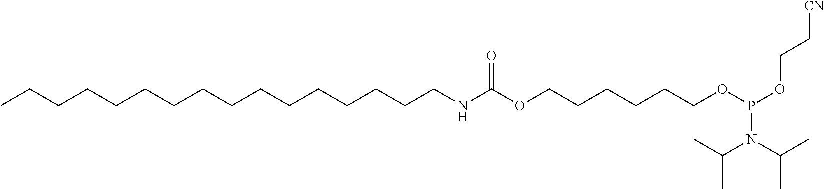 [SMILES: CCCCCCCCCCCCCCCCNC(=O)OCCCCCCOP(OCCC#N)N(C(C)C)C(C)C]

LP-430p

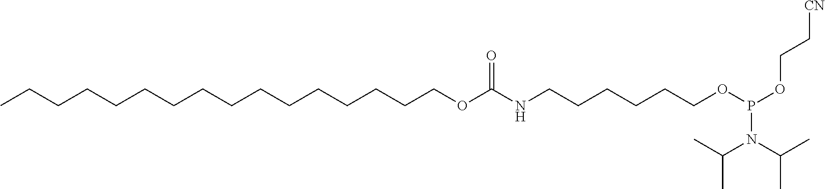 [SMILES: CCCCCCCCCCCCCCCCOC(=O)NCCCCCCOP(OCCC#N)N(C(C)C)C(C)C]

LP-431p

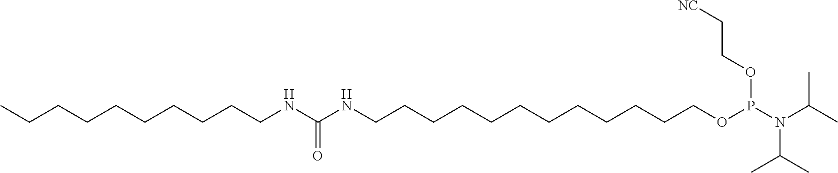 [SMILES: CCCCCCCCCCNC(=O)NCCCCCCCCCCCCOP(OCCC#N)N(C(C)C)C(C)C]

LP-435p

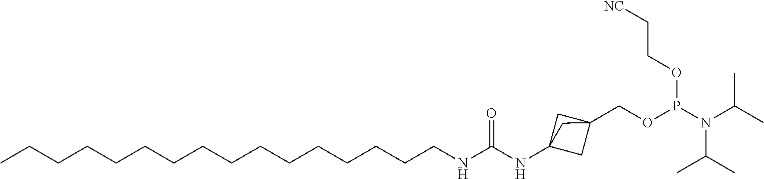 [SMILES: CCCCCCCCCCCCCCCCNC(=O)NC12CC(COP(OCCC#N)N(C(C)C)C(C)C)(C1)C2]

LP-439p

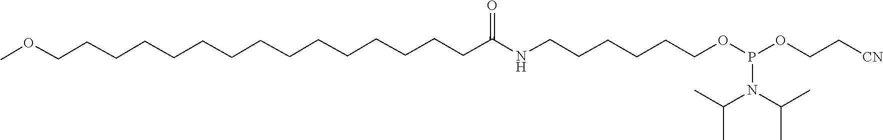 [SMILES: COCCCCCCCCCCCCCCCC(=O)NCCCCCCOP(OCCC#N)N(C(C)C)C(C)C]

LP-440p

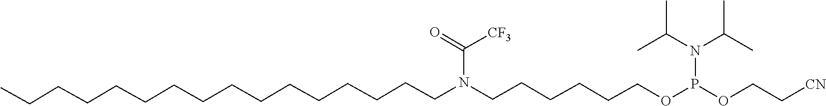 [SMILES: CCCCCCCCCCCCCCCCN(CCCCCCOP(OCCC#N)N(C(C)C)C(C)C)C(=O)C(F)(F)F]

LP-441p

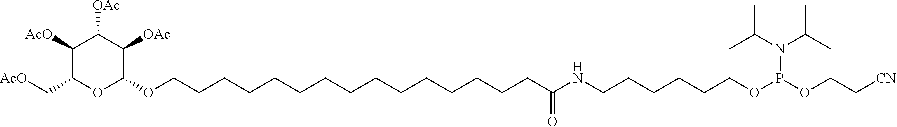 [SMILES: CC(=O)OC[C@H]1O[C@@H](OCCCCCCCCCCCCCCCC(=O)NCCCCCCOP(OCCC#N)N(C(C)C)C(C)C)[C@H](OC(C)=O)[C@@H](OC(C)=O)[C@@H]1OC(C)=O]

LP-456p

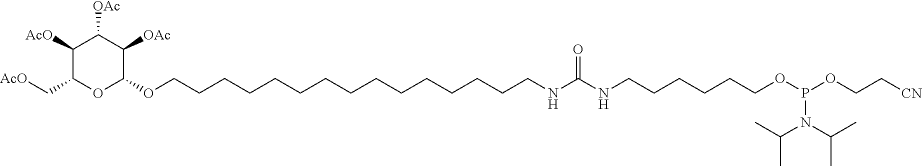 [SMILES: CC(=O)OC[C@H]1O[C@@H](OCCCCCCCCCCCCCCCNC(=O)NCCCCCCOP(OCCC#N)N(C(C)C)C(C)C)[C@H](OC(C)=O)[C@@H](OC(C)=O)[C@@H]1OC(C)=O]

LP-462p

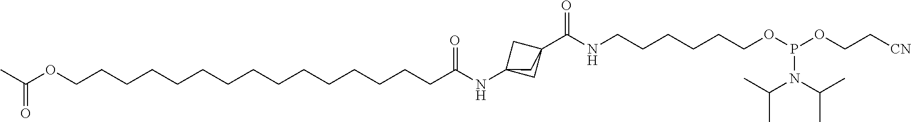 [SMILES: CC(=O)OCCCCCCCCCCCCCCCC(=O)NC12CC(C(=O)NCCCCCCOP(OCCC#N)N(C(C)C)C(C)C)(C1)C2]

LP-463p

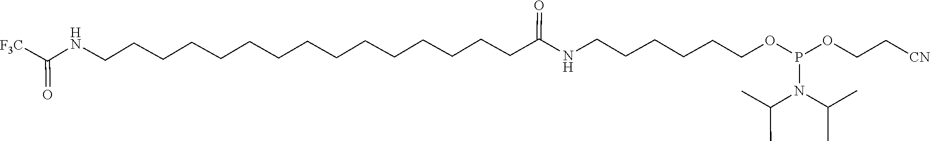 [SMILES: CC(C)N(C(C)C)P(OCCC#N)OCCCCCCNC(=O)CCCCCCCCCCCCCCCNC(=O)C(F)(F)F]

LP-464p

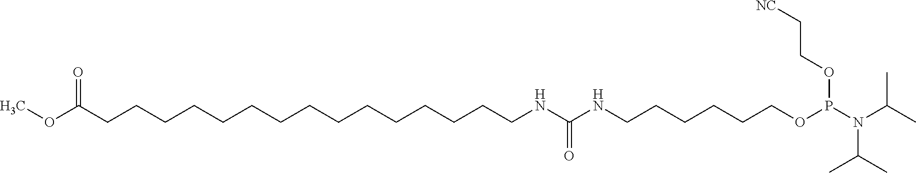 [SMILES: COC(=O)CCCCCCCCCCCCCCCNC(=O)NCCCCCCOP(OCCC#N)N(C(C)C)C(C)C]

LP-465p

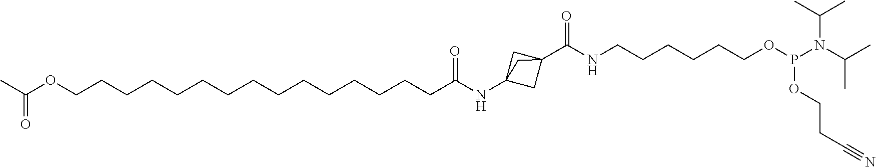 [SMILES: CC(=O)OCCCCCCCCCCCCCCCC(=O)NC12CC(C(=O)NCCCCCCOP(OCCC#N)N(C(C)C)C(C)C)(C1)C2]

LP-466p

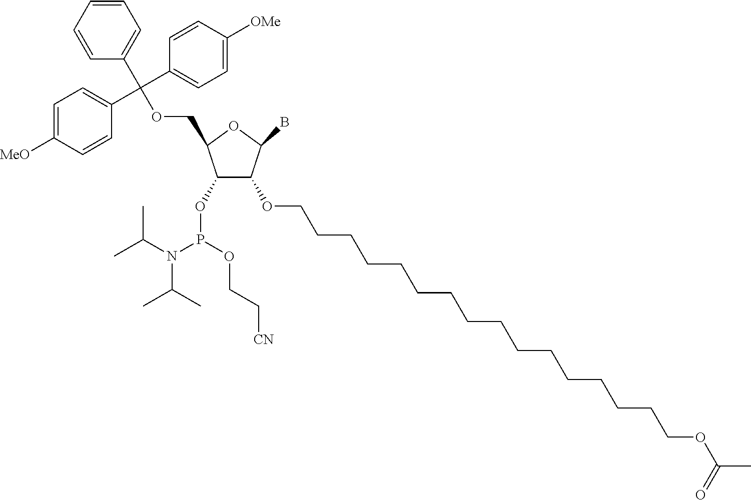

LP-493p (2' internal) (wherein B is a nucleobase)

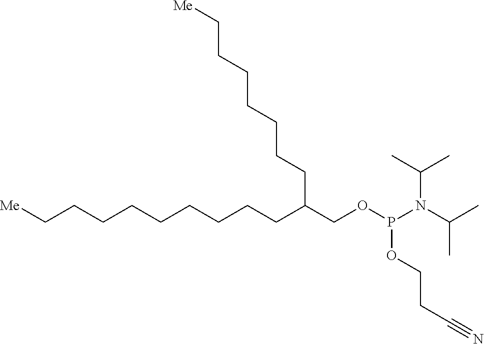 [SMILES: CCCCCCCCCCC(CCCCCCCC)COP(OCCC#N)N(C(C)C)C(C)C]

(2C8C12) phosphoramidite

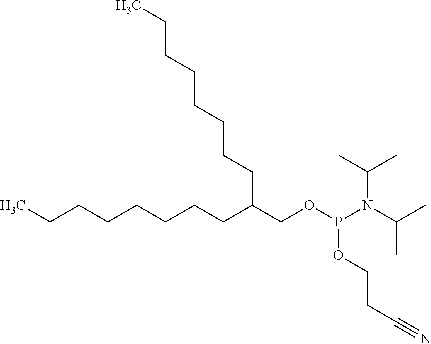 [SMILES: CCCCCCCCC(CCCCCCCC)COP(OCCC#N)N(C(C)C)C(C)C]

(2C6C10) phosphoramidite

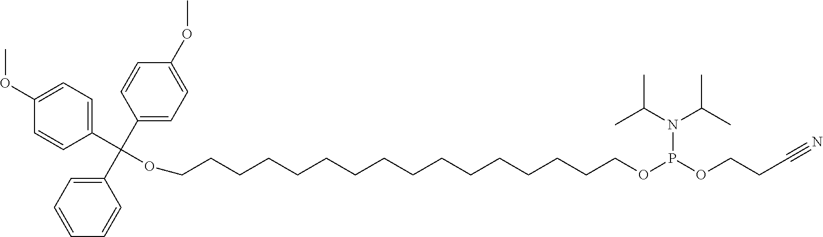 [SMILES: COc1ccc(C(OCCCCCCCCCCCCCCCCOP(OCCC#N)N(C(C)C)C(C)C)(c2ccccc2)c2ccc(OC)cc2)cc1]

HO-C16 phosphoramidite

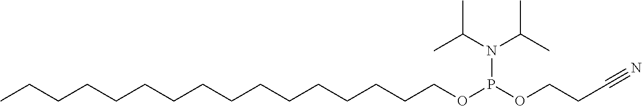 [SMILES: CCCCCCCCCCCCCCCCOP(OCCC#N)N(C(C)C)C(C)C]

C16 phosphoramidite

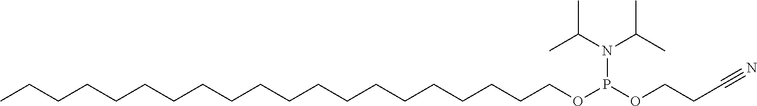 [SMILES: CCCCCCCCCCCCCCCCCCCCOP(OCCC#N)N(C(C)C)C(C)C]

C22 phosphoramidite

In some embodiments, one or more PK/PD modulators may be conjugated to RNAi agents described herein. In some embodiments, one, two, three, four, five, six, seven or more PK/PD modulators may be conjugated to RNAi agents described herein.

PK/PD modulator precursors may be conjugated to RNAi agents using any known method in the art. In some embodiments, PK/PD modulator precursors comprising an ester moiety may be reacted with RNAi agents comprising an amine to form a compound comprising a PK/PD modulator conjugated to an RNAi agent. In some embodiments, the amine may be on the 5′ or 3′ terminus of the RNAi agent. In some embodiments, the amine may be on the 5′ terminus of the RNAi agent. In some embodiments, the amine may be on the 3′ terminus of the RNAi agent. In some embodiments, PK/PD modulator precursors comprising a sulfonyl moiety may be reacted with RNAi agents comprising an amine to form a compound comprising a PK/PD modulator conjugated to an RNAi agent. In some embodiments, the amine may be on the 5′ or 3′ terminus of the RNAi agent. In some embodiments, the amine may be on the 5′ terminus of the RNAi agent. In some embodiments, the moiety may be on the 3′ terminus of the RNAi agent. In some embodiments, PK/PD modulator precursors comprising a phosphoramidite moiety may be reacted with RNAi agents comprising a hydroxyl moiety to form a compound comprising a PK/PD modulator conjugated to an RNAi agent. In some embodiments, the hydroxyl moiety may be on the 5′ or 3′ terminus of the RNAi agent. In some embodiments, the hydroxyl moiety may be on the 5′ terminus of the RNAi agent. In some embodiments, the hydroxyl moiety may be on the 3′ terminus of the RNAi agent.

In some embodiments, PK/PD modulators may be conjugated to the 5′ end of the sense or antisense strand, the 3′ end of the sense or antisense strand, or to an internal nucleotide of RNAi agents. In some embodiments, an RNAi agent is synthesized with a disulfide-containing moiety at the 3′ end of the sense strand, and a PK/PD modulator precursor may be conjugated to the 3′ end of the sense strand using any of the appropriate general synthetic schemes shown above.

In some embodiments, Lipid PK/PD modulators include compounds shown in Table 2.

TABLE 2
Lipid PK/PD modulators

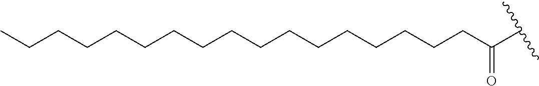 [SMILES: *C(=O)CCCCCCCCCCCCCCCCC]

LP-128a

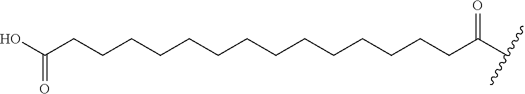 [SMILES: *C(=O)CCCCCCCCCCCCCCC(=O)O]

LP-132a

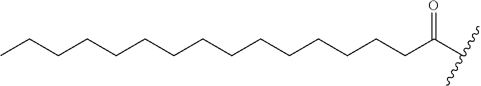 [SMILES: *C(=O)CCCCCCCCCCCCCCC]

LP-183a

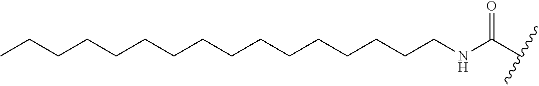 [SMILES: *C(=O)NCCCCCCCCCCCCCCCC]

LP-183r-a

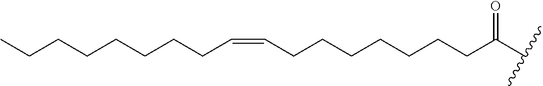 [SMILES: *C(=O)CCCCCCC/C=C\CCCCCCCC]

LP-200a

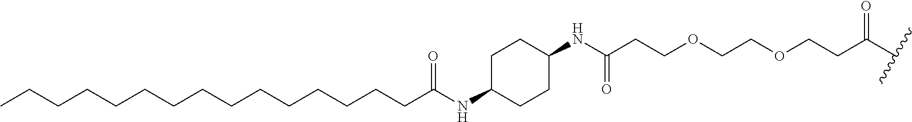 [SMILES: *C(=O)CCOCCOCCC(=O)N[C@H]1CC[C@@H](NC(=O)CCCCCCCCCCCCCCC)CC1]

LP-232a

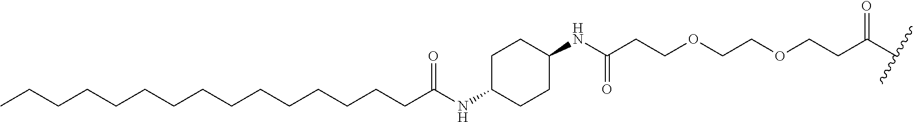 [SMILES: *C(=O)CCOCCOCCC(=O)N[C@H]1CC[C@H](NC(=O)CCCCCCCCCCCCCCC)CC1]

LP-233a

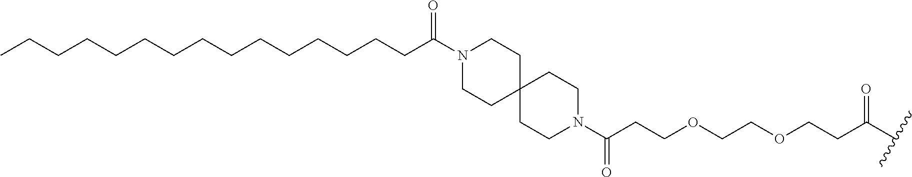 [SMILES: *C(=O)CCOCCOCCC(=O)N1CCC2(CCN(C(=O)CCCCCCCCCCCCCCC)CC2)CC1]

LP-242a

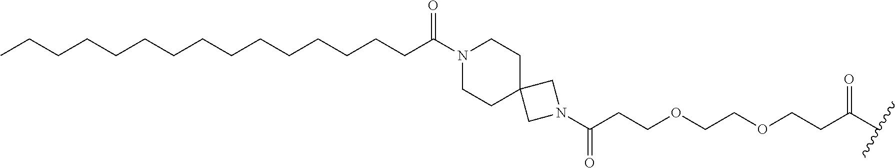 [SMILES: *C(=O)CCOCCOCCC(=O)N1CC2(CCN(C(=O)CCCCCCCCCCCCCCC)CC2)C1]

LP-243a

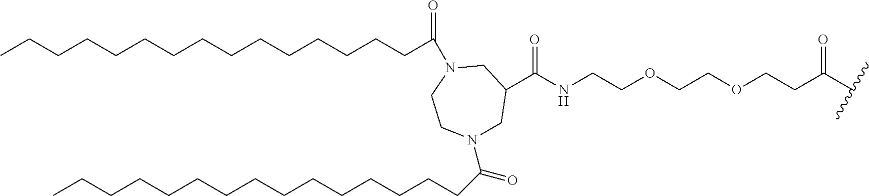 [SMILES: *C(=O)CCOCCOCCNC(=O)C1CN(C(=O)CCCCCCCCCCCCCCC)CCN(C(=O)CCCCCCCCCCCCCCC)C1]

LP-245a

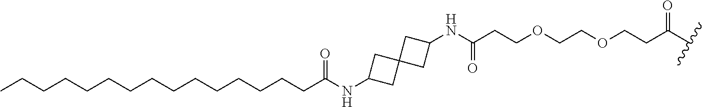 [SMILES: *C(=O)CCOCCOCCC(=O)NC1CC2(CC(NC(=O)CCCCCCCCCCCCCCC)C2)C1]

LP-249a

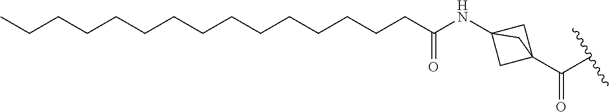 [SMILES: *C(=O)C12CC(NC(=O)CCCCCCCCCCCCCCC)(C1)C2]

LP-257a

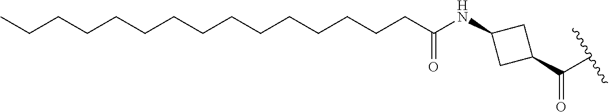 [SMILES: *C(=O)[C@H]1C[C@@H](NC(=O)CCCCCCCCCCCCCCC)C1]

LP-259a

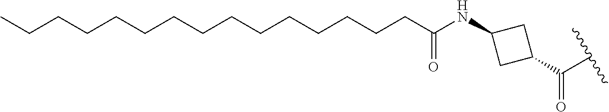 [SMILES: *C(=O)[C@H]1C[C@H](NC(=O)CCCCCCCCCCCCCCC)C1]

LP-260a

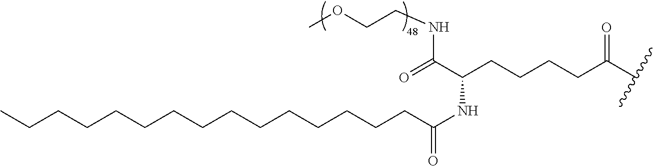

LP-262a

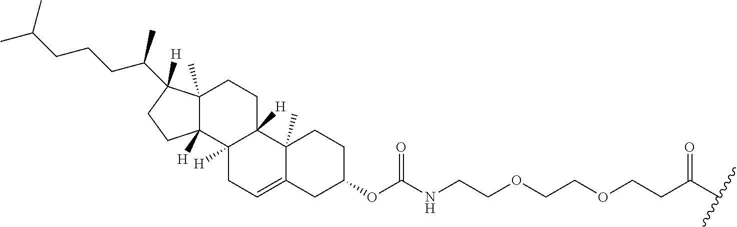 [SMILES: *C(=O)CCOCCOCCNC(=O)O[C@H]1CC[C@@]2(C)C(=CC[C@H]3[C@@H]4CC[C@H]([C@H](C)CCCC(C)C)[C@@]4(C)CC[C@@H]32)C1]

LP-269a

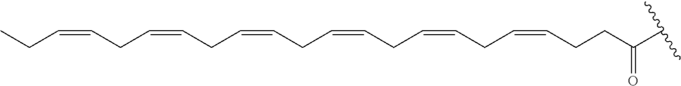 [SMILES: *C(=O)CC/C=C\C/C=C\C/C=C\C/C=C\C/C=C\C/C=C\CC]

LP-273a

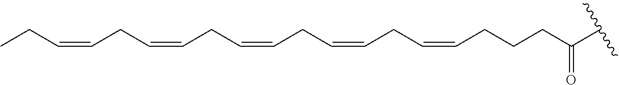 [SMILES: *C(=O)CCC/C=C\C/C=C\C/C=C\C/C=C\C/C=C\CC]

LP-274a

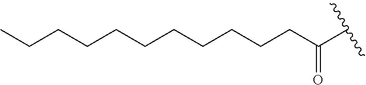 [SMILES: *C(=O)CCCCCCCCCCC]

LP-276a

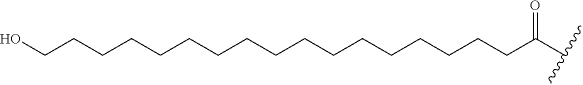 [SMILES: *C(=O)CCCCCCCCCCCCCCCCCO]

LP-283a

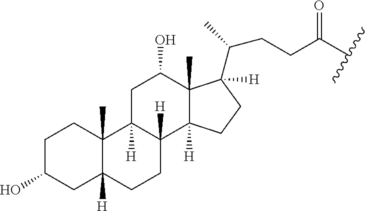

LP-286a

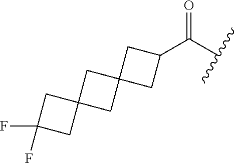 [SMILES: *C(=O)C1CC2(C1)CC1(CC(F)(F)C1)C2]

LP-287a

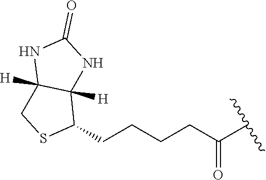 [SMILES: *C(=O)CCCC[C@@H]1SC[C@@H]2NC(=O)N[C@@H]21]

LP-289a

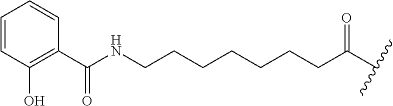 [SMILES: *C(=O)CCCCCCCNC(=O)c1ccccc1O]

LP-290a

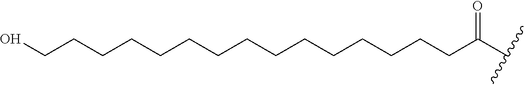 [SMILES: *C(=O)CCCCCCCCCCCCCCCO]

LP-293a

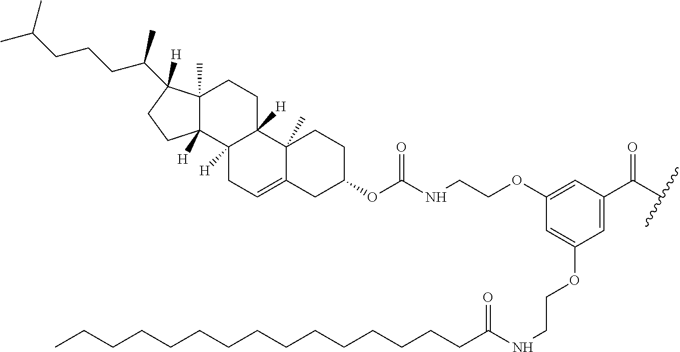 [SMILES: *C(=O)c1cc(OCCNC(=O)CCCCCCCCCCCCCCC)cc(OCCNC(=O)O[C@H]2CC[C@@]3(C)C(=CC[C@H]4[C@@H]5CC[C@H]([C@H](C)CCCC(C)C)[C@@]5(C)CC[C@@H]43)C2)c1]

LP-296a

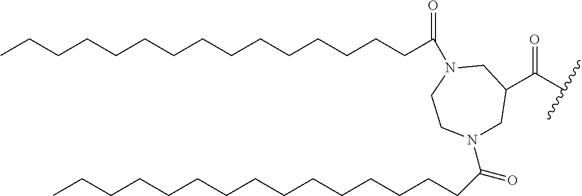 [SMILES: *C(=O)C1CN(C(=O)CCCCCCCCCCCCCCC)CCN(C(=O)CCCCCCCCCCCCCCC)C1]

LP-300a

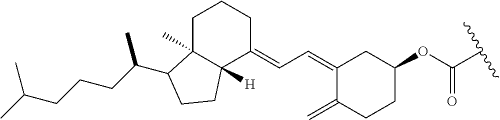 [SMILES: *C(=O)O[C@H]1CCC(=C)/C(=C\C=C2/CCC[C@]3(C)C([C@H](C)CCCC(C)C)CC[C@@H]23)C1]

LP-303a

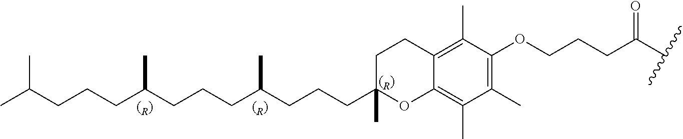

LP-304a

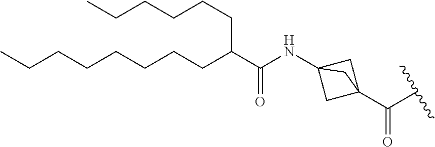 [SMILES: *C(=O)C12CC(NC(=O)C(CCCCCC)CCCCCCCC)(C1)C2]

LP-310a

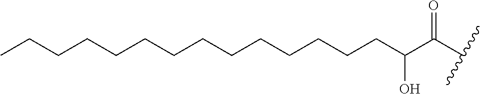 [SMILES: *C(=O)C(O)CCCCCCCCCCCCCC]

LP-383a

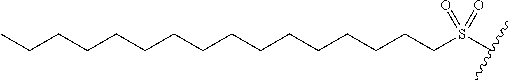 [SMILES: *S(=O)(=O)CCCCCCCCCCCCCCCC]

LP-395a

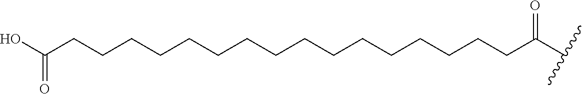 [SMILES: *C(=O)CCCCCCCCCCCCCCCCC(=O)O]

LP-396a

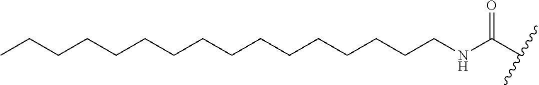 [SMILES: *C(=O)NCCCCCCCCCCCCCCCC]

LP-409a

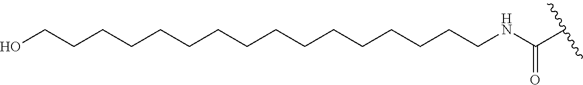 [SMILES: *C(=O)NCCCCCCCCCCCCCCCCO]

LP-429a

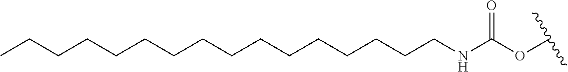 [SMILES: *OC(=O)NCCCCCCCCCCCCCCCC]

LP-430a

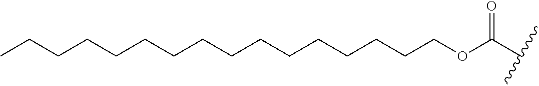 [SMILES: *C(=O)OCCCCCCCCCCCCCCCC]

LP-431a

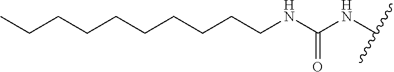 [SMILES: *NC(=O)NCCCCCCCCCC]

LP-435a

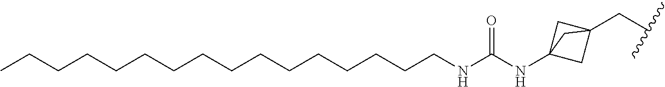 [SMILES: *CC12CC(NC(=O)NCCCCCCCCCCCCCCCC)(C1)C2]

LP-439a

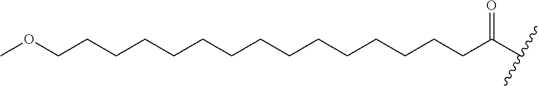 [SMILES: *C(=O)CCCCCCCCCCCCCCCOC]

LP-440a

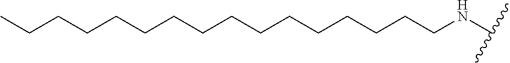 [SMILES: *NCCCCCCCCCCCCCCCC]

LP-441a

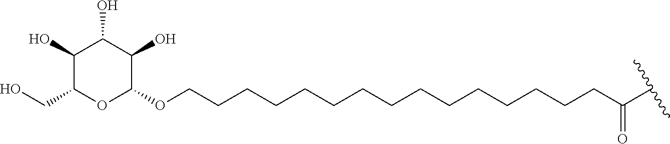 [SMILES: *C(=O)CCCCCCCCCCCCCCCO[C@@H]1O[C@H](CO)[C@@H](O)[C@H](O)[C@H]1O]

LP-456a

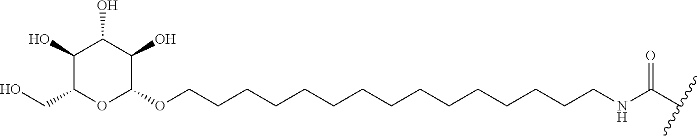 [SMILES: *C(=O)NCCCCCCCCCCCCCCCO[C@@H]1O[C@H](CO)[C@@H](O)[C@H](O)[C@H]1O]

LP-462a

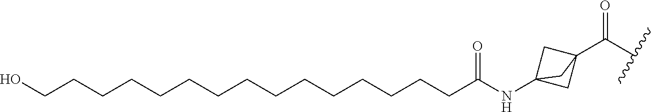 [SMILES: *C(=O)C12CC(NC(=O)CCCCCCCCCCCCCCCO)(C1)C2]

LP-463a

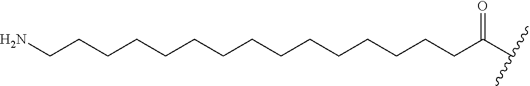 [SMILES: *C(=O)CCCCCCCCCCCCCCCN]

LP-464a

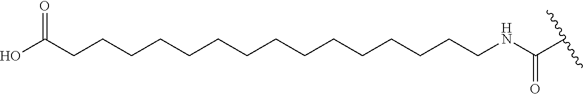 [SMILES: *C(=O)NCCCCCCCCCCCCCCCC(=O)O]

LP-465a

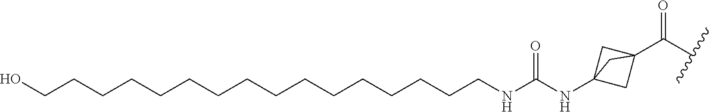 [SMILES: *C(=O)C12CC(NC(=O)NCCCCCCCCCCCCCCCCO)(C1)C2]

LP-466a

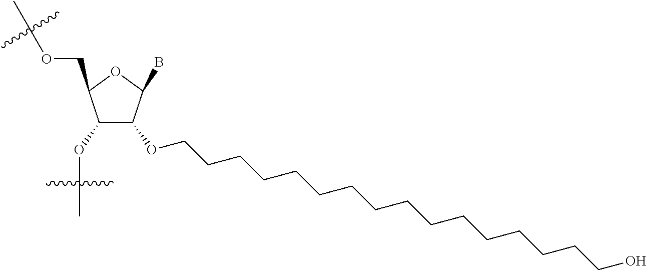

LP-493a (2' internal) (wherein B is a nucleobase)

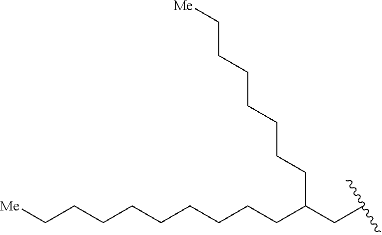 [SMILES: *CC(CCCCCCCC)CCCCCCCCCC]

(2C8C12)a

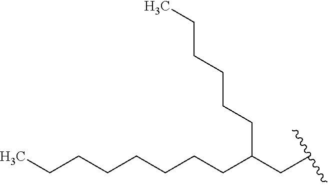 [SMILES: *CC(CCCCCC)CCCCCCCC]

(2C6C10)a

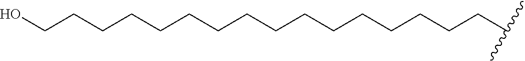 [SMILES: *CCCCCCCCCCCCCCCCO]

HO-C16a

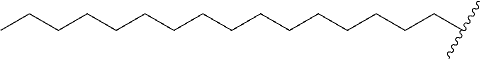 [SMILES: *CCCCCCCCCCCCCCCC]

C16a

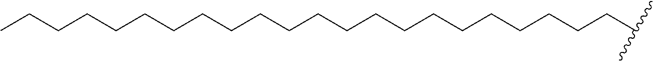 [SMILES: *CCCCCCCCCCCCCCCCCCCCCC]

C22a

wherein [symbol] indicates the point of connection to an oligonucleotide.

In some embodiments, lipid PK/PD modulators are represented by compounds having a formula shown in Table 3.

TABLE 3
Lipid PK/PD modulators

| | |
|---|---|
| 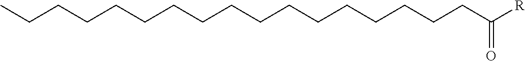 | LP-128b |
| 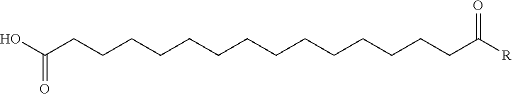 | LP-132b |
| 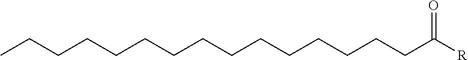 | LP-183b |
| 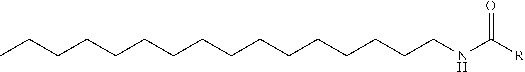 | LP-183r-b |
| 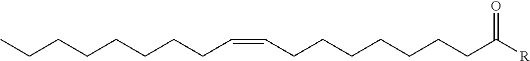 | LP-200b |
| 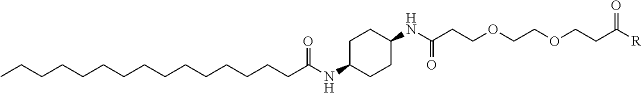 | LP-232b |
| 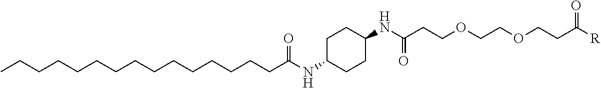 | LP-233b |
| 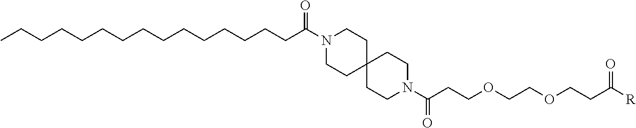 | LP-242b |
| 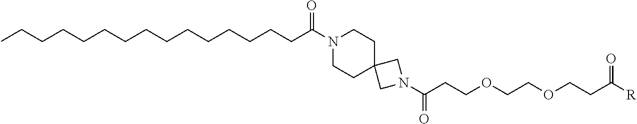 | LP-243b |
| 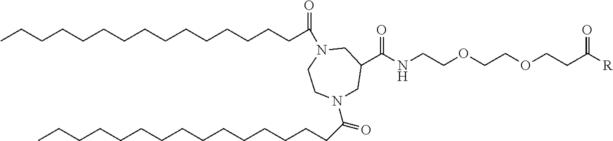 | LP-245b |
| 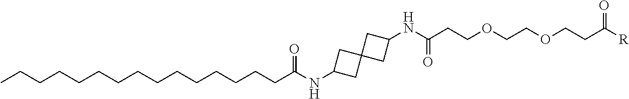 | LP-249b |
| 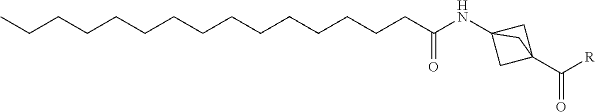 | LP-257b |
| 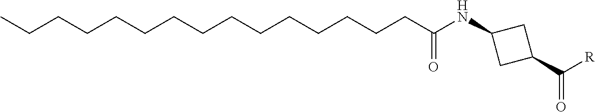 | LP-259b |
| 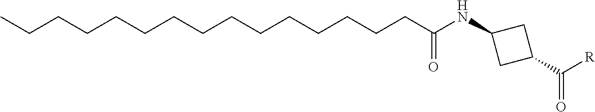 | LP-260b |
| 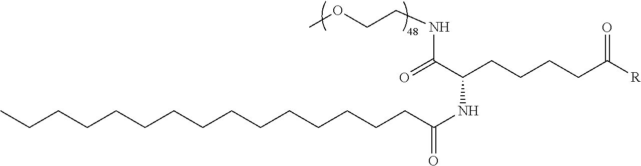 | LP-262b |
| 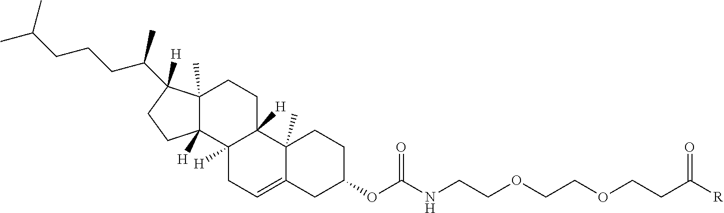 | LP-269b |
| 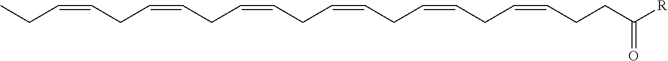 | LP-273b |
| 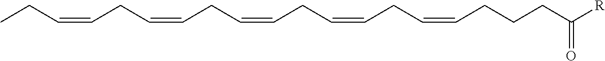 | LP-274b |
| 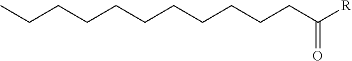 | LP-276b |
| 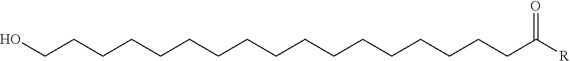 | LP-283b |
| 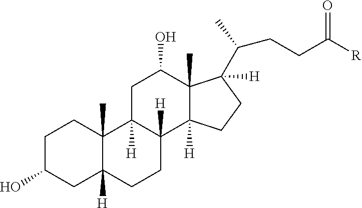 | LP-286b |
| 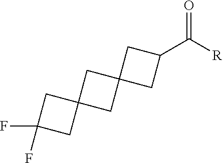 | LP-287b |
| 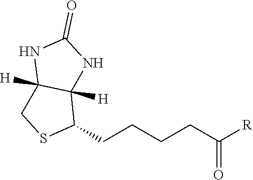 | LP-289b |
| 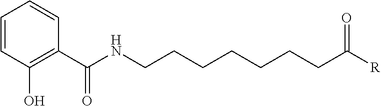 | LP-290b |
| 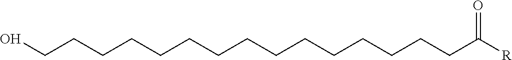 | LP-293b |
| 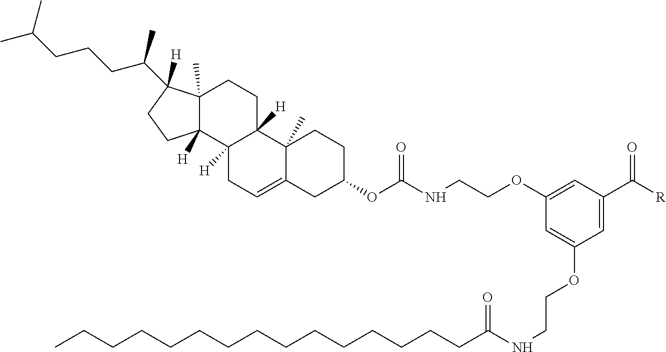 | LP-296b |
| 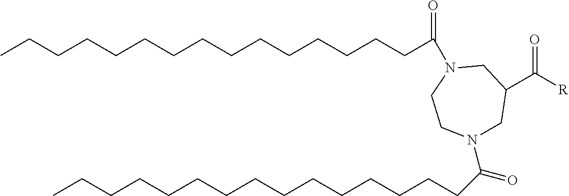 | LP-300b |
| 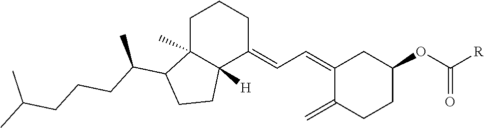 | LP-303b |
| 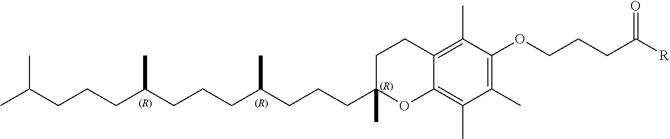 | LP-304b |
| 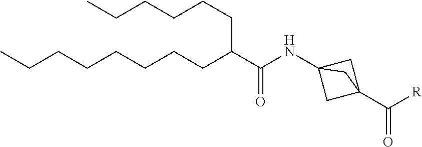 | LP-310b |
| 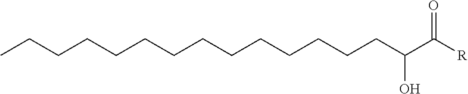 | LP-383b |
| 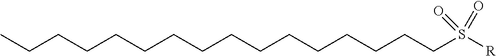 | LP-395b |
| 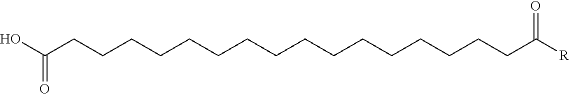 | LP-396b |
| 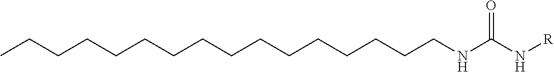 | LP-409b |
| 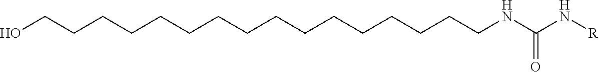 | LP-429b |
| 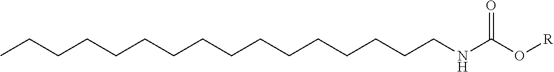 | LP-430b |
| 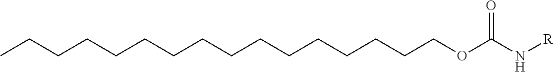 | LP-431b |
| 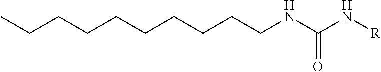 | LP-435b |
| 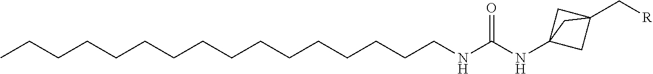 | LP-439b |
| 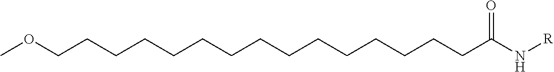 | LP-440b |
| 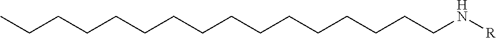 | LP-441b |
| 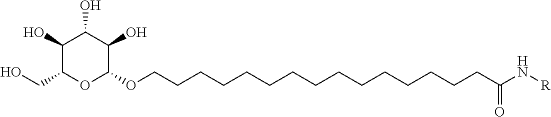 | LP-456b |
| 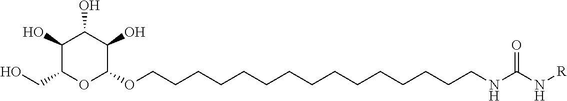 | LP-462b |
| 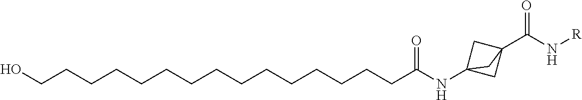 | LP-463b |
| 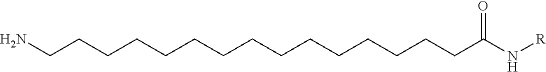 | LP-464b |
| 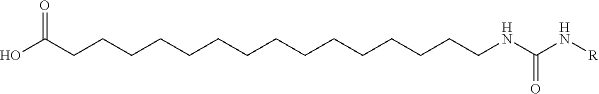 | LP-465b |
| 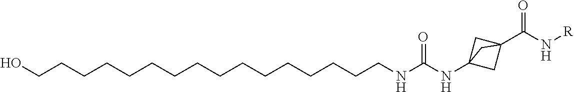 | LP-466b |
| 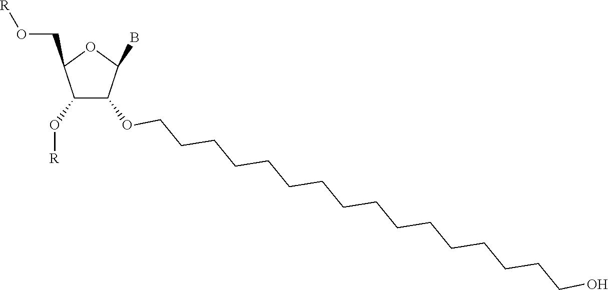 | LP-493b |
| (2′ internal) (wherein B is a nucleobase) | |
| 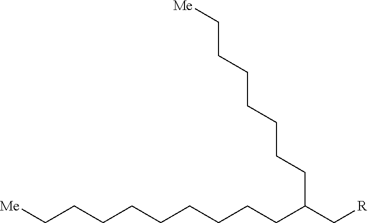 | (2C8C12)b |
| 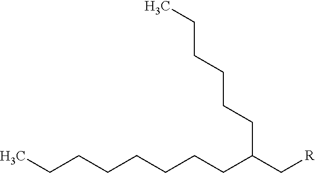 | (2C6C10)b |
| 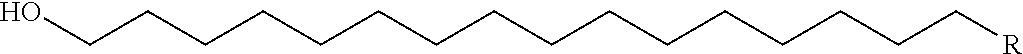 | HO-C16b |
| 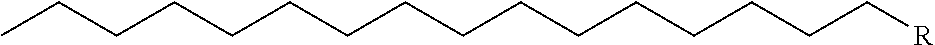 | C16b |
| 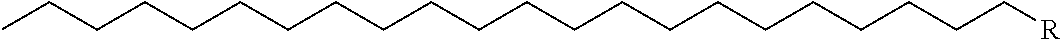 | C22b |

wherein R comprises an oligonucleotide.

In some embodiments, lipid PK/PD modulators comprise an aliphatic linker between the lipid component and an oligonucleotide. Example PK/PD modulators are represented by compounds having a formula shown in Table 3a.

TABLE 3a
TLipid PK/PD modulators comprising linkers

| | |
|---|---|
| 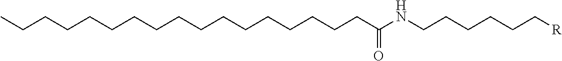 | LP-128c |
| 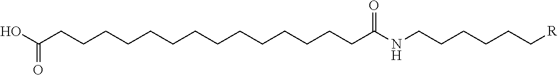 | LP-132c |
| 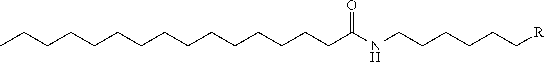 | LP-183c |
| 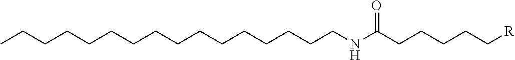 | LP-183r-c |
| 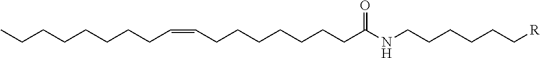 | LP-200c |
| 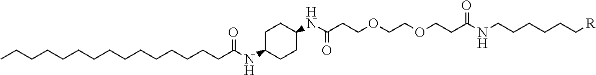 | LP-232c |
| 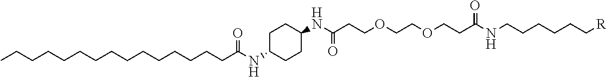 | LP-233c |
| 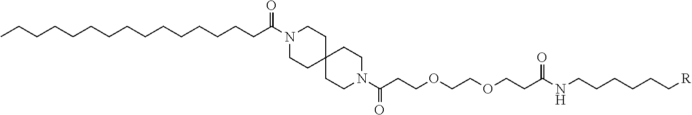 | LP-242c |
| 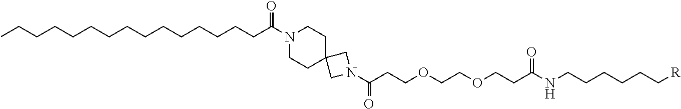 | LP-243c |
| 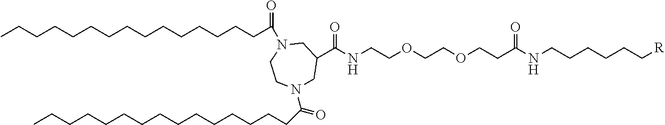 | LP-245c |
| 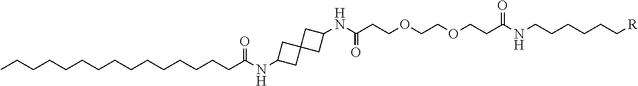 | LP-249c |
| 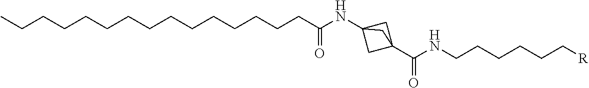 | LP-257c |
| 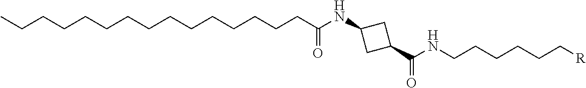 | LP-259c |
| 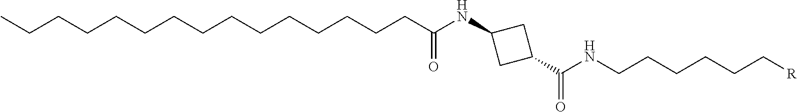 | LP-260c |
| 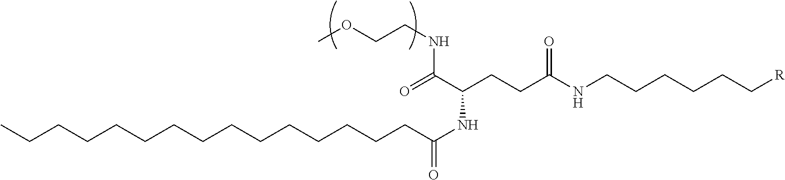 | LP-262c |
| 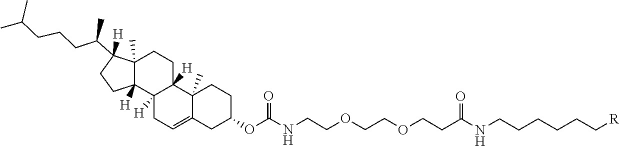 | LP-269c |
| 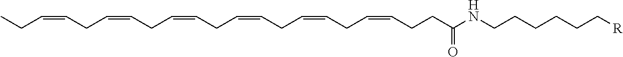 | LP-273c |
| 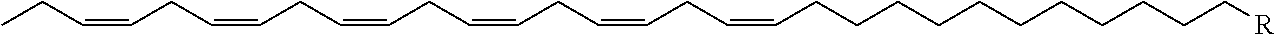 | LP-274c |
| 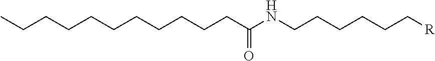 | LP-276c |
| 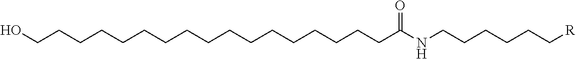 | LP-283c |
| 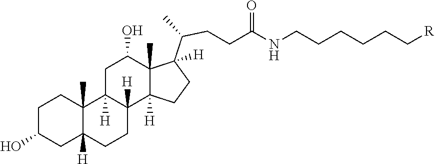 | LP-286c |
| 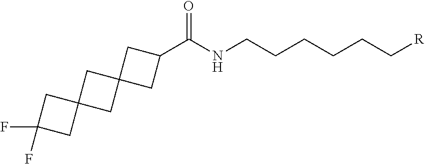 | LP-287c |
| 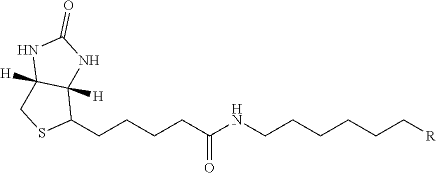 | LP-289c |
| 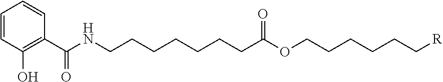 | LP-290c |
| 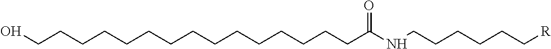 | LP-293c |
| 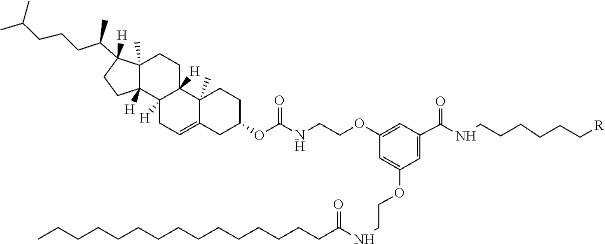 | LP-296c |
| 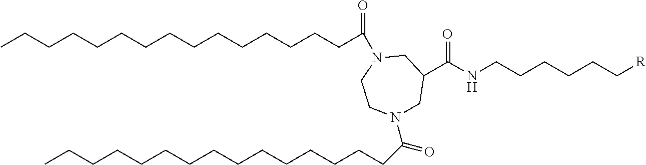 | LP-300c |
| 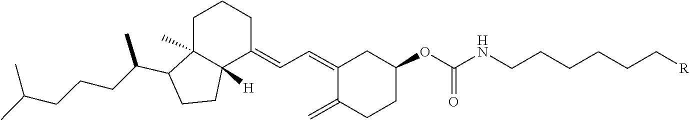 | LP-303c |
| 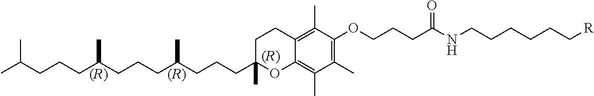 | LP-304c |
| 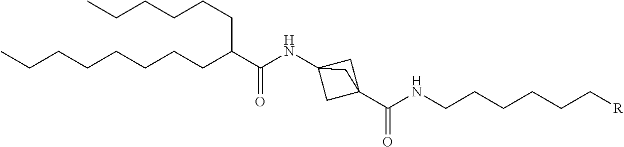 | LP-310c |
| 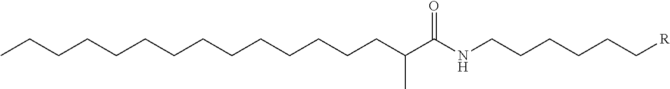 | LP-383c |
| 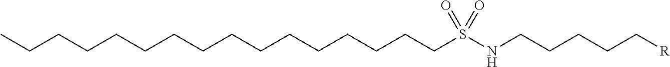 | LP-395c |
| 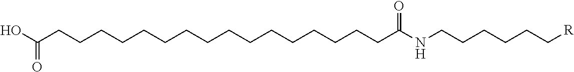 | LP-396c |
| 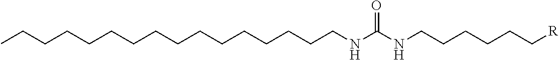 | LP-409p |
| 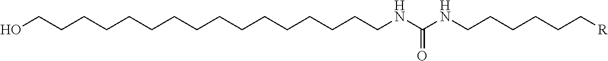 | LP-429c |
| 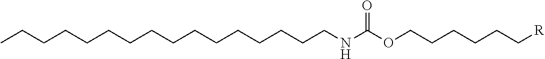 | LP-430c |
| 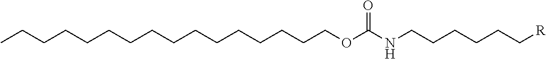 | LP-431c |
| 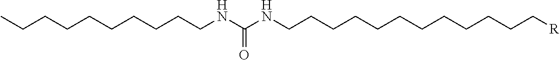 | LP-435c |
| 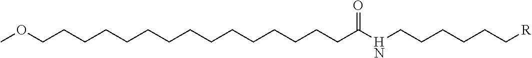 | LP-440c |
| 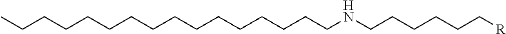 | LP-441c |
| 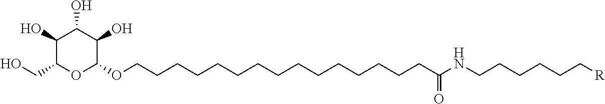 | LP-456c |
| 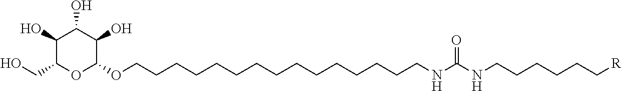 | LP-462c |
| 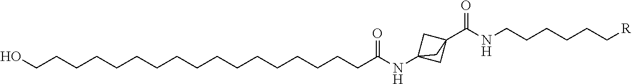 | LP-463c |
| 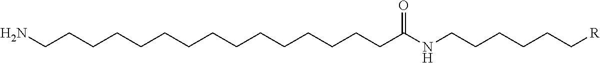 | LP-464c |
| 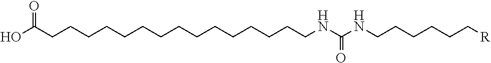 | LP-465c |
| 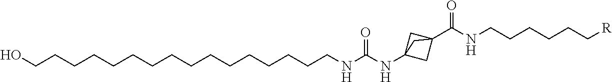 | LP-466c |
| 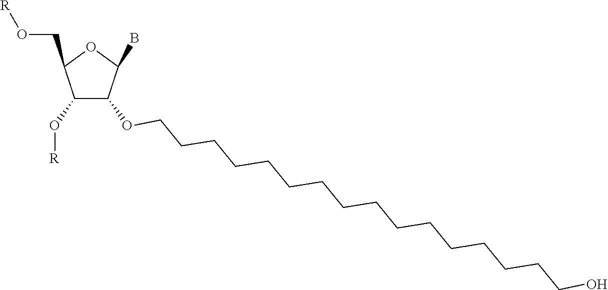 | LP-493c |
| (2′ internal) | |

wherein R comprises an oligonucleotide. In some embodiments, R is the point of connection to the 5′ terminal nucleotide of an oligonucleotide.

### Definitions

As used herein, the terms “oligonucleotide” and “polynucleotide” mean a polymer of linked nucleosides each of which can be independently modified or unmodified.

As used herein, an “RNAi agent” (also referred to as an “RNAi trigger”) means a composition that contains an RNA or RNA-like (e.g., chemically modified RNA) oligonucleotide molecule that is capable of degrading or inhibiting (e.g., degrades or inhibits under appropriate conditions) translation of messenger RNA (mRNA) transcripts of a target mRNA in a sequence specific manner. As used herein, RNAi agents may operate through the RNA interference mechanism (i.e., inducing RNA interference through interaction with the RNA interference pathway machinery (RNA-induced silencing complex or RISC) of mammalian cells), or by any alternative mechanism(s) or pathway(s). While it is believed that RNAi agents, as that term is used herein, operate primarily through the RNA interference mechanism, the disclosed RNAi agents are not bound by or limited to any particular pathway or mechanism of action. RNAi agents disclosed herein are comprised of a sense strand and an antisense strand, and include, but are not limited to: short (or small) interfering RNAs (siRNAs), double-stranded RNAs (dsRNA), micro RNAs (miRNAs), short hairpin RNAs (shRNA), and dicer substrates. The antisense strand of the RNAi agents described herein is at least partially complementary to the mRNA being targeted. RNAi agents can include one or more modified nucleotides and/or one or more non-phosphodiester linkages.

As used herein, the term “lipid” refers to moieties and molecules that are soluble in nonpolar solvents. The term lipid includes amphiphilic molecules comprising a polar, water-soluble head group and a hydrophobic tail. Lipids can be of natural or synthetic origin. Non-limiting examples of lipids include fatty acids (e.g., saturated fatty acids, monounsaturated fatty acids, and polyunsaturated fatty acids), glycerolipids (e.g., monoacylglycerols, diacylglycerols, and triacylglycerols), phospholipids (e.g., phosphatidylethanolamine, phosphatidylcholine, and phosphatidylserine), sphingolipids (e.g., sphingomyelin), and cholesterol esters. As used herein, the term “saturated lipid” refers to lipids that are free of any unsaturation. As used herein, the term “unsaturated lipid” refers to lipids that comprise at least one (1) degree of unsaturation. As used herein, the term “branched lipid” refers to lipids comprising more than one linear chain, wherein each liner chain is covalently attached to at least one other linear chain. As used herein, the term “straight chain lipid” refers to lipids that are free of any branching.

As used herein, the terms “silence,” “reduce,” “inhibit,” “down-regulate,” or “knockdown” when referring to expression of a given gene, mean that the expression of the gene, as measured by the level of RNA transcribed from the gene or the level of polypeptide, protein, or protein subunit translated from the mRNA in a cell, group of cells, tissue, organ, or subject in which the gene is transcribed, is reduced when the cell, group of cells, tissue, organ, or subject is treated with the RNAi agents described herein as compared to a second cell, group of cells, tissue, organ, or subject that has not or have not been so treated.

As used herein, the terms “sequence” and “nucleotide sequence” mean a succession or order of nucleobases or nucleotides, described with a succession of letters using standard nomenclature.

As used herein, a “base,” “nucleotide base,” or “nucleobase,” is a heterocyclic pyrimidine or purine compound that is a component of a nucleotide, and includes the primary purine bases adenine and guanine, and the primary pyrimidine bases cytosine, thymine, and uracil. A nucleobase may further be modified to include, without limitation, universal bases, hydrophobic bases, promiscuous bases, size-expanded bases, and fluorinated bases. (See, e.g., Modified Nucleosides in Biochemistry, Biotechnology and Medicine, Herdewijn, P. ed. Wiley-VCH, 2008). The synthesis of such modified nucleobases (including phosphoramidite compounds that include modified nucleobases) is known in the art.

As used herein, and unless otherwise indicated, the term “complementary,” when used to describe a first nucleobase or nucleotide sequence (e.g., RNAi agent sense strand or targeted mRNA) in relation to a second nucleobase or nucleotide sequence (e.g., RNAi agent antisense strand or a single-stranded antisense oligonucleotide), means the ability of an oligonucleotide or polynucleotide including the first nucleotide sequence to hybridize (form base pair hydrogen bonds under mammalian physiological conditions (or similar conditions in vitro)) and form a duplex or double helical structure under certain standard conditions with an oligonucleotide or polynucleotide including the second nucleotide sequence. Complementary sequences include Watson-Crick base pairs or non-Watson-Crick base pairs and include natural or modified nucleotides or nucleotide mimics, at least to the extent that the above hybridization requirements are fulfilled. Sequence identity or complementarity is independent of modification. For example, a and Af, as defined herein, are complementary to U (or T) and identical to A for the purposes of determining identity or complementarity.

As used herein, “perfectly complementary” or “fully complementary” means that in a hybridized pair of nucleobase or nucleotide sequence molecules, all (100%) of the bases in a contiguous sequence of a first oligonucleotide will hybridize with the same number of bases in a contiguous sequence of a second oligonucleotide. The contiguous sequence may comprise all or a part of a first or second nucleotide sequence.

As used herein, “partially complementary” means that in a hybridized pair of nucleobase or nucleotide sequence molecules, at least 70%, but not all, of the bases in a contiguous sequence of a first oligonucleotide will hybridize with the same number of bases in a contiguous sequence of a second oligonucleotide. The contiguous sequence may comprise all or a part of a first or second nucleotide sequence.

As used herein, “substantially complementary” means that in a hybridized pair of nucleobase or nucleotide sequence molecules, at least 85%, but not all, of the bases in a contiguous sequence of a first oligonucleotide will hybridize with the same number of bases in a contiguous sequence of a second oligonucleotide. The contiguous sequence may comprise all or a part of a first or second nucleotide sequence.

As used herein, the terms “complementary,” “fully complementary,” “partially complementary,” and “substantially complementary” are used with respect to the nucleobase or nucleotide matching between the sense strand and the antisense strand of an RNAi agent, or between the antisense strand of an RNAi agent and a sequence of a target mRNA.

As used herein, the term “substantially identical” or “substantial identity,” as applied to a nucleic acid sequence means the nucleotide sequence (or a portion of a nucleotide sequence) has at least about 85% sequence identity or more, e.g., at least 90%, at least 95%, or at least 99% identity, compared to a reference sequence. Percentage of sequence identity is determined by comparing two optimally aligned sequences over a comparison window. The percentage is calculated by determining the number of positions at which the same type of nucleic acid base occurs in both sequences to yield the number of matched positions, dividing the number of matched positions by the total number of positions in the window of comparison and multiplying the result by 100 to yield the percentage of sequence identity. The inventions disclosed herein encompass nucleotide sequences substantially identical to those disclosed herein.

As used herein, the terms “treat,” “treatment,” and the like, mean the methods or steps taken to provide relief from or alleviation of the number, severity, and/or frequency of one or more symptoms of a disease in a subject. As used herein, “treat” and “treatment” may include the preventative treatment, management, prophylactic treatment, and/or inhibition or reduction of the number, severity, and/or frequency of one or more symptoms of a disease in a subject.

As used herein, the phrase “introducing into a cell,” when referring to an RNAi agent, means functionally delivering the RNAi agent into a cell. The phrase “functional delivery,” means delivering the RNAi agent to the cell in a manner that enables the RNAi agent to have the expected biological activity, e.g., sequence-specific inhibition of gene expression.

As used herein, the term “isomers” refers to compounds that have identical molecular formulae, but that differ in the nature or the sequence of bonding of their atoms or in the arrangement of their atoms in space. Isomers that differ in the arrangement of their atoms in space are termed “stereoisomers.” Stereoisomers that are not mirror images of one another are termed “diastereomers,” and stereoisomers that are non-superimposable mirror images are termed “enantiomers,” or sometimes optical isomers. A carbon atom bonded to four non-identical substituents is termed a “chiral center.”

As used herein, unless specifically identified in a structure as having a particular conformation, for each structure in which asymmetric centers are present and thus give rise to enantiomers, diastereomers, or other stereoisomeric configurations, each structure disclosed herein is intended to represent all such possible isomers, including their optically pure and racemic forms. For example, the structures disclosed herein are intended to cover mixtures of diastereomers as well as single stereoisomers.

As used in a claim herein, the phrase “consisting of” excludes any element, step, or ingredient not specified in the claim. When used in a claim herein, the phrase “consisting essentially of” limits the scope of a claim to the specified materials or steps and those that do not materially affect the basic and novel characteristic(s) of the claimed invention.

The person of ordinary skill in the art would readily understand and appreciate that the compounds and compositions disclosed herein may have certain atoms (e.g., N, O, or S atoms) in a protonated or deprotonated state, depending upon the environment in which the compound or composition is placed. Accordingly, as used herein, the structures disclosed herein envisage that certain functional groups, such as, for example, OH, SH, or NH, may be protonated or deprotonated. The disclosure herein is intended to cover the disclosed compounds and compositions regardless of their state of protonation based on the environment (such as pH), as would be readily understood by the person of ordinary skill in the art.

As used herein, the term “linked” or “conjugated” when referring to the connection between two compounds or molecules means that two molecules are joined by a covalent bond or are associated via noncovalent bonds (e.g., hydrogen bonds or ionic bonds). In some examples, where the term “linked” or “conjugated” refers to the association between two molecules via noncovalent bonds, the association between the two different molecules has a K_{D} of less than 1×10⁻⁴ M (e.g., less than 1×10⁻⁵ M, less than 1×10⁻⁶ M, or less than 1×10⁻⁷ M) in physiologically acceptable buffer (e.g., buffered saline). Unless stated, the terms “linked” and “conjugated” as used herein may refer to the connection between a first compound and a second compound either with or without any intervening atoms or groups of atoms.

As used herein, a linking group is one or more atoms that connects one molecule or portion of a molecule to another to second molecule or second portion of a molecule. Similarly, as used in the art, the term scaffold is sometimes used interchangeably with a linking group. Linking groups may comprise any number of atoms or functional groups. In some embodiments, linking groups may not facilitate any biological or pharmaceutical response, and merely serve to link two biologically active molecules.

As used herein, the term “alkyl” refers to a saturated aliphatic hydrocarbon group containing 1-12 (e.g., 1-8, 1-6, 1-4, or 1-3) carbon atoms. An alkyl group can be straight or branched. Examples of alkyl groups include, but are not limited to, methyl, ethyl, propyl, isopropyl, butyl, isobutyl, sec-butyl, tert-butyl, n-pentyl, n-heptyl, or 2-ethylhexyl.

Unless stated otherwise, use of the symbol [symbol] as used herein means that any group or groups may be linked (or connected) thereto that is in accordance with the scope of the inventions described herein.

As used herein, the term “including” is used to herein mean, and is used interchangeably with, the phrase “including but not limited to.” The term “or” is used herein to mean, and is used interchangeably with, the term “and/or,” unless the context clearly indicates otherwise.

As used in a claim herein, the phrase “consisting of” excludes any element, step, or ingredient not specified in the claim. When used in a claim herein, the phrase “consisting essentially of” limits the scope of a claim to the specified materials or steps and those that do not materially affect the basic and novel characteristic(s) of the claimed invention.

### Oligonucleotide-Based Agents, Including RNAi Agents

As used herein, an “oligonucleotide-based agent” is a nucleotide sequence containing about 10-50 (e.g., 10 to 48, 10 to 46, 10 to 44, 10 to 42, 10 to 40, 10 to 38, 10 to 36, 10 to 34, 10 to 32, 10 to 30, 10 to 28, 10 to 26, 10 to 24, 10 to 22, 10 to 20, 10 to 18, 10 to 16, 10 to 14, 10 to 12, 12 to 50, 12 to 48, 12 to 46, 12 to 44, 12 to 42, 12 to 40, 12 to 38, 12 to 36, 12 to 34, 12 to 32, 12 to 30, 12 to 28, 12 to 26, 12 to 24, 12 to 22, 12 to 20, 12 to 18, 12 to 16, 12 to 14, 14 to 50, 14 to 48, 14 to 46, 14 to 44, 14 to 42, 14 to 40, 14 to 38, 14 to 36, 14 to 34, 14 to 32, 14 to 30, 14 to 28, 14 to 26, 14 to 24, 14 to 22, 14 to 20, 14 to 18, 14 to 16, 16 to 50, 16 to 48, 16 to 46, 16 to 44, 16 to 42, 16 to 40, 16 to 38, 16 to 36, 16 to 34, 16 to 32, 16 to 30, 16 to 28, 16 to 26, 16 to 24, 16 to 22, 16 to 20, 16 to 18, 18 to 50, 18 to 48, 18 to 46, 18 to 44, 18 to 42, 18 to 40, 18 to 38, 18 to 36, 18 to 34, 18 to 32, 18 to 30, 18 to 28, 18 to 26, 18 to 24, 18 to 22, 18 to 20, 20 to 50, 20 to 48, 20 to 46, 20 to 44, 20 to 42, 20 to 40, 20 to 38, 20 to 36, 20 to 34, 20 to 32, 20 to 30, 20 to 28, 20 to 26, 20 to 24, 20 to 22, 22 to 50, 22 to 48, 22 to 46, 22 to 44, 22 to 42, 22 to 40, 22 to 38, 22 to 36, 22 to 34, 22 to 32, 22 to 30, 22 to 28, 22 to 26, 22 to 24, 24 to 50, 24 to 48, 24 to 46, 24 to 44, 24 to 42, 24 to 40, 24 to 38, 24 to 36, 24 to 34, 24 to 32, 24 to 30, 24 to 28, 24 to 26, 26 to 50, 26 to 48, 26 to 46, 26 to 44, 26 to 42, 26 to 40, 26 to 38, 26 to 36, 26 to 34, 26 to 32, 26 to 30, 26 to 28, 28 to 50, 28 to 48, 28 to 46, 28 to 44, 28 to 42, 28 to 40, 28 to 38, 28 to 36, 28 to 34, 28 to 32, to 28 to 30, 30 to 50, 30 to 48, 30 to 46, 30 to 44, 30 to 42, 30 to 40, 30 to 38, 30 to 36, 30 to 34, 30 to 32, 32 to 50, 32 to 48, 32 to 46, 32 to 44, 32 to 42, 32 to 40, 32 to 38, 32 to 36, 32 to 34, 34 to 50, 34 to 48, 34 to 46, 34 to 44, 34 to 42, 34 to 40, 34 to 38, 34 to 36, 36 to 50, 36 to 48, 36 to 46, 36 to 44, 36 to 42, 36 to 40, 36 to 38, 38 to 50, 38 to 48, 38 to 46, 38 to 44, 38 to 42, 38 to 40, 40 to 50, 40 to 48, 40 to 46, 40 to 44, 40 to 42, 42 to 50, 42 to 48, 42 to 46, 42 to 44, 44 to 50, 44 to 48, 44 to 46, 46 to 50, 46 to 48, or 48 to 50) nucleotides or nucleotide base pairs. In some embodiments, an oligonucleotide-based agent has a nucleobase sequence that is at least partially complementary to a coding sequence in an expressed target nucleic acid or target gene within a cell. In some embodiments, the oligonucleotide-based agents, upon delivery to a cell expressing a gene, are able to inhibit the expression of the underlying gene, and are referred to herein as “expression-inhibiting oligonucleotide-based agents.” The gene expression can be inhibited in vitro or in vivo.

“Oligonucleotide-based agents” include, but are not limited to: single-stranded oligonucleotides, single-stranded antisense oligonucleotides, short interfering RNAs (siRNAs), double-strand RNAs (dsRNA), micro RNAs (miRNAs), short hairpin RNAs (shRNA), ribozymes, interfering RNA molecules, and dicer substrates. In some embodiments, an oligonucleotide-based agent is a single-stranded oligonucleotide, such as an antisense oligonucleotide. In some embodiments, an oligonucleotide-based agent is a double-stranded oligonucleotide. In some embodiments, an oligonucleotide-based agent is a double-stranded oligonucleotide that is an RNAi agent.

In some embodiments, the oligonucleotide-based agent is/are an “RNAi agent,” which as defined herein is a composition that contains an RNA or RNA-like (e.g., chemically modified RNA) oligonucleotide molecule that is capable of degrading or inhibiting translation of messenger RNA (mRNA) transcripts of a target mRNA in a sequence specific manner. As used herein, RNAi agents may operate through the RNA interference mechanism (i.e., inducing RNA interference through interaction with the RNA interference pathway machinery (RNA-induced silencing complex or RISC) of mammalian cells), or by any alternative mechanism(s) or pathway(s). While it is believed that RNAi agents, as that term is used herein, operate primarily through the RNA interference mechanism, the disclosed RNAi agents are not bound by or limited to any particular pathway or mechanism of action. RNAi agents disclosed herein are comprised of a sense strand and an antisense strand, and include, but are not limited to: short (or small) interfering RNAs (siRNAs), double-strand RNAs (dsRNA), micro RNAs (miRNAs), short hairpin RNAs (shRNA), and dicer substrates. The antisense strand of the RNAi agents described herein is at least partially complementary to the mRNA being targeted. RNAi agents can include one or more modified nucleotides and/or one or more non-phosphodiester linkages.

Typically, RNAi agents can be comprised of at least a sense strand (also referred to as a passenger strand) that includes a first sequence, and an antisense strand (also referred to as a guide strand) that includes a second sequence. The length of an RNAi agent sense and antisense strands can each be 16 to 49 nucleotides in length. In some embodiments, the sense and antisense strands of an RNAi agent are independently 17 to 26 nucleotides in length. In some embodiments, the sense and antisense strands are independently 19 to 26 nucleotides in length. In some embodiments, the sense and antisense strands are independently 21 to 26 nucleotides in length. In some embodiments, the sense and antisense strands are independently 21 to 24 nucleotides in length. The sense and antisense strands can be either the same length or different lengths. The RNAi agents include an antisense strand sequence that is at least partially complementary to a sequence in the target gene, and upon delivery to a cell expressing the target, an RNAi agent may inhibit the expression of one or more target genes in vivo or in vitro.

Oligonucleotide-based agents generally, and RNAi agents specifically, may be comprised of modified nucleotides and/or one or more non-phosphodiester linkages. As used herein, a “modified nucleotide” is a nucleotide other than a ribonucleotide (2′-hydroxyl nucleotide). In some embodiments, at least 50% (e.g., at least 60%, at least 70%, at least 80%, at least 90%, at least 95%, at least 97%, at least 98%, at least 99%, or 100%) of the nucleotides are modified nucleotides. As used herein, modified nucleotides include, but are not limited to, deoxyribonucleotides, nucleotide mimics, abasic nucleotides, 2′-modified nucleotides, 3′ to 3′ linkages (inverted) nucleotides, non-natural base-comprising nucleotides, bridged nucleotides, peptide nucleic acids, 2′,3′-seco nucleotide mimics (unlocked nucleobase analogues, locked nucleotides, 3′-O-methoxy (2′ intemucleoside linked) nucleotides, 2′-F-Arabino nucleotides, 5′-Me, 2′-fluoro nucleotide, morpholino nucleotides, vinyl phosphonate deoxyribonucleotides, vinyl phosphonate containing nucleotides, and cyclopropyl phosphonate containing nucleotides. 2′-modified nucleotides (i.e. a nucleotide with a group other than a hydroxyl group at the 2′ position of the five-membered sugar ring) include, but are not limited to, 2′-O-methyl nucleotides, 2′-deoxy-2′-fluoro nucleotides, 2′-deoxy nucleotides, 2′-methoxyethyl (2′-O-2-methoxylethyl) nucleotides, 2′-amino nucleotides, and 2′-alkyl nucleotides.

Moreover, one or more nucleotides of an oligonucleotide-based agent, such as an RNAi agent, may be linked by non-standard linkages or backbones (i.e., modified intemucleoside linkages or modified backbones). A modified intemucleoside linkage may be a non-phosphate-containing covalent intemucleoside linkage. Modified intemucleoside linkages or backbones include, but are not limited to, 5′-phosphorothioate groups, chiral phosphorothioates, thiophosphates, phosphorodithioates, phosphotriesters, aminoalkyl-phosphotriesters, alkyl phosphonates (e.g., methyl phosphonates or 3′-alkylene phosphonates), chiral phosphonates, phosphinates, phosphoramidates (e.g., 3′-amino phosphoramidate, aminoalkylphosphoramidates, or thionophosphoramidates), thionoalkyl-phosphonates, thionoalkylphosphotriesters, morpholino linkages, boranophosphates having normal 3′-5′ linkages, 2′-5′ linked analogs of boranophosphates, or boranophosphates having inverted polarity wherein the adjacent pairs of nucleoside units are linked 3′-5′ to 5′-3′ or 2′-5′ to 5′-2′.

It is not necessary for all positions in a given compound to be uniformly modified. Conversely, more than one modification may be incorporated in a single oligonucleotide-based agent or even in a single nucleotide thereof.

The RNAi agent sense strands and antisense strands may be synthesized and/or modified by methods known in the art. Additional disclosures related to RNAi agents may be found, for example, in the disclosure of modifications may be found, for example, in International Patent Application No. PCT/US2017/045446 (WO2018027106) to Arrowhead Pharmaceuticals, Inc., which also is incorporated by reference herein in its entirety.

### Modified Nucleotides

In some embodiments, an RNAi agent contains one or more modified nucleotides. As used herein, a “modified nucleotide” is a nucleotide other than a ribonucleotide (2′-hydroxyl nucleotide). In some embodiments, at least 50% (e.g., at least 60%, at least 70%, at least 80%, at least 90%, at least 95%, at least 97%, at least 98%, at least 99%, or 100%) of the nucleotides are modified nucleotides. As used herein, modified nucleotides can include, but are not limited to, deoxyribonucleotides, nucleotide mimics, abasic nucleotides (represented herein as Ab), 2′-modified nucleotides, 3′ to 3′ linkages (inverted) nucleotides (represented herein as invdN, invN, invn), modified nucleobase-comprising nucleotides, bridged nucleotides, peptide nucleic acids (PNAs), 2′,3′-seco nucleotide mimics (unlocked nucleobase analogues, represented herein as NUNA or NUNA), locked nucleotides (represented herein as NLNA or NLNA), 3′-O-methoxy (2′ intemucleoside linked) nucleotides (represented herein as 3′-OMen), 2′-F-Arabino nucleotides (represented herein as NfANA or Nf_{ANA}), 5′-Me, 2′-fluoro nucleotide (represented herein as 5Me-Nf), morpholino nucleotides, vinyl phosphonate deoxyribonucleotides (represented herein as vpdN), vinyl phosphonate containing nucleotides, and cyclopropyl phosphonate containing nucleotides (cPrpN). 2′-modified nucleotides (i.e., a nucleotide with a group other than a hydroxyl group at the 2′ position of the five-membered sugar ring) include, but are not limited to, 2′-O-methyl nucleotides (represented herein as a lower case letter ‘n’ in a nucleotide sequence), 2′-deoxy-2′-fluoro nucleotides (also referred to herein as 2′-fluoro nucleotide, and represented herein as Nf), 2′-deoxy nucleotides (represented herein as dN), 2′-methoxyethyl (2′-O-2-methoxylethyl) nucleotides (also referred to herein as 2′-MOE, and represented herein as NM), 2′-amino nucleotides, and 2′-alkyl nucleotides. It is not necessary for all positions in a given compound to be uniformly modified. Conversely, more than one modification can be incorporated in a single target RNAi agent or even in a single nucleotide thereof. The target RNAi agent sense strands and antisense strands can be synthesized and/or modified by methods known in the art. Modification at one nucleotide is independent of modification at another nucleotide.

Modified nucleobases include synthetic and natural nucleobases, such as 5-substituted pyrimidines, 6-azapyrimidines and N-2, N-6 and 0-6 substituted purines, (e.g., 2-aminopropyladenine, 5-propynyluracil, or 5-propynylcytosine), 5-methylcytosine (5-me-C), 5-hydroxymethyl cytosine, inosine, xanthine, hypoxanthine, 2-aminoadenine, 6-alkyl (e.g., 6-methyl, 6-ethyl, 6-isopropyl, or 6-n-butyl) derivatives of adenine and guanine, 2-alkyl (e.g., 2-methyl, 2-ethyl, 2-isopropyl, or 2-n-butyl) and other alkyl derivatives of adenine and guanine, 2-thiouracil, 2-thiothymine, 2-thiocytosine, 5-halouracil, cytosine, 5-propynyl uracil, 5-propynyl cytosine, 6-azo uracil, 6-azo cytosine, 6-azo thymine, 5-uracil (pseudouracil), 4-thiouracil, 8-halo, 8-amino, 8-sulfhydryl, 8-thioalkyl, 8-hydroxyl and other 8-substituted adenines and guanines, 5-halo (e.g., 5-brorno), 5-trifluoromethyl, and other 5-substituted uracils and cytosines, 7-methylguanine and 7-methyladenine, 8-azaguanine and 8-azaadenine, 7-deazaguanine, 7-deazaadenine, 3-deazaguanine, and 3-deazaadenine.

In some embodiments, all or substantially all of the nucleotides of an RNAi agent are modified nucleotides. As used herein, an RNAi agent wherein substantially all of the nucleotides present are modified nucleotides is an RNAi agent having four or fewer (i.e., 0, 1, 2, 3, or 4) nucleotides in both the sense strand and the antisense strand being ribonucleotides (i.e., unmodified). As used herein, a sense strand wherein substantially all of the nucleotides present are modified nucleotides is a sense strand having two or fewer (i.e., 0, 1, or 2) nucleotides in the sense strand being unmodified ribonucleotides. As used herein, an antisense sense strand wherein substantially all of the nucleotides present are modified nucleotides is an antisense strand having two or fewer (i.e., 0, 1, or 2) nucleotides in the sense strand being unmodified ribonucleotides. In some embodiments, one or more nucleotides of an RNAi agent is an unmodified ribonucleotide.

### Modified Internucleoside Linkages

In some embodiments, one or more nucleotides of an RNAi agent are linked by non-standard linkages or backbones (i.e., modified intemucleoside linkages or modified backbones). Modified internucleoside linkages or backbones include, but are not limited to, phosphorothioate groups (represented herein as a lower case “s”), chiral phosphorothioates, thiophosphates, phosphorodithioates, phosphotriesters, aminoalkyl-phosphotriesters, alkyl phosphonates (e.g., methyl phosphonates or 3′-alkylene phosphonates), chiral phosphonates, phosphinates, phosphoramidates (e.g., 3′-amino phosphoramidate, aminoalkylphosphoramidates, or thionophosphoramidates), thionoalkyl-phosphonates, thionoalkylphosphotriesters, morpholino linkages, boranophosphates having normal 3′-5′ linkages, 2′-5′ linked analogs of boranophosphates, or boranophosphates having inverted polarity wherein the adjacent pairs of nucleoside units are linked 3′-5′ to 5′-3′ or 2′-5′ to 5′-2′. In some embodiments, a modified internucleoside linkage or backbone lacks a phosphorus atom. Modified internucleoside linkages lacking a phosphorus atom include, but are not limited to, short chain alkyl or cycloalkyl inter-sugar linkages, mixed heteroatom and alkyl or cycloalkyl inter-sugar linkages, or one or more short chain heteroatomic or heterocyclic inter-sugar linkages. In some embodiments, modified internucleoside backbones include, but are not limited to, siloxane backbones, sulfide backbones, sulfoxide backbones, sulfone backbones, formacetyl and thioformacetyl backbones, methylene formacetyl and thioformacetyl backbones, alkene-containing backbones, sulfamate backbones, methyleneimino and methylenehydrazino backbones, sulfonate and sulfonamide backbones, amide backbones, and other backbones having mixed N, O, S, and CH₂ components.

In some embodiments, a sense strand of an RNAi agent can contain 1, 2, 3, 4, 5, or 6 phosphorothioate linkages, an antisense strand of an RNAi agent can contain 1, 2, 3, 4, 5, or 6 phosphorothioate linkages, or both the sense strand and the antisense strand independently can contain 1, 2, 3, 4, 5, or 6 phosphorothioate linkages. In some embodiments, a sense strand of an RNAi agent can contain 1, 2, 3, or 4 phosphorothioate linkages, an antisense strand of an RNAi agent can contain 1, 2, 3, or 4 phosphorothioate linkages, or both the sense strand and the antisense strand independently can contain 1, 2, 3, or 4 phosphorothioate linkages.

In some embodiments, an RNAi agent sense strand contains at least two phosphorothioate intemucleoside linkages. In some embodiments, the at least two phosphorothioate intemucleoside linkages are between the nucleotides at positions 1-3 from the 3′ end of the sense strand. In some embodiments, one phosphorothioate intemucleoside linkage is at the 5′ end of the sense strand, and another phosphorothioate linkage is at the 3′ end of the sense strand. In some embodiments, two phosphorothioate intemucleoside linkage are located at the 5′ end of the sense strand, and another phosphorothioate linkage is at the 3′ end of the sense strand. In some embodiments, the sense strand does not include any phosphorothioate intemucleoside linkages between the nucleotides, but contains one, two, or three phosphorothioate linkages between the terminal nucleotides on both the 5′ and 3′ ends and the optionally present inverted abasic residue terminal caps. In some embodiments, the targeting ligand is linked to the sense strand via a phosphorothioate linkage.

In some embodiments, an RNAi agent antisense strand contains four phosphorothioate intemucleoside linkages. In some embodiments, the four phosphorothioate intemucleoside linkages are between the nucleotides at positions 1-3 from the 5′ end of the antisense strand and between the nucleotides at positions 19-21, 20-22, 21-23, 22-24, 23-25, or 24-26 from the 5′ end. In some embodiments, three phosphorothioate intemucleoside linkages are located between positions 1-4 from the 5′ end of the antisense strand, and a fourth phosphorothioate intemucleoside linkage is located between positions 20-21 from the 5′ end of the antisense strand. In some embodiments, an RNAi agent contains at least three or four phosphorothioate intemucleoside linkages in the antisense strand.

In some embodiments, an RNAi agent contains one or more modified nucleotides and one or more modified intemucleoside linkages. In some embodiments, a 2′-modified nucleoside is combined with modified intemucleoside linkage.

### Linking Groups and Delivery Agents

In some embodiments, the oligonucleotide-based agent, such as RNAi agents described herein, contains or is conjugated to one or more non-nucleotide groups including, but not limited to a linking group or a delivery agent. The non-nucleotide group can enhance targeting, delivery, or attachment of the RNAi agent. Examples of linking groups are provided in Table 4. The non-nucleotide group can be covalently linked to the 3′ and/or 5′ end of either the sense strand and/or the antisense strand. In some embodiments, an RNAi agent contains a non-nucleotide group linked to the 3′ and/or 5′ end of the sense strand. In some embodiments, a non-nucleotide group is linked to the 5′ end of an RNAi agent sense strand. A non-nucleotide group can be linked directly or indirectly to the RNAi agent via a linker/linking group. In some embodiments, a non-nucleotide group is linked to the RNAi agent via a labile, cleavable, or reversible bond or linker.

In some embodiments, a non-nucleotide group enhances the pharmacokinetic or biodistribution properties of an RNAi agent or conjugate to which it is attached to improve cell- or tissue-specific distribution and cell-specific uptake of the conjugate. In some embodiments, a non-nucleotide group enhances endocytosis of the RNAi agent.

The RNAi agents described herein can be synthesized having a reactive group, such as an amino group (also referred to herein as an amine), at the 5′-terminus and/or the 3′-terminus. The reactive group can be used subsequently to attach a targeting moiety using methods typical in the art.

For example, in some embodiments, the RNAi agents disclosed herein are synthesized having an NH₂-C₆ group at the 5′-terminus of the sense strand of the RNAi agent. The terminal amino group subsequently can be reacted to form a conjugate with, for example, a group that includes a compound having affinity for one or more integrins (i.e., and integrin targeting ligand) or a PK enhancer. In some embodiments, the RNAi agents disclosed herein are synthesized having one or more alkyne groups at the 5′-terminus of the sense strand of the RNAi agent. The terminal alkyne group(s) can subsequently be reacted to form a conjugate with, for example, a group that includes a targeting ligand.

In some embodiments, the RNAi agent is synthesized having present a linking group, which can then facilitate covalent linkage of the RNAi agent to a targeting ligand, a targeting group, a PK/PD modulator, or another type of delivery agent. The linking group can be linked to the 3′ and/or the 5′ end of the RNAi agent sense strand or antisense strand. In some embodiments, the linking group is linked to the RNAi agent sense strand. In some embodiments, the linking group is conjugated to the 5′ or 3′ end of an RNAi agent sense strand. In some embodiments, a linking group is conjugated to the 5′ end of an RNAi agent sense strand. Examples of linking groups, include, but are not limited to: C6-SS-Alk-Me, reactive groups such a primary amines and alkynes, alkyl groups, abasic residues/nucleotides, amino acids, trialkyne functionalized groups, ribitol, and/or PEG groups.

A linker or linking group is a connection between two atoms that links one chemical group (such as an RNAi agent) or segment of interest to another chemical group (such as a targeting ligand, targeting group, PK/PD modulator, or delivery agent) or segment of interest via one or more covalent bonds. A labile linkage contains a labile bond. A linkage can optionally include a spacer that increases the distance between the two joined atoms. A spacer may further add flexibility and/or length to the linkage. Spacers include, but are not be limited to, alkyl groups, alkenyl groups, alkynyl groups, aryl groups, aralkyl groups, aralkenyl groups, and aralkynyl groups; each of which can contain one or more heteroatoms, heterocycles, amino acids, nucleotides, and saccharides. Spacer groups are well known in the art and the preceding list is not meant to limit the scope of the description.

In some embodiments, targeting groups are linked to the RNAi agents without the use of an additional linker. In some embodiments, the targeting group is designed to have a linker readily present to facilitate the linkage to an RNAi agent. In some embodiments, when two or more RNAi agents are included in a composition, the two or more RNAi agents can be linked to their respective targeting groups using the same linkers. In some embodiments, when two or more RNAi agents are included in a composition, the two or more RNAi agents are linked to their respective targeting groups using different linkers.

RNAi agents whether modified or unmodified, may contain 3′ and/or 5′ targeting group(s), linking group(s), and/or may be conjugated with, or comprise, PK/PD modulator(s). Any of the RNAi agent sequences or are otherwise described herein, which contain a 3′ or 5′ targeting ligand, targeting group, PK/PD modulator, or linking group, can alternatively contain no 3′ or 5′ targeting ligand, targeting group, linking group, or PK/PD modulator, or can contain a different 3′ or 5′ targeting ligand, targeting group, linking group, or PK/PD modulator including, but not limited to, those depicted in Tables 2 and 3. Any of the RNAi agent duplexes listed in Table A, whether modified or unmodified, can further comprise a targeting ligand, targeting group, linking group, or PK/PD modulator, and the targeting group or linking group can be attached to the 3′ or 5′ terminus of either the sense strand or the antisense strand of the RNAi agent duplex.

In some embodiments, a linking group may be conjugated synthetically to the 5′ or 3′ end of the sense strand of an RNAi agent described herein. In some embodiments, a linking group is conjugated synthetically to the 5′ end of the sense strand of an RNAi agent. In some embodiments, a linking group conjugated to an RNAi agent may be a trialkyne linking group.

Examples of certain modified nucleotides, Capping Moieties and linking groups, are provided in Table 4.

Table 4: Structures Representing Various Modified Nucleotides, Capping Moieties and Linking Groups.

TABLE 4
Structures Representing Various Modified Nucleotides, Capping Moieties and Linking Groups.

| 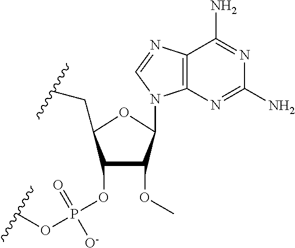 | | a_2N |
|---|---|---|
| 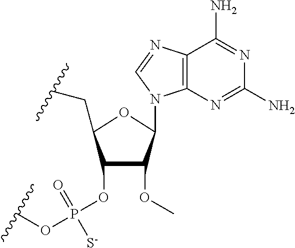 | | a_2Ns |
| 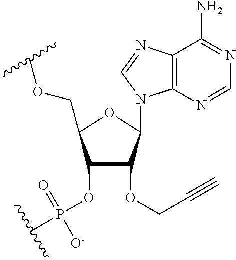 | | aAlk |
| 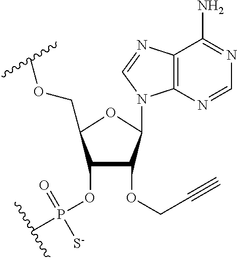 | | aAlks |
| 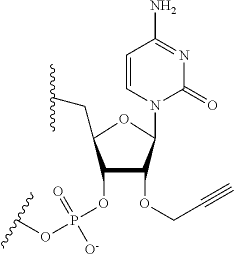 | | cAlk |
| 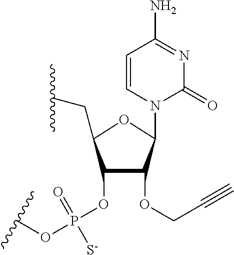 | | cAlks |
| 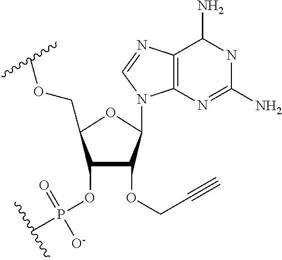 | | gAlk |
| 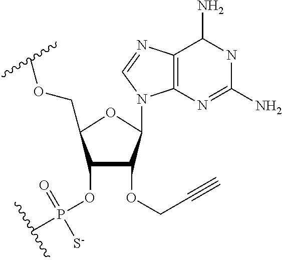 | | gAlks |
| 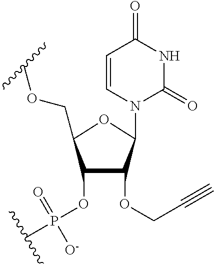 | | uAlk |
| 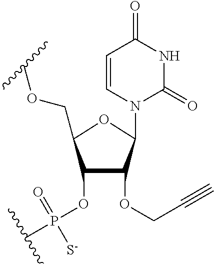 | | aAlks |
| 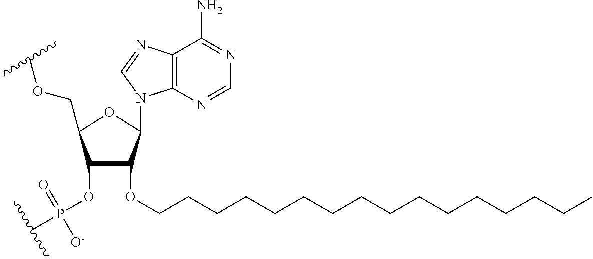 | | aC16 |
| 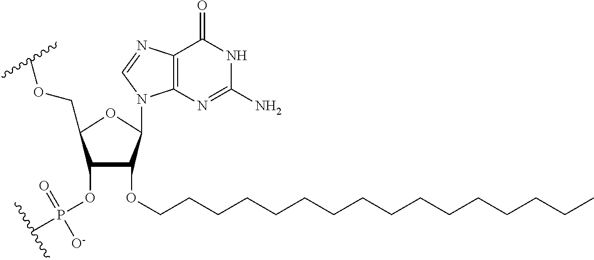 | | gC16 |
| 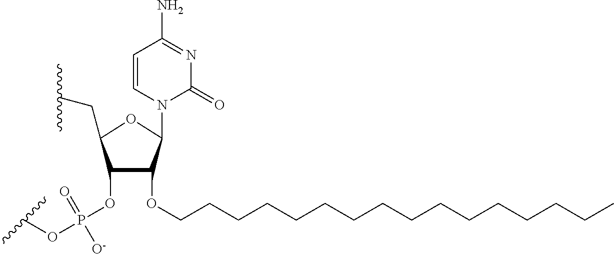 | | cC16 |
| 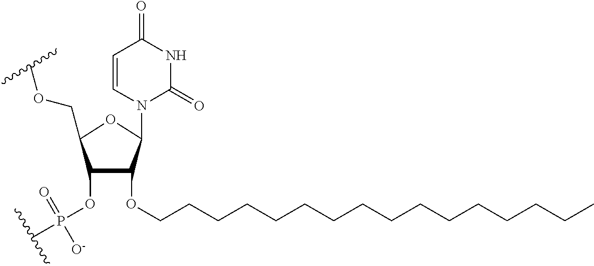 | | uC16 |
| 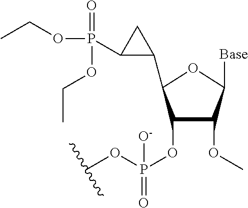 | | cPrp |
| When positioned internally in oligonucleotide: | | |
| linkage towards 5′ end of oligonucleotide | | |
| 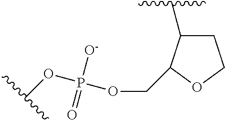 [SMILES: *OP(=O)([O-])OCC1OCCC1*] | | (invAb) |
| linkage towards 3′ end of | | |
| oligonucleotide | | |
| When positioned internally in oligonucleotide: | | |
| linkage towards 5′ end of oligonucleotide | | |
| 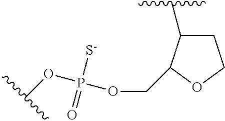 [SMILES: *OP(=O)([S-])OCC1OCCC1*] | | (invAb)s |
| linkage towards 3′ end of | | |
| oligonucleotide | | |
| When positioned at the 3′ terminal end of oligonucleotide: | | |
| linkage towards 5′ end of oligonucleotide | | |
| 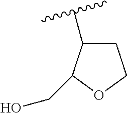 [SMILES: *C1CCOC1CO] | | (invAb) |
| When positioned at the 3′ terminal end of oligonucleotide: | | |
| 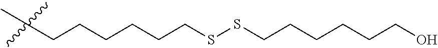 [SMILES: *CCCCCCSSCCCCCCO] | | (C6-SS-C6) |
| When positioned internally in oligonucleotide: | | |
| linkage towards 5′ end of | linkage towards 3′ end of | |
| oligonucleotide | oligonucleotide | |
| 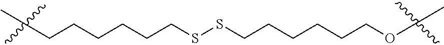 [SMILES: *CCCCCCSSCCCCCCO*] | | (C6-SS-C6) |
| linkage towards 5′ end of | linkage towards 3′ end of | |
| oligonucleotide | oligonucleotide | |
| 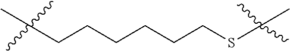 [SMILES: *CCCCCCS*] | | (C6-S) |
| When positioned at the 3′ terminal end of oligonucleotide: | | |
| 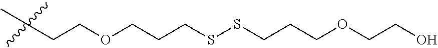 [SMILES: *CCOCCCSSCCCOCCO] | | (6-SS-6) |
| When positioned internally in oligonucleotide: | | |
| linkage towards 5′ end of | linkage towards 3′ end of | |
| oligonucleotide | oligonucleotide | |

| | |
|---|---|
| 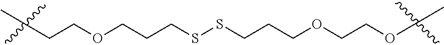 [SMILES: *CCOCCCSSCCCOCCO*] | (6-SS-6) |
| 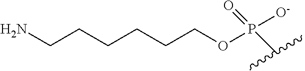 [SMILES: *P(=O)([O-])OCCCCCCN] | (NH2-C6) |
| 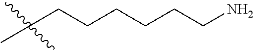 [SMILES: *CCCCCCN] | (C6-NH2) |
| 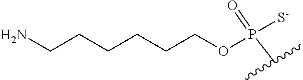 [SMILES: *P(=O)([S-])OCCCCCCN] | (NH2-C6)s |
| 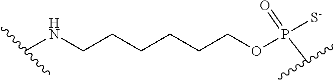 [SMILES: *NCCCCCCOP(*)(=O)[S-]] | (NH-C6)s |
| 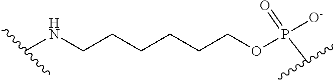 [SMILES: *NCCCCCCOP(*)(=O)[O-]] | (NH-C6) |
| 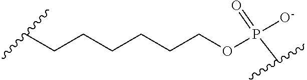 [SMILES: *CCCCCCOP(*)(=O)[O-]] | (C6) |
| 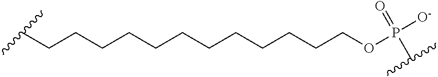 [SMILES: *CCCCCCCCCCCCOP(*)(=O)[O-]] | (C12) |
| 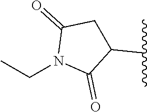 [SMILES: *C1CC(=O)N(CC)C1=O] | |
| nEm or NEM (N-ethylmaleimide) | |
| 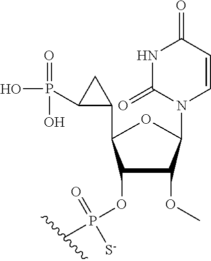 | cPrpus |
| 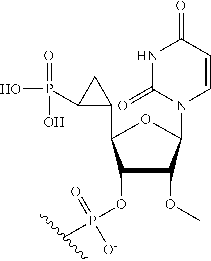 | cPrpu |
| 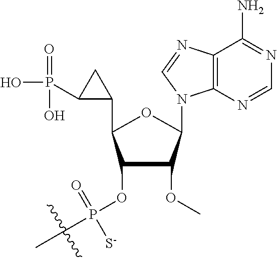 | cPrpas |
| 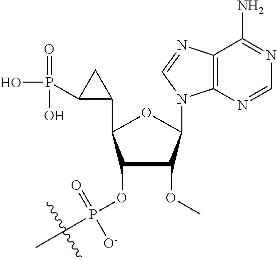 | cPrpa |

Alternatively, other linking groups known in the art may be used.

In addition or alternatively to linking an RNAi agent to one or more targeting ligands, targeting groups, and/or PK/PD modulators, in some embodiments, a delivery agent may be used to deliver an RNAi agent to a cell or tissue. A delivery agent is a compound that can improve delivery of the RNAi agent to a cell or tissue, and can include, or consist of, but is not limited to: a polymer, such as an amphipathic polymer, a membrane active polymer, a peptide, a melittin peptide, a melittin-like peptide (MLP), a lipid, a reversibly modified polymer or peptide, or a reversibly modified membrane active polyamine.

In some embodiments, the RNAi agents can be combined with lipids, nanoparticles, polymers, liposomes, micelles, DPCs or other delivery systems available in the art. The RNAi agents can also be chemically conjugated to targeting groups, lipids (including, but not limited to cholesterol and cholesteryl derivatives), nanoparticles, polymers, liposomes, micelles, DPCs (see, for example WO 2000/053722, WO 2008/022309, WO 2011/104169, and WO 2012/083185, WO 2013/032829, WO 2013/158141, each of which is incorporated herein by reference), or other delivery systems available in the art.

### Pharmaceutical Compositions

In some embodiments, the present disclosure provides pharmaceutical compositions that include, consist of, or consist essentially of, one or more compounds of LP-128a, LP-132a, LP-183a, LP-183r-a, LP-200a, LP-232a, LP-233a, LP-242a, LP-243a, LP-245a, LP-249a, LP-257a, LP-259a, LP-260a, LP-262a, LP-269a, LP-273a, LP-274a, LP-276a, LP-283a, LP-286a, LP-287a, LP-289a, LP-290a, LP-293a, LP-296a, LP-300a, LP-303a, LP-304a, LP-310a, LP-383a, LP-395a, LP-396a, LP-409a, LP-429a, LP-430a, LP-431a, LP-435a, LP-439a, LP-440a, LP-441a, LP-456a, LP-462a, LP-463a, LP-464a, LP-465a, LP-466a, LP-493a (2′ internal), (2C8C12)a, (2C6C10)a, HO-C16a, C16a, C22a, LP-128b, LP-132b, LP-183b, LP-183r-b, LP-200b, LP-232b, LP-233b, LP-242b, LP-243b, LP-245b, LP-249b, LP-257b, LP-259b, LP-260b, LP-262b, LP-269b, LP-273b, LP-274b, LP-276b, LP-283b, LP-286b, LP-287b, LP-289b, LP-290b, LP-293b, LP-296b, LP-300b, LP-303b, LP-304b, LP-310b, LP-383b, LP-395b, LP-396b, LP-409b, LP-429b, LP-430b, LP-431b, LP-435b, LP-439b, LP-440b, LP-441b, LP-456b, LP-462b, LP-463b, LP-464b, LP-465b, LP-466b, LP-493b (2′ internal), (2C8C12)b, (2C6C10)b, HO-C16b, C16b, C22b, LP-128c, LP-132c, LP-183c, LP-183r-c, LP-200c, LP-232c, LP-233c, LP-242c, LP-243c, LP-245c, LP-249c, LP-257c, LP-259c, LP-260c, LP-262c, LP-269c, LP-273c, LP-274c, LP-276c, LP-283c, LP-286c, LP-287c, LP-289c, LP-290c, LP-293c, LP-296c, LP-300c, LP-303c, LP-304c, LP-310c, LP-383c, LP-395c, LP-396c, LP-409c, LP-429c, LP-430c, LP-431c, LP-435c, LP-440c, LP-441c, LP-456c, LP-462c, LP-463c, LP-464c, LP-465c, LP-466c, and LP-493c (2′ internal).

As used herein, a “pharmaceutical composition” comprises a pharmacologically effective amount of an Active Pharmaceutical Ingredient (API), and optionally one or more pharmaceutically acceptable excipients. Pharmaceutically acceptable excipients (excipients) are substances other than the Active Pharmaceutical ingredient (API, therapeutic product) that are intentionally included in the drug delivery system. Excipients do not exert or are not intended to exert a therapeutic effect at the intended dosage. Excipients may act to a) aid in processing of the drug delivery system during manufacture, b) protect, support or enhance stability, bioavailability or patient acceptability of the API, c) assist in product identification, and/or d) enhance any other attribute of the overall safety, effectiveness, of delivery of the API during storage or use. A pharmaceutically acceptable excipient may or may not be an inert substance.

Excipients include, but are not limited to: absorption enhancers, anti-adherents, anti-foaming agents, anti-oxidants, binders, buffering agents, carriers, coating agents, colors, delivery enhancers, delivery polymers, dextran, dextrose, diluents, disintegrants, emulsifiers, extenders, fillers, flavors, glidants, humectants, lubricants, oils, polymers, preservatives, saline, salts, solvents, sugars, suspending agents, sustained release matrices, sweeteners, thickening agents, tonicity agents, vehicles, water-repelling agents, and wetting agents.

The pharmaceutical compositions described herein can contain other additional components commonly found in pharmaceutical compositions. In some embodiments, the additional component is a pharmaceutically-active material. Pharmaceutically-active materials include, but are not limited to: anti-pruritics, astringents, local anesthetics, or anti-inflammatory agents (e.g., antihistamine, diphenhydramine, etc.), small molecule drug, antibody, antibody fragment, aptamers, and/or vaccines.

The pharmaceutical compositions may also contain preserving agents, solubilizing agents, stabilizing agents, wetting agents, emulsifiers, sweeteners, colorants, odorants, salts for the variation of osmotic pressure, buffers, coating agents, or antioxidants. They may also contain other agent with a known therapeutic benefit.

The pharmaceutical compositions can be administered in a number of ways depending upon whether local or systemic treatment is desired and upon the area to be treated. Administration can be made by any way commonly known in the art, such as, but not limited to, topical (e.g., by a transdermal patch), pulmonary (e.g., by inhalation or insufflation of powders or aerosols, including by nebulizer, intratracheal, intranasal), epidermal, transdermal, oral or parenteral. Parenteral administration includes, but is not limited to, intravenous, intraarterial, subcutaneous, intraperitoneal or intramuscular injection or infusion; subdermal (e.g., via an implanted device), intracranial, intraparenchymal, intrathecal, and intraventricular, administration. In some embodiments, the pharmaceutical compositions described herein are administered by subcutaneous injection. The pharmaceutical compositions may be administered orally, for example in the form of tablets, coated tablets, dragées, hard or soft gelatin capsules, solutions, emulsions or suspensions. Administration can also be carried out rectally, for example using suppositories; locally or percutaneously, for example using ointments, creams, gels, or solutions; or parenterally, for example using injectable solutions.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor® EL (BASF, Parsippany, NJ) or phosphate buffered saline. It should be stable under the conditions of manufacture and storage and should be preserved against the contaminating action of microorganisms such as bacteria and fungi. The carrier can be a solvent or dispersion medium containing, for example, water, ethanol, polyol (for example, glycerol, propylene glycol, and liquid polyethylene glycol), and suitable mixtures thereof. The proper fluidity can be maintained, for example, by the use of a coating such as lecithin, by the maintenance of the required particle size in the case of dispersion and by the use of surfactants. In many cases, it will be preferable to include isotonic agents, for example, sugars, polyalcohols such as mannitol, sorbitol, and sodium chloride in the composition. Prolonged absorption of the injectable compositions can be brought about by including in the composition an agent which delays absorption, for example, aluminum monostearate and gelatin.

Sterile injectable solutions can be prepared by incorporating the active compound in the required amount in an appropriate solvent with one or a combination of ingredients enumerated above, as required, followed by filter sterilization. Generally, dispersions are prepared by incorporating the active compound into a sterile vehicle which contains a basic dispersion medium and the required other ingredients from those enumerated above. In the case of sterile powders for the preparation of sterile injectable solutions, methods of preparation include vacuum drying and freeze-drying which yields a powder of the active ingredient plus any additional desired ingredient from a previously sterile-filtered solution thereof.

Formulations suitable for intra-articular administration can be in the form of a sterile aqueous preparation of any of the ligands described herein that can be in microcrystalline form, for example, in the form of an aqueous microcrystalline suspension. Liposomal formulations or biodegradable polymer systems can also be used to present any of the ligands described herein for both intra-articular and ophthalmic administration.

The active compounds can be prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. Liposomal suspensions can also be used as pharmaceutically acceptable carriers. These can be prepared according to methods known to those skilled in the art, for example, as described in U.S. Pat. No. 4,522,811.

A pharmaceutical composition can contain other additional components commonly found in pharmaceutical compositions. Such additional components include, but are not limited to: anti-pruritics, astringents, local anesthetics, or anti-inflammatory agents (e.g., antihistamine, diphenhydramine, etc.). As used herein, “pharmacologically effective amount,” “therapeutically effective amount,” or simply “effective amount” refers to that amount of an the pharmaceutically active agent to produce a pharmacological, therapeutic or preventive result.

Medicaments containing compounds of Formula LP-128a, LP-132a, LP-183a, LP-183r-a, LP-200a, LP-232a, LP-233a, LP-242a, LP-243a, LP-245a, LP-249a, LP-257a, LP-259a, LP-260a, LP-262a, LP-269a, LP-273a, LP-274a, LP-276a, LP-283a, LP-286a, LP-287a, LP-289a, LP-290a, LP-293a, LP-296a, LP-300a, LP-303a, LP-304a, LP-310a, LP-383a, LP-395a, LP-396a, LP-409a, LP-429a, LP-430a, LP-431a, LP-435a, LP-439a, LP-440a, LP-441a, LP-456a, LP-462a, LP-463a, LP-464a, LP-465a, LP-466a, LP-493a (2′ internal), (2C8C12)a, (2C6C10)a, HO-C16a, C16a, C22a, LP-128b, LP-132b, LP-183b, LP-183r-b, LP-200b, LP-232b, LP-233b, LP-242b, LP-243b, LP-245b, LP-249b, LP-257b, LP-259b, LP-260b, LP-262b, LP-269b, LP-273b, LP-274b, LP-276b, LP-283b, LP-286b, LP-287b, LP-289b, LP-290b, LP-293b, LP-296b, LP-300b, LP-303b, LP-304b, LP-310b, LP-383b, LP-395b, LP-396b, LP-409b, LP-429b, LP-430b, LP-431b, LP-435b, LP-439b, LP-440b, LP-441b, LP-456b, LP-462b, LP-463b, LP-464b, LP-465b, LP-466b, LP-493b (2′ internal), (2C8C12)b, (2C6C10)b, HO-C16b, C16b, C22b, LP-128c, LP-132c, LP-183c, LP-183r-c, LP-200c, LP-232c, LP-233c, LP-242c, LP-243c, LP-245c, LP-249c, LP-257c, LP-259c, LP-260c, LP-262c, LP-269c, LP-273c, LP-274c, LP-276c, LP-283c, LP-286c, LP-287c, LP-289c, LP-290c, LP-293c, LP-296c, LP-300c, LP-303c, LP-304c, LP-310c, LP-383c, LP-395c, LP-396c, LP-409c, LP-429c, LP-430c, LP-431c, LP-435c, LP-440c, LP-441c, LP-456c, LP-462c, LP-463c, LP-464c, LP-465c, LP-466c, and LP-493c (2′ internal), are also an object of the present invention, as are processes for the manufacture of such medicaments, which processes comprise bringing one or more compound of LP-128a, LP-132a, LP-183a, LP-183r-a, LP-200a, LP-232a, LP-233a, LP-242a, LP-243a, LP-245a, LP-249a, LP-257a, LP-259a, LP-260a, LP-262a, LP-269a, LP-273a, LP-274a, LP-276a, LP-283a, LP-286a, LP-287a, LP-289a, LP-290a, LP-293a, LP-296a, LP-300a, LP-303a, LP-304a, LP-310a, LP-383a, LP-395a, LP-396a, LP-409a, LP-429a, LP-430a, LP-431a, LP-435a, LP-439a, LP-440a, LP-441a, LP-456a, LP-462a, LP-463a, LP-464a, LP-465a, LP-466a, LP-493a (2′ internal), (2C8C12)a, (2C6C10)a, HO-C16a, C16a, C22a, LP-128b, LP-132b, LP-183b, LP-183r-b, LP-200b, LP-232b, LP-233b, LP-242b, LP-243b, LP-245b, LP-249b, LP-257b, LP-259b, LP-260b, LP-262b, LP-269b, LP-273b, LP-274b, LP-276b, LP-283b, LP-286b, LP-287b, LP-289b, LP-290b, LP-293b, LP-296b, LP-300b, LP-303b, LP-304b, LP-310b, LP-383b, LP-395b, LP-396b, LP-409b, LP-429b, LP-430b, LP-431b, LP-435b, LP-439b, LP-440b, LP-441b, LP-456b, LP-462b, LP-463b, LP-464b, LP-465b, LP-466b, LP-493b (2′ internal), (2C8C12)b, (2C6C10)b, HO-C16b, C16b, C22b, LP-128c, LP-132c, LP-183c, LP-183r-c, LP-200c, LP-232c, LP-233c, LP-242c, LP-243c, LP-245c, LP-249c, LP-257c, LP-259c, LP-260c, LP-262c, LP-269c, LP-273c, LP-274c, LP-276c, LP-283c, LP-286c, LP-287c, LP-289c, LP-290c, LP-293c, LP-296c, LP-300c, LP-303c, LP-304c, LP-310c, LP-383c, LP-395c, LP-396c, LP-409c, LP-429c, LP-430c, LP-431c, LP-435c, LP-440c, LP-441c, LP-456c, LP-462c, LP-463c, LP-464c, LP-465c, LP-466c, and LP-493c (2′ internal), and, if desired, one or more other substances with a known therapeutic benefit, into a pharmaceutically acceptable form.

The described compounds of Formula LP-128a, LP-132a, LP-183a, LP-183r-a, LP-200a, LP-232a, LP-233a, LP-242a, LP-243a, LP-245a, LP-249a, LP-257a, LP-259a, LP-260a, LP-262a, LP-269a, LP-273a, LP-274a, LP-276a, LP-283a, LP-286a, LP-287a, LP-289a, LP-290a, LP-293a, LP-296a, LP-300a, LP-303a, LP-304a, LP-310a, LP-383a, LP-395a, LP-396a, LP-409a, LP-429a, LP-430a, LP-431a, LP-435a, LP-439a, LP-440a, LP-441a, LP-456a, LP-462a, LP-463a, LP-464a, LP-465a, LP-466a, LP-493a (2′ internal), (2C8C12)a, (2C6C10)a, HO-C16a, C16a, C22a, LP-128b, LP-132b, LP-183b, LP-183r-b, LP-200b, LP-232b, LP-233b, LP-242b, LP-243b, LP-245b, LP-249b, LP-257b, LP-259b, LP-260b, LP-262b, LP-269b, LP-273b, LP-274b, LP-276b, LP-283b, LP-286b, LP-287b, LP-289b, LP-290b, LP-293b, LP-296b, LP-300b, LP-303b, LP-304b, LP-310b, LP-383b, LP-395b, LP-396b, LP-409b, LP-429b, LP-430b, LP-431b, LP-435b, LP-439b, LP-440b, LP-441b, LP-456b, LP-462b, LP-463b, LP-464b, LP-465b, LP-466b, LP-493b (2′ internal), (2C8C12)b, (2C6C10)b, HO-C16b, C16b, C22b, LP-128c, LP-132c, LP-183c, LP-183r-c, LP-200c, LP-232c, LP-233c, LP-242c, LP-243c, LP-245c, LP-249c, LP-257c, LP-259c, LP-260c, LP-262c, LP-269c, LP-273c, LP-274c, LP-276c, LP-283c, LP-286c, LP-287c, LP-289c, LP-290c, LP-293c, LP-296c, LP-300c, LP-303c, LP-304c, LP-310c, LP-383c, LP-395c, LP-396c, LP-409c, LP-429c, LP-430c, LP-431c, LP-435c, LP-440c, LP-441c, LP-456c, LP-462c, LP-463c, LP-464c, LP-465c, LP-466c, and LP-493c (2′ internal), and pharmaceutical compositions comprising compounds of LP-128a, LP-132a, LP-183a, LP-183r-a, LP-200a, LP-232a, LP-233a, LP-242a, LP-243a, LP-245a, LP-249a, LP-257a, LP-259a, LP-260a, LP-262a, LP-269a, LP-273a, LP-274a, LP-276a, LP-283a, LP-286a, LP-287a, LP-289a, LP-290a, LP-293a, LP-296a, LP-300a, LP-303a, LP-304a, LP-310a, LP-383a, LP-395a, LP-396a, LP-409a, LP-429a, LP-430a, LP-431a, LP-435a, LP-439a, LP-440a, LP-441a, LP-456a, LP-462a, LP-463a, LP-464a, LP-465a, LP-466a, LP-493a (2′ internal), (2C8C12)a, (2C6C10)a, HO-C16a, C16a, C22a, LP-128b, LP-132b, LP-183b, LP-183r-b, LP-200b, LP-232b, LP-233b, LP-242b, LP-243b, LP-245b, LP-249b, LP-257b, LP-259b, LP-260b, LP-262b, LP-269b, LP-273b, LP-274b, LP-276b, LP-283b, LP-286b, LP-287b, LP-289b, LP-290b, LP-293b, LP-296b, LP-300b, LP-303b, LP-304b, LP-310b, LP-383b, LP-395b, LP-396b, LP-409b, LP-429b, LP-430b, LP-431b, LP-435b, LP-439b, LP-440b, LP-441b, LP-456b, LP-462b, LP-463b, LP-464b, LP-465b, LP-466b, LP-493b (2′ internal), (2C8C12)b, (2C6C10)b, HO-C16b, C16b, C22b, LP-128c, LP-132c, LP-183c, LP-183r-c, LP-200c, LP-232c, LP-233c, LP-242c, LP-243c, LP-245c, LP-249c, LP-257c, LP-259c, LP-260c, LP-262c, LP-269c, LP-273c, LP-274c, LP-276c, LP-283c, LP-286c, LP-287c, LP-289c, LP-290c, LP-293c, LP-296c, LP-300c, LP-303c, LP-304c, LP-310c, LP-383c, LP-395c, LP-396c, LP-409c, LP-429c, LP-430c, LP-431c, LP-435c, LP-440c, LP-441c, LP-456c, LP-462c, LP-463c, LP-464c, LP-465c, LP-466c, and LP-493c (2′ internal), disclosed herein may be packaged or included in a kit, container, pack, or dispenser. The compounds of Formula LP-128a, LP-132a, LP-183a, LP-183r-a, LP-200a, LP-232a, LP-233a, LP-242a, LP-243a, LP-245a, LP-249a, LP-257a, LP-259a, LP-260a, LP-262a, LP-269a, LP-273a, LP-274a, LP-276a, LP-283a, LP-286a, LP-287a, LP-289a, LP-290a, LP-293a, LP-296a, LP-300a, LP-303a, LP-304a, LP-310a, LP-383a, LP-395a, LP-396a, LP-409a, LP-429a, LP-430a, LP-431a, LP-435a, LP-439a, LP-440a, LP-441a, LP-456a, LP-462a, LP-463a, LP-464a, LP-465a, LP-466a, LP-493a (2′ internal), (2C8C12)a, (2C6C10)a, HO-C16a, C16a, C22a, LP-128b, LP-132b, LP-183b, LP-183r-b, LP-200b, LP-232b, LP-233b, LP-242b, LP-243b, LP-245b, LP-249b, LP-257b, LP-259b, LP-260b, LP-262b, LP-269b, LP-273b, LP-274b, LP-276b, LP-283b, LP-286b, LP-287b, LP-289b, LP-290b, LP-293b, LP-296b, LP-300b, LP-303b, LP-304b, LP-310b, LP-383b, LP-395b, LP-396b, LP-409b, LP-429b, LP-430b, LP-431b, LP-435b, LP-439b, LP-440b, LP-441b, LP-456b, LP-462b, LP-463b, LP-464b, LP-465b, LP-466b, LP-493b (2′ internal), (2C8C12)b, (2C6C1O)b, HO-C16b, C16b, C22b, LP-128c, LP-132c, LP-183c, LP-183r-c, LP-200c, LP-232c, LP-233c, LP-242c, LP-243c, LP-245c, LP-249c, LP-257c, LP-259c, LP-260c, LP-262c, LP-269c, LP-273c, LP-274c, LP-276c, LP-283c, LP-286c, LP-287c, LP-289c, LP-290c, LP-293c, LP-296c, LP-300c, LP-303c, LP-304c, LP-310c, LP-383c, LP-395c, LP-396c, LP-409c, LP-429c, LP-430c, LP-431c, LP-435c, LP-440c, LP-441c, LP-456c, LP-462c, LP-463c, LP-464c, LP-465c, LP-466c, and LP-493c (2′ internal), and pharmaceutical compositions comprising the compounds of Formula LP-128a, LP-132a, LP-183a, LP-183r-a, LP-200a, LP-232a, LP-233a, LP-242a, LP-243a, LP-245a, LP-249a, LP-257a, LP-259a, LP-260a, LP-262a, LP-269a, LP-273a, LP-274a, LP-276a, LP-283a, LP-286a, LP-287a, LP-289a, LP-290a, LP-293a, LP-296a, LP-300a, LP-303a, LP-304a, LP-310a, LP-383a, LP-395a, LP-396a, LP-409a, LP-429a, LP-430a, LP-431a, LP-435a, LP-439a, LP-440a, LP-441a, LP-456a, LP-462a, LP-463a, LP-464a, LP-465a, LP-466a, LP-493a (2′ internal), (2C8C12)a, (2C6C10)a, HO-C16a, C16a, C22a, LP-128b, LP-132b, LP-183b, LP-183r-b, LP-200b, LP-232b, LP-233b, LP-242b, LP-243b, LP-245b, LP-249b, LP-257b, LP-259b, LP-260b, LP-262b, LP-269b, LP-273b, LP-274b, LP-276b, LP-283b, LP-286b, LP-287b, LP-289b, LP-290b, LP-293b, LP-296b, LP-300b, LP-303b, LP-304b, LP-310b, LP-383b, LP-395b, LP-396b, LP-409b, LP-429b, LP-430b, LP-431b, LP-435b, LP-439b, LP-440b, LP-441b, LP-456b, LP-462b, LP-463b, LP-464b, LP-465b, LP-466b, LP-493b (2′ internal), (2C8C12)b, (2C6C10)b, HO-C16b, C16b, C22b, LP-128c, LP-132c, LP-183c, LP-183r-c, LP-200c, LP-232c, LP-233c, LP-242c, LP-243c, LP-245c, LP-249c, LP-257c, LP-259c, LP-260c, LP-262c, LP-269c, LP-273c, LP-274c, LP-276c, LP-283c, LP-286c, LP-287c, LP-289c, LP-290c, LP-293c, LP-296c, LP-300c, LP-303c, LP-304c, LP-310c, LP-383c, LP-395c, LP-396c, LP-409c, LP-429c, LP-430c, LP-431c, LP-435c, LP-440c, LP-441c, LP-456c, LP-462c, LP-463c, LP-464c, LP-465c, LP-466c, and LP-493c (2′ internal), may be packaged in pre-filled syringes or vials.

### Methods of Treatment and Inhibition of Expression

The compounds of Formula LP-128a, LP-132a, LP-183a, LP-183r-a, LP-200a, LP-232a, LP-233a, LP-242a, LP-243a, LP-245a, LP-249a, LP-257a, LP-259a, LP-260a, LP-262a, LP-269a, LP-273a, LP-274a, LP-276a, LP-283a, LP-286a, LP-287a, LP-289a, LP-290a, LP-293a, LP-296a, LP-300a, LP-303a, LP-304a, LP-310a, LP-383a, LP-395a, LP-396a, LP-409a, LP-429a, LP-430a, LP-431a, LP-435a, LP-439a, LP-440a, LP-441a, LP-456a, LP-462a, LP-463a, LP-464a, LP-465a, LP-466a, LP-493a (2′ internal), (2C8C12)a, (2C6C10)a, HO-C16a, C16a, C22a, LP-128b, LP-132b, LP-183b, LP-183r-b, LP-200b, LP-232b, LP-233b, LP-242b, LP-243b, LP-245b, LP-249b, LP-257b, LP-259b, LP-260b, LP-262b, LP-269b, LP-273b, LP-274b, LP-276b, LP-283b, LP-286b, LP-287b, LP-289b, LP-290b, LP-293b, LP-296b, LP-300b, LP-303b, LP-304b, LP-310b, LP-383b, LP-395b, LP-396b, LP-409b, LP-429b, LP-430b, LP-431b, LP-435b, LP-439b, LP-440b, LP-441b, LP-456b, LP-462b, LP-463b, LP-464b, LP-465b, LP-466b, LP-493b (2′ internal), (2C8C12)b, (2C6C10)b, HO-C16b, C16b, C22b, LP-128c, LP-132c, LP-183c, LP-183r-c, LP-200c, LP-232c, LP-233c, LP-242c, LP-243c, LP-245c, LP-249c, LP-257c, LP-259c, LP-260c, LP-262c, LP-269c, LP-273c, LP-274c, LP-276c, LP-283c, LP-286c, LP-287c, LP-289c, LP-290c, LP-293c, LP-296c, LP-300c, LP-303c, LP-304c, LP-310c, LP-383c, LP-395c, LP-396c, LP-409c, LP-429c, LP-430c, LP-431c, LP-435c, LP-440c, LP-441c, LP-456c, LP-462c, LP-463c, LP-464c, LP-465c, LP-466c, and LP-493c (2′ internal), disclosed herein can be used to treat a subject (e.g., a human or other mammal) having a disease or disorder that would benefit from administration of such compounds. In some embodiments, the compounds of Formula LP-128a, LP-132a, LP-183a, LP-183r-a, LP-200a, LP-232a, LP-233a, LP-242a, LP-243a, LP-245a, LP-249a, LP-257a, LP-259a, LP-260a, LP-262a, LP-269a, LP-273a, LP-274a, LP-276a, LP-283a, LP-286a, LP-287a, LP-289a, LP-290a, LP-293a, LP-296a, LP-300a, LP-303a, LP-304a, LP-310a, LP-383a, LP-395a, LP-396a, LP-409a, LP-429a, LP-430a, LP-431a, LP-435a, LP-439a, LP-440a, LP-441a, LP-456a, LP-462a, LP-463a, LP-464a, LP-465a, LP-466a, LP-493a (2′ internal), (2C8C12)a, (2C6C10)a, HO-C16a, C16a, C22a, LP-128b, LP-132b, LP-183b, LP-183r-b, LP-200b, LP-232b, LP-233b, LP-242b, LP-243b, LP-245b, LP-249b, LP-257b, LP-259b, LP-260b, LP-262b, LP-269b, LP-273b, LP-274b, LP-276b, LP-283b, LP-286b, LP-287b, LP-289b, LP-290b, LP-293b, LP-296b, LP-300b, LP-303b, LP-304b, LP-310b, LP-383b, LP-395b, LP-396b, LP-409b, LP-429b, LP-430b, LP-431b, LP-435b, LP-439b, LP-440b, LP-441b, LP-456b, LP-462b, LP-463b, LP-464b, LP-465b, LP-466b, LP-493b (2′ internal), (2C8C12)b, (2C6C10)b, HO-C16b, C16b, C22b, LP-128c, LP-132c, LP-183c, LP-183r-c, LP-200c, LP-232c, LP-233c, LP-242c, LP-243c, LP-245c, LP-249c, LP-257c, LP-259c, LP-260c, LP-262c, LP-269c, LP-273c, LP-274c, LP-276c, LP-283c, LP-286c, LP-287c, LP-289c, LP-290c, LP-293c, LP-296c, LP-300c, LP-303c, LP-304c, LP-310c, LP-383c, LP-395c, LP-396c, LP-409c, LP-429c, LP-430c, LP-431c, LP-435c, LP-440c, LP-441c, LP-456c, LP-462c, LP-463c, LP-464c, LP-465c, LP-466c, and LP-493c (2′ internal), disclosed herein can be used to treat a subject (e.g., a human) that would benefit from reduction and/or inhibition in expression of a target mRNA and/or protein levels, for example, a subject that has been diagnosed with or is suffering from symptoms related to a CNS disease or disorder.

In some embodiments, the subject is administered a therapeutically effective amount of one or more compounds of Formula LP-128a, LP-132a, LP-183a, LP-183r-a, LP-200a, LP-232a, LP-233a, LP-242a, LP-243a, LP-245a, LP-249a, LP-257a, LP-259a, LP-260a, LP-262a, LP-269a, LP-273a, LP-274a, LP-276a, LP-283a, LP-286a, LP-287a, LP-289a, LP-290a, LP-293a, LP-296a, LP-300a, LP-303a, LP-304a, LP-310a, LP-383a, LP-395a, LP-396a, LP-409a, LP-429a, LP-430a, LP-431a, LP-435a, LP-439a, LP-440a, LP-441a, LP-456a, LP-462a, LP-463a, LP-464a, LP-465a, LP-466a, LP-493a (2′ internal), (2C8C12)a, (2C6C10)a, HO-C16a, C16a, C22a, LP-128b, LP-132b, LP-183b, LP-183r-b, LP-200b, LP-232b, LP-233b, LP-242b, LP-243b, LP-245b, LP-249b, LP-257b, LP-259b, LP-260b, LP-262b, LP-269b, LP-273b, LP-274b, LP-276b, LP-283b, LP-286b, LP-287b, LP-289b, LP-290b, LP-293b, LP-296b, LP-300b, LP-303b, LP-304b, LP-310b, LP-383b, LP-395b, LP-396b, LP-409b, LP-429b, LP-430b, LP-431b, LP-435b, LP-439b, LP-440b, LP-441b, LP-456b, LP-462b, LP-463b, LP-464b, LP-465b, LP-466b, LP-493b (2′ internal), (2C8C12)b, (2C6C1O)b, HO-C16b, C16b, C22b, LP-128c, LP-132c, LP-183c, LP-183r-c, LP-200c, LP-232c, LP-233c, LP-242c, LP-243c, LP-245c, LP-249c, LP-257c, LP-259c, LP-260c, LP-262c, LP-269c, LP-273c, LP-274c, LP-276c, LP-283c, LP-286c, LP-287c, LP-289c, LP-290c, LP-293c, LP-296c, LP-300c, LP-303c, LP-304c, LP-310c, LP-383c, LP-395c, LP-396c, LP-409c, LP-429c, LP-430c, LP-431c, LP-435c, LP-440c, LP-441c, LP-456c, LP-462c, LP-463c, LP-464c, LP-465c, LP-466c, and LP-493c (2′ internal), disclosed herein. Treatment of a subject can include therapeutic and/or prophylactic treatment. The subject is administered a therapeutically effective amount of one or more compounds of Formula LP-128a, LP-132a, LP-183a, LP-183r-a, LP-200a, LP-232a, LP-233a, LP-242a, LP-243a, LP-245a, LP-249a, LP-257a, LP-259a, LP-260a, LP-262a, LP-269a, LP-273a, LP-274a, LP-276a, LP-283a, LP-286a, LP-287a, LP-289a, LP-290a, LP-293a, LP-296a, LP-300a, LP-303a, LP-304a, LP-310a, LP-383a, LP-395a, LP-396a, LP-409a, LP-429a, LP-430a, LP-431a, LP-435a, LP-439a, LP-440a, LP-441a, LP-456a, LP-462a, LP-463a, LP-464a, LP-465a, LP-466a, LP-493a (2′ internal), (2C8C12)a, (2C6C10)a, HO-C16a, C16a, C22a, LP-128b, LP-132b, LP-183b, LP-183r-b, LP-200b, LP-232b, LP-233b, LP-242b, LP-243b, LP-245b, LP-249b, LP-257b, LP-259b, LP-260b, LP-262b, LP-269b, LP-273b, LP-274b, LP-276b, LP-283b, LP-286b, LP-287b, LP-289b, LP-290b, LP-293b, LP-296b, LP-300b, LP-303b, LP-304b, LP-310b, LP-383b, LP-395b, LP-396b, LP-409b, LP-429b, LP-430b, LP-431b, LP-435b, LP-439b, LP-440b, LP-441b, LP-456b, LP-462b, LP-463b, LP-464b, LP-465b, LP-466b, LP-493b (2′ internal), (2C8C12)b, (2C6C10)b, HO-C16b, C16b, C22b, LP-128c, LP-132c, LP-183c, LP-183r-c, LP-200c, LP-232c, LP-233c, LP-242c, LP-243c, LP-245c, LP-249c, LP-257c, LP-259c, LP-260c, LP-262c, LP-269c, LP-273c, LP-274c, LP-276c, LP-283c, LP-286c, LP-287c, LP-289c, LP-290c, LP-293c, LP-296c, LP-300c, LP-303c, LP-304c, LP-310c, LP-383c, LP-395c, LP-396c, LP-409c, LP-429c, LP-430c, LP-431c, LP-435c, LP-440c, LP-441c, LP-456c, LP-462c, LP-463c, LP-464c, LP-465c, LP-466c, and LP-493c (2′ internal), described herein. The subject can be a human, patient, or human patient. The subject may be an adult, adolescent, child, or infant. Administration of a pharmaceutical composition described herein can be to a human being or animal.

The compounds of Formula LP-128a, LP-132a, LP-183a, LP-183r-a, LP-200a, LP-232a, LP-233a, LP-242a, LP-243a, LP-245a, LP-249a, LP-257a, LP-259a, LP-260a, LP-262a, LP-269a, LP-273a, LP-274a, LP-276a, LP-283a, LP-286a, LP-287a, LP-289a, LP-290a, LP-293a, LP-296a, LP-300a, LP-303a, LP-304a, LP-310a, LP-383a, LP-395a, LP-396a, LP-409a, LP-429a, LP-430a, LP-431a, LP-435a, LP-439a, LP-440a, LP-441a, LP-456a, LP-462a, LP-463a, LP-464a, LP-465a, LP-466a, LP-493a (2′ internal), (2C8C12)a, (2C6C10)a, HO-C16a, C16a, C22a, LP-128b, LP-132b, LP-183b, LP-183r-b, LP-200b, LP-232b, LP-233b, LP-242b, LP-243b, LP-245b, LP-249b, LP-257b, LP-259b, LP-260b, LP-262b, LP-269b, LP-273b, LP-274b, LP-276b, LP-283b, LP-286b, LP-287b, LP-289b, LP-290b, LP-293b, LP-296b, LP-300b, LP-303b, LP-304b, LP-310b, LP-383b, LP-395b, LP-396b, LP-409b, LP-429b, LP-430b, LP-431b, LP-435b, LP-439b, LP-440b, LP-441b, LP-456b, LP-462b, LP-463b, LP-464b, LP-465b, LP-466b, LP-493b (2′ internal), (2C8C12)b, (2C6C10)b, HO-C16b, C16b, C22b, LP-128c, LP-132c, LP-183c, LP-183r-c, LP-200c, LP-232c, LP-233c, LP-242c, LP-243c, LP-245c, LP-249c, LP-257c, LP-259c, LP-260c, LP-262c, LP-269c, LP-273c, LP-274c, LP-276c, LP-283c, LP-286c, LP-287c, LP-289c, LP-290c, LP-293c, LP-296c, LP-300c, LP-303c, LP-304c, LP-310c, LP-383c, LP-395c, LP-396c, LP-409c, LP-429c, LP-430c, LP-431c, LP-435c, LP-440c, LP-441c, LP-456c, LP-462c, LP-463c, LP-464c, LP-465c, LP-466c, and LP-493c (2′ internal), described herein can be used to treat at least one symptom in a subject having a disease or disorder related to a target gene, or having a disease or disorder that is mediated at least in part by the expression of the target gene. In some embodiments, the compounds of Formula LP-128a, LP-132a, LP-183a, LP-183r-a, LP-200a, LP-232a, LP-233a, LP-242a, LP-243a, LP-245a, LP-249a, LP-257a, LP-259a, LP-260a, LP-262a, LP-269a, LP-273a, LP-274a, LP-276a, LP-283a, LP-286a, LP-287a, LP-289a, LP-290a, LP-293a, LP-296a, LP-300a, LP-303a, LP-304a, LP-310a, LP-383a, LP-395a, LP-396a, LP-409a, LP-429a, LP-430a, LP-431a, LP-435a, LP-439a, LP-440a, LP-441a, LP-456a, LP-462a, LP-463a, LP-464a, LP-465a, LP-466a, LP-493a (2′ internal), (2C8C12)a, (2C6C10)a, HO-C16a, C16a, C22a, LP-128b, LP-132b, LP-183b, LP-183r-b, LP-200b, LP-232b, LP-233b, LP-242b, LP-243b, LP-245b, LP-249b, LP-257b, LP-259b, LP-260b, LP-262b, LP-269b, LP-273b, LP-274b, LP-276b, LP-283b, LP-286b, LP-287b, LP-289b, LP-290b, LP-293b, LP-296b, LP-300b, LP-303b, LP-304b, LP-310b, LP-383b, LP-395b, LP-396b, LP-409b, LP-429b, LP-430b, LP-431b, LP-435b, LP-439b, LP-440b, LP-441b, LP-456b, LP-462b, LP-463b, LP-464b, LP-465b, LP-466b, LP-493b (2′ internal), (2C8C12)b, (2C6C10)b, HO-C16b, C16b, C22b, LP-128c, LP-132c, LP-183c, LP-183r-c, LP-200c, LP-232c, LP-233c, LP-242c, LP-243c, LP-245c, LP-249c, LP-257c, LP-259c, LP-260c, LP-262c, LP-269c, LP-273c, LP-274c, LP-276c, LP-283c, LP-286c, LP-287c, LP-289c, LP-290c, LP-293c, LP-296c, LP-300c, LP-303c, LP-304c, LP-310c, LP-383c, LP-395c, LP-396c, LP-409c, LP-429c, LP-430c, LP-431c, LP-435c, LP-440c, LP-441c, LP-456c, LP-462c, LP-463c, LP-464c, LP-465c, LP-466c, and LP-493c (2′ internal), are used to treat or manage a clinical presentation of a subject with a disease or disorder that would benefit from or be mediated at least in party by a reduction in mRNA of a target gene. The subject is administered a therapeutically effective amount of one or more of the compounds of Formula LP-128a, LP-132a, LP-183a, LP-183r-a, LP-200a, LP-232a, LP-233a, LP-242a, LP-243a, LP-245a, LP-249a, LP-257a, LP-259a, LP-260a, LP-262a, LP-269a, LP-273a, LP-274a, LP-276a, LP-283a, LP-286a, LP-287a, LP-289a, LP-290a, LP-293a, LP-296a, LP-300a, LP-303a, LP-304a, LP-310a, LP-383a, LP-395a, LP-396a, LP-409a, LP-429a, LP-430a, LP-431a, LP-435a, LP-439a, LP-440a, LP-441a, LP-456a, LP-462a, LP-463a, LP-464a, LP-465a, LP-466a, LP-493a (2′ internal), (2C8C12)a, (2C6C10)a, HO-C16a, C16a, C22a, LP-128b, LP-132b, LP-183b, LP-183r-b, LP-200b, LP-232b, LP-233b, LP-242b, LP-243b, LP-245b, LP-249b, LP-257b, LP-259b, LP-260b, LP-262b, LP-269b, LP-273b, LP-274b, LP-276b, LP-283b, LP-286b, LP-287b, LP-289b, LP-290b, LP-293b, LP-296b, LP-300b, LP-303b, LP-304b, LP-310b, LP-383b, LP-395b, LP-396b, LP-409b, LP-429b, LP-430b, LP-431b, LP-435b, LP-439b, LP-440b, LP-441b, LP-456b, LP-462b, LP-463b, LP-464b, LP-465b, LP-466b, LP-493b (2′ internal), (2C8C12)b, (2C6C10)b, HO-C16b, C16b, C22b, LP-128c, LP-132c, LP-183c, LP-183r-c, LP-200c, LP-232c, LP-233c, LP-242c, LP-243c, LP-245c, LP-249c, LP-257c, LP-259c, LP-260c, LP-262c, LP-269c, LP-273c, LP-274c, LP-276c, LP-283c, LP-286c, LP-287c, LP-289c, LP-290c, LP-293c, LP-296c, LP-300c, LP-303c, LP-304c, LP-310c, LP-383c, LP-395c, LP-396c, LP-409c, LP-429c, LP-430c, LP-431c, LP-435c, LP-440c, LP-441c, LP-456c, LP-462c, LP-463c, LP-464c, LP-465c, LP-466c, and LP-493c (2′ internal), or compositions described herein. In some embodiments, the methods disclosed herein comprise administering a composition comprising a compound of Formula LP-128a, LP-132a, LP-183a, LP-183r-a, LP-200a, LP-232a, LP-233a, LP-242a, LP-243a, LP-245a, LP-249a, LP-257a, LP-259a, LP-260a, LP-262a, LP-269a, LP-273a, LP-274a, LP-276a, LP-283a, LP-286a, LP-287a, LP-289a, LP-290a, LP-293a, LP-296a, LP-300a, LP-303a, LP-304a, LP-310a, LP-383a, LP-395a, LP-396a, LP-409a, LP-429a, LP-430a, LP-431a, LP-435a, LP-439a, LP-440a, LP-441a, LP-456a, LP-462a, LP-463a, LP-464a, LP-465a, LP-466a, LP-493a (2′ internal), (2C8C12)a, (2C6C10)a, HO-C16a, C16a, C22a, LP-128b, LP-132b, LP-183b, LP-183r-b, LP-200b, LP-232b, LP-233b, LP-242b, LP-243b, LP-245b, LP-249b, LP-257b, LP-259b, LP-260b, LP-262b, LP-269b, LP-273b, LP-274b, LP-276b, LP-283b, LP-286b, LP-287b, LP-289b, LP-290b, LP-293b, LP-296b, LP-300b, LP-303b, LP-304b, LP-310b, LP-383b, LP-395b, LP-396b, LP-409b, LP-429b, LP-430b, LP-431b, LP-435b, LP-439b, LP-440b, LP-441b, LP-456b, LP-462b, LP-463b, LP-464b, LP-465b, LP-466b, LP-493b (2′ internal), (2C8C12)b, (2C6C10)b, HO-C16b, C16b, C22b, LP-128c, LP-132c, LP-183c, LP-183r-c, LP-200c, LP-232c, LP-233c, LP-242c, LP-243c, LP-245c, LP-249c, LP-257c, LP-259c, LP-260c, LP-262c, LP-269c, LP-273c, LP-274c, LP-276c, LP-283c, LP-286c, LP-287c, LP-289c, LP-290c, LP-293c, LP-296c, LP-300c, LP-303c, LP-304c, LP-310c, LP-383c, LP-395c, LP-396c, LP-409c, LP-429c, LP-430c, LP-431c, LP-435c, LP-440c, LP-441c, LP-456c, LP-462c, LP-463c, LP-464c, LP-465c, LP-466c, and LP-493c (2′ internal), described herein to a subject to be treated. In some embodiments, the subject is administered a prophylactically effective amount of any one or more of the described compounds of Formula LP-128a, LP-132a, LP-183a, LP-183r-a, LP-200a, LP-232a, LP-233a, LP-242a, LP-243a, LP-245a, LP-249a, LP-257a, LP-259a, LP-260a, LP-262a, LP-269a, LP-273a, LP-274a, LP-276a, LP-283a, LP-286a, LP-287a, LP-289a, LP-290a, LP-293a, LP-296a, LP-300a, LP-303a, LP-304a, LP-310a, LP-383a, LP-395a, LP-396a, LP-409a, LP-429a, LP-430a, LP-431a, LP-435a, LP-439a, LP-440a, LP-441a, LP-456a, LP-462a, LP-463a, LP-464a, LP-465a, LP-466a, LP-493a (2′ internal), (2C8C12)a, (2C6C10)a, HO-C16a, C16a, C22a, LP-128b, LP-132b, LP-183b, LP-183r-b, LP-200b, LP-232b, LP-233b, LP-242b, LP-243b, LP-245b, LP-249b, LP-257b, LP-259b, LP-260b, LP-262b, LP-269b, LP-273b, LP-274b, LP-276b, LP-283b, LP-286b, LP-287b, LP-289b, LP-290b, LP-293b, LP-296b, LP-300b, LP-303b, LP-304b, LP-310b, LP-383b, LP-395b, LP-396b, LP-409b, LP-429b, LP-430b, LP-431b, LP-435b, LP-439b, LP-440b, LP-441b, LP-456b, LP-462b, LP-463b, LP-464b, LP-465b, LP-466b, LP-493b (2′ internal), (2C8C12)b, (2C6C10)b, HO-C16b, C16b, C22b, LP-128c, LP-132c, LP-183c, LP-183r-c, LP-200c, LP-232c, LP-233c, LP-242c, LP-243c, LP-245c, LP-249c, LP-257c, LP-259c, LP-260c, LP-262c, LP-269c, LP-273c, LP-274c, LP-276c, LP-283c, LP-286c, LP-287c, LP-289c, LP-290c, LP-293c, LP-296c, LP-300c, LP-303c, LP-304c, LP-310c, LP-383c, LP-395c, LP-396c, LP-409c, LP-429c, LP-430c, LP-431c, LP-435c, LP-440c, LP-441c, LP-456c, LP-462c, LP-463c, LP-464c, LP-465c, LP-466c, and LP-493c (2′ internal), thereby treating the subject by preventing or inhibiting the at least one symptom.

In certain embodiments, the present disclosure provides methods for treatment of diseases, disorders, conditions, or pathological states mediated at least in part by target gene expression, in a patient in need thereof, wherein the methods include administering to the patient any of the compounds of Formula LP-128a, LP-132a, LP-183a, LP-183r-a, LP-200a, LP-232a, LP-233a, LP-242a, LP-243a, LP-245a, LP-249a, LP-257a, LP-259a, LP-260a, LP-262a, LP-269a, LP-273a, LP-274a, LP-276a, LP-283a, LP-286a, LP-287a, LP-289a, LP-290a, LP-293a, LP-296a, LP-300a, LP-303a, LP-304a, LP-310a, LP-383a, LP-395a, LP-396a, LP-409a, LP-429a, LP-430a, LP-431a, LP-435a, LP-439a, LP-440a, LP-441a, LP-456a, LP-462a, LP-463a, LP-464a, LP-465a, LP-466a, LP-493a (2′ internal), (2C8C12)a, (2C6C10)a, HO-C16a, C16a, C22a, LP-128b, LP-132b, LP-183b, LP-183r-b, LP-200b, LP-232b, LP-233b, LP-242b, LP-243b, LP-245b, LP-249b, LP-257b, LP-259b, LP-260b, LP-262b, LP-269b, LP-273b, LP-274b, LP-276b, LP-283b, LP-286b, LP-287b, LP-289b, LP-290b, LP-293b, LP-296b, LP-300b, LP-303b, LP-304b, LP-310b, LP-383b, LP-395b, LP-396b, LP-409b, LP-429b, LP-430b, LP-431b, LP-435b, LP-439b, LP-440b, LP-441b, LP-456b, LP-462b, LP-463b, LP-464b, LP-465b, LP-466b, LP-493b (2′ internal), (2C8C12)b, (2C6C10)b, HO-C16b, C16b, C22b, LP-128c, LP-132c, LP-183c, LP-183r-c, LP-200c, LP-232c, LP-233c, LP-242c, LP-243c, LP-245c, LP-249c, LP-257c, LP-259c, LP-260c, LP-262c, LP-269c, LP-273c, LP-274c, LP-276c, LP-283c, LP-286c, LP-287c, LP-289c, LP-290c, LP-293c, LP-296c, LP-300c, LP-303c, LP-304c, LP-310c, LP-383c, LP-395c, LP-396c, LP-409c, LP-429c, LP-430c, LP-431c, LP-435c, LP-440c, LP-441c, LP-456c, LP-462c, LP-463c, LP-464c, LP-465c, LP-466c, and LP-493c (2′ internal), described herein.

In some embodiments, the gene expression level and/or mRNA level of a target gene in a subject to whom a compound of Formula LP-128a, LP-132a, LP-183a, LP-183r-a, LP-200a, LP-232a, LP-233a, LP-242a, LP-243a, LP-245a, LP-249a, LP-257a, LP-259a, LP-260a, LP-262a, LP-269a, LP-273a, LP-274a, LP-276a, LP-283a, LP-286a, LP-287a, LP-289a, LP-290a, LP-293a, LP-296a, LP-300a, LP-303a, LP-304a, LP-310a, LP-383a, LP-395a, LP-396a, LP-409a, LP-429a, LP-430a, LP-431a, LP-435a, LP-439a, LP-440a, LP-441a, LP-456a, LP-462a, LP-463a, LP-464a, LP-465a, LP-466a, LP-493a (2′ internal), (2C8C12)a, (2C6C10)a, HO-C16a, C16a, C22a, LP-128b, LP-132b, LP-183b, LP-183r-b, LP-200b, LP-232b, LP-233b, LP-242b, LP-243b, LP-245b, LP-249b, LP-257b, LP-259b, LP-260b, LP-262b, LP-269b, LP-273b, LP-274b, LP-276b, LP-283b, LP-286b, LP-287b, LP-289b, LP-290b, LP-293b, LP-296b, LP-300b, LP-303b, LP-304b, LP-310b, LP-383b, LP-395b, LP-396b, LP-409b, LP-429b, LP-430b, LP-431b, LP-435b, LP-439b, LP-440b, LP-441b, LP-456b, LP-462b, LP-463b, LP-464b, LP-465b, LP-466b, LP-493b (2′ internal), (2C8C12)b, (2C6C10)b, HO-C16b, C16b, C22b, LP-128c, LP-132c, LP-183c, LP-183r-c, LP-200c, LP-232c, LP-233c, LP-242c, LP-243c, LP-245c, LP-249c, LP-257c, LP-259c, LP-260c, LP-262c, LP-269c, LP-273c, LP-274c, LP-276c, LP-283c, LP-286c, LP-287c, LP-289c, LP-290c, LP-293c, LP-296c, LP-300c, LP-303c, LP-304c, LP-310c, LP-383c, LP-395c, LP-396c, LP-409c, LP-429c, LP-430c, LP-431c, LP-435c, LP-440c, LP-441c, LP-456c, LP-462c, LP-463c, LP-464c, LP-465c, LP-466c, and LP-493c (2′ internal), described herein is administered is reduced by at least about 30%, 35%, 40%, 45%, 50%, 55%, 60%, 65%, 70%, 75%, 80%, 85%, 95%, 96%, 97%, 98%, 99%, or greater than 99% relative to the subject prior to being administered the compound or to a subject not receiving the compound. The gene expression level and/or mRNA level in the subject may be reduced in a cell, group of cells, and/or tissue of the subject.

In some embodiments, the target protein level in a subject to whom a compound of Formula LP-128a, LP-132a, LP-183a, LP-183r-a, LP-200a, LP-232a, LP-233a, LP-242a, LP-243a, LP-245a, LP-249a, LP-257a, LP-259a, LP-260a, LP-262a, LP-269a, LP-273a, LP-274a, LP-276a, LP-283a, LP-286a, LP-287a, LP-289a, LP-290a, LP-293a, LP-296a, LP-300a, LP-303a, LP-304a, LP-310a, LP-383a, LP-395a, LP-396a, LP-409a, LP-429a, LP-430a, LP-431a, LP-435a, LP-439a, LP-440a, LP-441a, LP-456a, LP-462a, LP-463a, LP-464a, LP-465a, LP-466a, LP-493a (2′ internal), (2C8C12)a, (2C6C10)a, HO-C16a, C16a, C22a, LP-128b, LP-132b, LP-183b, LP-183r-b, LP-200b, LP-232b, LP-233b, LP-242b, LP-243b, LP-245b, LP-249b, LP-257b, LP-259b, LP-260b, LP-262b, LP-269b, LP-273b, LP-274b, LP-276b, LP-283b, LP-286b, LP-287b, LP-289b, LP-290b, LP-293b, LP-296b, LP-300b, LP-303b, LP-304b, LP-310b, LP-383b, LP-395b, LP-396b, LP-409b, LP-429b, LP-430b, LP-431b, LP-435b, LP-439b, LP-440b, LP-441b, LP-456b, LP-462b, LP-463b, LP-464b, LP-465b, LP-466b, LP-493b (2′ internal), (2C8C12)b, (2C6C10)b, HO-C16b, C16b, C22b, LP-128c, LP-132c, LP-183c, LP-183r-c, LP-200c, LP-232c, LP-233c, LP-242c, LP-243c, LP-245c, LP-249c, LP-257c, LP-259c, LP-260c, LP-262c, LP-269c, LP-273c, LP-274c, LP-276c, LP-283c, LP-286c, LP-287c, LP-289c, LP-290c, LP-293c, LP-296c, LP-300c, LP-303c, LP-304c, LP-310c, LP-383c, LP-395c, LP-396c, LP-409c, LP-429c, LP-430c, LP-431c, LP-435c, LP-440c, LP-441c, LP-456c, LP-462c, LP-463c, LP-464c, LP-465c, LP-466c, and LP-493c (2′ internal), described herein has been administered is reduced by at least about 30%, 35%, 40%, 45%, 50%, 55%, 60%, 65%, 70%, 75%, 80%, 85%, 90%, 95%, 96%, 97%, 98%, 99%, or greater than 99% relative to the subject prior to being administered the compound or to a subject not receiving the compound. The protein level in the subject may be reduced in a cell, group of cells, tissue, blood, and/or other fluid of the subject.

A reduction in target mRNA levels and/or target protein levels can be assessed by any methods known in the art. As used herein, a reduction or decrease in target mRNA level and/or protein level are collectively referred to herein as a reduction or decrease in target gene and/or protein levels or inhibiting or reducing the expression of a target gene.

In some embodiments, compounds of Formula LP-128a, LP-132a, LP-183a, LP-183r-a, LP-200a, LP-232a, LP-233a, LP-242a, LP-243a, LP-245a, LP-249a, LP-257a, LP-259a, LP-260a, LP-262a, LP-269a, LP-273a, LP-274a, LP-276a, LP-283a, LP-286a, LP-287a, LP-289a, LP-290a, LP-293a, LP-296a, LP-300a, LP-303a, LP-304a, LP-310a, LP-383a, LP-395a, LP-396a, LP-409a, LP-429a, LP-430a, LP-431a, LP-435a, LP-439a, LP-440a, LP-441a, LP-456a, LP-462a, LP-463a, LP-464a, LP-465a, LP-466a, LP-493a (2′ internal), (2C8C12)a, (2C6C10)a, HO-C16a, C16a, C22a, LP-128b, LP-132b, LP-183b, LP-183r-b, LP-200b, LP-232b, LP-233b, LP-242b, LP-243b, LP-245b, LP-249b, LP-257b, LP-259b, LP-260b, LP-262b, LP-269b, LP-273b, LP-274b, LP-276b, LP-283b, LP-286b, LP-287b, LP-289b, LP-290b, LP-293b, LP-296b, LP-300b, LP-303b, LP-304b, LP-310b, LP-383b, LP-395b, LP-396b, LP-409b, LP-429b, LP-430b, LP-431b, LP-435b, LP-439b, LP-440b, LP-441b, LP-456b, LP-462b, LP-463b, LP-464b, LP-465b, LP-466b, LP-493b (2′ internal), (2C8C12)b, (2C6C10)b, HO-C16b, C16b, C22b, LP-128c, LP-132c, LP-183c, LP-183r-c, LP-200c, LP-232c, LP-233c, LP-242c, LP-243c, LP-245c, LP-249c, LP-257c, LP-259c, LP-260c, LP-262c, LP-269c, LP-273c, LP-274c, LP-276c, LP-283c, LP-286c, LP-287c, LP-289c, LP-290c, LP-293c, LP-296c, LP-300c, LP-303c, LP-304c, LP-310c, LP-383c, LP-395c, LP-396c, LP-409c, LP-429c, LP-430c, LP-431c, LP-435c, LP-440c, LP-441c, LP-456c, LP-462c, LP-463c, LP-464c, LP-465c, LP-466c, and LP-493c (2′ internal), described herein may be used in the preparation of a pharmaceutical composition for use in the treatment of a disease, disorder, or symptom that is mediated at least in part by target gene expression. In some embodiments, the disease, disorder, or symptom that is mediated at least in part by target gene expression is a CNS disease or disorder.

In some embodiments, methods of treating a subject are dependent on the body weight of the subject. In some embodiments, compounds of Formula LP-128a, LP-132a, LP-183a, LP-183r-a, LP-200a, LP-232a, LP-233a, LP-242a, LP-243a, LP-245a, LP-249a, LP-257a, LP-259a, LP-260a, LP-262a, LP-269a, LP-273a, LP-274a, LP-276a, LP-283a, LP-286a, LP-287a, LP-289a, LP-290a, LP-293a, LP-296a, LP-300a, LP-303a, LP-304a, LP-310a, LP-383a, LP-395a, LP-396a, LP-409a, LP-429a, LP-430a, LP-431a, LP-435a, LP-439a, LP-440a, LP-441a, LP-456a, LP-462a, LP-463a, LP-464a, LP-465a, LP-466a, LP-493a (2′ internal), (2C8C12)a, (2C6C10)a, HO-C16a, C16a, C22a, LP-128b, LP-132b, LP-183b, LP-183r-b, LP-200b, LP-232b, LP-233b, LP-242b, LP-243b, LP-245b, LP-249b, LP-257b, LP-259b, LP-260b, LP-262b, LP-269b, LP-273b, LP-274b, LP-276b, LP-283b, LP-286b, LP-287b, LP-289b, LP-290b, LP-293b, LP-296b, LP-300b, LP-303b, LP-304b, LP-310b, LP-383b, LP-395b, LP-396b, LP-409b, LP-429b, LP-430b, LP-431b, LP-435b, LP-439b, LP-440b, LP-441b, LP-456b, LP-462b, LP-463b, LP-464b, LP-465b, LP-466b, LP-493b (2′ internal), (2C8C12)b, (2C6C10)b, HO-C16b, C16b, C22b, LP-128c, LP-132c, LP-183c, LP-183r-c, LP-200c, LP-232c, LP-233c, LP-242c, LP-243c, LP-245c, LP-249c, LP-257c, LP-259c, LP-260c, LP-262c, LP-269c, LP-273c, LP-274c, LP-276c, LP-283c, LP-286c, LP-287c, LP-289c, LP-290c, LP-293c, LP-296c, LP-300c, LP-303c, LP-304c, LP-310c, LP-383c, LP-395c, LP-396c, LP-409c, LP-429c, LP-430c, LP-431c, LP-435c, LP-440c, LP-441c, LP-456c, LP-462c, LP-463c, LP-464c, LP-465c, LP-466c, and LP-493c (2′ internal), may be administered at a dose of about 0.05 mg/kg to about 40.0 mg/kg of body weight of the subject. In other embodiments compounds of Formula Formula LP-128a, LP-132a, LP-183a, LP-183r-a, LP-200a, LP-232a, LP-233a, LP-242a, LP-243a, LP-245a, LP-249a, LP-257a, LP-259a, LP-260a, LP-262a, LP-269a, LP-273a, LP-274a, LP-276a, LP-283a, LP-286a, LP-287a, LP-289a, LP-290a, LP-293a, LP-296a, LP-300a, LP-303a, LP-304a, LP-310a, LP-383a, LP-395a, LP-396a, LP-409a, LP-429a, LP-430a, LP-431a, LP-435a, LP-439a, LP-440a, LP-441a, LP-456a, LP-462a, LP-463a, LP-464a, LP-465a, LP-466a, LP-493a (2′ internal), (2C8C12)a, (2C6C10)a, HO-C16a, C16a, C22a, LP-128b, LP-132b, LP-183b, LP-183r-b, LP-200b, LP-232b, LP-233b, LP-242b, LP-243b, LP-245b, LP-249b, LP-257b, LP-259b, LP-260b, LP-262b, LP-269b, LP-273b, LP-274b, LP-276b, LP-283b, LP-286b, LP-287b, LP-289b, LP-290b, LP-293b, LP-296b, LP-300b, LP-303b, LP-304b, LP-310b, LP-383b, LP-395b, LP-396b, LP-409b, LP-429b, LP-430b, LP-431b, LP-435b, LP-439b, LP-440b, LP-441b, LP-456b, LP-462b, LP-463b, LP-464b, LP-465b, LP-466b, LP-493b (2′ internal), (2C8C12)b, (2C6C10)b, HO-C16b, C16b, C22b, LP-128c, LP-132c, LP-183c, LP-183r-c, LP-200c, LP-232c, LP-233c, LP-242c, LP-243c, LP-245c, LP-249c, LP-257c, LP-259c, LP-260c, LP-262c, LP-269c, LP-273c, LP-274c, LP-276c, LP-283c, LP-286c, LP-287c, LP-289c, LP-290c, LP-293c, LP-296c, LP-300c, LP-303c, LP-304c, LP-310c, LP-383c, LP-395c, LP-396c, LP-409c, LP-429c, LP-430c, LP-431c, LP-435c, LP-440c, LP-441c, LP-456c, LP-462c, LP-463c, LP-464c, LP-465c, LP-466c, and LP-493c (2′ internal), may be administered at a dose of about 5 mg/kg to about 20 mg/kg of body weight of the subject.

In some embodiments, compounds of Formula Formula LP-128a, LP-132a, LP-183a, LP-183r-a, LP-200a, LP-232a, LP-233a, LP-242a, LP-243a, LP-245a, LP-249a, LP-257a, LP-259a, LP-260a, LP-262a, LP-269a, LP-273a, LP-274a, LP-276a, LP-283a, LP-286a, LP-287a, LP-289a, LP-290a, LP-293a, LP-296a, LP-300a, LP-303a, LP-304a, LP-310a, LP-383a, LP-395a, LP-396a, LP-409a, LP-429a, LP-430a, LP-431a, LP-435a, LP-439a, LP-440a, LP-441a, LP-456a, LP-462a, LP-463a, LP-464a, LP-465a, LP-466a, LP-493a (2′ internal), (2C8C12)a, (2C6C10)a, HO-C16a, C16a, C22a, LP-128b, LP-132b, LP-183b, LP-183r-b, LP-200b, LP-232b, LP-233b, LP-242b, LP-243b, LP-245b, LP-249b, LP-257b, LP-259b, LP-260b, LP-262b, LP-269b, LP-273b, LP-274b, LP-276b, LP-283b, LP-286b, LP-287b, LP-289b, LP-290b, LP-293b, LP-296b, LP-300b, LP-303b, LP-304b, LP-310b, LP-383b, LP-395b, LP-396b, LP-409b, LP-429b, LP-430b, LP-431b, LP-435b, LP-439b, LP-440b, LP-441b, LP-456b, LP-462b, LP-463b, LP-464b, LP-465b, LP-466b, LP-493b (2′ internal), (2C8C12)b, (2C6C10)b, HO-C16b, C16b, C22b, LP-128c, LP-132c, LP-183c, LP-183r-c, LP-200c, LP-232c, LP-233c, LP-242c, LP-243c, LP-245c, LP-249c, LP-257c, LP-259c, LP-260c, LP-262c, LP-269c, LP-273c, LP-274c, LP-276c, LP-283c, LP-286c, LP-287c, LP-289c, LP-290c, LP-293c, LP-296c, LP-300c, LP-303c, LP-304c, LP-310c, LP-383c, LP-395c, LP-396c, LP-409c, LP-429c, LP-430c, LP-431c, LP-435c, LP-440c, LP-441c, LP-456c, LP-462c, LP-463c, LP-464c, LP-465c, LP-466c, and LP-493c (2′ internal), may be administered in a split dose, meaning that two doses are given to a subject in a short (for example, less than 24 hour) time period. In some embodiments, about half of the desired daily amount is administered in an initial administration, and the remaining about half of the desired daily amount is administered approximately four hours after the initial administration.

In some embodiments, compounds of Formula Formula LP-128a, LP-132a, LP-183a, LP-183r-a, LP-200a, LP-232a, LP-233a, LP-242a, LP-243a, LP-245a, LP-249a, LP-257a, LP-259a, LP-260a, LP-262a, LP-269a, LP-273a, LP-274a, LP-276a, LP-283a, LP-286a, LP-287a, LP-289a, LP-290a, LP-293a, LP-296a, LP-300a, LP-303a, LP-304a, LP-310a, LP-383a, LP-395a, LP-396a, LP-409a, LP-429a, LP-430a, LP-431a, LP-435a, LP-439a, LP-440a, LP-441a, LP-456a, LP-462a, LP-463a, LP-464a, LP-465a, LP-466a, LP-493a (2′ internal), (2C8C12)a, (2C6C10)a, HO-C16a, C16a, C22a, LP-128b, LP-132b, LP-183b, LP-183r-b, LP-200b, LP-232b, LP-233b, LP-242b, LP-243b, LP-245b, LP-249b, LP-257b, LP-259b, LP-260b, LP-262b, LP-269b, LP-273b, LP-274b, LP-276b, LP-283b, LP-286b, LP-287b, LP-289b, LP-290b, LP-293b, LP-296b, LP-300b, LP-303b, LP-304b, LP-310b, LP-383b, LP-395b, LP-396b, LP-409b, LP-429b, LP-430b, LP-431b, LP-435b, LP-439b, LP-440b, LP-441b, LP-456b, LP-462b, LP-463b, LP-464b, LP-465b, LP-466b, LP-493b (2′ internal), (2C8C12)b, (2C6C10)b, HO-C16b, C16b, C22b, LP-128c, LP-132c, LP-183c, LP-183r-c, LP-200c, LP-232c, LP-233c, LP-242c, LP-243c, LP-245c, LP-249c, LP-257c, LP-259c, LP-260c, LP-262c, LP-269c, LP-273c, LP-274c, LP-276c, LP-283c, LP-286c, LP-287c, LP-289c, LP-290c, LP-293c, LP-296c, LP-300c, LP-303c, LP-304c, LP-310c, LP-383c, LP-395c, LP-396c, LP-409c, LP-429c, LP-430c, LP-431c, LP-435c, LP-440c, LP-441c, LP-456c, LP-462c, LP-463c, LP-464c, LP-465c, LP-466c, and LP-493c (2′ internal), described herein may be administered once a week (i.e., weekly). In other embodiments, compounds of Formula Formula LP-128a, LP-132a, LP-183a, LP-183r-a, LP-200a, LP-232a, LP-233a, LP-242a, LP-243a, LP-245a, LP-249a, LP-257a, LP-259a, LP-260a, LP-262a, LP-269a, LP-273a, LP-274a, LP-276a, LP-283a, LP-286a, LP-287a, LP-289a, LP-290a, LP-293a, LP-296a, LP-300a, LP-303a, LP-304a, LP-310a, LP-383a, LP-395a, LP-396a, LP-409a, LP-429a, LP-430a, LP-431a, LP-435a, LP-439a, LP-440a, LP-441a, LP-456a, LP-462a, LP-463a, LP-464a, LP-465a, LP-466a, LP-493a (2′ internal), (2C8C12)a, (2C6C10)a, HO-C16a, C16a, C22a, LP-128b, LP-132b, LP-183b, LP-183r-b, LP-200b, LP-232b, LP-233b, LP-242b, LP-243b, LP-245b, LP-249b, LP-257b, LP-259b, LP-260b, LP-262b, LP-269b, LP-273b, LP-274b, LP-276b, LP-283b, LP-286b, LP-287b, LP-289b, LP-290b, LP-293b, LP-296b, LP-300b, LP-303b, LP-304b, LP-310b, LP-383b, LP-395b, LP-396b, LP-409b, LP-429b, LP-430b, LP-431b, LP-435b, LP-439b, LP-440b, LP-441b, LP-456b, LP-462b, LP-463b, LP-464b, LP-465b, LP-466b, LP-493b (2′ internal), (2C8C12)b, (2C6C10)b, HO-C16b, C16b, C22b, LP-128c, LP-132c, LP-183c, LP-183r-c, LP-200c, LP-232c, LP-233c, LP-242c, LP-243c, LP-245c, LP-249c, LP-257c, LP-259c, LP-260c, LP-262c, LP-269c, LP-273c, LP-274c, LP-276c, LP-283c, LP-286c, LP-287c, LP-289c, LP-290c, LP-293c, LP-296c, LP-300c, LP-303c, LP-304c, LP-310c, LP-383c, LP-395c, LP-396c, LP-409c, LP-429c, LP-430c, LP-431c, LP-435c, LP-440c, LP-441c, LP-456c, LP-462c, LP-463c, LP-464c, LP-465c, LP-466c, and LP-493c (2′ internal), described herein may be administered biweekly (once every other week).

In some embodiments, compounds of Formula LP-128a, LP-132a, LP-183a, LP-183r-a, LP-200a, LP-232a, LP-233a, LP-242a, LP-243a, LP-245a, LP-249a, LP-257a, LP-259a, LP-260a, LP-262a, LP-269a, LP-273a, LP-274a, LP-276a, LP-283a, LP-286a, LP-287a, LP-289a, LP-290a, LP-293a, LP-296a, LP-300a, LP-303a, LP-304a, LP-310a, LP-383a, LP-395a, LP-396a, LP-409a, LP-429a, LP-430a, LP-431a, LP-435a, LP-439a, LP-440a, LP-441a, LP-456a, LP-462a, LP-463a, LP-464a, LP-465a, LP-466a, LP-493a (2′ internal), (2C8C12)a, (2C6C10)a, HO-C16a, C16a, C22a, LP-128b, LP-132b, LP-183b, LP-183r-b, LP-200b, LP-232b, LP-233b, LP-242b, LP-243b, LP-245b, LP-249b, LP-257b, LP-259b, LP-260b, LP-262b, LP-269b, LP-273b, LP-274b, LP-276b, LP-283b, LP-286b, LP-287b, LP-289b, LP-290b, LP-293b, LP-296b, LP-300b, LP-303b, LP-304b, LP-310b, LP-383b, LP-395b, LP-396b, LP-409b, LP-429b, LP-430b, LP-431b, LP-435b, LP-439b, LP-440b, LP-441b, LP-456b, LP-462b, LP-463b, LP-464b, LP-465b, LP-466b, LP-493b (2′ internal), (2C8C12)b, (2C6C10)b, HO-C16b, C16b, C22b, LP-128c, LP-132c, LP-183c, LP-183r-c, LP-200c, LP-232c, LP-233c, LP-242c, LP-243c, LP-245c, LP-249c, LP-257c, LP-259c, LP-260c, LP-262c, LP-269c, LP-273c, LP-274c, LP-276c, LP-283c, LP-286c, LP-287c, LP-289c, LP-290c, LP-293c, LP-296c, LP-300c, LP-303c, LP-304c, LP-310c, LP-383c, LP-395c, LP-396c, LP-409c, LP-429c, LP-430c, LP-431c, LP-435c, LP-440c, LP-441c, LP-456c, LP-462c, LP-463c, LP-464c, LP-465c, LP-466c, and LP-493c (2′ internal), described herein or compositions containing such compounds may be used for the treatment of a disease, disorder, or symptom that is mediated at least in part by target gene expression. In some embodiments, the disease, disorder or symptom that is mediated at least in part by target gene expression is a CNS disease or disorder.

Another aspect of the invention provides for a method of reducing a target gene expression in vivo, the method comprising introducing to a cell a compound of Formula Formula LP-128a, LP-132a, LP-183a, LP-183r-a, LP-200a, LP-232a, LP-233a, LP-242a, LP-243a, LP-245a, LP-249a, LP-257a, LP-259a, LP-260a, LP-262a, LP-269a, LP-273a, LP-274a, LP-276a, LP-283a, LP-286a, LP-287a, LP-289a, LP-290a, LP-293a, LP-296a, LP-300a, LP-303a, LP-304a, LP-310a, LP-383a, LP-395a, LP-396a, LP-409a, LP-429a, LP-430a, LP-431a, LP-435a, LP-439a, LP-440a, LP-441a, LP-456a, LP-462a, LP-463a, LP-464a, LP-465a, LP-466a, LP-493a (2′ internal), (2C8C12)a, (2C6C10)a, HO-C16a, C16a, C22a, LP-128b, LP-132b, LP-183b, LP-183r-b, LP-200b, LP-232b, LP-233b, LP-242b, LP-243b, LP-245b, LP-249b, LP-257b, LP-259b, LP-260b, LP-262b, LP-269b, LP-273b, LP-274b, LP-276b, LP-283b, LP-286b, LP-287b, LP-289b, LP-290b, LP-293b, LP-296b, LP-300b, LP-303b, LP-304b, LP-310b, LP-383b, LP-395b, LP-396b, LP-409b, LP-429b, LP-430b, LP-431b, LP-435b, LP-439b, LP-440b, LP-441b, LP-456b, LP-462b, LP-463b, LP-464b, LP-465b, LP-466b, LP-493b (2′ internal), (2C8C12)b, (2C6C10)b, HO-C16b, C16b, C22b, LP-128c, LP-132c, LP-183c, LP-183r-c, LP-200c, LP-232c, LP-233c, LP-242c, LP-243c, LP-245c, LP-249c, LP-257c, LP-259c, LP-260c, LP-262c, LP-269c, LP-273c, LP-274c, LP-276c, LP-283c, LP-286c, LP-287c, LP-289c, LP-290c, LP-293c, LP-296c, LP-300c, LP-303c, LP-304c, LP-310c, LP-383c, LP-395c, LP-396c, LP-409c, LP-429c, LP-430c, LP-431c, LP-435c, LP-440c, LP-441c, LP-456c, LP-462c, LP-463c, LP-464c, LP-465c, LP-466c, and LP-493c (2′ internal), described herein, wherein the compound comprises an RNAi agent at least substantially complementary to the target gene. In some embodiments, the cell is a CNS cell. In some embodiments, the cell is within a subject. In some embodiments, the subject has been diagnosed with a disease or disorder that is treated, prevented or ameliorated by reducing expression of the target gene. In some embodiments, the disease or disorder is a CNS disease or disorder selected from the group consisting of Alzheimer's Disease, Parkinson's Disease, Huntington's Disease, Amyotrophic lateral sclerosis (ALS), Spinal muscular atrophy (SMA), and Lewy body disease.

Another aspect of the invention provides for the use of any one of the lipid PK/PD modulators conjugated to an oligonucleotide-based agent described herein for the treatment, prevention, or amelioration of a disease or disorder. In some embodiments, the disease or disorder is a CNS disease or disorder selected from the group consisting of Alzheimer's Disease, Parkinson's Disease, Huntington's Disease, Amyotrophic lateral sclerosis (ALS), Spinal muscular atrophy (SMA), and Lewy body disease.

### Cells, Tissues, and Non-Human Organisms

Cells, tissues, and non-human organisms that include at least one of the compounds of Formula Formula LP-128a, LP-132a, LP-183a, LP-183r-a, LP-200a, LP-232a, LP-233a, LP-242a, LP-243a, LP-245a, LP-249a, LP-257a, LP-259a, LP-260a, LP-262a, LP-269a, LP-273a, LP-274a, LP-276a, LP-283a, LP-286a, LP-287a, LP-289a, LP-290a, LP-293a, LP-296a, LP-300a, LP-303a, LP-304a, LP-310a, LP-383a, LP-395a, LP-396a, LP-409a, LP-429a, LP-430a, LP-431a, LP-435a, LP-439a, LP-440a, LP-441a, LP-456a, LP-462a, LP-463a, LP-464a, LP-465a, LP-466a, LP-493a (2′ internal), (2C8C12)a, (2C6C10)a, HO-C16a, C16a, C22a, LP-128b, LP-132b, LP-183b, LP-183r-b, LP-200b, LP-232b, LP-233b, LP-242b, LP-243b, LP-245b, LP-249b, LP-257b, LP-259b, LP-260b, LP-262b, LP-269b, LP-273b, LP-274b, LP-276b, LP-283b, LP-286b, LP-287b, LP-289b, LP-290b, LP-293b, LP-296b, LP-300b, LP-303b, LP-304b, LP-310b, LP-383b, LP-395b, LP-396b, LP-409b, LP-429b, LP-430b, LP-431b, LP-435b, LP-439b, LP-440b, LP-441b, LP-456b, LP-462b, LP-463b, LP-464b, LP-465b, LP-466b, LP-493b (2′ internal), (2C8C12)b, (2C6C10)b, HO-C16b, C16b, C22b, LP-128c, LP-132c, LP-183c, LP-183r-c, LP-200c, LP-232c, LP-233c, LP-242c, LP-243c, LP-245c, LP-249c, LP-257c, LP-259c, LP-260c, LP-262c, LP-269c, LP-273c, LP-274c, LP-276c, LP-283c, LP-286c, LP-287c, LP-289c, LP-290c, LP-293c, LP-296c, LP-300c, LP-303c, LP-304c, LP-310c, LP-383c, LP-395c, LP-396c, LP-409c, LP-429c, LP-430c, LP-431c, LP-435c, LP-440c, LP-441c, LP-456c, LP-462c, LP-463c, LP-464c, LP-465c, LP-466c, and LP-493c (2′ internal), described herein is contemplated. The cell, tissue, or non-human organism is made by delivering the compound of Formula Formula LP-128a, LP-132a, LP-183a, LP-183r-a, LP-200a, LP-232a, LP-233a, LP-242a, LP-243a, LP-245a, LP-249a, LP-257a, LP-259a, LP-260a, LP-262a, LP-269a, LP-273a, LP-274a, LP-276a, LP-283a, LP-286a, LP-287a, LP-289a, LP-290a, LP-293a, LP-296a, LP-300a, LP-303a, LP-304a, LP-310a, LP-383a, LP-395a, LP-396a, LP-409a, LP-429a, LP-430a, LP-431a, LP-435a, LP-439a, LP-440a, LP-441a, LP-456a, LP-462a, LP-463a, LP-464a, LP-465a, LP-466a, LP-493a (2′ internal), (2C8C12)a, (2C6C10)a, HO-C16a, C16a, C22a, LP-128b, LP-132b, LP-183b, LP-183r-b, LP-200b, LP-232b, LP-233b, LP-242b, LP-243b, LP-245b, LP-249b, LP-257b, LP-259b, LP-260b, LP-262b, LP-269b, LP-273b, LP-274b, LP-276b, LP-283b, LP-286b, LP-287b, LP-289b, LP-290b, LP-293b, LP-296b, LP-300b, LP-303b, LP-304b, LP-310b, LP-383b, LP-395b, LP-396b, LP-409b, LP-429b, LP-430b, LP-431b, LP-435b, LP-439b, LP-440b, LP-441b, LP-456b, LP-462b, LP-463b, LP-464b, LP-465b, LP-466b, LP-493b (2′ internal), (2C8C12)b, (2C6C10)b, HO-C16b, C16b, C22b, LP-128c, LP-132c, LP-183c, LP-183r-c, LP-200c, LP-232c, LP-233c, LP-242c, LP-243c, LP-245c, LP-249c, LP-257c, LP-259c, LP-260c, LP-262c, LP-269c, LP-273c, LP-274c, LP-276c, LP-283c, LP-286c, LP-287c, LP-289c, LP-290c, LP-293c, LP-296c, LP-300c, LP-303c, LP-304c, LP-310c, LP-383c, LP-395c, LP-396c, LP-409c, LP-429c, LP-430c, LP-431c, LP-435c, LP-440c, LP-441c, LP-456c, LP-462c, LP-463c, LP-464c, LP-465c, LP-466c, and LP-493c (2′ internal), to the cell, tissue, or non-human organism by any means available in the art. In some embodiments, the cell is a mammalian cell, including, but not limited to, a human cell. In some embodiments the cell is a CNS cell.

The above provided embodiments and items are now illustrated with the following, non-limiting examples.

### EXAMPLES

The following examples are not limiting and are intended to illustrate certain embodiments disclosed herein.

Unless expressly stated otherwise, numerals used to refer to compounds of a given example are only made with reference to that particular example and not any other examples disclosed herein. For example, compound 1 of “Synthesis of LP-183 phosphoramidite” in Example 2 is different from, and does not refer to, compound 1 of “Synthesis of LP-232p” in Example 2. Similarly, it will be appreciated that a particular compound disclosed herein may be identified by different numerals in different examples. Compounds that are disclosed in various tables throughout the detailed description (i.e., LPXXa, LPXXb, and LPXX-p, wherein XX is a number) are referred to consistently throughout the examples herein.

It will be appreciated that, unless expressly stated otherwise, use of the term “EDC” in the examples herein refers to the EDC hydrochloride salt which is commercially available.

### Example 1. Syntheses of RNAi Agents and Compositions

The following describes the general procedures for the syntheses of certain RNAi agents, and conjugates thereof, that are illustrated in the non-limiting Examples set forth herein.

Synthesis of RNAi Agents. RNAi agents can be synthesized using methods generally known in the art. For the synthesis of the RNAi agents illustrated in the Examples set forth herein, the sense and antisense strands of the RNAi agents were synthesized according to solid phase phosphoramidite technology used in oligonucleotide synthesis. Depending on the scale, a MerMade96E® (Bioautomation), a MerMadel2® (Bioautomation), or an Oligopilot 100 (GE Healthcare) was used. Syntheses were performed on a solid support made of controlled pore glass (CPG, 500 Å or 600 Å, obtained from Prime Synthesis, Aston, PA, USA) or polystyrene (obtained from Kinovate, Oceanside, CA, USA). All RNA and 2′-modified RNA phosphoramidites were purchased from Thermo Fisher Scientific (Milwaukee, WI, USA), ChemGenes (Wilmington, MA, USA), or Hongene Biotech (Morrisville, NC, USA). Specifically, the following 2′-O-methyl phosphoramidites that were used include the following. (5′-O-dimethoxytrityl-N⁶-(benzoyl)-2′-O-methyl-adenosine-3′-O-(2-cyanoethyl-N,N-diisopropylamino) phosphoramidite, 5′-O-dimethoxy-trityl-N⁴-(acetyl)-2′-O-methyl-cytidine-3′-O-(2-cyanoethyl-N,N-diisopropyl-amino) phosphoramidite, (5′-O-dimethoxytrityl-N²-(isobutyryl)-2′-O-methyl-guanosine-3′-O-(2-cyanoethyl-N,N-diisopropylamino) phosphoramidite, and 5′-O-dimethoxytrityl-2′-O-methyl-uridine-3′-O-(2-cyanoethyl-N,N-diisopropylamino) phosphoramidite. The 2′-deoxy-2′-fluoro-phosphoramidites and 2′-O-propargyl phosphoramidites carried the same protecting groups as the 2′-O-methyl phosphoramidites. 5′-dimethoxytrityl-2′-O-methyl-inosine-3′-O-(2-cyanoethyl-N,N-diisopropylamino) phosphoramidites were purchased from Glen Research (Virginia). The inverted abasic (3′-O-dimethoxytrityl-2′-deoxyribose-5′-O-(2-cyanoethyl-N,N-diisopropylamino) phosphoramidites were purchased from ChemGenes. The following UNA phosphoramidites that were used included the following: 5′-(4,4′-Dimethoxytrityl)-N6-(benzoyl)-2′,3′-seco-adenosine, 2′-benzoyl-3′-[(2-cyanoethyl)-(N,N-diisopropyl)]-phosphoramidite, 5′-(4,4′-Dimethoxytrityl)-N-acetyl-2′,3′-seco-cytosine, 2′-benzoyl-3′-[(2-cyanoethyl)-(N,N-diiso-propyl)]-phosphoramidite, 5′-(4,4′-Dimethoxytrityl)-N-isobutyryl-2′,3′-seco-guanosine, 2′-benzoyl-3′-[(2-cyanoethyl)-(N,N-diisopropyl)]-phosphoramidite, and 5′-(4,4′-Dimethoxy-trityl)-2′,3′-seco-uridine, 2′-benzoyl-3′-[(2-cyanoethyl)-(N,N- di-iso-propyl)]-phosphoramidite. In order to introduce phosphorothioate linkages, a 100 mM solution of 3-phenyl 1,2,4-dithiazoline-5-one (POS, obtained from PolyOrg, Inc., Leominster, MA, USA) in anhydrous acetonitrile or a 200 mM solution of xanthane hydride (TCI America, Portland, OR, USA) in pyridine was employed.

TFA aminolink phosphoramidites were also commercially purchased (ThermoFisher) to introduce the (NH2-C6) reactive group linkers. TFA aminolink phosphoramidite was dissolved in anhydrous acetonitrile (50 mM) and molecular sieves (3 Å) were added. 5-Benzylthio-1H-tetrazole (BTT, 250 mM in acetonitrile) or 5-Ethylthio-1H-tetrazole (ETT, 250 mM in acetonitrile) was used as activator solution. Coupling times were 10 min (RNA), 90 sec (2′ 0-Me), and 60 sec (2′ F). Trialkyne-containing phosphoramidites were synthesized to introduce the respective (TriAlk #) linkers. When used in connection with the RNAi agents presented in certain Examples herein, trialkyne-containing phosphoramidites were dissolved in anhydrous dichloromethane or anhydrous acetonitrile (50 mM), while all other amidites were dissolved in anhydrous acetonitrile (50 mM), and molecular sieves (3 Å) were added. 5-Benzylthio-1H-tetrazole (BTT, 250 mM in acetonitrile) or 5-Ethylthio-1H-tetrazole (ETT, 250 mM in acetonitrile) was used as activator solution. Coupling times were 10 min (RNA), 90 sec (2′ O-Me), and 60 sec (2′ F).

For some RNAi agents, a linker, such as a C6-SS-C6 or a 6-SS-6 group, was introduced at the 3′ terminal end of the sense strand. Pre-loaded resin was commercially acquired with the respective linker. Alternatively, for some sense strands, a dT resin was used and the respectively linker was then added via standard phosphoramidite synthesis.

Cleavage and deprotection of support bound oligomer. After finalization of the solid phase synthesis, the dried solid support was treated with a 1:1 volume solution of 40 weight (wt.) % methylanine in water and 28% to 31% ammonium hydroxide solution (Aldrich) for 1.5 hours at 30° C. The solution was evaporated and the solid residue was reconstituted in water (see below).

Purification. Crude oligomers were purified by anionic exchange HPLC using a TSKgel® SuperQ-5PW 13 μm column (available from Tosoh Biosciences) and Shimadzu LC-8 system. Buffer A was 20 mM Tris, 5 mM EDTA, pH 9.0 and contained 20% Acetonitrile and buffer B was the same as buffer A with the addition of 1.5 M sodium chloride. UV traces at 260 nm were recorded. Appropriate fractions were pooled then run on size exclusion HPLC using a GE Healthcare XK 16/40 column packed with Sephadex@G25 fine (available from Sigman Aldrich) with a running buffer of 100 mM ammoniun bicarbonate, pH 6.7 and 20% Acetonitrile or filtered water.

Annealing. Complementary strands were mixed by combining equimolar RNA solutions (sense and antisense) in 1× PBS (Phosphate-Buffered Saline, 1×, Coming, Cellgro) to form the RNAi agents. Some RNAi agents were lyophilized and stored at −15 to −25° C. Duplex concentration was determined by measuring the solution absorbance on a UV-Vis spectrometer in 1× PBS. The solution absorbance at 260 nm was then multiplied by a conversion factor and the dilution factor to determine the duplex concentration. The conversion factor used was either 0.037 mg/(mL-cm) or was calculated from an experimentally determined extinction coefficient.

### Example 2. Synthesis of Lipid PK/PD Modulator Precursors

### Synthesis of LP-183 phosphoramidite

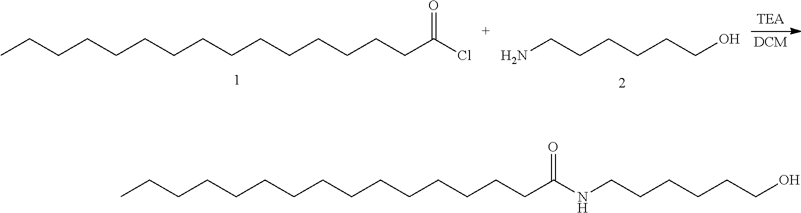 [SMILES: CCCCCCCCCCCCCCCC(=O)Cl ; NCCCCCCO ; CCCCCCCCCCCCCCCC(=O)NCCCCCCO]

To a solution of compound 2 (2.00 g) in DCM was added TEA (2.27 mL) followed by compound 1 (4.931 g) dropwise at room temperature. Then the mixture was stirred at room temperature for 2 h. The mixture was then filtered. The white solid was dried overnight. Product is as white solid, yield, 4.267 g, 74%. LC-MS: calculated [M+H]356.35, found 356.63.

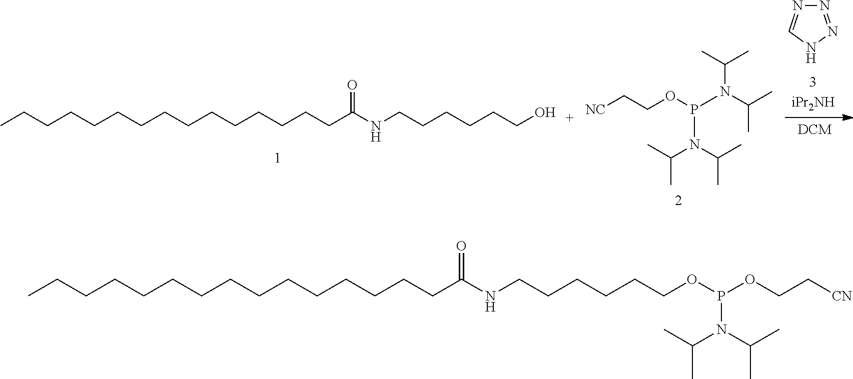 [SMILES: c1nnn[nH]1 ; CCCCCCCCCCCCCCCC(=O)NCCCCCCO ; CC(C)N(C(C)C)P(OCCC#N)N(C(C)C)C(C)C ; CCCCCCCCCCCCCCCC(=O)NCCCCCCOP(OCCC#N)N(C(C)C)C(C)C]

To a mixture of compound 1 (2.54 g) in 120 mL DCM was added compound 3 (0.61 g) followed by compound 2 (5.37 g) dropwise at room temperature. Then the mixture was stirred at room temperature overnight. 5 mL TEA was added followed by Celite. After removing solvent in vacuo, the residue was loaded on a 40 g column by dry method. Hexanes (2% TEA) to 50% EtOAc (2% TEA) in Hexanes (2% TEA) as gradient was used to purify the product. Product is a white waxy solid, yield 3.462 g, 87%. LC-MS: calculated [M+H]556.46, found 556.64.

### Synthesis of LP-183r-p

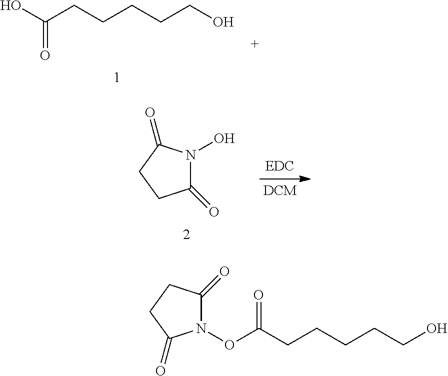 [SMILES: O=C(O)CCCCCO ; O=C1CCC(=O)N1O ; O=C(CCCCCO)ON1C(=O)CCC1=O]

To a solution of Compound 1 (312 mg) in 10 mL DCM was added Compound 2 (299 mg) and EDC (498 mg) at RT. The reaction mixture was stirred at RT for 1 h. After removing solvent in vacuo, the residue was dry loaded on a 12 g column. Hexanes to EtOAc was used as the mobile phase. Product is a clear oil, 408 mg, 75% yield. LC-MS: calculated [M+H]230.10, found 230.34.

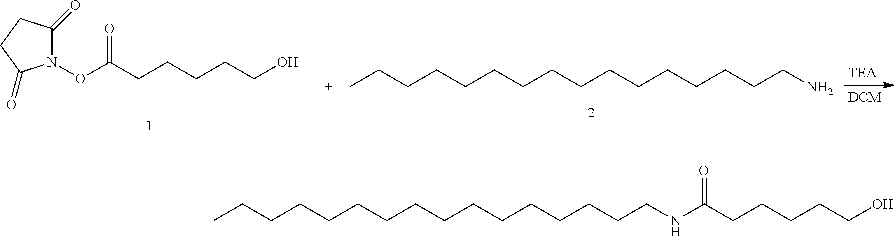 [SMILES: O=C(CCCCCO)ON1C(=O)CCC1=O ; CCCCCCCCCCCCCCCCN ; CCCCCCCCCCCCCCCCNC(=O)CCCCCO]

To a solution of compound 1 (408 mg) in 20 mL DCM was added compound 2 (516 mg) and TEA (0.745 mL) at RT. The reaction mixture was stirred at RT overnight. After removing solvent in vacuo, the residue was recrystalized in MeOH. Product is a white solid, 555 mg, 88% yield. LC-MS: calculated [M+H]356.35, found 356.45.

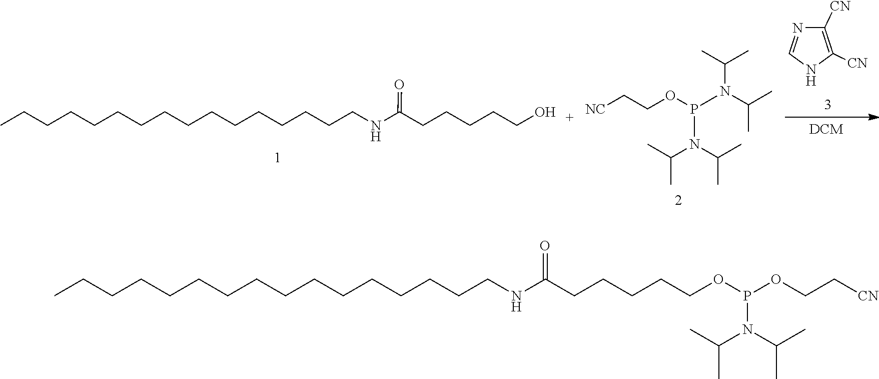 [SMILES: CCCCCCCCCCCCCCCCNC(=O)CCCCCO ; CC(C)N(C(C)C)P(OCCC#N)N(C(C)C)C(C)C ; N#Cc1nc[nH]c1C#N ; CCCCCCCCCCCCCCCCNC(=O)CCCCCOP(OCCC#N)N(C(C)C)C(C)C]

To a mixture of compound 1 (200 mg) in 10 mL DCM was added compound 3 (33.2 mg) followed by compound 2 (339 mg) dropwise at RT. Then the mixture was stirred at RT overnight. 1 mL TEA was added followd by some Celite®. After removing solvent in vacuo, the residue was dry loaded on a 4 g column. Hexanes (2% TEA) to 50% EtOAc (2% TEA) in Hexanes (2% TEA) as gradient was used as the mobile phase. Product is a white wax solid, 95 mg, 30% yield. LC-MS: calculated [M+H]556.46, found 556.82.

### Synthesis of LP-232p

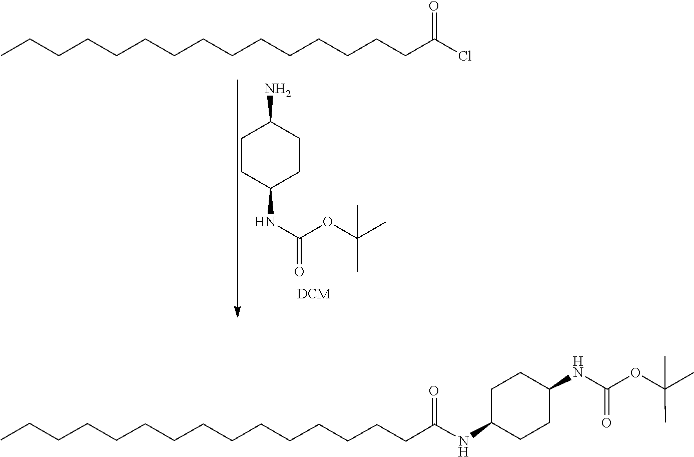

Palmitoyl chloride (100 mg) was stirred in a solution of cis-4-(boc-amino)cyclohexylamine (0.0819 g) in 5 mL DCM. After stirring the suspension overnight, water was added and the organics were extracted using DCM and dried over Na₂SO₄. After filtration, the solvent was concentrated to dryness and the crude product was purified by cloumn (Hexanes to EtOAc). Product is 52 mg, 31%.

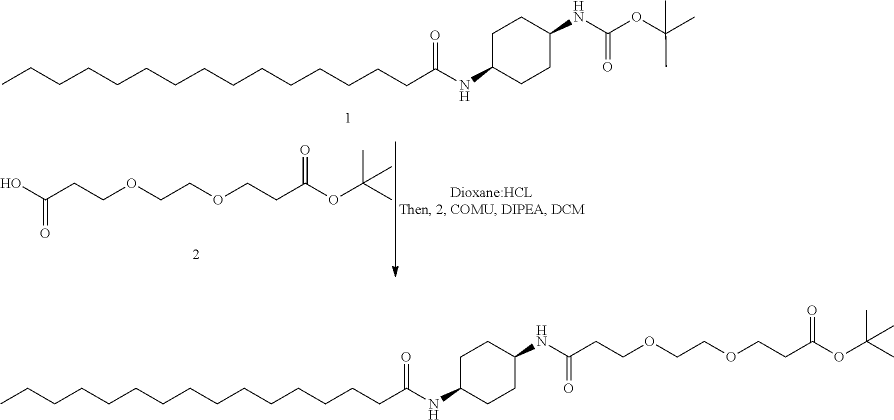

To 1 (0.0520 g) was added 2 mL Dioxane:HCl (4N) until boc deprotection was complete. After removing solvent in vacuo, to the residue was stirred in a solution of 2 (0.0316 g), DIPEA (0.0445 g) and COMU (0.0620 g) in 5 mL DCM. After stirring the suspension overnight, water was added and the organics were extracted using DCM and dried over Na₂SO₄. After filtration, the solvent was concentrated to dryness and the crude product was purified by column (DCM to 20% MeOH in DCM). Product was 45 mg. 65%.

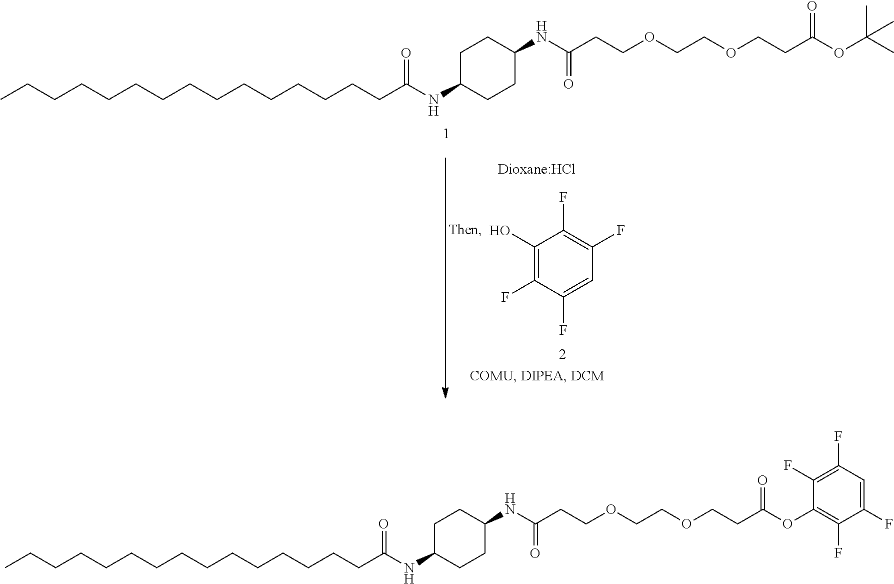

To 1 (0.0449 g) was added 2 mL Dioxane:HCl (4N) until OtBu deprotection was complete. After removing solvent in vacuo, to the residue was stirred in a solution of 2 (0.0217 g), DIPEA (0.039 mL) and COMU (0.0425 g) in 5 mL DCM. After stirring the suspension overnight, water was added and the organics were extracted using DCM and dried over Na₂SO₄. After filtration, the solvent was concentrated to dryness and the crude product was purified by column (DCM to 20% MeOH in DCM). Product was 30 mg, 58%.

### Synthesis of LP-233p

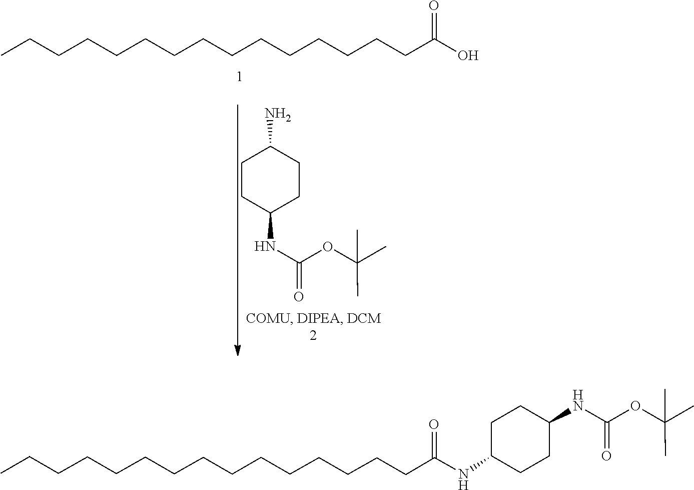 [SMILES: CCCCCCCCCCCCCCCC(=O)O ; CC(C)(C)OC(=O)N[C@H]1CC[C@H](N)CC1 ; CCCCCCCCCCCCCCCC(=O)N[C@H]1CC[C@H](NC(=O)OC(C)(C)C)CC1]

Palmitic acid 1 (0.100 g) was stirred in a solution of 2 (0.0693 g), COMU (0.166 g), DIPEA (0.16 mL), in 5 mL DCM. After stirring the suspension overnight (heated at 40° C.), water was added and the organics were extracted using DCM and dried over Na₂SO₄. After filtration, the solvent was concentrated to dryness and the crude product was purified by column (Hexanes to EtOAc). Product was 96 mg, 69%.

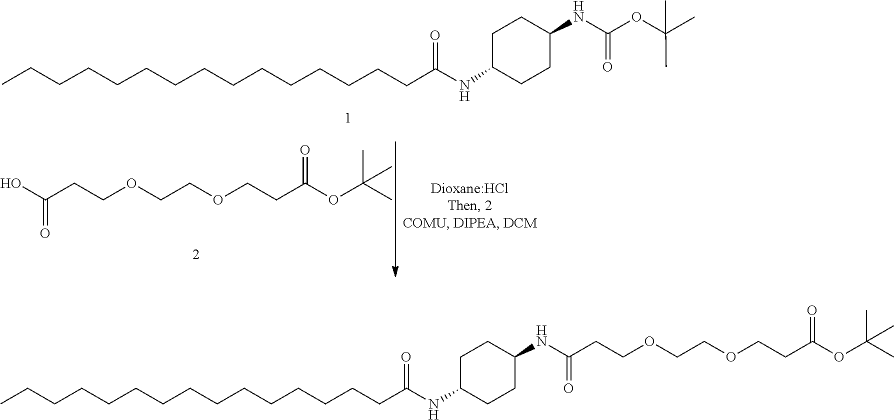

To 1 (0.0955 g) was added 2 mL Dioxane:HCl (4N) until boc deprotection was complete. After removing solvent in vacuo, to the residue was stirred in a solution of 2 (0.0581 g), DIPEA (0.11 mL) and COMU (0.114 g) in 5 mL DCM. After stirring the suspension overnight, water was added and the organics were extracted using DCM and dried over Na₂SO₄. After filtration, the solvent was concentrated to dryness and the crude product was purified by column (DCM to 20% MeOH in DCM). Product was 68 mg, 54%.

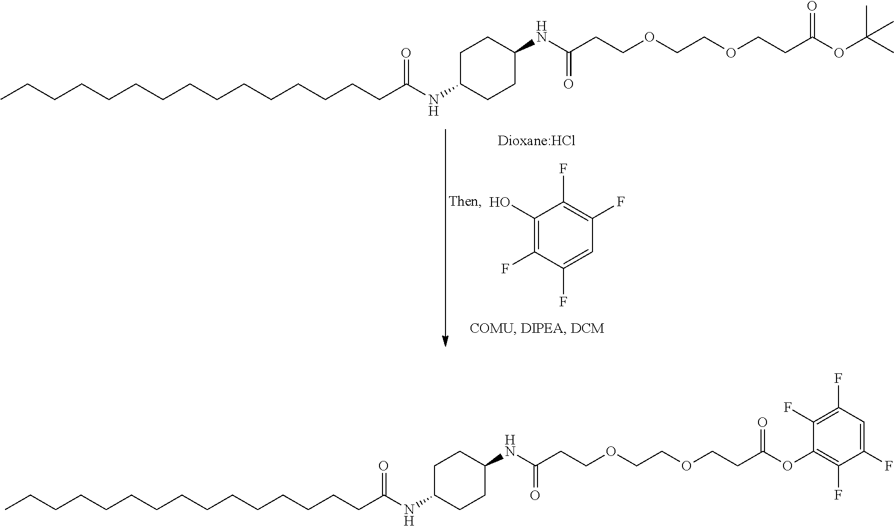

To 1 (0.068 g) was added 2 mL Dioxane:HCl (4N) until otBu deprotection was complete. After removing solvent in vacuo, to the residue was stirred in a solution of tetrafluorophenol (0.021 g), DIPEA (0.059 mL) and COMU (0.064 g) in 5 mL DCM. After stirring the suspension overnight, water was added and the organics were extracted using DCM and dried over Na₂SO₄. After filtration, the solvent was concentrated to dryness and the crude product was purified by column (DCM to 20% MeOH in DCM). Product was 22 mg, 28%.

### Synthesis of LP-242p

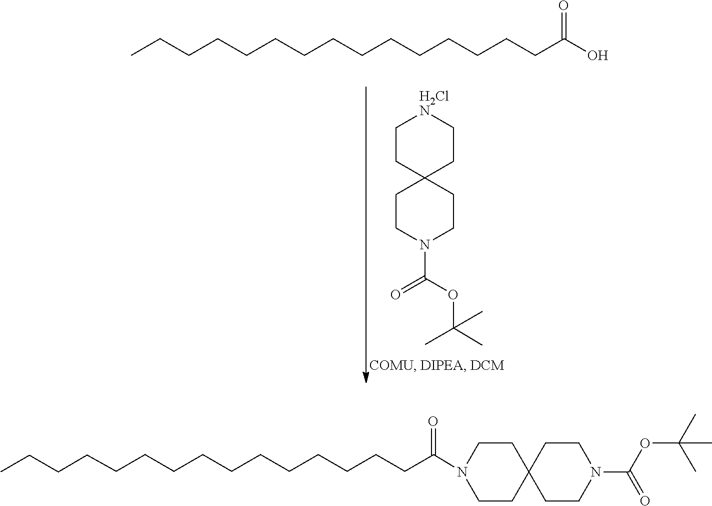

Palmitic acid (0.100 g) was stirred in a solution of tBu-3,9diazaspiro[5,5]undecane-3-carboxylate HCl (0.073 g), COMU (0.166 g), DIPEA (0.16 mL), in 5 mL DCM. After stirring the suspension overnight (heated at 40° C.), water was added and the organics were extracted using DCM and dried over Na₂SO₄. After filtration, the solvent was concentrated to dryness and the crude product was purified by flash chromatography.

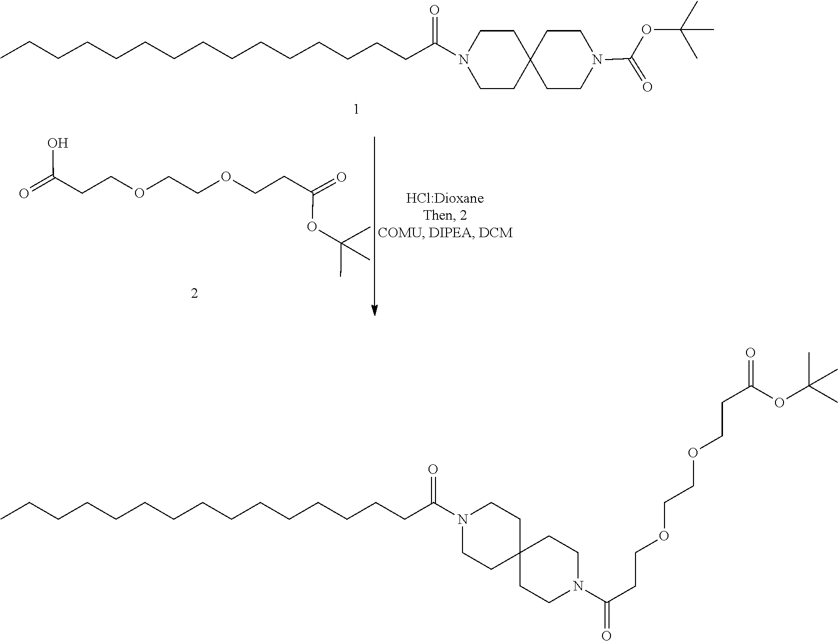

1 (0.017 g) was treated with HCl:Dioxane and after 1 h, crude reaction was dried in vacuo. To this was added a solution of 2 (0.0095 g), COMU (0.0186 g), DIPEA (0.0134 g), in 5 mL DCM. After stirring the suspension, water was added and the organics were extracted using DCM and dried over Na₂SO₄. After filtration, the solvent was concentrated to dryness and the crude product was purified by flash chromatography.

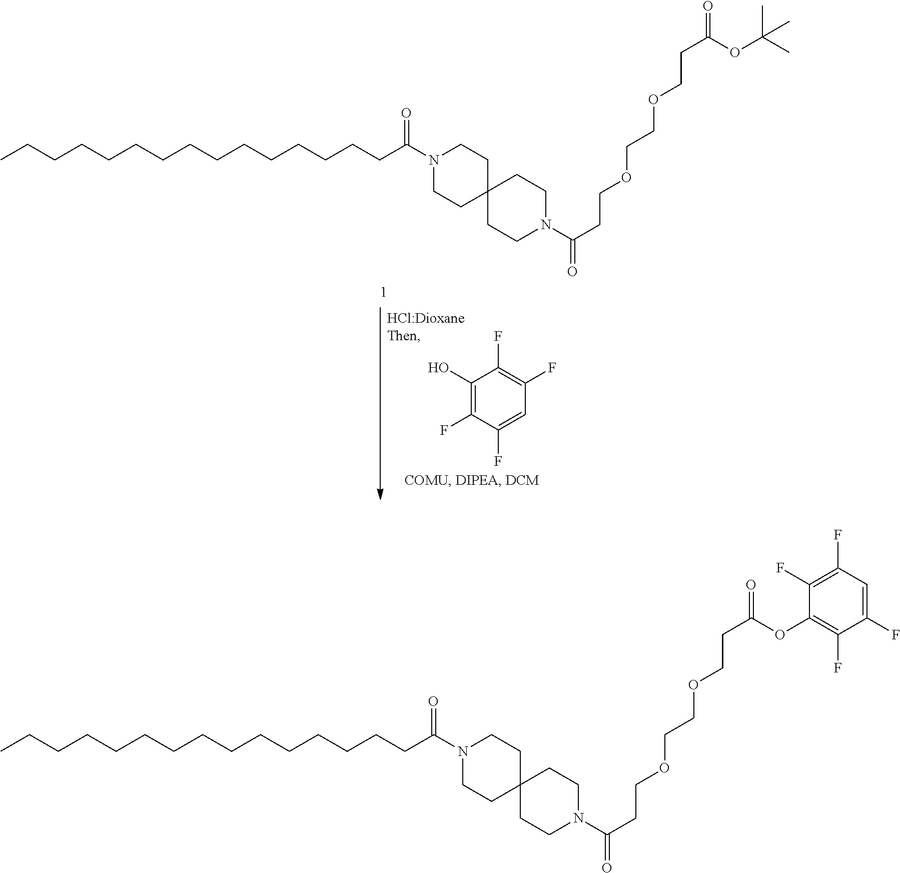

To 1 (0.121 G) was added 2 mL Dioxane:HCl (4N) until otBu deprotection was complete. After removing the solvent in vacuo, to crude 1 was stirred in a solution of tetrafluorophenol (0.0585 g), DIPEA (0.11 mL) and COMU (0.115 g) in 5 mL DCM. After stirring the suspension overnight, water was added and the organics were extracted using DCM and dried over Na₂SO₄. After filtration, the solvent was concentrated to dryness and the crude product was purified by flash chromatography.

### Synthesis of LP-243p

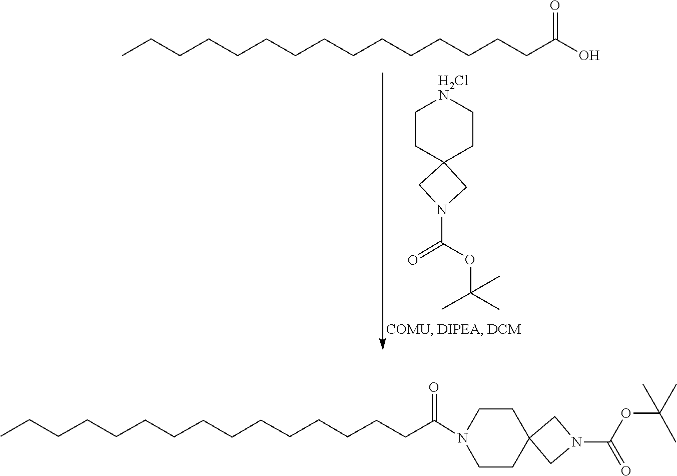

To palmitic acid (0.100 g) was stirred in a solution of tBu-3,9diazaspiro[5,5]undecane-3-carboxylate HCl (0.0732 g), COMU (0.166 g), DIPEA (0.161 mL), in 5 mL DCM. After stirring the suspension overnight (heated at 40° C.), water was added and the organics were extracted using DCM and dried over Na₂SO₄. After filtration, the solvent was concentrated to dryness and the crude product was purified by flash chromatography.

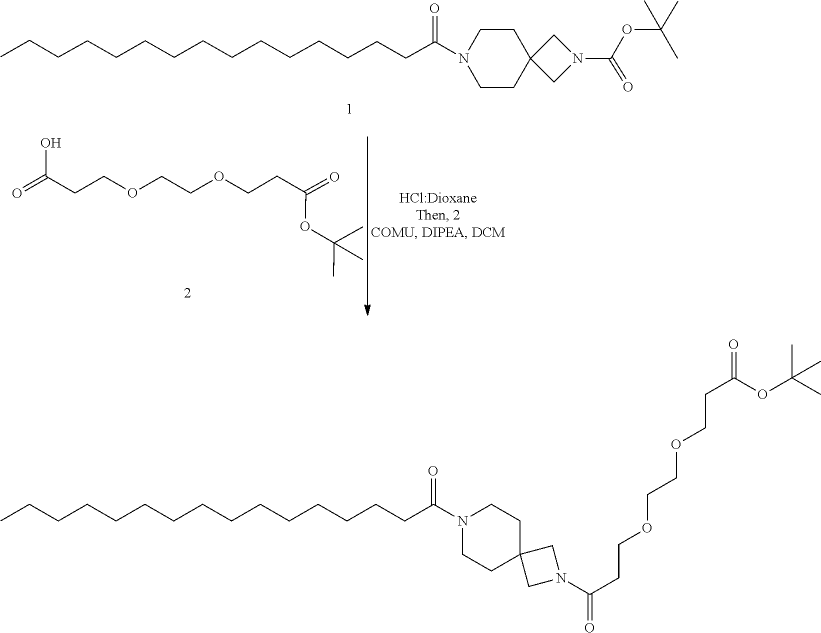

1 (0.0200 g) was treated with HCl:Dioxane and after 1 h, crude reaction was dried in vacuo. To this was added a solution of 2 (0.0119 g), COMU (0.0232 g), DIPEA (0.022 mL), in 5 mL DCM. After stirring the suspension, water was added and the organics were extracted using DCM and dried over Na₂SO₄. After filtration, the solvent was concentrated to dryness and the crude product was purified by flash chromatography.

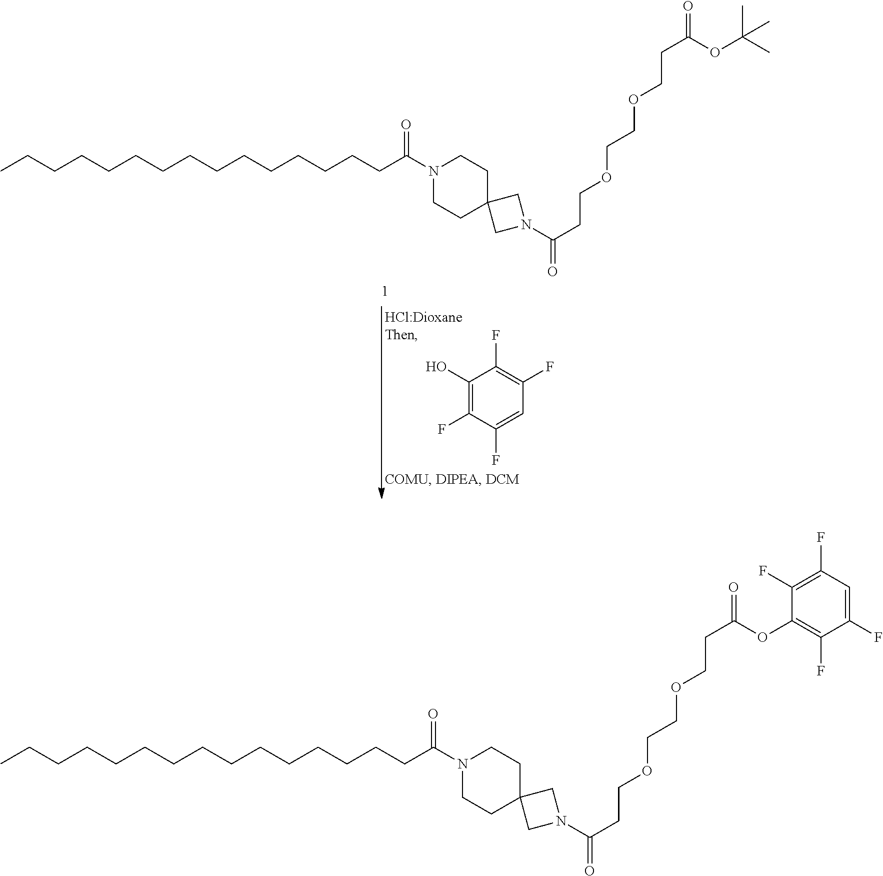

To 1 (0.121 g) was added 2 mL Dioxane:HCl (4N) until otBu deprotection was complete. After removing the solvent in vacuo, crude 1 was stirred in a solution of tetrafluorophenol (0.0363 g), DIPEA (0.104 mL) and COMU (0.112 g) in 5 mL DCM. After stirring the suspension overnight, water was added and the organics were extracted using DCM and dried over Na₂SO₄. After filtration, the solvent was concentrated to dryness and the crude product was purified by flash chromatography.

### Synthesis of LP-245p

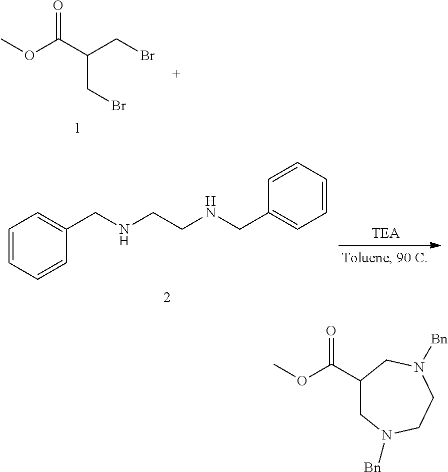

To a mixture of 1 (2.08 g) and 2 (1.98 g) in 50 mL toluene was added TEA at room temperature. The reaction mixture was stirred at 90° C. overnight. After cooling to room temperature, EtOAc and water were added for workup. Purification was on a 40 g column. Hexanes to 30% EtOAc in Hexanes as gradient was used to purify. Product was a light yellow oil, 1388 mg, 51%. LC-MS: calculated [M+H]339.21, found 339.62.

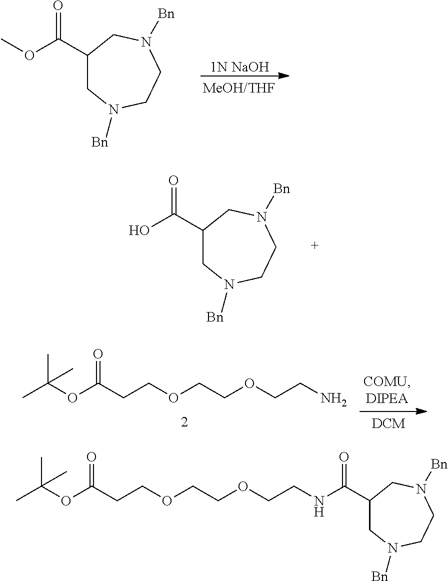

To a mixture of 1 (0.241 g) in MeOH/THF (4 mL/4 mL) was added 1N NaOH (6 mL) at room temperature. The reaction mixture was stirred at 60° C. for 1 h. After removing the organic solvent in vacuo, 1N HCl was added to adjust the mixture to pH ˜1. Then NaHCO₃ was added to adjust pH between 7-8. DCM was added to workup. After removing DCM in vacuo, the residue was placed on high vacuum for 2 h. The residue was diluted by DCM, then DIPEA (0.248 mL), COMU (0.336 g) and 2 (0.166 g) were added. The reaction mixture was sitrred at room temperature for 2 h. The reaction mixture was washed with 1N HCl, NaHCO₃ and brine. Purification was on a 12 g column. Hexanes to EtOAc as gradient was used to purify. Product was a brown oil, 285 mg, 74%. LC-MS: calculated [M+H]540.34, found 541.07.

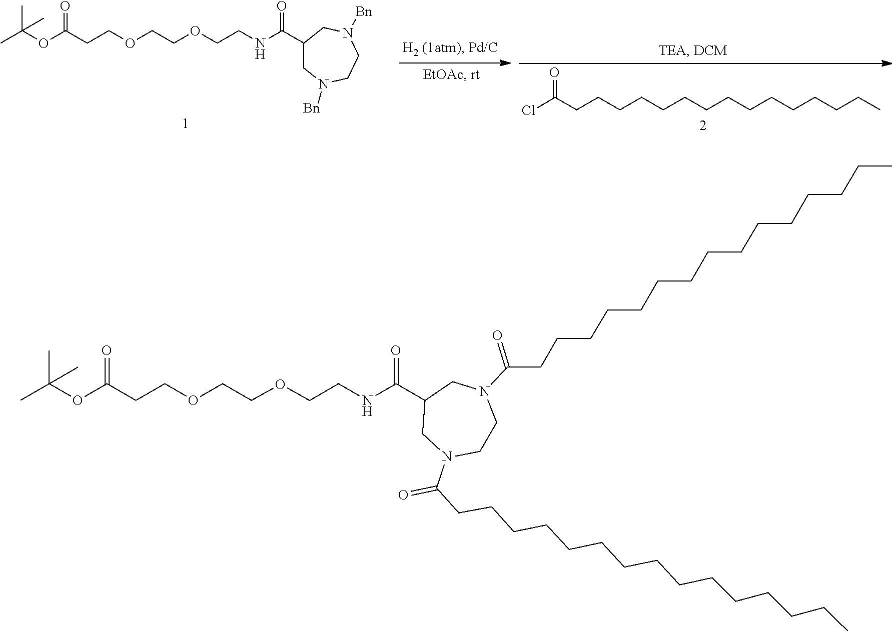 [SMILES: CC(C)(C)OC(=O)CCOCCOCCNC(=O)C1CN(Cc2ccccc2)CCN(Cc2ccccc2)C1 ; CCCCCCCCCCCCCCCC(=O)Cl ; CCCCCCCCCCCCCCCC(=O)N1CCN(C(=O)CCCCCCCCCCCCCCC)CC(C(=O)NCCOCCOCCC(=O)OC(C)(C)C)C1]

To a mixture of 1 (0.0740 g) and Pd/C in EtOAc was charged with H₂ (1 atm) at room temperature. The reaction mixture was stirred at room temperature for 4 h. The reaction mixture was filtered by a Celite® pad. After removing EtOAc in vacuo, the residue was under high vacuum for 1 h. The residue was dissolved in 3 mL DCM, 2 (0.166 mL) and TEA (0.115 mL) were added at room temperature. The mixture was stirred at room temperature for 2 h. Water was added for workup. Purification was on a 12 g column. DCM to 20% MeOH in DCM as gradient was used to purify. Product was a clear oil, 43 mg, 37%. LC-MS: calculated [M+H]836.71,

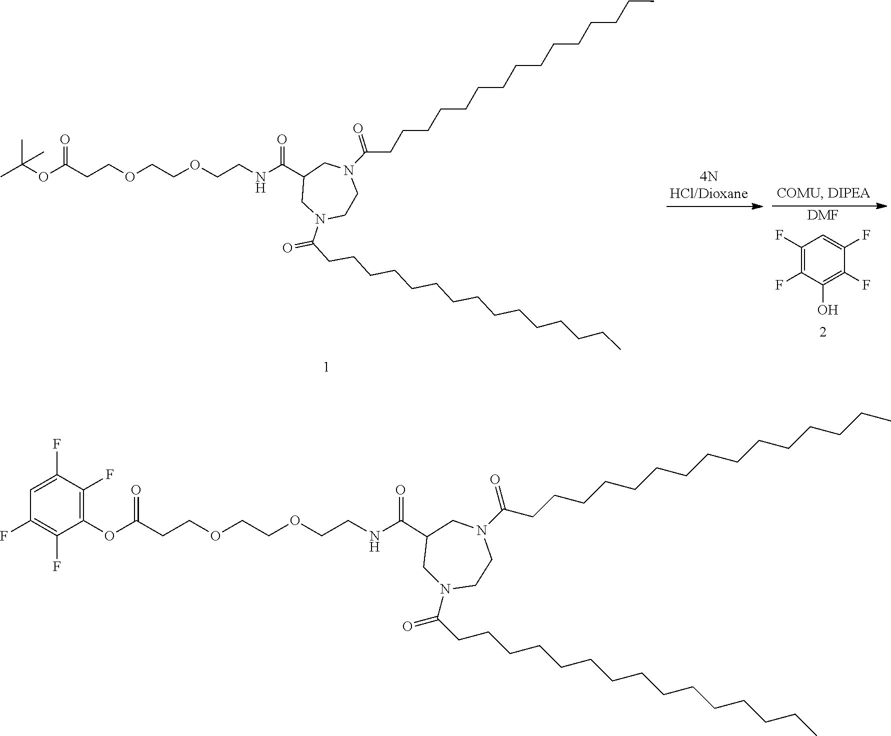

A solution of 1 (0.0430 g) in 4N HCl/Dioxane (3 mL) was stirred at room temperature overnight. After removing solvent in vacuo, the residue was placed under high vacuum for 3 h. The residue was dissolved in 3 mL DMF, then, DIPEA (0.027 g), COMU (0.0660 g) and 2 (0.017 g) were added. The mixture was stirred at room temperature for 2 h. After removing solvent in vacuo, the residue was loaded on a 4 g column. DCM to 20% MeOH in DCM as gradient was used to purify. Product was a light yellow oil, 34 mg, 37%. LC-MS: calculated [M+H]928.64, found 929.59.

### Synthesis of LP-249p

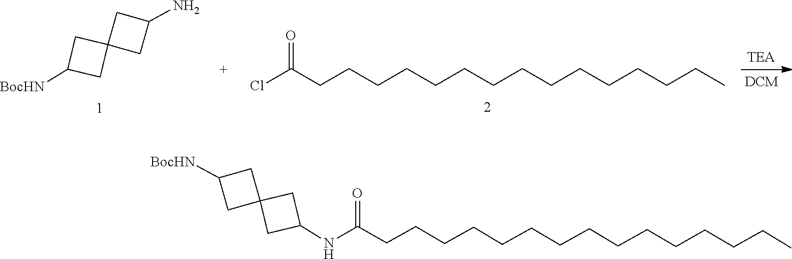 [SMILES: CC(C)(C)OC(=O)NC1CC2(CC(N)C2)C1 ; CCCCCCCCCCCCCCCC(=O)Cl ; CCCCCCCCCCCCCCCC(=O)NC1CC2(C1)CC(NC(=O)OC(C)(C)C)C2]

To a mixture of 1 (0.0600 g) and 2 (0.161 mL) in 4 mL DCM was added TEA (0.111 mL) at room temperature. The reaction mixture was stirred at room temperature for 2 h. Water was added for workup. Purification was on a 4 g column. Hexanes to EtOAc as gradient was used to purify. Product was a white solid, 74 mg, 60%. LC-MS: calculated [M+H]465.41, found 465.91.

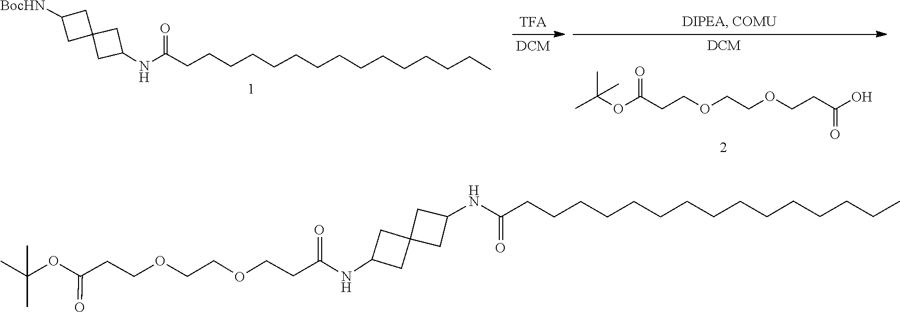 [SMILES: CCCCCCCCCCCCCCCC(=O)NC1CC2(C1)CC(NC(=O)OC(C)(C)C)C2 ; CC(C)(C)OC(=O)CCOCCOCCC(=O)O ; CCCCCCCCCCCCCCCC(=O)NC1CC2(C1)CC(NC(=O)CCOCCOCCC(=O)OC(C)(C)C)C2]

To a solution of 1 (0.0740 g) in DCM was added TFA (50% in DCM) at room temperature. The reaction mixture was stirred at room temperature for 0.5 h. The solvent was removed in vacuo, then the residue was under high vacuum for 2 h. The residue was dissolved in DMF, then 2 (0.0420 g), DIPEA (0.084 mL) and COMU (0.102 g) were added at room temperature. The mixture was stirred at room temperature for 2 h. The solvent was removed in vacuo. Purification was on a 12 g column. DCM to 20% MeOH in DCM as gradient was used to purify. Product was a white solid, 56 mg, 58%. LC-MS: calculated [M+H]609.48, found 610.29.

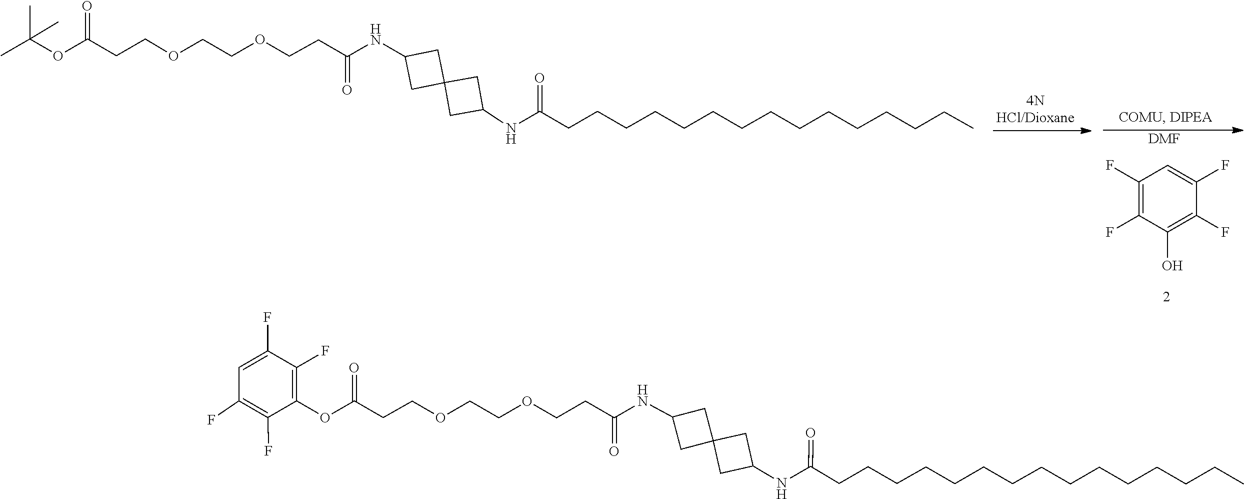

The solution of 1 (0.0560 g) in 4N HCl/Dioxane (3 mL) was stirred at room temperature overnight. After removing solvent in vacuo, the residue was under high vacuum for 3 h. The residue was dissolved in 2 mL DMF, then, DIPEA (0.048 mL), COMU (0.118 g) and 2 (0.031 g) were added. The mixture was stirred at room temperature for 2 h. After removing solvent in vacuo, the residue was loaded on a 4 g column. DCM to 20% MeOH in DCM as gradient was used to purify. Product was an off-white solid, 16 mg, 25%. LC-MS: calculated [M+H]701.42, found 702.20.

### Synthesis of LP-257p

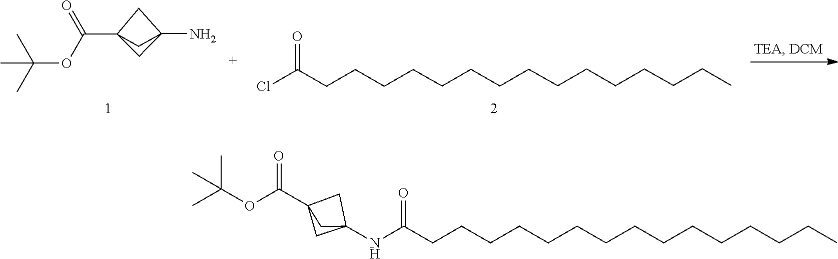 [SMILES: CC(C)(C)OC(=O)C12CC(N)(C1)C2 ; CCCCCCCCCCCCCCCC(=O)Cl ; CCCCCCCCCCCCCCCC(=O)NC12CC(C(=O)OC(C)(C)C)(C1)C2]

The solution of 1 (0.100 g) in 3 mL DCM was added 2 (0.331 mL) and TEA (0.304 mL) at room temperature. The reaction was stirred at room temperature for 1 h. EtOAc was added to dilute, then the mixture was washed with 1N HCl, NaHCO₃ and brine. After removing the solvent in vacuo, the residue was loaded on a 4 g column. Hexanes to EtOAc as gradient was used to purify. Product was a white solid, 134 mg, 58%. LC-MS: calculated [M+H]: 422.36, found 422.79.

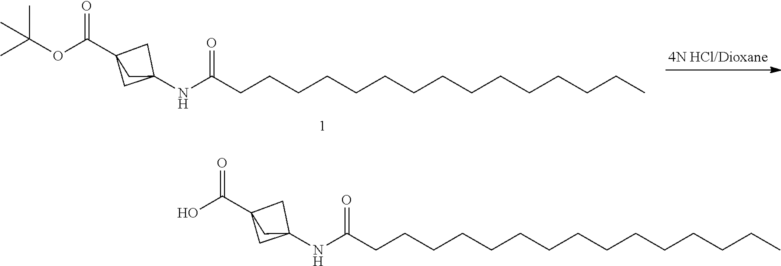 [SMILES: CCCCCCCCCCCCCCCC(=O)NC12CC(C(=O)OC(C)(C)C)(C1)C2 ; CCCCCCCCCCCCCCCC(=O)NC12CC(C(=O)O)(C1)C2]

The solution of 1 (0.134 g) in 4N HCl/Dioxane (8 mL) was stirred at room temperature overnight. After removing solvent in vacuo, the residue was under high vacuum for 3 h. Product was a white solid, 118 mg, which would be used for next step without further purification. LC-MS: calculated [M+H]366.30, found 366.62.

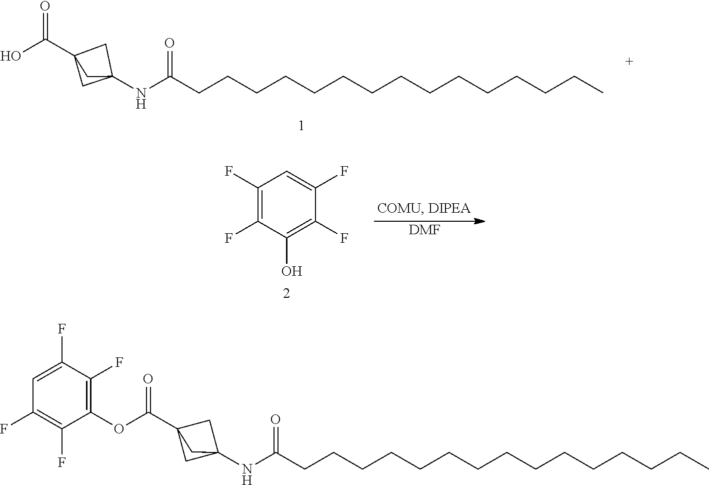

The solution of 1 (0.0490 g) in 3 mL DMF was added COMU (0.086 g), DIPEA (0.047 mL) and 2 (0.045 g) at room temperature. The mixture was stirred at room temperature for 1 h. The reaction mixture was diluted with EtOAc, then was washed with 1N HCl, NaHCO₃ and brine. After removing solvent in vacuo, the residue was loaded on a 4 g column. Hexanes to EtOAc as gradient was used to purify. Product was a white solid, 23 mg, 33%. LC-MS: calculated [M+H]514.29, found 514.79.

### Synthesis of LP-259p

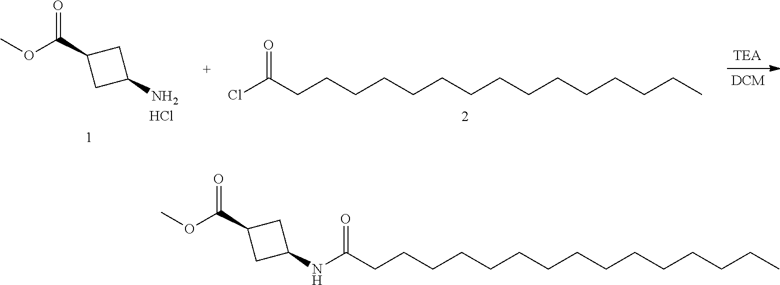

The solution of 1 (0.100 g) in 3 mL DCM was added 2 (0.366 mL) and TEA (0.337 mL) at room temperature. The reaction was stirred at room temperature for 1 h. The reaction mixture was loaded on a 12 g column. Hexanes to EtOAc as gradient was used to purify. Product was a white solid, 183 mg, 82%. LC-MS: calculated [M+H]: 368.32, found 368.60.

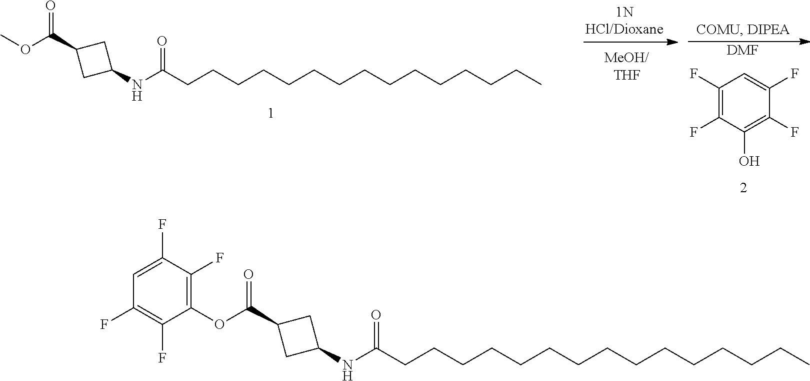

The solution of 1 (0.0900 g) in MeOH/THF/1N NaOH (3 mL/3 mL/3 mL) was stirred at 60° C. for 1 h. After cooling to room temperature, the MeOH/THF was removed in vacuo. The pH was adjusted to ˜1 with 1N HCl. EtOAc and water were added to workup. After removing EtOAc in vacuo, the residue was under high vacuum for 3 h. The residue was dissolved in 3 mL DMF, then COMU (0.136 g), DIPEA (0.085 mL) and 2 (0.053 g) were added at room temperature. The reaction was stirred at room temperature for 1 h. EtOAc was added to dilute the reaction mixture. The reaction mixture was washed with 1N HCl, NaHCO₃ and brine. After removing EtOAc in vacuo, the residue was loaded on a 12 g column. Hexanes to EtOAc as gradient was used to purify. Product was a white solid, 87 mg, 71%. LC-MS: calculated [M+H]: 502.29, found 502.72.

### Synthesis of LP-260p

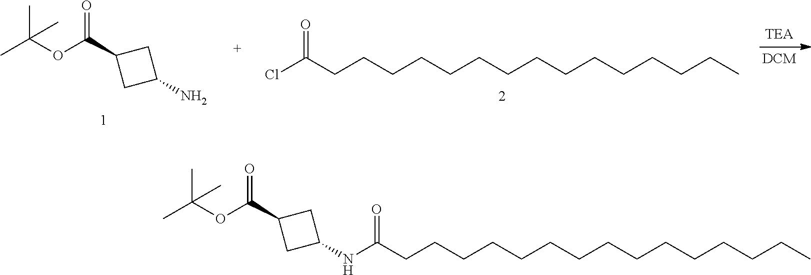 [SMILES: CC(C)(C)OC(=O)[C@H]1C[C@H](N)C1 ; CCCCCCCCCCCCCCCC(=O)Cl ; CCCCCCCCCCCCCCCC(=O)N[C@H]1C[C@H](C(=O)OC(C)(C)C)C1]

The solution of 1 (0.100 g) in DDC was added 2 (0.354 mL) and TEA (0.326 mL) at room temperature. The reaction was stirred at room temperature for 1 h. The reaction mixture was loaded on a 12 g column. Hexanes to EtOAc as gradient was used to purify. Product was a white solid, 208 mg, 87%. LC-MS: calculated [M+H]: 410.36, found 410.73.

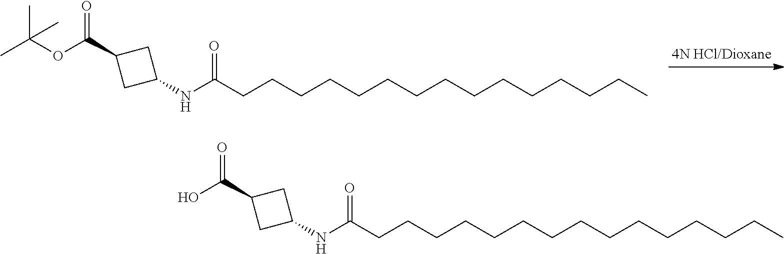 [SMILES: CCCCCCCCCCCCCCCC(=O)N[C@H]1C[C@H](C(=O)OC(C)(C)C)C1 ; CCCCCCCCCCCCCCCC(=O)N[C@H]1C[C@H](C(=O)O)C1]

The solution of 1 (0.208 g) in 4N HCl/Dioxane (8 mL) was stirred at room temperature overnight. After removing solvent in vacuo, the residue was under high vacuum for 3 h. Product was a white solid, 179 mg, which would be used for next step without further purification. LC-MS: calculated [M+H]354.30, found 354.65.

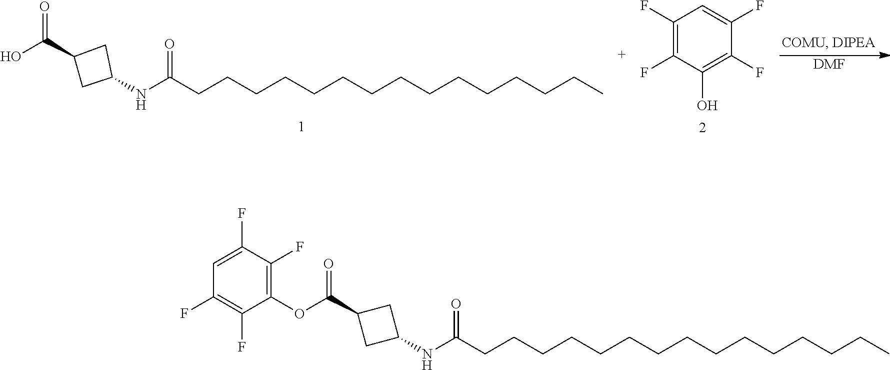

The solution of 1 (0.0760 g) in 3 mL DMF was added COMU (0.120 g), DIPEA (0.072 mL) and 2 (0.0460 g) at room temperature. The mixture was stirred at room temperature for 1 h. The reaction mixture was diluted with EtOAc, then was washed with 1N HCl, NaHCO₃ and brine. After removing solvent in vacuo, the residue was loaded on a 12 g column. Hexanes to EtOAc as gradient was used to purify. Product was a white solid, 55 mg, 51%. LC-MS: calculated [M+H]502.29, found 502.72.

### Synthesis of LP-262p

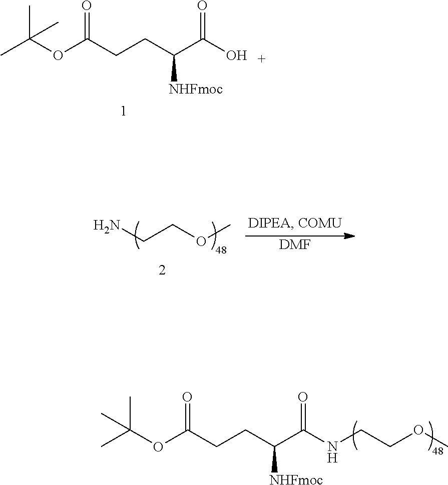

To a solution of 1 (0.0220 g) and 2 (0.100 g) and DIPEA (0.017 mL) in 2 mL DMF was added COMU (0.0240 g) at room temperature. The mixture was stirred at room temperature for 2 h. The reaction mixture was diluted with DCM. Then it was washed with 1N HCl, saturated NaHCO₃ and brine. Purification was perfomed on a 4 g column. DCM to 20% MeOH in DCM as gradient was used to purify. Product was a clear solid, 77 mg, 65%. LC-MS: calculated [M+2H]+H2O: 1294.76, found 1295.29; calculated [M+3H]+H2O: 869.51, found 869.45;calculated [M+4H]: 638.88, found 638.54.

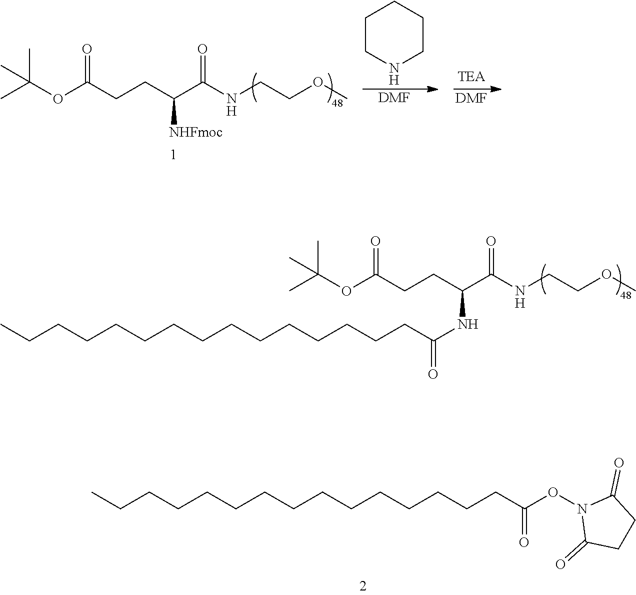

A solution of 1 (0.077 g) in DMF/piperidine (0.8 mL/0.2 mL) was stirred at room temperature for 1 h. After removing the solvent in vacuo, the residue was placed under high vacuum for 3 h. The residue was dissovled in 3 mL DMF, then 2 (0.016 g) and TEA (0.013 mL) were added at room temperature. The reaction was stirred at room temperature for 1.5 h. After removing the solvent in vacuo, the residue was loaded on a 4 g column. DCM to 20% MeOH in DCM as gradient was used to purify. Product was a white solid, 61 mg, 78%. LC-MS: calculated [M+2H]+H2O: 1302.84, found 1303.81; calculated [M+4H]: 642.92, found 642.62.

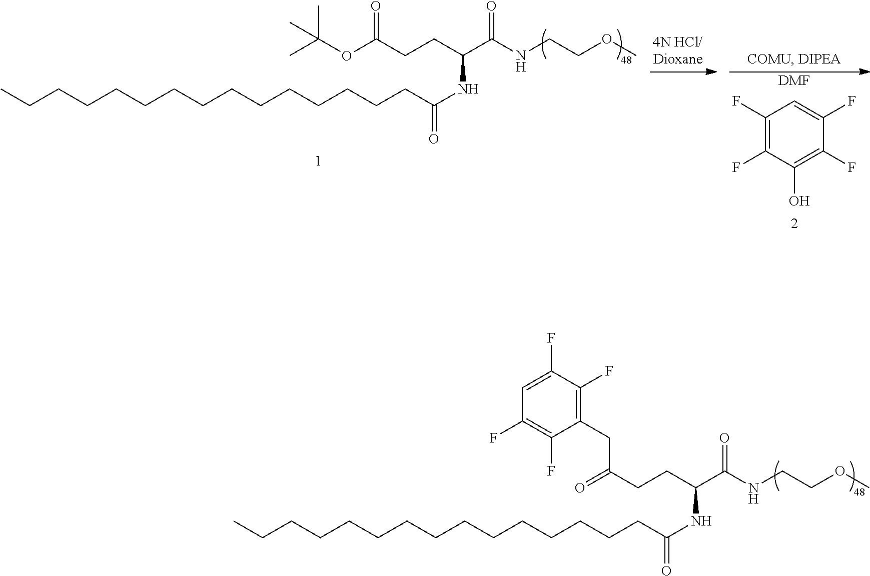

The solution of 1 (0.0610 g) in 4N HCl/Dioxane (5 mL) was stirred at room temperature overnight. After removing the solvent in vacuo, the residue was placed under high vacuum for 3 h. The residue was dissovled in 3 mL DMF, then COMU (0.0152 g), DIPEA (0.009 mL) and 2 (0.0060 g) were added at room temperature. The reaction was stirred at room temperature for 1.5 h. After removing the solvent in vacuo, the residue was loaded on a 4 g column. DCM to 20% MeOH in DCM as gradient was used to purify. Product was a white solid, 13 mg, 21%. LC-MS: calculated [M+2H]+H2O: 1348.80, found 1348.94; calculated [M+3H]+H2O: 905.54, found 905.09.

### Synthesis of LP-269p

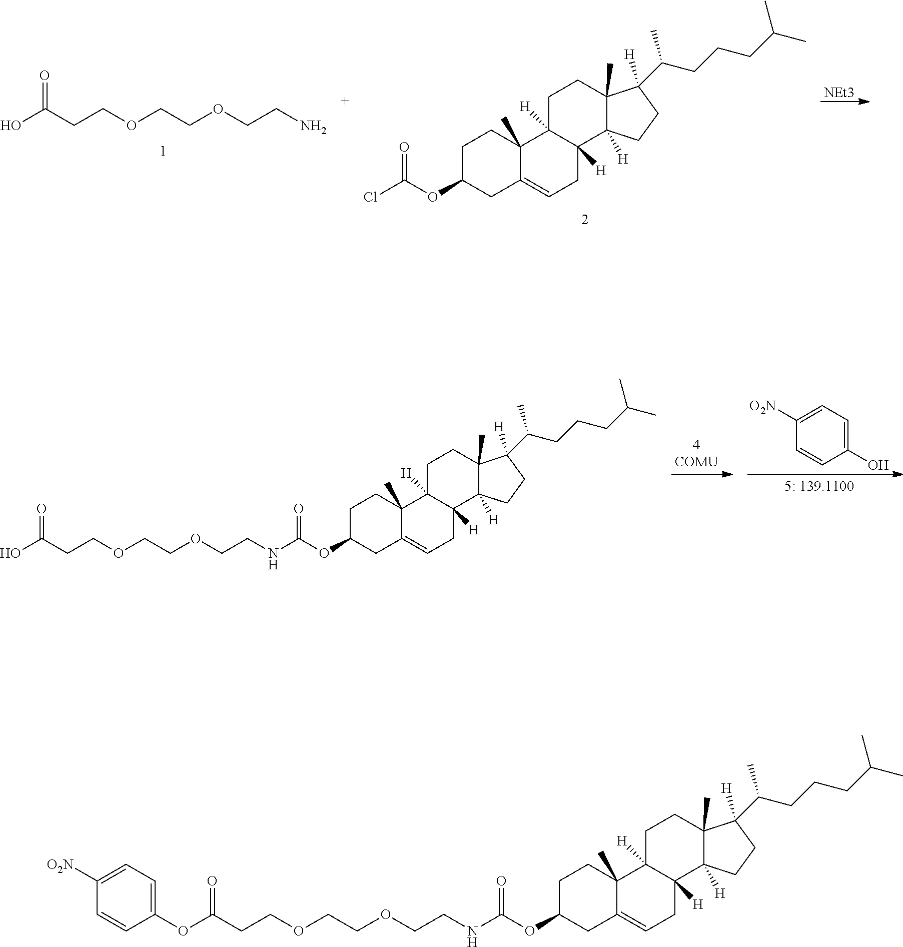 [SMILES: NCCOCCOCCC(=O)O ; CC(C)CCC[C@@H](C)[C@H]1CC[C@H]2[C@@H]3CC=C4C[C@@H](OC(=O)Cl)CC[C@]4(C)[C@H]3CC[C@]12C ; CC(C)CCC[C@@H](C)[C@H]1CC[C@H]2[C@@H]3CC=C4C[C@@H](OC(=O)NCCOCCOCCC(=O)O)CC[C@]4(C)[C@H]3CC[C@]12C ; O=[N+]([O-])c1ccc(O)cc1 ; "NEt3" ; CC(C)CCC[C@@H](C)[C@H]1CC[C@H]2[C@@H]3CC=C4C[C@@H](OC(=O)NCCOCCOCCC(=O)Oc5ccc([N+](=O)[O-])cc5)CC[C@]4(C)[C@H]3CC[C@]12C]

To a solution of 1 (88.6 mg, 0.500 mmol, 1.0 eqv.) and 2 (93.7 mg, 0.600 mmol, 1.20 eqv.) in 20 mL DCM was added TEA (0.418 mL, 3.000 mmol, 6.0 eqv.) under ambient conditions. Reaction was stirred at r.t. for 3 hours followed by adding COMU (257 mg, 0.600 mmol, 1.20 eqv.) then 4-nitrophenol (166.1 mg, 1.000 mmol, 2.0 eqv.). The reaction was stirred at r.t. overnight. The reaction mixture was washed with 1N HCl, then brine. The mixture was then dried with Na₂SO₄ and concentrated. The residue was purified by CombiFlash® using silica gel as the stationary phase with a gradient of EA to Hex 0-100%. 72 mg product was obtained (19% yield).

### Synthesis of LP-273p

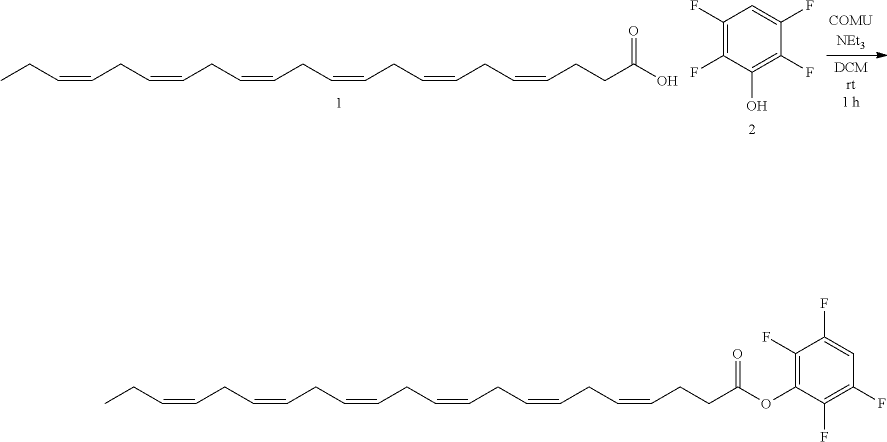

To a solution of compound 1 (0.200 g), NEt₃ (0.255 mL), and COMU (0.261 g) in DCM was added 2 (0.152 g) under ambient conditions. The reaction was stirred until full conversion was observed by LC-MS. The reaction mixture was directly concentrated for isolation. The residue was purified by CombiFlash® via DCM liquid-load onto a 12-g column with a gradient hexanes to 100% EtOAc, in which product eluted at 28% B. The product was concentrated under vacuum to provide a clear and lightly yellow oil. MS m/z: calculated [M+H]+ 477.23 m/z, observed 477.52 m/z.

### Synthesis of LP-274p

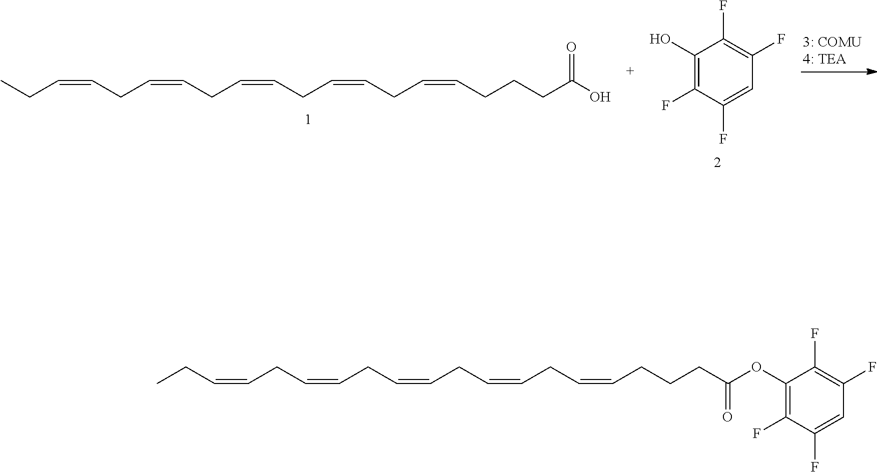 [SMILES: CC/C=C\C/C=C\C/C=C\C/C=C\C/C=C\CCCC(=O)O ; Oc1c(F)c(F)cc(F)c1F ; CC/C=C\C/C=C\C/C=C\C/C=C\C/C=C\CCCC(=O)Oc1c(F)c(F)cc(F)c1F]

To a solution of EPA 1 (60.5 mg, 0.200 mmol, 1 eqv.) and 2 (36.5 mg, 0.220 mmol, 1.10 eqv.) in 20 mL DCM was added COMU (94.2 mg, 0.220 mmol, 1.10 eqv.) and then TEA (0.084 mL, 0.600 mmol, 3.0 eqv.) under ambient conditions. The reaction was stirred until full conversion was observed by LC-MS. The reaction mixture was washed with 1N HCl, then brine. The mixture was then dried with Na₂SO₄ and concentrated. The reaction mixture was purified by CombiFlash® using silica gel as the stationary phase with a gradient of EA to Hex 0-50%. 69 mg product was obtained (76% yield).

### Synthesis of LP-283p

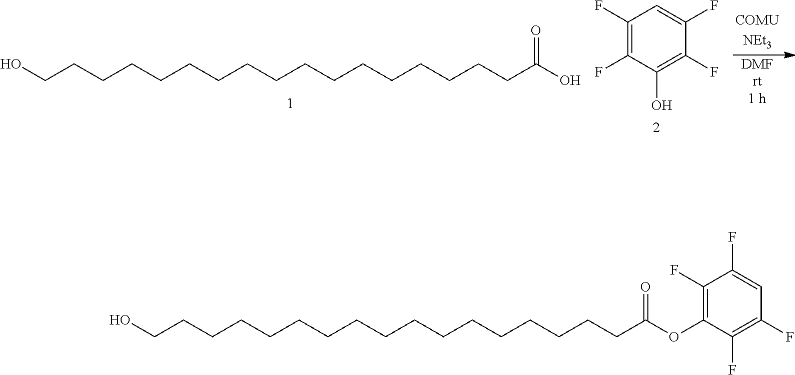

To a solution of compound 1 (49 mg), NEt₃ (0.068 mL), and COMU (76.8 mg) in DMF was added compound 2 (29.8 mg) under ambient conditions. The reaction was stirred until full conversion was observed by LC-MS. Conversion was not able to be clearly observed by LC-MS, and instead, reaction was allowed to stir for 30 min. until bright yellow color (before the addition of compound 2) transitioned to a honey orange color and all material was observed to be mainly dissolved. The reaction mixture was washed with water, extracted with DCM, dried over Na₂SO₄, filtered, and concentrated under vacuum. The residue was purified by CombiFlash® via DCM liquid-load onto a 12-g column with a gradient hexanes to 100% EtOAc in which product eluted at 31% B. The product was concentrated under vacuum to provide a white solid residue and confirmed by 1H NMR in CDCl₃.

### Synthesis of LP-286p

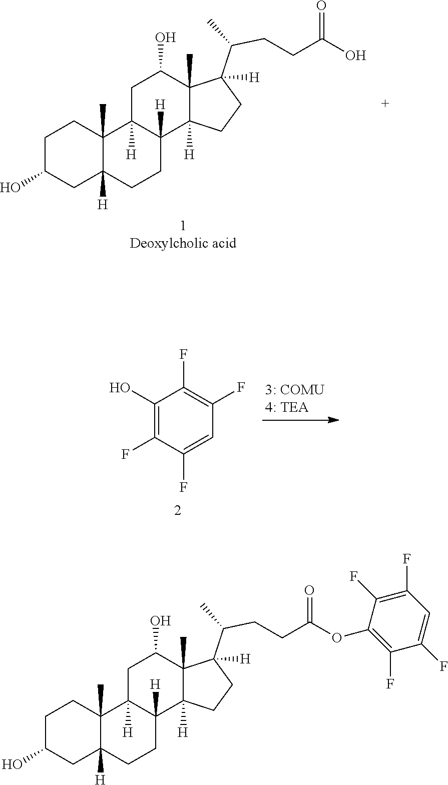

To a solution of 1 (78.5 mg, 0.200 mmol, 1 eqv.) and 2 (36.5 mg, 0.220 mmol, 1.10 eqv.) in 20 mL DCM was added COMU (94.2 mg, 0.220 mmol, 1.10 eqv.) and then TEA (0.084 mL, 0.600 mmol, 3.0 eqv.) under ambient conditions. The reaction was stirred until full conversion was observed by LC-MS. The reaction mixture was washed with 1N HCl, then brine. The mixture was dried with Na₂SO₄ and concentrated. The reaction mixture was purified by CombiFlash® using silica gel as the stationary phase with a gradient of EA to Hex 0-50%. 69 mg product was obtained (57% yield).

### Synthesis of LP-287p

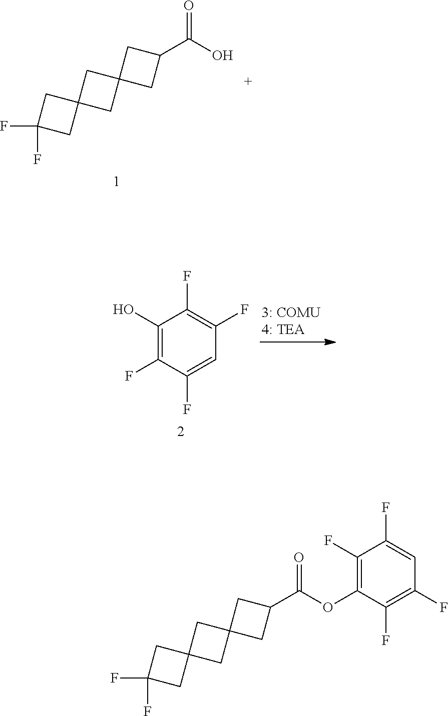 [SMILES: O=C(O)C1CC2(C1)CC1(CC(F)(F)C1)C2 ; Oc1c(F)c(F)cc(F)c1F ; O=C(Oc1c(F)c(F)cc(F)c1F)C1CC2(C1)CC1(CC(F)(F)C1)C2]

To a solution of 1 (43.3 mg, 0.200 mmol, 1 eqv.) and 2 (36.5 mg, 0.220 mmol, 1.10 eqv.) in 20 mL DCM was added COMU (94.2 mg, 0.220 mmol, 1.10 eqv.) and then TEA (0.084 mL, 0.600 mmol, 3.0 eqv.) under ambient conditions. The reaction was stirred until full conversion was observed by LC-MS. The reaction mixture was washed with 1N HCl, then brine. The mixture was dried with Na₂SO₄ and concentrated. The reaction mixture was purified by CombiFlash® using silica gel as the stationary phase with a gradient of EA to Hex 0-50%. 52 mg product was obtained (71% yield).

### Synthesis of LP-290p

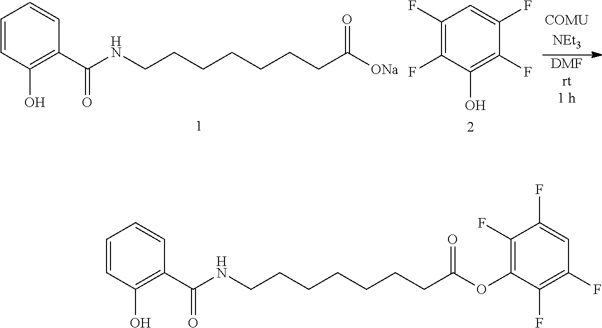

To a solution of compound 1 (0.0540 g), NEt₃ (0.075 mL), and COMU (0.084 g) in DMF was added 2 (0.0327 g) under ambient conditions. The reaction was stirred for 30 min. until bright yellow color (pre-addition of 2) transitioned to a honey orange color and all material was observed to be mostly dissolved. The reaction mixture was washed with water, extracted with DCM, dried over Na₂SO₄, filtered, and concentrated under vacuum. The residue was purified by CombiFlash® via DCM liquid-load onto a 12-g column with a gradient hexanes to 100% EtOAc in which product eluted at 31% B. The product was concentrated under vacuum to provide a white solid residue and confirmed by 1H NMR in CDCl₃. LC-MS: calculated [M+H]+ 428.14 m/z, observed 428.46 m/z.

### Synthesis of LP-293p

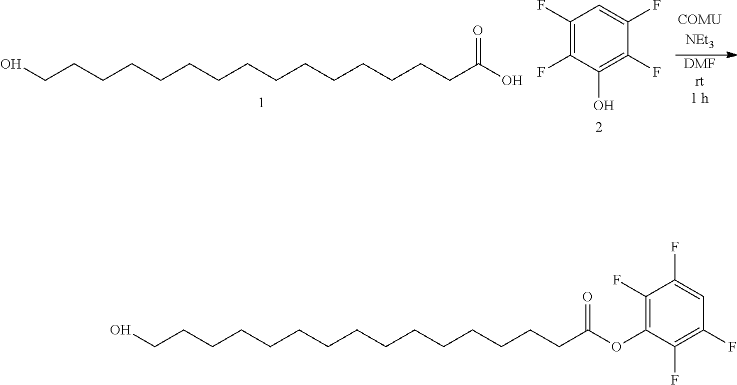

To a solution of compound 1 (73 mg), NEt₃ (0.112 mL), and COMU (126 mg) in DMF was added compound 2 (48.9 mg) under ambient conditions. The reaction was stirred until full conversion was observed by LC-MS. Conversion was not able to be clearly observed by LC-MS, and instead, reaction was allowed to stir for 30 min. until bright yellow color (before the addition of compound 2) transitioned to a honey orange color and all material was observed to be mainly dissolved. The reaction mixture was then washed with water, extracted with DCM, dried over Na₂SO₄, filtered, and concentrated under vacuum. The residue was purified by CombiFlash® via DCM liquid-load onto a 12-g column with a gradient hexanes to 100% EtOAc in which product eluted at 30% B. The product was concentrated under vacuum to provide a white solid residue and confirmed by 1H NMR in CDCl₃.

### Synthesis of LP-296p

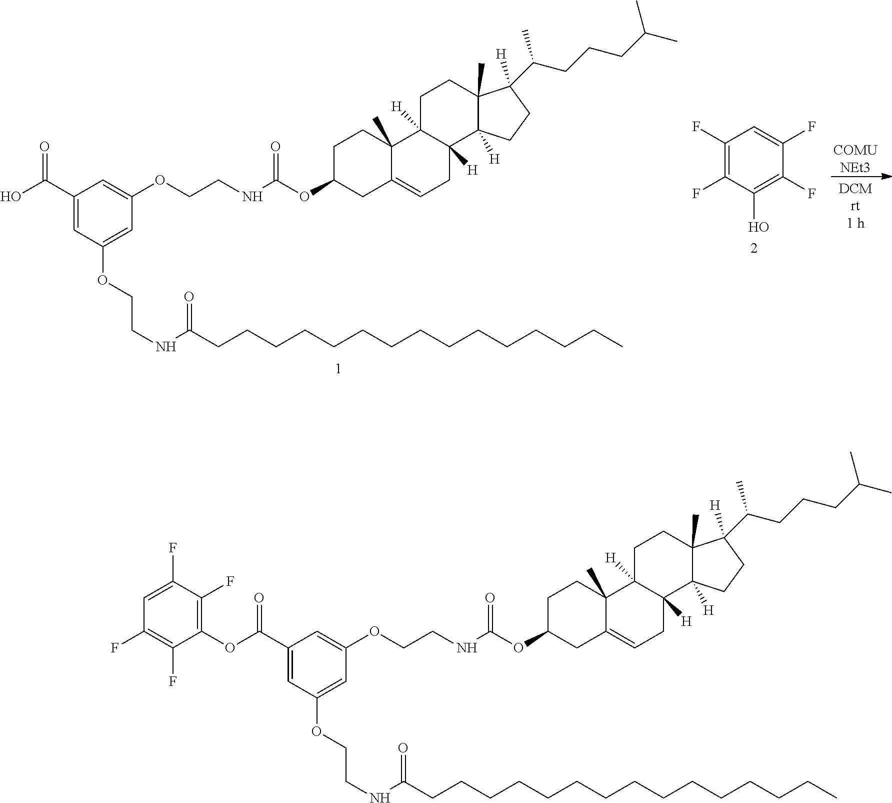

To a solution of compound 1 (0.0344 g), NEt₃ (0.0117 g), and COMU (0.0182 g) in DCM was added 2 (0.0071 g) under ambient conditions. The reaction was allowed to stir for 30 min. until bright yellow color (pre-addition of 2) transitioned to a honey orange color and all material was observed to be mainly dissolved. The reaction mixture was directly concentrated for isolation. The residue was purified by CombiFlash® via DCM liquid-load onto a 4-g column with a DCM to 20% MeOH/DCM (0% B to 20% B, to 40% B, to 50% B, then to 100% B), in which product eluted at 23% B. The product was concentrated under vacuum to provide a clear and colorless oil and confirmed by 1H NMR in CDCl₃. MS m/z: calculated [M+H]+ 1039.67 m/z; observed 1040.36, 671.78 m/z.

### Synthesis of LP-300p

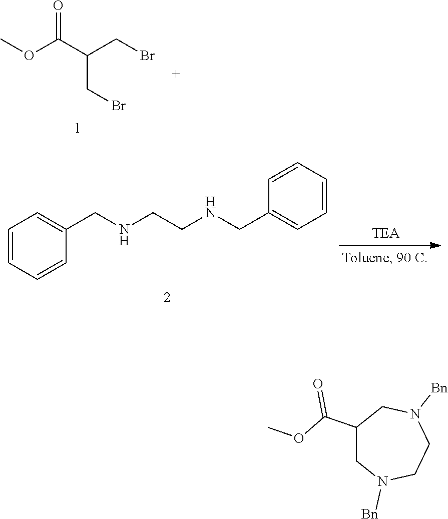

To a solution of 2 (5.29 g) in 100 mL toluene was added TEA (8.4 mL) at room temperature, then 1 (5.20 g) was added dropwise. The reaction mixture was stirred at 90° C. for 16 h. After cooling down to room temperature, EtOAc and water were added to workup. Purification was performed on a 120 g column. Hexanes to 30% EtOAc in Hexanes as gradient was used to purify. Product was a light yellow oil, 3658 mg, 54%. LC-MS: calculated [M+H]339.21, found 339.17.

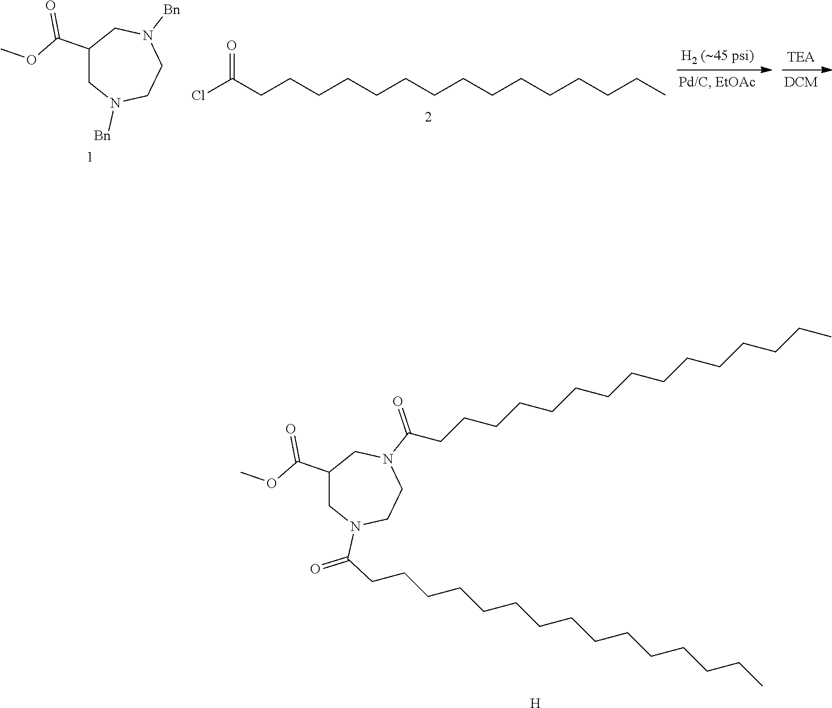

The mixture of 1 (0.113 g) and 10% Pd/C (0.0036 g) in 10 mL EtOAc was charged with H₂ (˜45 psi). The reaction mixture was stirred at room temperature for 4 h. After filtration, the solvent was removed in vacuo. Then the residue was placed under high vacuum for 1 h. The residue was dissolved in 10 mL DCM, then TEA (0.279 mL) and 2 (0.405 mL) were added at room temperature. The reaction mixture was stirred at room temperature for 1 h. Purification was performed on a 12 g column. Hexanes to 50% EtOAc in Hexanes as gradient was used to purify. Product was a white solid, 141 mg, 66%. LC-MS: calculated [M+H]635.57, found 635.95.

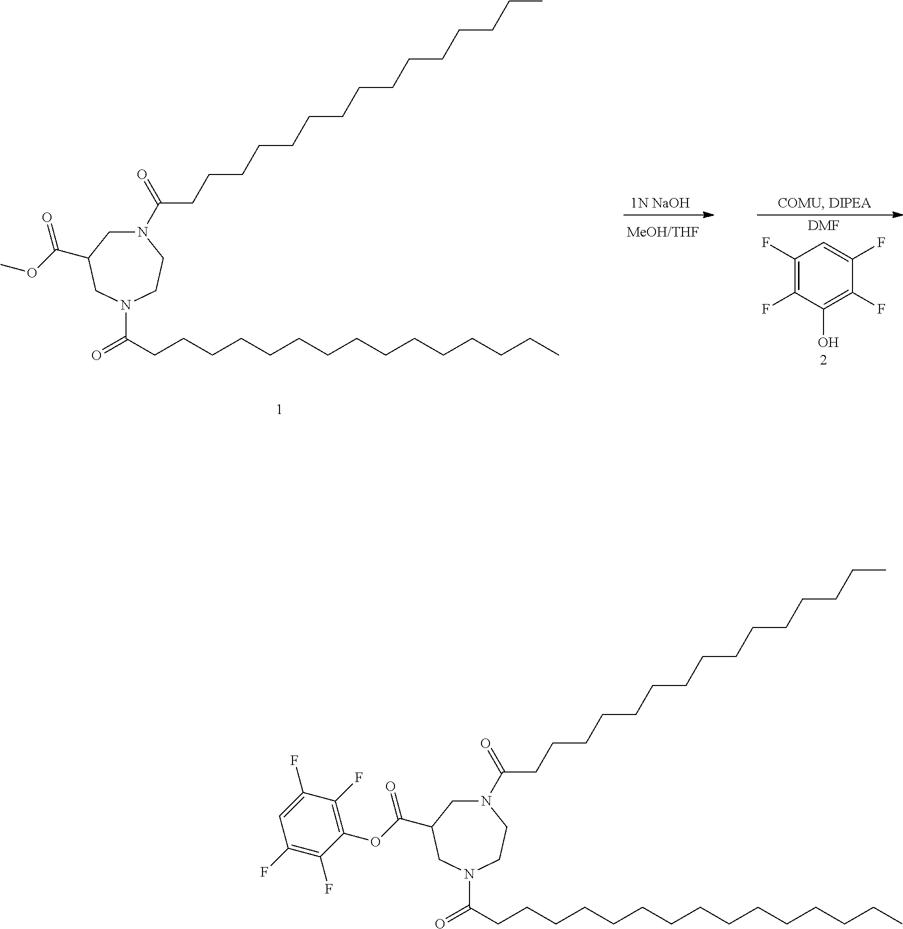

The solution of 1 (0.141 g) in MeOH/THF (3 mL/3 mL) was added 1N NaOH (3 mL) at room temperature. The mixture was stirred at room temperature for 2 h. After removing organic solvent in vacuo, the residue was acidified with conc. HCl to pH ˜1. EtOAc was added to extract the product. After removing solvent in vacuo, the residue was placed under high vacuum for 3 h. The residue was dissolved in DMF/DCM (5 mL/5 mL), then DIPEA (0.077 mL), COMU (0.143 g) and 2 (0.074 g) were added. The mixture was stirred at room temperature for 2 h. The reaction mixture was diluted with EtOAc, then was washed with 1N HCl and Brine. After removing solvent in vacuo, the residue was loaded on a 12 g column. Hexanes to 30% EtOAc in Hexanes as gradient was used to purify. Product was a white solid, 80 mg, 47%. LC-MS: calculated [M+H]769.55, found 769.98.

### Synthesis of LP-303p

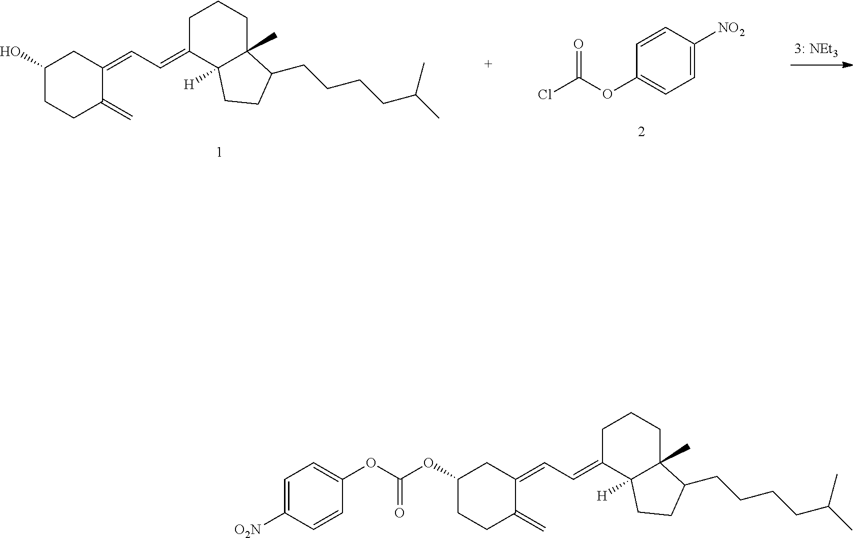 [SMILES: C=C1CC[C@H](O)C/C1=C/C=C1\CCC[C@]2(C)C(CCCCC(C)C)CC[C@@H]12 ; O=C(Cl)Oc1ccc([N+](=O)[O-])cc1 ; C=C1CC[C@H](OC(=O)Oc2ccc([N+](=O)[O-])cc2)C/C1=C/C=C1\CCC[C@]2(C)C(CCCCC(C)C)CC[C@@H]12]

To a solution of Vitamin D 1 (185 mg, 0.500 mmol, 1 eqv.) and 2 (111 mg, 0.550 mmol, 1.10 eqv.) in 30 mL DCM was added TEA (0.139 mL, 1.00 mmol, 2.0 eqv.) under ambient conditions. The reaction was stirred at r.t for 8 hours. The reaction mixture was washed with 1N HCl, then brine. The mixture was dried with Na₂SO₄ and concentrated. The residue was purified by CombiFlash® using silica gel as the stationary phase with a gradient of EA to Hex 0-100%, 95 mg product was obtained (35% yield).

### Synthesis of LP-304p

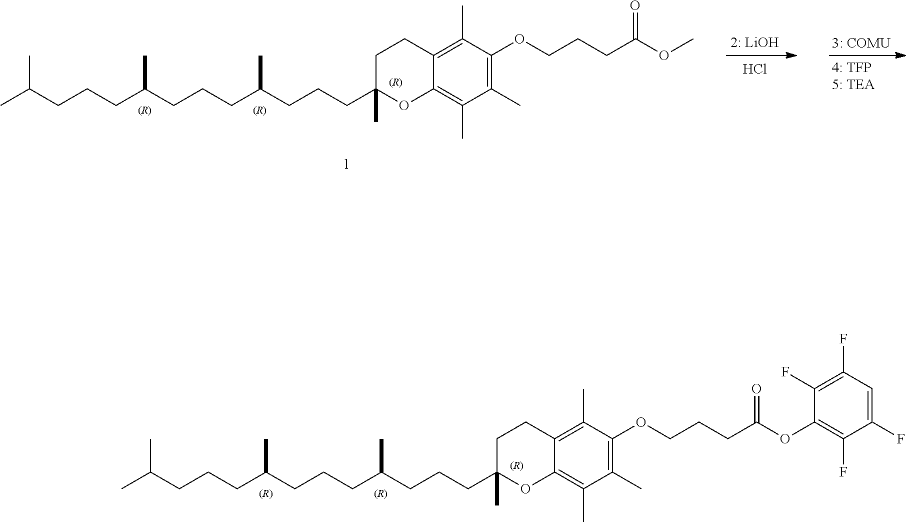

1 (200 mg, 0.377 mmol, 1.0 eqv.) was hydrolyzed with LiOH (151 mg, 3.77 mmol, 10.0 eqv.) in MeOH/TFH/H2O(1:1:1, 90 mL). After removing all organic solvent, the aqueous phase was acidified to pH=3 with 1N HCl. The reaction mixture extracted with ethyl acetate (100 mL×3). The organic phases were combined, dried with Na₂SO₄ and concentrated to get crude acid.

To a solution of above crude acid and tetrafluorophenol 4 (68.9 mg, 0.415 mmol, 1.10 eqv.) in 30 mL DCM was added COMU (194 mg, 0.453 mmol, 1.20 eqv.) and then TEA (0.158 mL, 1.13 mmol, 3.0 eqv.) under ambient conditions. The reaction was stirred until full conversion was observed by LC-MS. The reaction mixture was washed with 1N HCl, then brine. Dry with Na₂SO₄ and concentrated. The reaction mixture was purified by CombiFlash® using silica gel as the stationary phase with a gradient of EA to Hex 0-100%. 170 mg product was obtained (85% yield).

### Synthesis of LP-310p

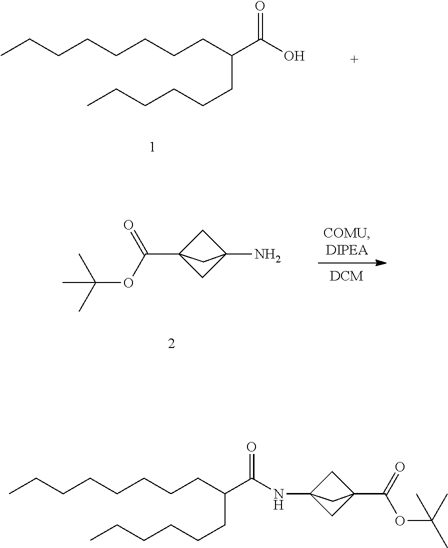 [SMILES: CCCCCCCCC(CCCCCC)C(=O)O ; CC(C)(C)OC(=O)C12CC(N)(C1)C2 ; CCCCCCCCC(CCCCCC)C(=O)NC12CC(C(=O)OC(C)(C)C)(C1)C2]

To the solution of 1 in DCM was added DIPEA (0.057 mL), COMU (0.077 g) and 2 (0.0300 g) at room temperature. After stirring at room temperature for 2 h, the reaction was quenched with 0.1N HCl. The organic layer was washed with brine. After removing the solvent, the residue was loaded on a 4 g column. Hexanes to 50% Hexanes in EtOAc as gradient was used to purify. Product was a white solid, 46 mg, 44%. LC-MS: calculated [M+H]422.36, found 422.61.

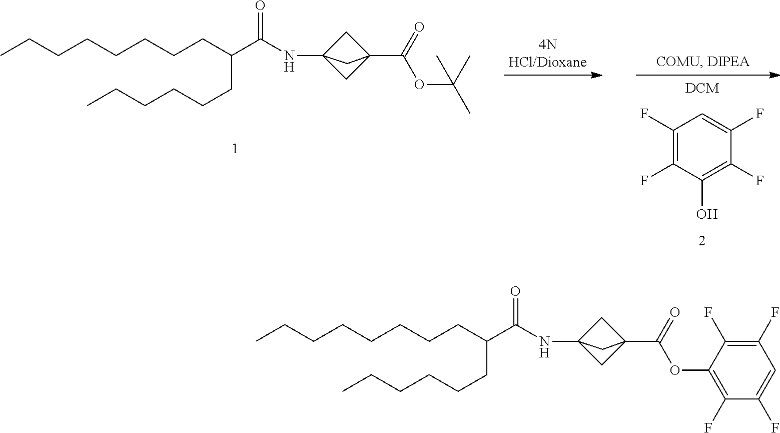

The solution of 1 (0.046 g) in 4N HCl/Dioxane (2 mL) was stirred at room temperature overnight. After removing the solvent in vacuo, the residue was placed under high vacuum for 3 h. Then the residue was dissolved in DCM at room temperature, then COMU (0.0700 g), DIPEA (0.038 mL) and 2 (0.036 g) were added at room temperature. After stirring at room temperature for 2 h, the solvent was removed in vacuo. The residue was loaded on a 4 g column. Hexanes to 50% Hexanes in EtOAc as gradient was used to purify. Product was a white solid, 21 mg, 38%. LC-MS: calculated [M+H]514.29, found 514.61.

### Synthesis of LP-383p

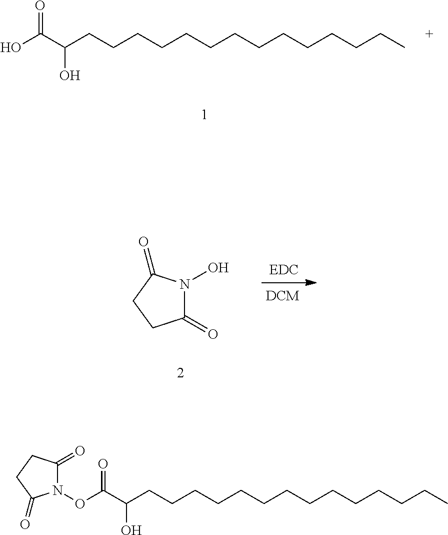 [SMILES: CCCCCCCCCCCCCCC(O)C(=O)O ; O=C1CCC(=O)N1O ; CCCCCCCCCCCCCCC(O)C(=O)ON1C(=O)CCC1=O]

To the solution of compound 1 (0.050 g) in 5 mL DCM was added compound 2 (0.023 g) and EDC (0.039 g) at room temperature. The mixture was stirred at room temperature for 1 h. After removing the solvent in vacuo, the residue was loaded on a 4 g column by dry method. Hexanes to 50% EtOAc in Hexanes was used to purify the product. Pdt is a white solid, yield, 29 mg. LC-MS: calculated [M+H+H2O]388.27, found 388.03.

### Synthesis of LP-409p

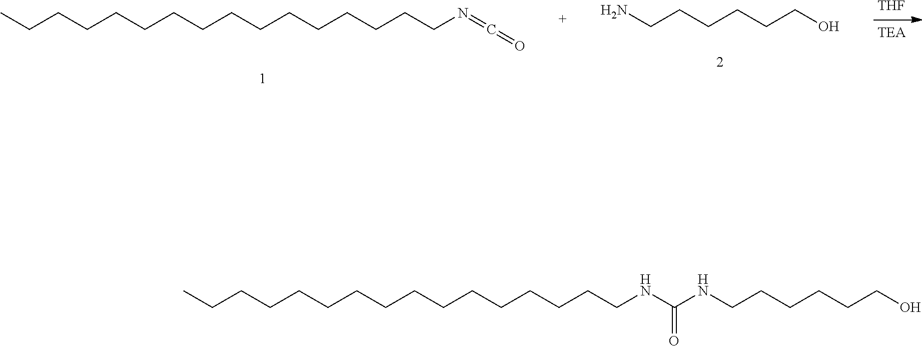 [SMILES: CCCCCCCCCCCCCCCCN=C=O ; NCCCCCCO ; "THF" ; CCCCCCCCCCCCCCCCNC(=O)NCCCCCCO]

Compounds 1 (1.40 g) and 2 (0.613 g) were dissolved in 100 mL THF, then TEA (2.01 mL) was added. The reaction was stirred at 60° C. until full conversion was confirmed via LC-MS (2-3 hours). The reaction was cooled down to room temperature. Product obtained as whilte precipitate, which was filtered and washed with Acetone (20 mL). Compound structure was verified using ¹H and ¹³P NMR.

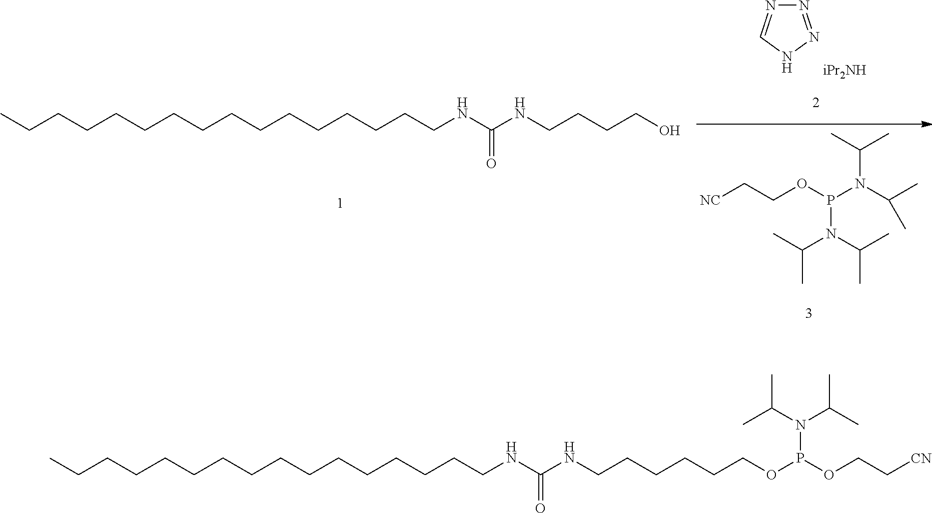 [SMILES: "iPr2NH" ; c1nnn[nH]1 ; CCCCCCCCCCCCCCCCNC(=O)NCCCCO ; CC(C)N(C(C)C)P(OCCC#N)N(C(C)C)C(C)C ; CCCCCCCCCCCCCCCCNC(=O)NCCCCCCOP(OCCC#N)N(C(C)C)C(C)C]

Compounds 1 (1.9 g), 2 (0.846 g) and 3 (2.98 g) were dissolved in 100 mL DCM then heated to 40° C. The reaction was stirred until the solution became clear. The reaction was cooled down to room temperature and stirred overnight. After removing all DCM, the product was dry loaded onto a 24 g column. Product was obtained as a white solid using 0-50% (EA/Hex, 1% TEA added) as mobile phase.

### Synthesis of LP-429p

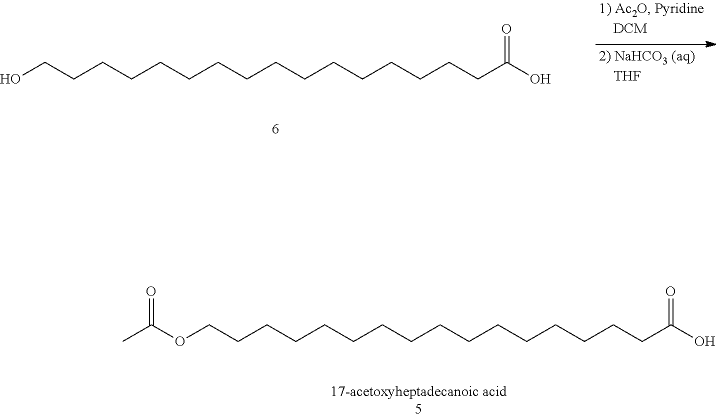 [SMILES: O=C(O)CCCCCCCCCCCCCCCCO ; CC(=O)OCCCCCCCCCCCCCCCCC(=O)O]

17-hydroxyhexadecanoic acid (6) (3.53 g, 12.3 mmol) was added to a 500 mL RBF. The flask was purged with nitrogen, then DCM (150 mL) was added followed by acetic anhydride (18.6 mL, 197 mmol) and pyridine (30.8 mL, 382 mmol). The reaction was stirred overnight. The reaction mixture was concentrated and azeotroped 3 times with toluene to remove residual pyridine, acetic acid, acetic anhydride. The residue was then stirred in 100 mL of a 1:1 THF/aq. NaHCO₃ mixture for 24 hours. About half of the THF was removed via rotary evaporator and the mixture was diluted with water and acidified with 3 M HCl until a pH of 1. The mixture became very foamy during the acidification. The product was collected by filtration and dried in vacuo to yield 3.22 g (80% yield) of compound 5 as a white solid. The product was not purified further.

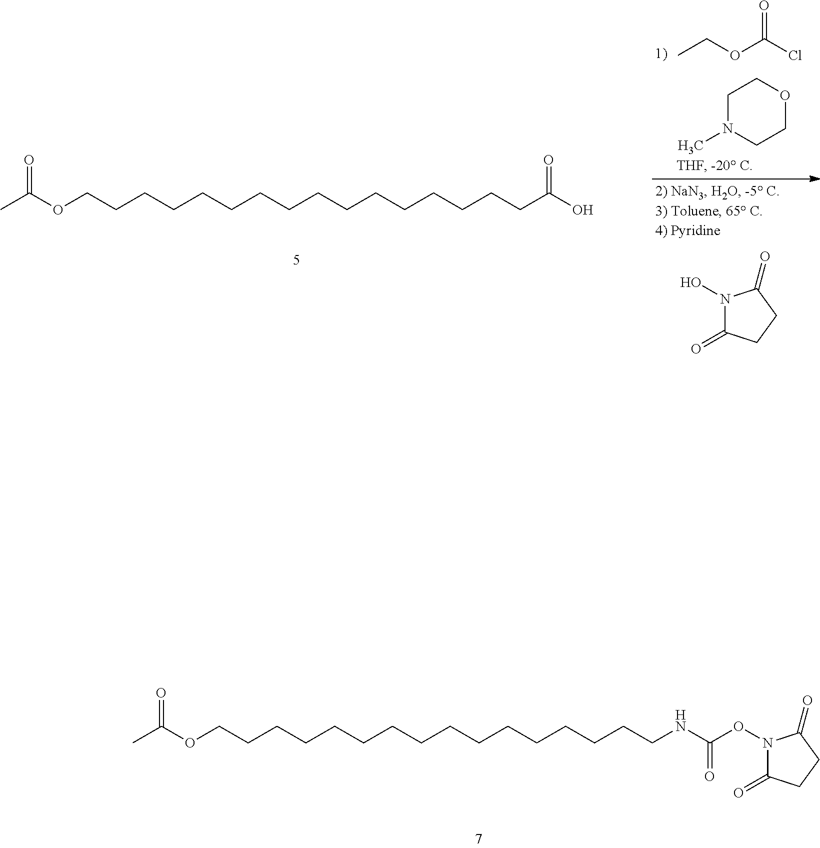

Compound 5 (3.47 g, 10.6 mmol) was dissolved in THF (55 mL) and cooled to −15 to −20° C. in a methanol/ice bath. Once cooled, N-methyl morpholine (1.4 mL, 12.7 mmol) and ethyl chloroformate (1.2 mL, 12.7 mmol) were added. The reaction was stirred at −15 for 30 minutes. After 30 minutes a solution of sodium azide (1.72 grams, 26.4 mmol) in water (6.6 mL) was added and the reaction was stirred for 30 minutes at −5°-0° C. in a water/salt/ice bath. The reaction mixture was diluted with EtOAc (20 mL) and water (20 mL). The layers were separated and the aqueous layer was extracted with EtOAc (2×50 mL), the combined organic layers were washed with water (50 mL), brine (50 mL), dried over sodium sulfate and concentrated to a white solid. Proton NMR showed no remaining starting material based on protons alpha to the carbonyl. The solid was dissolved in toluene (55 mL) and heated to 65° C. until gas evolution stopped (about 30 minutes). The reaction was cooled to room temperature and N-hydroxy succinimide (1.22 g, 10.5 mmol) was added followed by pyridine (0.85 mL, 10.5 mmol). Proton NMR indicated not all the isocyanate was consumed after 2 hours, additional 2 eq of N-hydroxy succinimide (2.43 g, 21.1 mmol) was added. The reaction was stirred overnight. No isocyanate remained by proton NMR after stirring overnight. The reaction mixture was concentrated, the resulting white powder was dissolved in EtOAc (100 mL) and poured into 300 mL hexanes. The percipitate was collected by filtration. Proton NMR of the product showed residual N-hydroxy succinimide. The product was dissolved in DCM and purified by silica gel chromatography 65:35 Hexanes:EtOAc to 0:100 Hexanes:EtOAc. Product began eluting at 50% EtOAc and dragged on the column. Fractions containing product were combined to yield 2.25 g (48% yield) of compound 7 as a white solid.

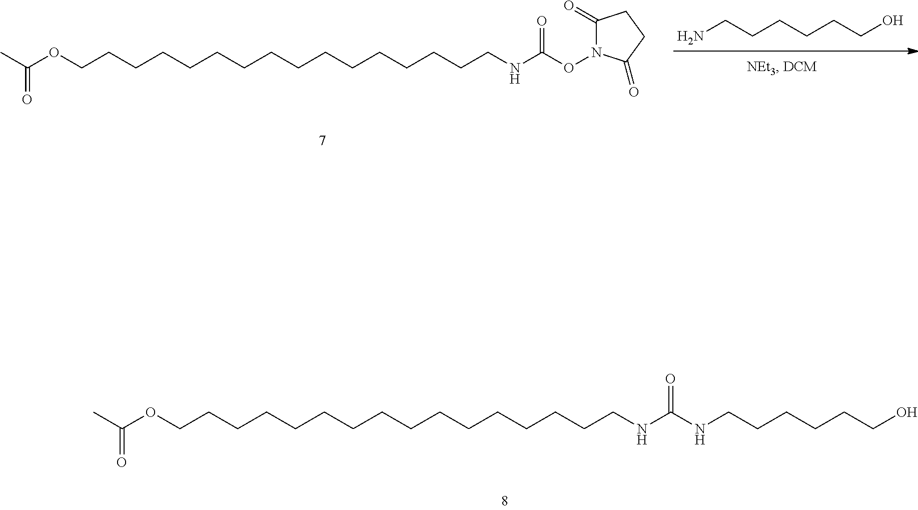 [SMILES: CC(=O)OCCCCCCCCCCCCCCCCNC(=O)ON1C(=O)CCC1=O ; NCCCCCCO ; CC(=O)OCCCCCCCCCCCCCCCCNC(=O)NCCCCCCO]

Compound 7 (1.00 g, 2.27 mmol) was added to a solution of 6-amino-1-hexanol (0.266 g, 2.27 mmol) and NEt₃ (0.95 mL, 6.81 mmol) in DCM (50 mL). A white ppt formed. No SM remained by LC-MS after 18 hours. The reaction was concentrated by rotary evaporator, te residue was dissolved in about 8 mL of ethyl acetate and was cool to −20° C. in a freezer. A precipitate formed and settled at the bottom of the flask. The EtOAc was decanted off twice and the precipitate was collected and dried under vacuum to yield 0.95 grams (94% yield) of compound 8 as a white powder.

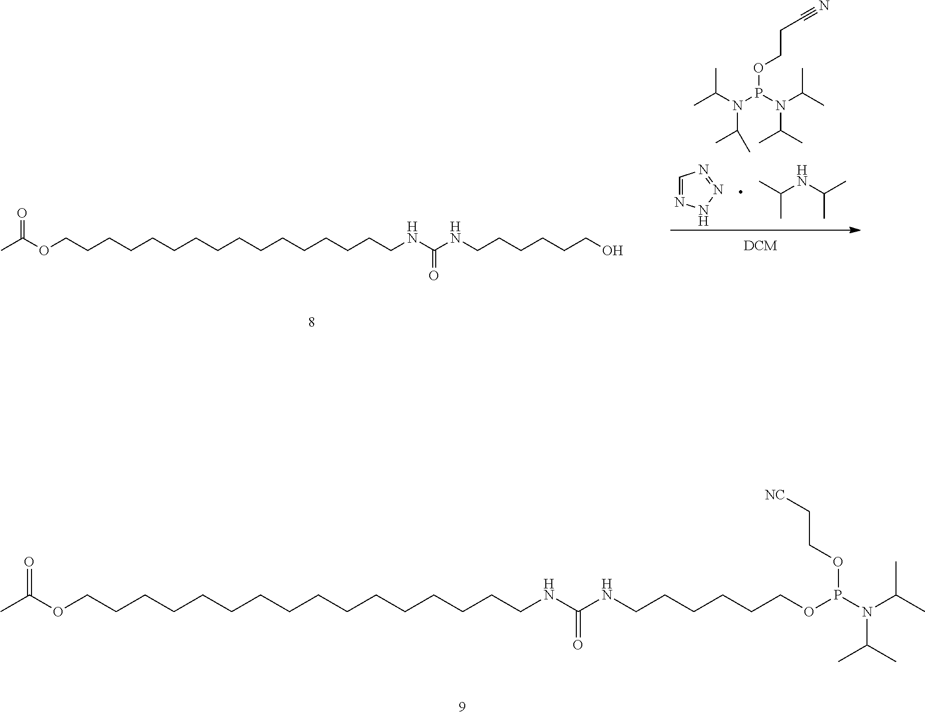

In a 100 mL RBF compound 8 (0.95 g, 2.14 mmol) was dried by 3 successive evaporations of toluene. Diisopropylammonium tetrazolide (0.146 g, 0.86 mmol) and 4 angstrom molecular sieves were added to the flask. The flask was purged and backfilled with nitrogen 3 times, and the solids were dissolved in DCM (50 mL). The mixture was stirred for 30 minutes. After 30 minutes 2-cyanoethyl N,N,N′,N′-tetraisopropylphosphorodiamidite (0.98 g, 3.25 mmol) was added and the reaction was stirred for 18 hours. After 18 hours, LC-MS indicated no starting alcohol remained. The reaction was transferred to a separatory funnel, washed with sat. aq. NaHCO₃ (2×40 mL), water (40 mL), brine (40 mL), dried over magnesium sulfate and concentrated to dryness. Hexanes was added to the flask and the residue was stirred in hexanes for 2 hours to yield a white precipitate. The white solid was collected by filtration, washed with hexanes (2×20 mL), and dried under vacuum to yield 1.2 grams (87% yield) of compound 9 as a white solid.

### Synthesis of LP-430p

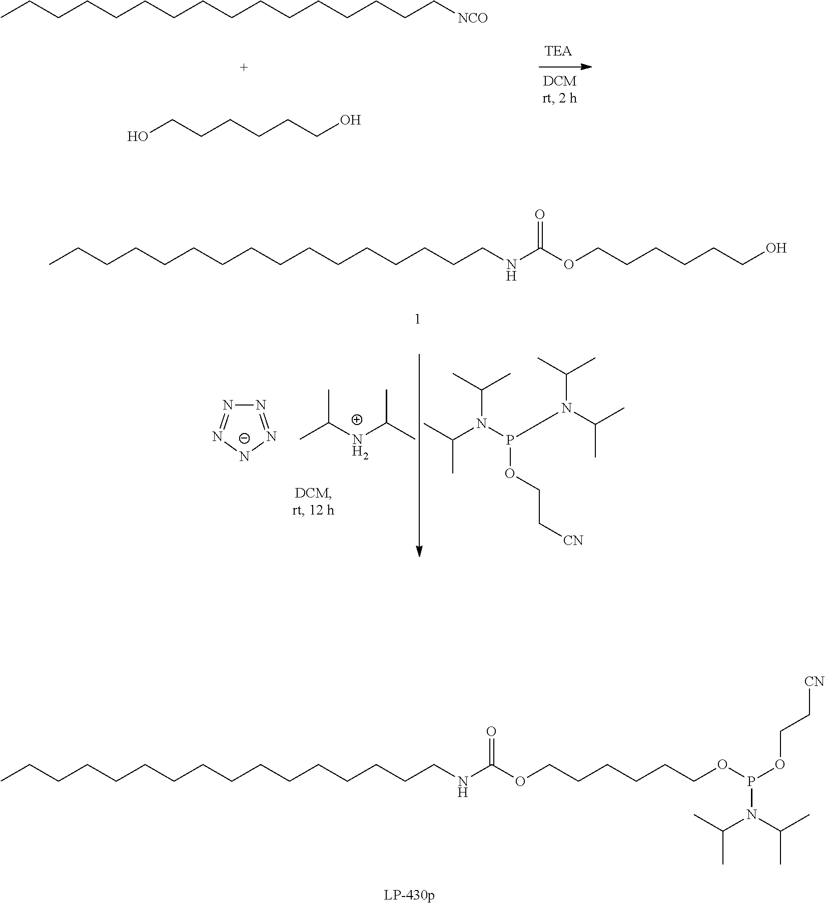

To a round bottom flask with hexadecyl isocyanate (1 eq) in DCM (5 mL) was added a solution of 1,6-hexanediol (1 eq) and TEA (2 eq) in DCM (5 mL). This mixture was stirred at room temperature for 2 hours. Then, the mixture was concentrated under reduced pressure and purified via CombiFlash chromatography using 2% MeOH in DCM to give compound 1 as an off-white solid in 20% yield. LC-MS [M+H]+ 386.3634 m/z, observed 386.3642 m/z.

Compound 1 (1 eq) was dried by two evaporations of toluene. Then, it was dissolved in anhydrous DCM (10 mL) and diisopropylammonium tetrazolide (1.4 eq) was added followed by activated molecular sieves (100 mg). The mixture was stirred under N₂ gas at room temperature for 30 minutes. Then, 2-cyanoethyl N,N,N′,N′-tetraisopropylphosphorodiamidite (1.6 eq) was added and stirring was continued at room temperature for 12 hours. After, 0.3 mL of TEA was added to quench the reaction and the mixture was directly loaded onto celite. CombiFlash chromatography using hexanes: ethyl acetate +1% TEA (70:30) to give pure product as a waxy, off-white solid in 41.7% yield. LC-MS [M+H]+ 586.4713 m/z, observed 586.4720 m/z.

### Synthesis of LP-431p

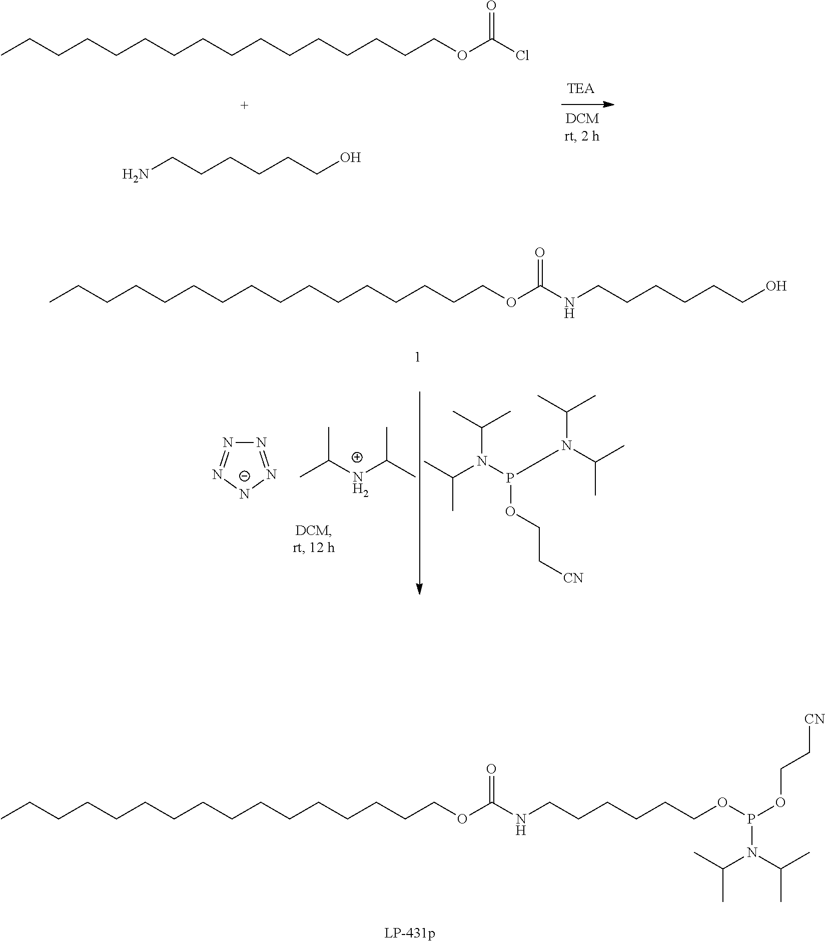

To around bottom flask containing 6-amino-1-hexanol (1.2 eq) and TEA (2 eq) in DCM (5 mL) was added a solution of hexadecyl chloroformate (1 eq) in DCM (5 mL). The reaction mixture was stirred at room temperature for 2 hours. Then, the mixture was concentrated under reduced pressure and purified via CombiFlash chromatography using 2% MeOH in DCM to give compound 1 as an off-white solid in 20% yield. LC-MS [M+H]+ 386.3634 m/z, observed 386.3638 m/z.

Compound 1 (1 eq) was dried by two evaporations of toluene. Then, it was dissolved in anhydrous DCM (10 mL) and diisopropylammonium tetrazolide (1.4 eq) was added followed by activated molecular sieves (100 mg). The mixture was stirred under N₂ gas at room temperature for 30 minutes. Then, 2-cyanoethyl N,N,N′,N′-tetraisopropylphosphorodiamidite (1.6 eq) was added and stirring was continued at room temperature for 12 hours. After, 0.3 mL of TEA was added to quench the reaction and the mixture was directly loaded onto celite. CombiFlash chromatography using hexanes: ethyl acetate +1% TEA (70:30) to give pure product as a waxy, off-white solid in 82.3% yield. LC-MS [M+H]+ 586.4713 m/z, observed 586.4705 m/z.

### Synthesis of LP-435p

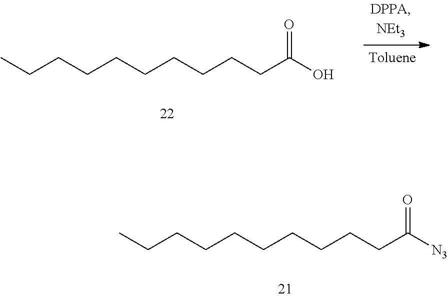

Undecanoic acid (2.0 g, 10.7 mmol) was dissolved in toluene (30 mL) and triethylamine (3.0 mL, 21.5 mmol) and diphenylphosphoryl azide (3.84 g, 14.0 mmol) were added. The reaction was stirred overnight. The acyl azide was observed by mass spec under basic conditions. The mixture was concentrated and the crude product was purified buy silica gel chromatorgraphy (0:100 EtOAc:Hexanes to 20:80 EtOAc:Hexanes) The product eluted at 10% EtOAc. Fractions containing product were concentrated to yield 0.975 g (43% yield) of compound 21 as a clear liquid.

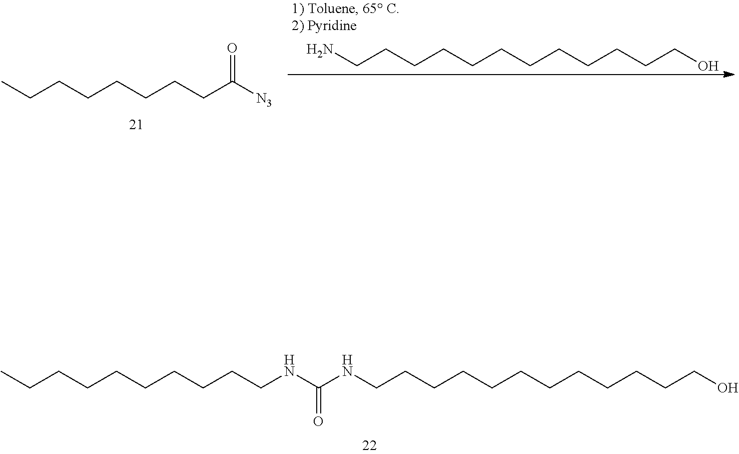 [SMILES: CCCCCCCCC(=O)N=[N+]=[N-] ; NCCCCCCCCCCCCO ; CCCCCCCCCCNC(=O)NCCCCCCCCCCCCO]

Compound 21 (0.975, 5.2 mmol) was dissolved in toluene (40 mL) and heated to 65° C. for 1 hour. Gas evolution was observed upon reaching 65° C. and stopped after approx. 30 min. The reaction mixture was cooled to room temperature. In a separate flask 1-amino-12-dodecanol (1.05 g, 5.2 mmol) was dissolved in THF (20 mL) and pyridine (0.85 mL, 10.5 mmol). The toluene solution was added to the THF solution and a white ppt rapidly formed. The reaction was stirred overnight. The reaction mixture was concentrated, and the crude product was recrystallized from isopropanol to yield 1.5558 g (77% yield) of compound 22 as a white solid.

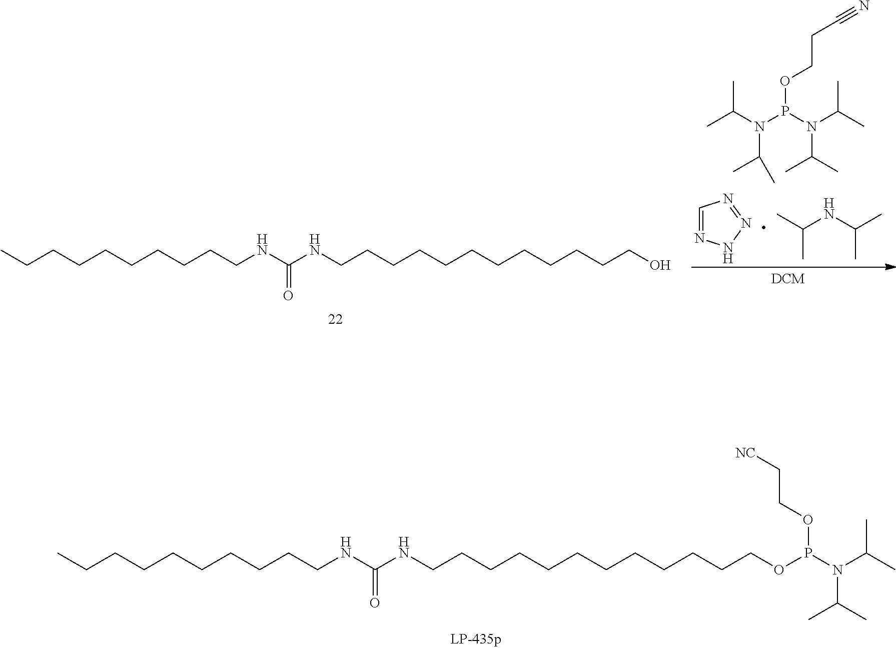

In a 100 mL RBF compound 22 (1.55 g, 4.0 mmol) was dried by 2 successive evaporations of toluene. Diisopropylammonium tetrazolide (0.277 g, 1.6 mmol) and 4 angstrom molecular sieves were added to the flask. The flask was purged and backfilled with nitrogen 3 times, and the solids were suspended in DCM (20 mL). The solids only partially dissolved. To the mixture 2-cyanoethyl N,N,N′,N′-tetraisopropylphosphorodiamidite (1.88 g, 6.2 mmol) was added and the reaction was stirred for 18 hours. LC-MS indicated no starting alcohol remained The reaction was transferred to a separatory funnel, washed with sat. aq. NaHCO₃ (2×40 mL), water (40 mL), brine (40 mL), dried over sodium sulfate and concentrated to dryness. Hexanes was added to the flask and the residue was stirred in hexanes for 1 hour to yield a white precipitate. The white solid was collected by filtration, washed with hexanes (2×20 mL), and dried under vacuum to yield 1.103 grams of a white powder. Proton NMR indicated a large amount of water remained, and a significant amount of the material was insoluble chloroform and DCM. The mixture was suspended in DCM, dried over magnesium sulfate, filtered through an additional pad of magnesium sulfate, and concentrated to yield 0.46 g (19% yield) of compound LP-435p as an off-white powder.

### Synthesis of LP-439p

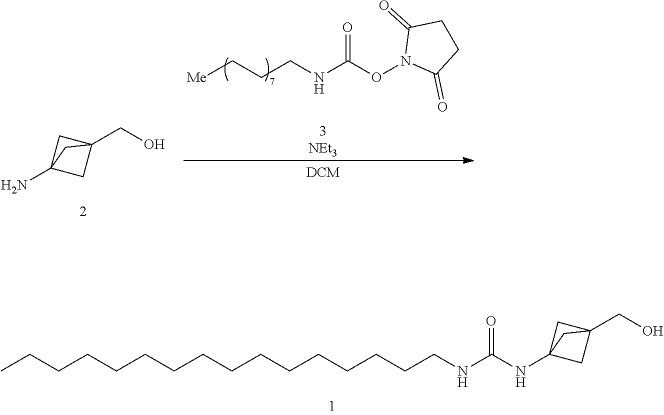

(3-aminobicyclo[1.1.1]pentan-1-yl)methanol (2) (0.20 g, 1.77 mmol) and 2,5-dioxopyrrolidin-1-yl hexadecylcarbamate (3) (0.67 g, 1.75 mmol) were dissolved in DCM (40 mL) followed by the addition oftriethylamine (0.72 mL, 5.3 mmol). The reaction was stirred overnight. After 18 hours a precipitate was observed. The precipitate was collected by filtration and washed with DCM (2×10 mL). The precipitate was dried in vacuo to yield 0.325 g (48% yield) of a white solid. Proton NMR analysis was consistent with product and crude material was of acceptable purity to proceed to the next step.

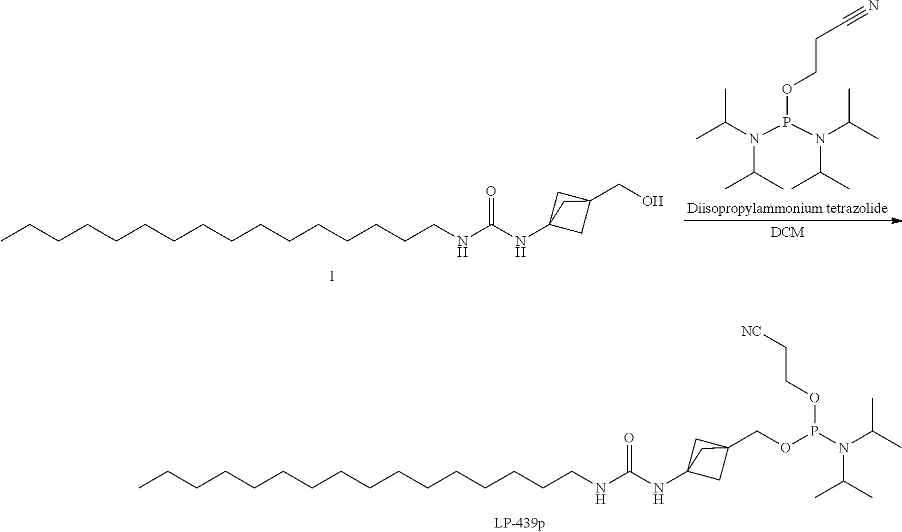 [SMILES: CCCCCCCCCCCCCCCCNC(=O)NC12CC(CO)(C1)C2 ; CC(C)N(C(C)C)P(OCCC#N)N(C(C)C)C(C)C ; CCCCCCCCCCCCCCCCNC(=O)NC12CC(COP(OCCC#N)N(C(C)C)C(C)C)(C1)C2]

Compound 1 (0.3 grams, 0.79 mmol) was dried by 4 successive evaporations with toluene then diisopropyl ammonium tetrazolide (0.054 g, 0.315 mmol) was added to the flask. The flask was purged and backfilled with nitrogen 3 times, the solids were suspended in DCM (20 mL) and 2-cyanoethyl N,N,N′,N′-tetraisopropylphosphorodiamidite (0.39 mL, 1.214 mmol) was added and the reaction was stirred for 18 hours. LC-MS analysis indicated no starting alcohol remained after 18 hours. The reaction was transferred to a separatory funnel, washed with sat. aq. NaHCO₃ (2×40 mL), water (40 mL), and concentrated to dryness. Hexanes was added to the residue, and the mixture was stirred for 1 hour to yield a white precipitate. The precipitate was collected by filtration, washed with hexanes, and dried under vacuum to yield 0.395 g (86% yield) of LP-439p as a white solid.

### Synthesis of LP-440p

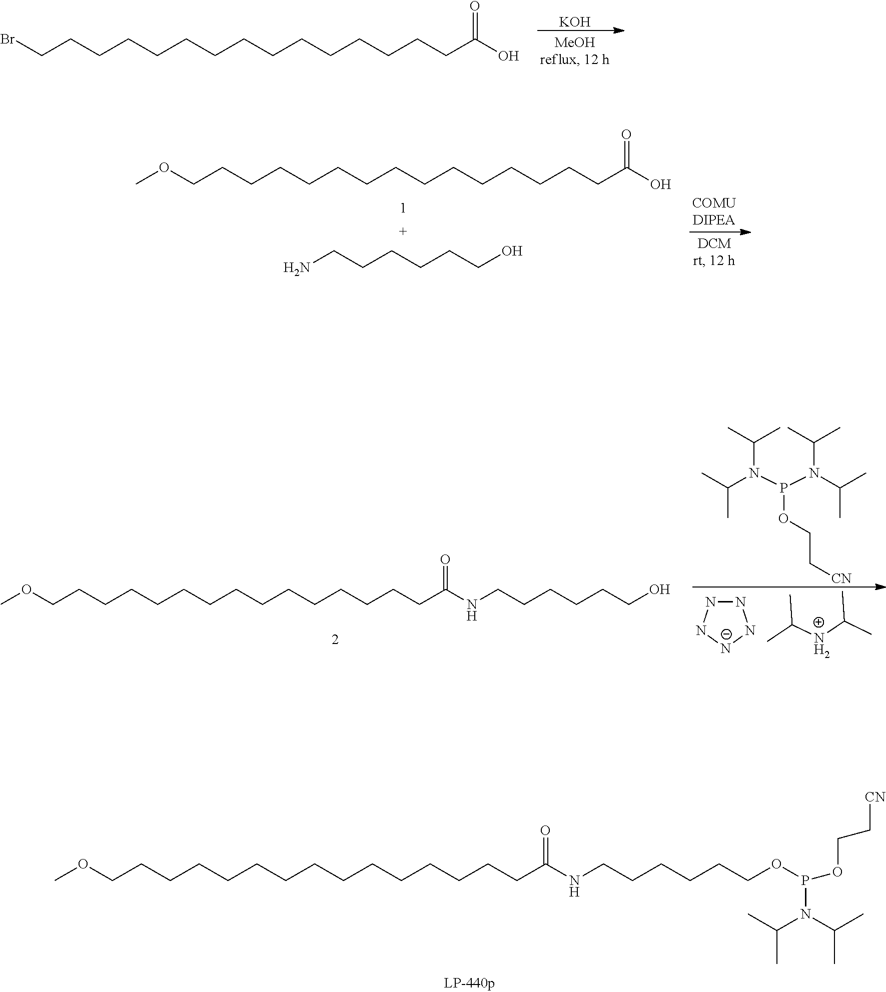

Anhydrous MeOH (8 mL) was cooled to 0° C., potassium hydroxide (3 eq) added, and the solution stirred for 30 min. A solution of 16-Bromohexadecanoic acid (1 eq) in anhydrous MeOH (7 mL) was then added via syringe. The reaction mixture was heated to reflux temperatures and stirred overnight. After cooling to room temperature, MeOH was removed in vacuo and the resulting crude mixture reconstituted in 1 N HCl (25 mL) and diethyl ether (5 mL). The crude product was extracted using diethyl ether (4×30 mL), the combined organic layers were washed with brine (30 mL) and dried over Na₂SO₄, and then the solvent removed in vacuo. Product was then purified on silica gel via column chromatography using hexanes: ethyl acetate (85:15) to give compound 1 as an oil in 86% yield. LC-MS [M+H]+ 287.2586 m/z, observed 287.2590.

To a solution of compound 1 (1 eq) in DCM (50 mL) was added COMU (1.2 eq) and DIPEA (2 eq). This mixture was stirred at room temperature for 30 minutes. Then, 6-amino-1-hexanol (1.2 eq) was added and the reaction mixture was stirred at room temperature for 12 hours. Then, the mixture was washed thrice with 1 M HCl (3×50 mL), once with brine (50 mL), dried over Na₂SO₄, and concentrated under reduced pressure. To the crude product was added ACN (100 mL) and carefully heated using the heatgun until all solids were soluble. This mixture was then left at room temperature which gave white crystals to form. The precipitate was then collected via vacuum filtration and washed several times with ACN to get rid of residual pink color. Compound 2 was obtained as white solid in 74% yield. LC-MS [M+H]+ 386.3634 m/z, observed 386.3626.

Compound 3 (1 eq) was dried by two evaporations of toluene. Then, it was dissolved in anhydrous DCM (10 mL) and diisopropylammonium tetrazolide (0.4 eq) was added followed by activated molecular sieves (100 mg). The mixture was stirred under N₂ gas at room temperature for 30 minutes. Then, 2-cyanoethyl N,N,N′,N′-tetraisopropylphosphorodiamidite (1.5 eq) was added and stirring was continued at room temperature for 12 hours. After, 0.3 mL of TEA was added to quench the reaction and the mixture was directly loaded onto celite. CombiFlash chromatography using hexanes: ethyl acetate +1% TEA (70:30) to give pure product as a waxy, off-white solid in 86% yield. LC-MS [M+H]+ 586.4713 m/z, observed 586.4705.

### Synthesis of LP-441p

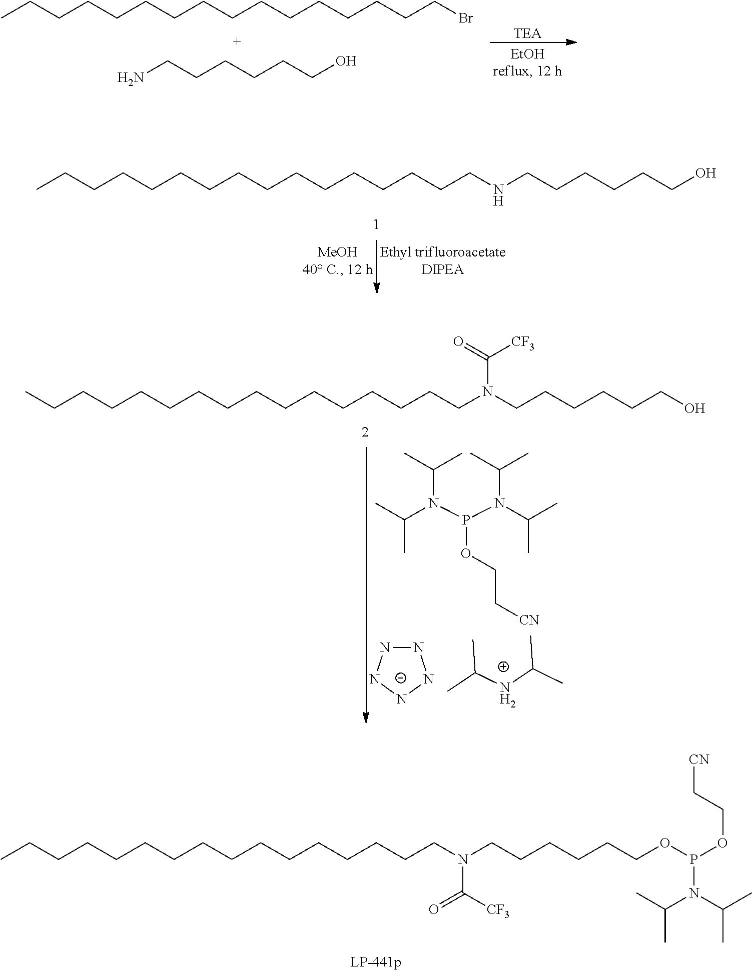

To a round bottom flask contain 6-amino-1-hexanol (2 eq) in EtOH (20 mL) was added 1-bromohexadecane (1 eq) and TEA (1.1 eq). This mixture was refluxed for 12 hours. Then, the solution was allowed to cool to room temperature and the solvent was removed in vacuo. Next, the crude was dissolved in H2O (20 mL) and extracted thrice with CH₃Cl (3×25 mL). The combined organics were washed once with brine (20 mL), dried over Na₂SO₄, and concentrated undeer reduced pressure. The crude mixture was purfied by CombiFlash chromatography using 10% MeOH in DCM +1% TEA to give compound 1 as an oil in 44% yield. LC-MS [M+H]+ 342.3736 m/z, observed 342.3728.

In a round bottom flask containing compound 1 (1 eq) in MeOH (25 mL) was added ethyl trifluoroacetate (5 eq) and DIPEA (2 eq). The reaction mixture was stirred at 40° C. for 12 hours. After, the solvent was removed under reduced pressure and the crude was purified via CombiFlash chromatography using 4%-6% MeOH in DCM to give compound 2 as an oil in 73% yield. LC-MS [M+H]+ 438.3559 m/z, observed 438.3551.

Compound 2 (1 eq) was dried by two evaporations of toluene. Then, it was dissolved in anhydrous DCM (10 mL) and diisopropylammonium tetrazolide (0.4 eq) was added followed by activated molecular sieves (100 mg). The mixture was stirred under N₂ gas at room temperature for 30 minutes. Then, 2-cyanoethyl N,N,N′,N′-tetraisopropylphosphorodiamidite (1.5 eq) was added and stirring was continued at room temperature for 12 hours. After, 0.3 mL of TEA was added to quench the reaction and the mixture was directly loaded onto celite. CombiFlash chromatography using hexanes: ethyl acetate +1% TEA (70:30) to give pure product as a waxy, off-white solid in 56% yield. LC-MS [M+H]+ 638.4637 m/z, observed 638.4629.

### Synthesis of LP-456p

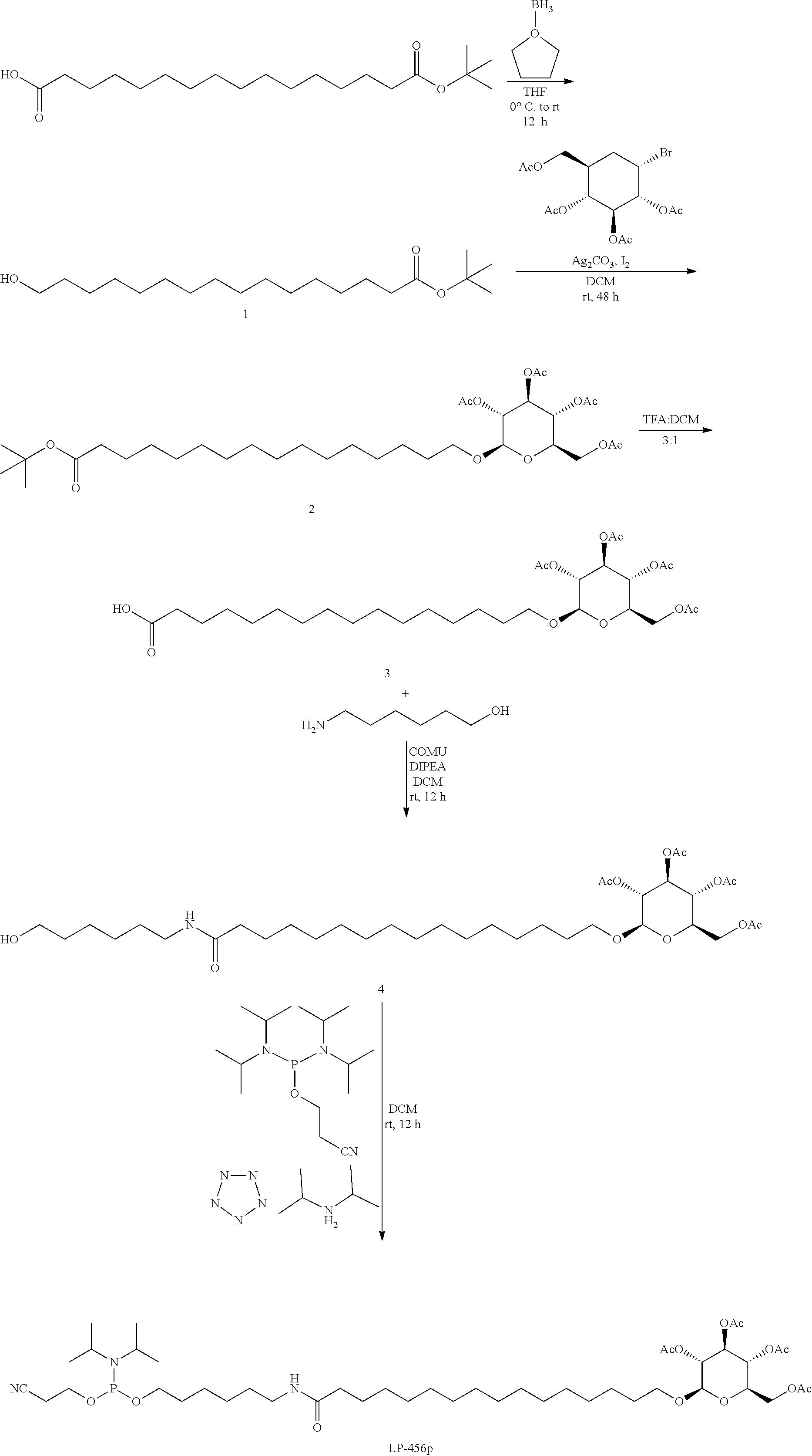

A 1 M solution of borane-tetrahydrofuran complex in tetrahydrofuran (1.5 eq) was added dropwise to a solution of 16(tert-r (1 eq) in dry tetrahydrofuran (20 mL) at 0° C. under nitrogen. The resulting solution was stirred at 0° C. for 2 hours, then the cooling bath was removed, and the mixture stirred at room temperature overnight. A saturated aqueous solution of sodium bicarbonate (50 mL) was added to quench the reaction. Then, the mixture was diluted with water (50 mL) and extracted thrice with DCM (3×50 mL). The combined organics were dried over Na₂SO₄ and concentrated under reduced pressure. The crude was purified via CombiFlash chromatography using hexane: ethyl acetate (80:20) to give compound 1 as an oil in 82% yield. LC-MS [M+H]+ 329.3056 m/z, observed 329.5060.

A mixture of compound 1 (1 eq), silver carbonate (3 eq), and a catalytic amount of iodine in DCM (5 mL) was stirred with molecular sieves for 15 min. To the mixture was added 2,3,4,6-Tetra-O-acetyl-alpha-D-glucopyranosyl bromide (1.5 eq) in DCM (5 mL) (also stirred with molecular sieves for 15 min). The resulting mixture was covered with aluminum foil and stirred at room temperature for 48 hours, then filtered through celite with EtOAc washing. The filtrate was concentrated, and the crude was purified via CombiFlash column chromatography using hexanes: ethyl acetate (80:20) to give compound 2 as an oil in 33% yield. LC-MS: [M+H2O]⁺ 676.4034 m/z, observed 676.4041.

To a solution of compound 2 in DCM (5 mL) was added TFA (15 mL). The solution was stirred for 2 hours at room temperature. After, the mixture was carefully poured into 100 mL of saturated NaHCO₃ (aq) solution. Once neutralized, the aqueous phase was extracted thrice with DCM (3×100 mL). The combined organics were dried over Na₂SO₄ and concentrated under reduced pressure to give compound 3 as a white solid in 97% yield. LC-MS: [M+H]+ 603.3381 m/z, observed 603.3388.

To a solution of compound 3 (1 eq) in DCM (10 mL) was added COMU (1.2 eq) and DIPEA (2 eq). This mixture was stirred at room temperature for 30 minutes. Then, 6-amino-1-hexanol (1.2 eq) was added and the reaction mixture was stirred at room temperature for 12 hours. Then, the mixture was washed thrice with 1 M HCl (3×10 mL), once with brine (10 mL), dried over Na₂SO₄, and concentrated under reduced pressure. The crude was purified via CombiFlash chromatography using 0-100% hexanes:ethyl acetate over 40 minutes to give compound 4 as an oil in 83% yield. LC-MS [M+H]+ 702.4429 m/z, observed 702.4421.

Compound 4 (1 eq) was concentrated by rotarty evaporator twice with toluene before charging anhydrous DCM (10 mL) to the reaction flask. The suspension was stirred 900 RPM under N₂ at ambient temperature with molecular sieves. 2-Cyanoethyl N,N,N′,N′-tetraisopropylphosphordiamidite (1.5 eq) was added to the suspension, followed by diisopropylammonium tetrazolide (0.4 eq). After 12 hours, TEA (300 uL) was added, and the reaction mixture was dry loaded onto celite. The product was purified using hexanes: ethyl acetate +1% TEA (60:40) to give LP-456p as an oil in 64% yield. LC-MS [M+H]+ 902.5507 m/z, observed 902.5517.

### Synthesis of LP-462p

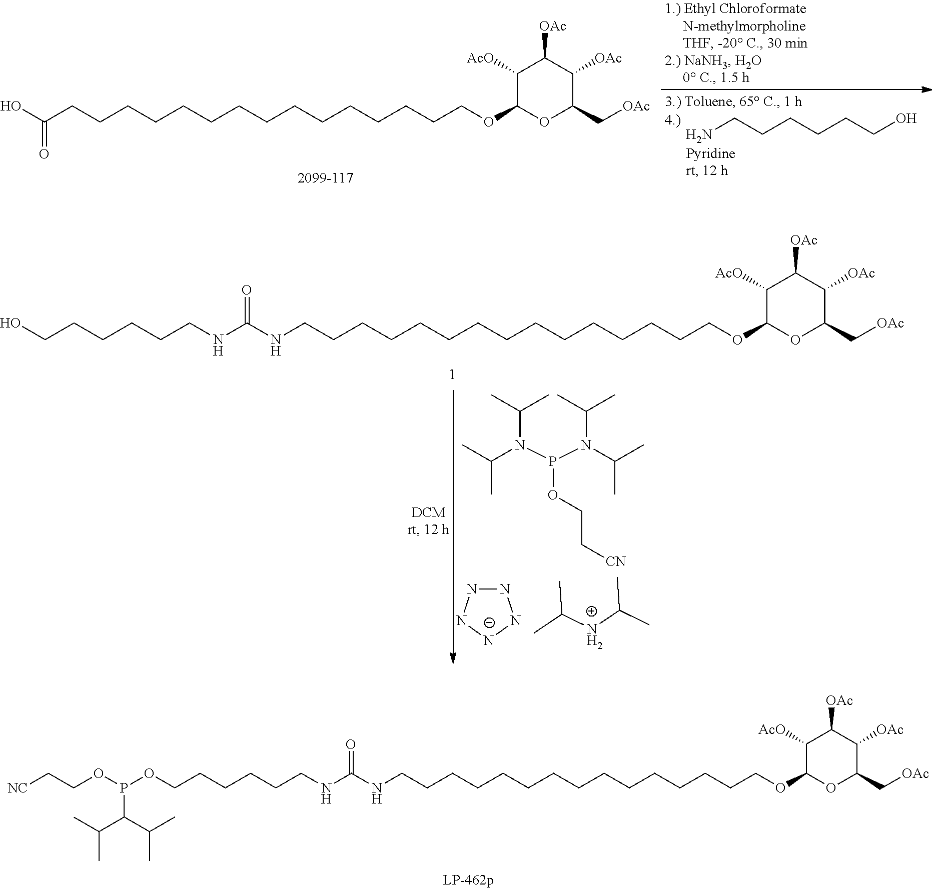

To a round bottom flask containing 2099-117 (1 eq) was added anhydrous THF (30 mL) and the solution was cooled to −20° C. Ethyl chloroformate (1.2) and N-methylmorpholine (1.2 eq) were added to the solution and the solution was stirred at −20° C. to −10° C. for 30 minutes. A solution of sodium azide (2.5 eq) in 1.5 mL of water was added to the reaction and the reaction was stirred at −7° C. for 90 minutes. The reaction was diluted with EtOAc. The aq. layer was separated and extracted 2 additional times with EtOAc. The combined organic layers were washed with brine, dried over Na₂SO₄, and concentrated to a clear liquid. The liquid was dissolved in toluene (30 mL) and heated to 65° C. for 1 hour, when no additional nitrogen gas formation was observed. Next, the solution was concentrated under reduced pressure and then dissolved in 30 mL of anhydrous DCM. 6-amino-1-hexanol (3 eq) and pyridine (1 eq) were added to the reaction mixture and stirring was continued for 12 hours. The mixture was concentrated under reduced pressure onto celite and purified via CombiFlash chromatography using 5% methanol in 95% DCM to give compound 1 as an oil in 51% yield. LC-MS [M+H2O]⁺ 717.4538 m/z, observed 717.4530.

Compound 1 (1 eq) was rotovaped twice with toluene before charging anhydrous DCM (10 mL) to the reaction flask. The suspension was stirred 900 RPM under N₂ at ambient temperature with molecular sieves. 2-Cyanoethyl N,N,N′,N′-tetraisopropylphosphordiamidite (1.5 eq) was added to the suspension, followed by diisopropylammonium tetrazolide (0.4 eq). After 12 hours, TEA (300 uL) was added, and the reaction mixture was dry loaded onto celite. The product was purified using hexanes: ethyl acetate +1% TEA (60:40) to give LP-462p as an oil in 64% yield. LC-MS [M+H]⁺ 916.5538 m/z, observed 916.5543.

### Synthesis of LP-463p

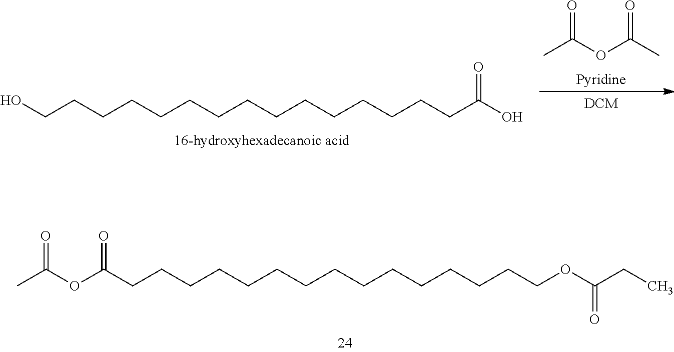

To a solution of 16-hydroxyhexadecanoic acid (1.5 g, 5.5 mmol) in DCM (60 mL) was added acetic anhydride (8.3 mL, 88 mmol) followed by pyridine (13.75 mL, 171 mmol) at room temperature. The mixture was stirred at room temperature overnight. After removing solvent in vacuo, the residue was redissolved in DCM and dry-loaded on a 80 g column. Hexanes to 50% EtOAc in Hexanes was used to purify. Compound 24 was obtained as a white solid, 1.22 g, 62%. LC-MS: calculated [M+H+H2O]375.27, found 374.80.

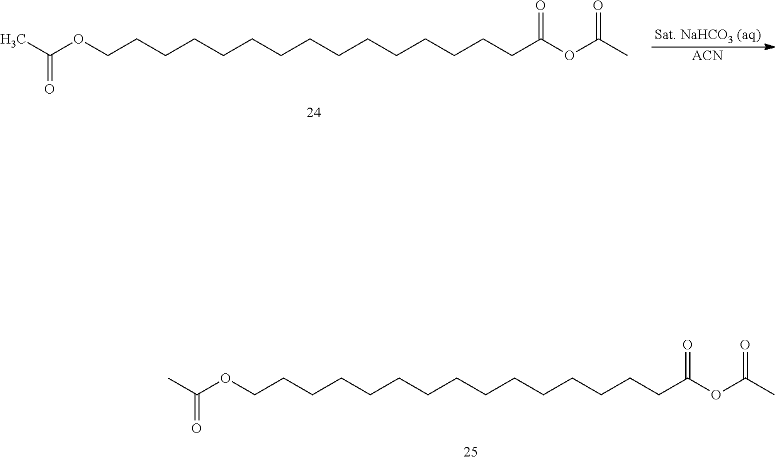

A suspension of compound 24 (1.22 g, 3.4 mmol) in ACN (40 mL) and sat. aq. NaHCO₃ (10 mL) was stirred at room temperature overnight. The pH was adjusted to 1 with 1N HCl. The precipitate was collected by suction filtration and was washed with H2O and air dried to yield 1.15 g (107% yield) of compound 25 is as a white solid. Greater than 100% yield due to residual water as determined by ¹H NMR. LC-MS: calculated [M+H]315.25, found 315.59.

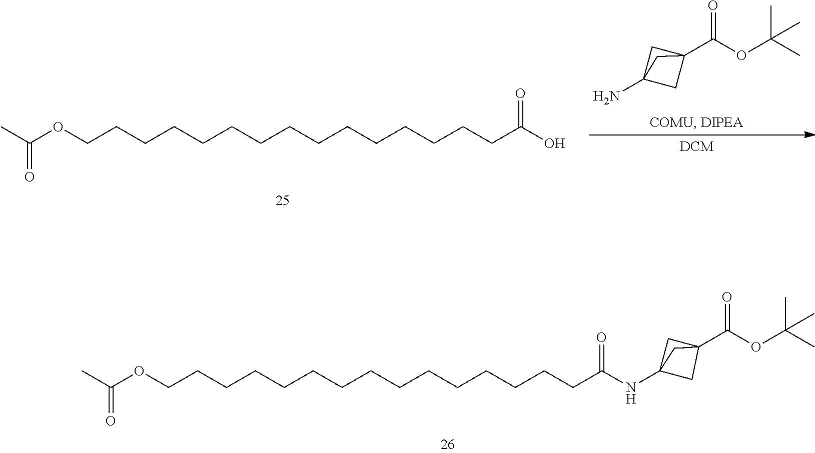 [SMILES: CC(=O)OCCCCCCCCCCCCCCCC(=O)O ; CC(C)(C)OC(=O)C12CC(N)(C1)C2 ; CC(=O)OCCCCCCCCCCCCCCCC(=O)NC12CC(C(=O)OC(C)(C)C)(C1)C2]

To a solution of compound 25 (1.15 g, 3.66 mmol) and diisopropylethylamine (1.28 mL, 7.3 mmol) in DCM (40 mL) was added COMU (1.8 g, 4.4 mmol) and tert-butyl 3-aminobicyclo[1.1.1]pentane-1-carboxylate (0.81 g, 4.4 mmol) at room temp. The mixture was stirred at room temp for 2 hours. The reaction mixture was concentrated onto silica gel and purified by column chromatography, 100% Hexanes:0% EtOAc to 0% Hexanes:100% EtOAc. Fractions containing product were combined and solvent was removed via rotary evaporator to yield 1.66 g (94% yield) of compound 26 as a brown solid. LC-MS: calculated [M+H]480.37, found 480.76.

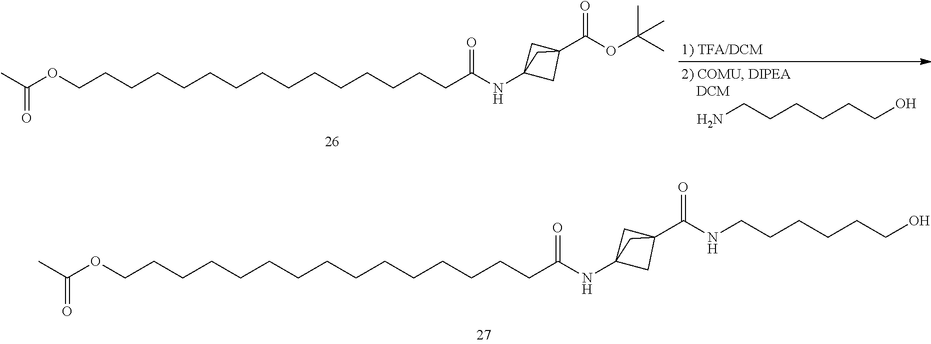

To a solution of compound 26 in DCM (10 mL) TFA (10 mL) was added, and the reaction was stirred at room temperature for 1.5 hours. After removing solvent in vacuo, the residue was dried under high vacuum for 2 hours. The residue was dissolved in DCM (30 mL) and diisopropylethylamine (1.2 mL, 6.9 mmol). After the residue was dissolved, COMU (1.77 g, 4.1 mmol) and 6-amino-1-hexanol (0.49 g, 4.1 mmol) were added at room temperature. The mixture was stirred at room temperature for 2.5 hours. After removing part of the solvent in vacuo, the residue was recrystallized with ACN. Product was collected by suction filtration and dried in vacuo to yield 1.48 grams (82% yield) of compound 27 as an off-white solid. LC-MS: calculated [M+H]523.41, found 524.06.

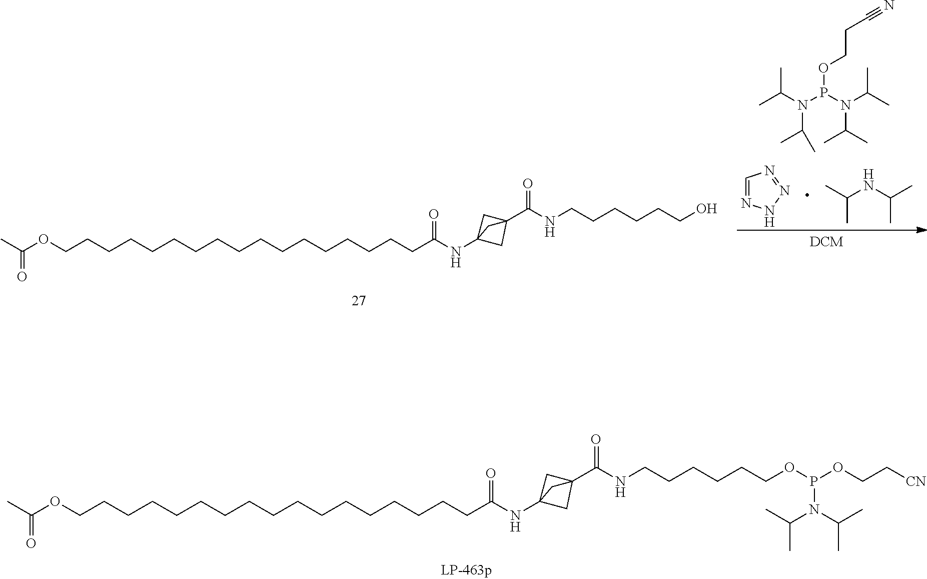

To a mixture of compound 27 (0.3 g, 0.57 mmol) in DCM (20 mL) was added Diisopropylammonium tetrazolide (0.039 g, 0.23 mmol) followed by drop wise addition of 2-cyanoethyl N,N,N′,N′-tetraisopropylphosphorodiamidite (0.277 g, 0.92 mmol) at room temperature. Then the mixture was refluxed 2 hours. After cooling to room temperature, the mixture was washed by sat. NaHCO₃ (aq) twice followed by H2O. After removing almost all solvent in vacuo, the residue was added to stirred hexanes and a white gel precipitate formed. After filtration, the white solid was collected by suction filtration and washed twice with hexanes. The white solid was dried under high vacuum to yield 0.305 g (73% yield) of compound LP-463p as a white solid. LC-MS: calculated [M+H]723.52, found 724.23.

### Synthesis of LP-464p

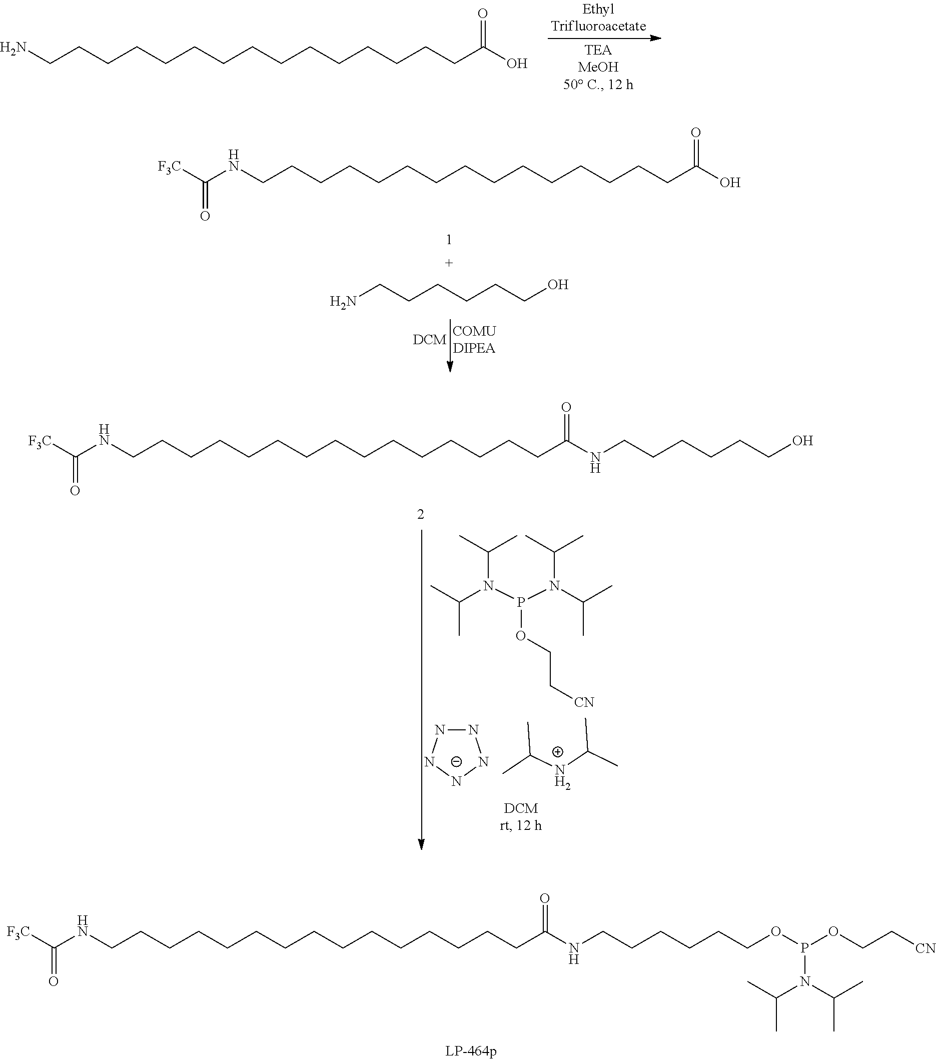

To a solution of 16-amino-hexadecanoic acid (1 eq) in anhydrous MeOH (20 mL) was added ethyl trifluoroacetate (1.5 eq) and TEA (1.1 eq). The reaction was stirred under nitrogen atmosphere for 12 hours at 50° C. Then, the mixture was concentrated under reduced pressure, diluted with EtOAc (30 mL) and washed twice with saurated KHSO₄ (15 mL), once with brine (15 mL), dried over Na₂SO₄, and concentrated under reduced pressure to give compound 1 as a white solid in 79% yield. LC-MS [M+H]⁺ 368.2412 m/z, observed 368.2419.

To a solution of compound 1 (1 eq) in DCM (30 mL) was added COMU (1.2 eq) and DIPEA (2 eq). This mixture was stirred at room temperature for 30 minutes. Then, 6-amino-1-hexanol (1.2 eq) was added and the reaction mixture was stirred at room temperature for 12 hours. Then, the mixture was washed thrice with 1 M HCl (3×15 mL), once with brine (15 mL), dried over Na₂SO₄, and concentrated under reduced pressure. To the crude product was added ACN (100 mL) and carefully heated using the heatgun until all solids were soluble. This mixture was then left at room temperature which gave white crystals to form. The precipitate was then collected via vacuum filtration and washed several times with ACN to get rid of residual pink color. Compound 2 was obtained as white solid in 82% yield. LC-MS [M+H]⁺ 467.3461 m/z, observed 467.3457.

Compound 2 (1 eq) was concentrated on rotary evaporator twice with toluene before charging anhydrous DCM (10 mL) to the reaction flask. The suspension was stirred 900 RPM under N₂ at ambient temperature with molecular sieves. 2-Cyanoethyl N,N,N′,N′-tetraisopropylphosphordiamidite (1.5 eq) was added to the suspension, followed by diisopropylammonium tetrazolide (0.4 eq). After 12 hours, TEA (300 uL) was added, and the reaction mixture was dry loaded onto celite. The product was purified using hexanes: ethyl acetate +1% TEA (60:40) to give LP-464p as waxy solid in 77% yield. LC-MS [M+H]⁺ 667.4539 m/z, observed 667.4544.

### Synthesis of LP-465p

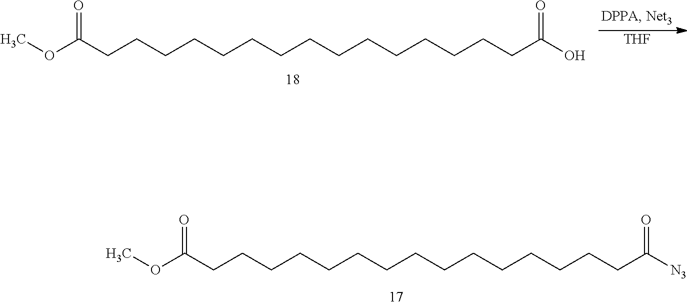 [SMILES: COC(=O)CCCCCCCCCCCCCCCC(=O)O ; COC(=O)CCCCCCCCCCCCCCCC(=O)N=[N+]=[N-] ; "THF"]

17-methoxy-17-oxohexadecanoic acid (1.0 g, 3.2 mmol) was dissolved in THF (50 mL) and triethylamine (0.89 mL, 6.4 mmol) and DPPA (0.75 mL, 3.5 mmol) were added. The reaction was stirred overnight. The reaction mixture was concentrated and the crude product was purified buy silica gel chromatorgraphy (20:80 EtOAc:Hexanes to 100:0 EtOAc:Hexanes). The product eluted at 10% EtOAc. Fractions 1-4 were found to contain product were concentrated to yield 0.60 g (56% yield) of compound 17 as a white solid.

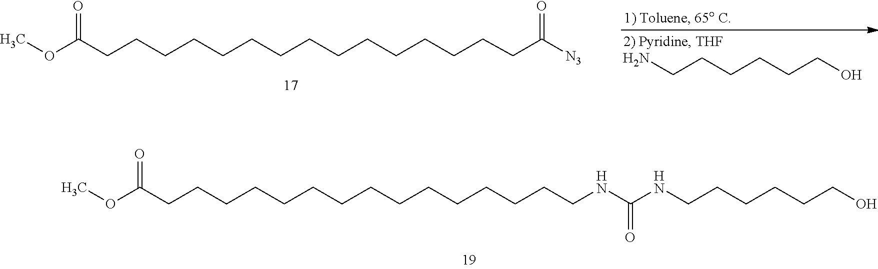 [SMILES: COC(=O)CCCCCCCCCCCCCCCC(=O)N=[N+]=[N-] ; NCCCCCCO ; COC(=O)CCCCCCCCCCCCCCCNC(=O)NCCCCCCO]

Compound 17 (0.58 g, 1.7 mmol) was dissolved in toluene (20 mL) and was heated to 65° C. until no more gas evolution was observed (30 minutes). The solution was cooled to room temperature then added to a solution of 6-amino-1-hexanol (0.2 g, 1.7 mmol) and pyridine (0.14, 1.7 mmol) in THF (20 mL). The reaction mixture was diluted with acetonitrile and the precipitate was collected by suction filtration, rinsed with acetonitrile, hexanes and dried in vacuo to yield 0.614 g (84% yield) of compound 19 as a white solid.

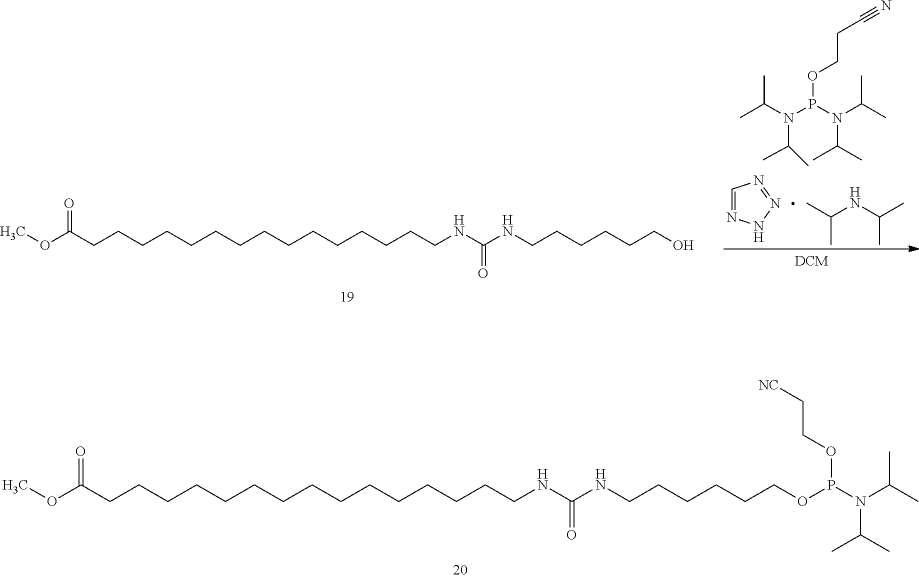

In a 100 mL RBF compound 19 (0.60 g, 1.4 mmol) was dried by 3 successive evaporations of toluene. Diisopropylammonium tetrazolide (0.096 g, 0.56 mmol) and 4 angstrom molecular sieves were added to the flask. The flask was purged and backfilled with nitrogen 3 times, and the solids were suspended in DCM (40 mL). The solids only partially dissolved. To the mixture, 2-cyanoethyl N,N,N′,N′-tetraisopropylphosphorodiamidite (0.65 g, 2.2 mmol) was added and the reaction was stirred for 18 hours. LC-MS after 18 hours indicated no starting alcohol remained. The reaction was transferred to a separatory funnel, washed with sat. aq. NaHCO₃ (2×40 mL), water (40 mL), and concentrated to dryness. Hexanes was added to the flask and the residue was stirred in hexanes for 2 hours to yield a white precipitate. The white solid was collected by filtration, washed with hexanes (2×20 mL), and dried under vacuum to yield 0.678 grams (77% yield) of LP-465p as a white solid.

### Synthesis of LP-466p

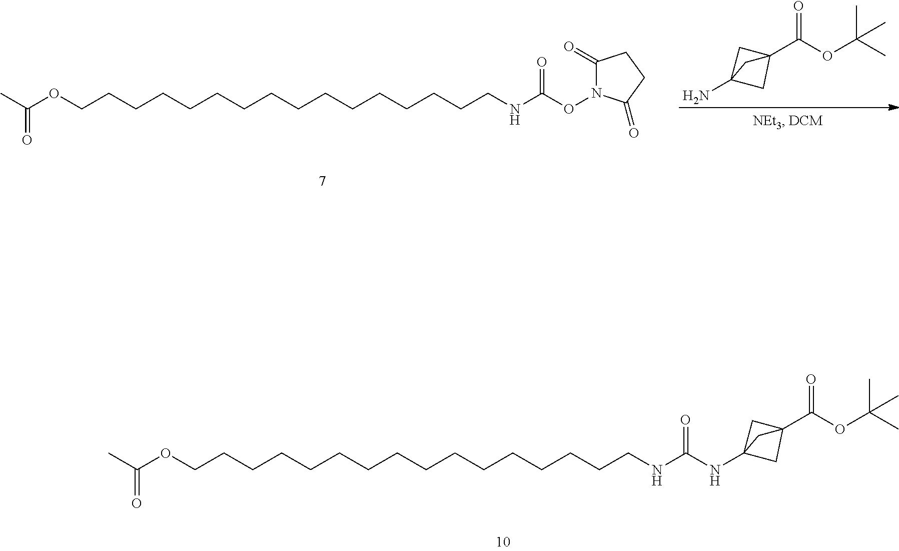 [SMILES: CC(=O)OCCCCCCCCCCCCCCCCNC(=O)ON1C(=O)CCC1=O ; CC(C)(C)OC(=O)C12CC(N)(C1)C2 ; CC(=O)OCCCCCCCCCCCCCCCCNC(=O)NC12CC(C(=O)OC(C)(C)C)(C1)C2]

Compound 7 (0.22 g, 0.50 mmol) and tert-butyl 3-aminobicyclo[1.1.1]pentane-1-carboxylate (0.0915 g, 0.50 mmol) were dissolved in DCM (10 mL) and triethylamine (0.21 mL, 1.5 mmol) was added. After 18 hours, <2% of the starting NHS ester remained by LC-MS. The reaction mixture was concentrated and loaded directly on to a silica gel column for purification. The product was purified by column chromatography 0% EtOAc/100% hexanes to 50% EtOAc 50% hexanes. Fractions 3-5 were combined to yield 0.23 g (89% yield) of compound 10 as a white solid.

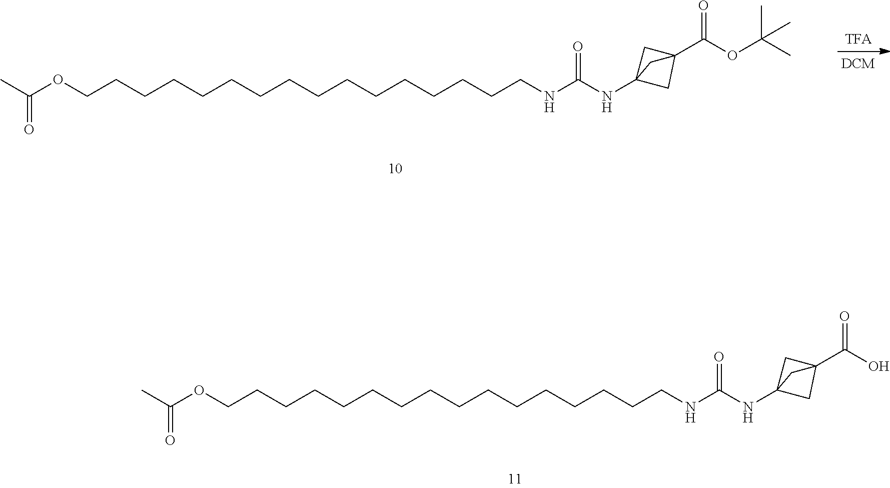

Compound 10 (0.23 g, 0.45 mmol) was dissolved in DCM (3 mL) and trifluoroacetic acid (3 mL) was added. The solution was stirred overnight. No SM was present by LC-MS after 18 hours. The reaction mixture was concentrated and the residual TFA was removed by 2 co-evaporations with toluene to yield 0.189 mg (93%) of compound 11 as a white solid.

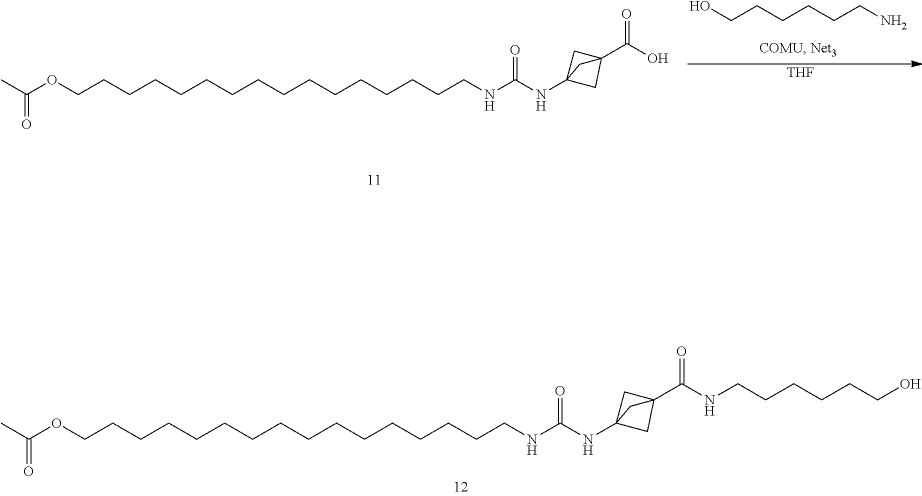 [SMILES: CC(=O)OCCCCCCCCCCCCCCCCNC(=O)NC12CC(C(=O)O)(C1)C2 ; NCCCCCCO ; CC(=O)OCCCCCCCCCCCCCCCCNC(=O)NC12CC(C(=O)NCCCCCCO)(C1)C2 ; "THF"]

Compound 11 (0.189 g, 0.48 mmol) and COMU (0.215 g, 0.5 mmol) were dissolved in DCM (10 mL) and triethylamine (0.333 mL, 2.4 mmol) was added. The reaction was stirred for about 5 minutes, then 6-amino-1-hexanol (0.059 g, 0.5 mmol) was added. After 1 hour, no starting material remained by LC-MS. The reaction mixture was concentrated, and water was added to the residue. The mixture was sonicated until all of the material was suspended in water and the precipitate was collected by filtration and washed 3 times with water. The precipitate was dried in vacuo to yield 0.166 g (70% yield) of compound 12 as a white solid.

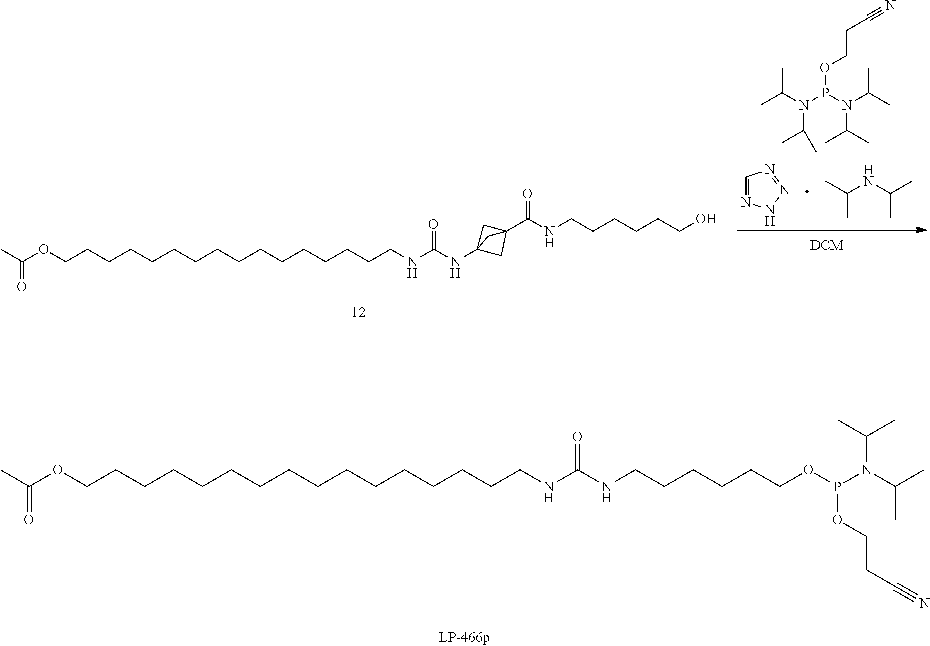

In a 100 mL RBF compound 12 (0.166 g, 0.3 mmol) was dried by 2 successive evaporations of toluene. Diisopropylammonium tetrazolide (0.02 g, 0.12 mmol) and 4 angstrom molecular sieves were added to the flask. The flask was purged and backfilled with nitrogen 3 times, and the solids were suspended in DCM (20 mL). The solids only partially dissolved. To the mixture 2-cyanoethyl N,N,N′,N′-tetraisopropylphosphorodiamidite (0.14 g, 0.46 mmol) was added and the reaction was stirred for 18 hours. LC-MS indicated no starting alcohol remained after 18 hours. The reaction was transferred to a separatory funnel, washed with sat. aq. NaHCO₃ (2×40 mL), water (40 mL), brine (40 mL), dried over magnesium sulfate and concentrated to dryness. Hexanes was added to the flask and the residue was stirred in hexanes for 1 hour to yield a white precipitate. The white solid was collected by filtration, washed with hexanes (2×20 mL), and dried under vacuum to yield 0.116 grams (51% yield) of LP-466p as a white waxy solid.

### Synthesis of LP-493p (shown as LP-493p uridine)

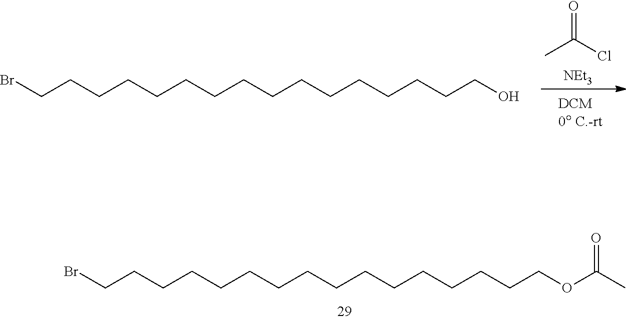

To a solution of 1-bromohexadecan-16-ol (6.0 g, 18.7 mmol) in DCM (90 mL) was added triethylamine (2.9 mL, 20.5 mmol). The resulting solution was cooled to 0° C. in an ice/water bath. After cooling, acetyl chloride (1.46 mL, 20.5 mmol) was added dropwise. The reaction was stirred at 0° C. for 1 hour after the addition was complete then allowed to warm to room temperature and stirred overnight. After about 18 hours, the reaction mixture was washed with sat. NaHCO₃ (20 mL), water, 1 M HCl (20 mL), water (2×20 mL), brine (20 mL), dried over sodium sulfate and concentrated to a white solid. The crude product was purified buy silica gel chromatorgraphy (0:100 EtOAc:Hexanes to 20:80 EtOAc:Hexanes) The product eluted at 10% EtOAc. Fractions 5-12 were concentrated to yield 6.02 g (89% yield) of compound 29 as a white powder.

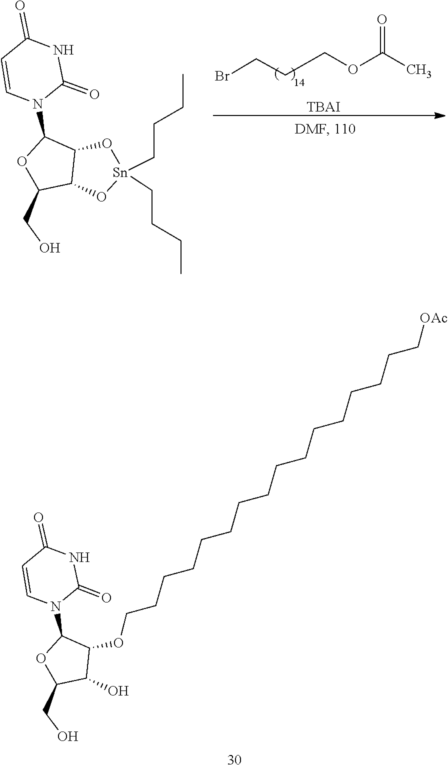

Compound 31 was prepared according to the literature procedure. Compound 31 (1.0 g, 2.1 mmol), Compound 29 (1.53 g, 4.2 mmol), and tetrabutyl ammonium iodide (1.6 g, 0.42 mmol) were placed in an oven dried flask. The flask was evacuated and purged with nitrogen three times, then dry DMF (10 mL) was added to the flask. The solution was heated to 110° C. for 18 hours. After 18 hours the reaction was cooled to room temperature and the solvent was removed in vacuo. The residue was resuspended in DCM/MeOH and concentrated onto silica gel for purification. The column was eluted with 3% MeOH/97% DCM to 20% MeOH/80% DCM. Fractions containing the 2′ and 3′ addition products were pooled and concentrated to yield 0.236 g (21% yield) of compound 30 plus the 3′ addition product.

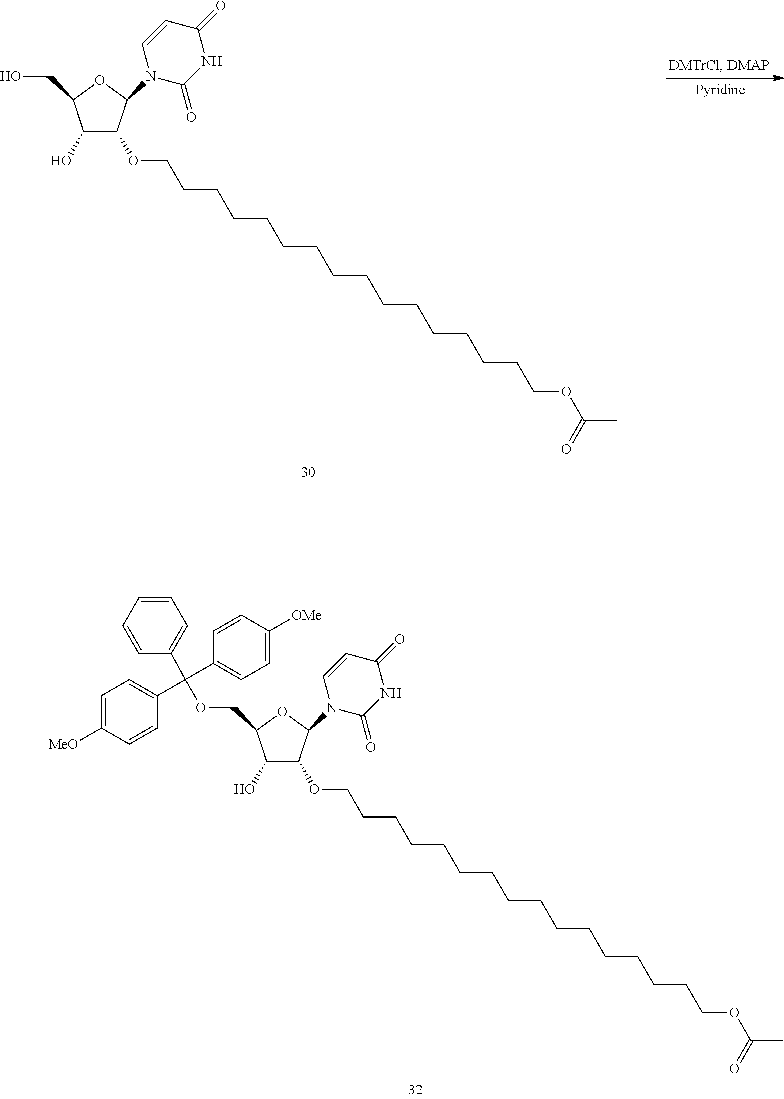 [SMILES: CC(=O)OCCCCCCCCCCCCCCCCO[C@@H]1[C@H](O)[C@@H](CO)O[C@H]1n1ccc(=O)[nH]c1=O ; COc1ccc(C(OC[C@H]2O[C@@H](n3ccc(=O)[nH]c3=O)[C@H](OCCCCCCCCCCCCCCCCOC(C)=O)[C@@H]2O)(c2ccccc2)c2ccc(OC)cc2)cc1]

Compound 30+3′ addition product (0.23 g, 0.44 mmol) was dried by successive evaporations of toluene and anhydrous pyridine using a rotary evaporator. DMAP (0.003 g, 0.022 mmol) and dimethoxytrityl chloride (0.165 g, 0.49 mmol) were added to the flask and the flask was evacuated and purged with nitrogen 3 times. The solids were dissolved in of pyridine (10 mL). The reaction was stirred overnight at room temperature. All volatiles were removed, residual pyridine was removed by co-distillation with toluene. The residue was partitioned between DCM (20 mL) and aqueous NaHCO₃ (20 mL). The organic phase was separated, the aqueous was extracted with DCM (20 mL), combined organic phases were dried (Na₂SO₄) and concentrated. The crude product was purified by silica gel chromatography. Silica was pretreated with a 50:50 mixture of Hexanes/EtOAc+2% v/v triethylamine. The product was isolated on CombiFlash using 40 g column, eluent: hexane-ethyl acetate +1% of Et₃N, 20-60% Compound eluted at 60% EtOAc. Late fractions were contaminated with 3′ alkylated product. Fractions containing pure 2′ alkylated product were combined and concentrated to yield 0.107 g (27% yield) of compound 32 as a white solid.

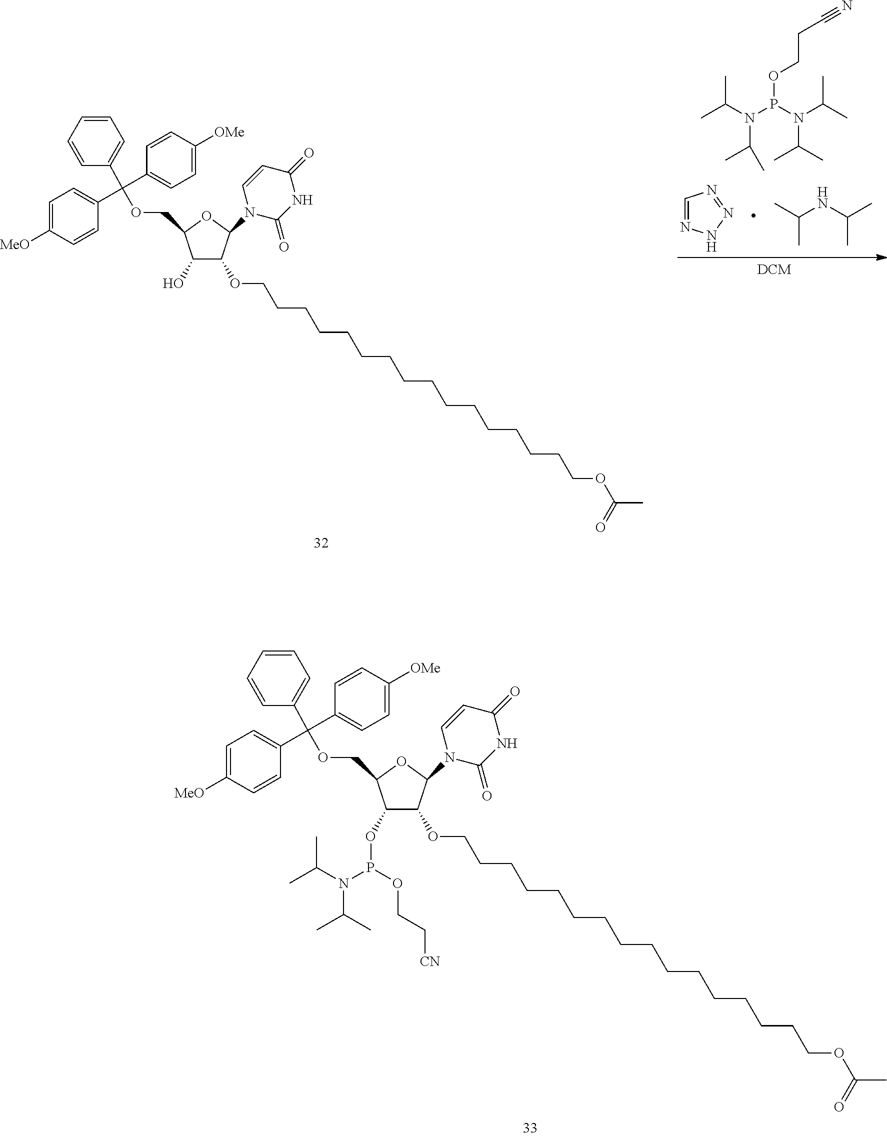

In a 25 mL RBF, Compound 32 (0.150 g, 0.18 mmol) and diisopropylamonium tetrazolide (0.043 g, 0.25 mmol), and 4 angstrom molecular sieves, were placed and the flask was evacuated purged with nitrogen 3 times. DCM (5 mL) was added, followed by the dropwise addition of 2-cyanoethyl N,N,N′,N-tetraisopropylphosphorodiamidite (0.092 mL, 0.29 mmol). The reaction was stirred overnight. The reaction mixture was quenched with −2 mL Sat. NaHCO₃, filtered into a separatory funnel, the layers were separated and the NaHCO₃ layer was extracted 1 additional time with DCM (10 mL). The combined organic layers were dried over Na₂SO₄ and concentrated to a thick viscous liquid. The crude product was purified buy silica gel chromatography (0:100 EtOAc:Hexanes to 100:0 EtOAc:Hexanes.) Silica was pretreated with a 50:50 mixture of Hexanes/EtOAc+2% v/v triethylamine. The product eluted at 45% EtOAc. Fractions 15-35 were found to contain product with little oxidized product contamination and were combined to yield 0.088 g (47% yield) of compound 33 as a sticky colorless solid. Fractions 36-50 were combined to yield 44 mg of a sticky colorless solid and contained product with more oxidized material.

### Synthesis of (2C8C12) phosphoramidite

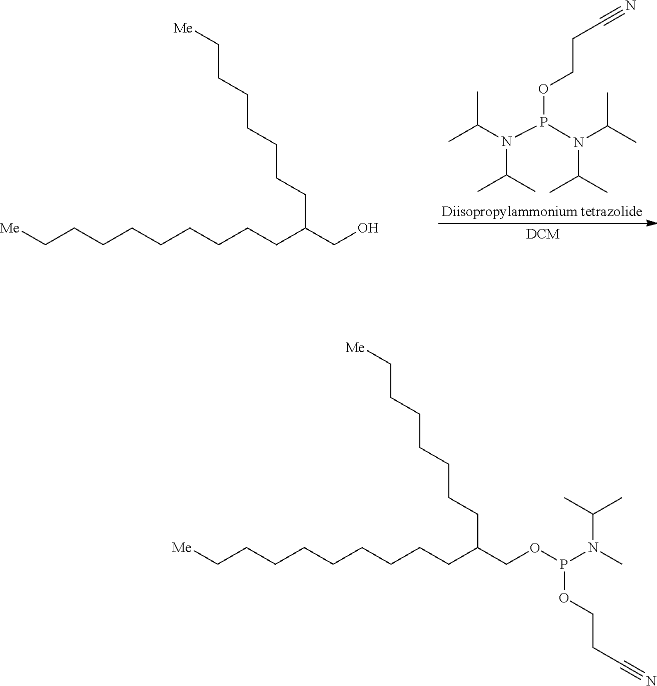 [SMILES: CCCCCCCCCCC(CO)CCCCCCCC ; CC(C)N(C(C)C)P(OCCC#N)N(C(C)C)C(C)C ; CCCCCCCCCCC(CCCCCCCC)COP(OCCC#N)N(C)C(C)C]

2-ocytyl-1-decanol (1.00 grams, 3.35 mmol) and diisopropylamonium tetrazolide (0.2868 grams, 1.68 mmol) were placed in a flask and the flask was purged with nitrogen. DCM (50 mL) was added to the mixture and 2-Cyanoethyl N,N,N′N′-tetraisopropylphosphorodiamidite (2.66 mL, 8.37 mmol) was added dropwise. Upon completion of the reaction, 3 mL of triethylamine was added to the reaction and the reaction was concentrated directly onto celite for purification. The crude product was purified buy silica gel chromatography (0:100 EtOAc:hexanes +2% triethylamine to 100:0 EtOAc:Hexanes +2% triethylamine) The product eluted with 100% Hexanes. Fractions containing product were concentrated to 1.268 g (76% yield) of a clear liquid.

### Synthesis of (2C6C10) phosphoramidite

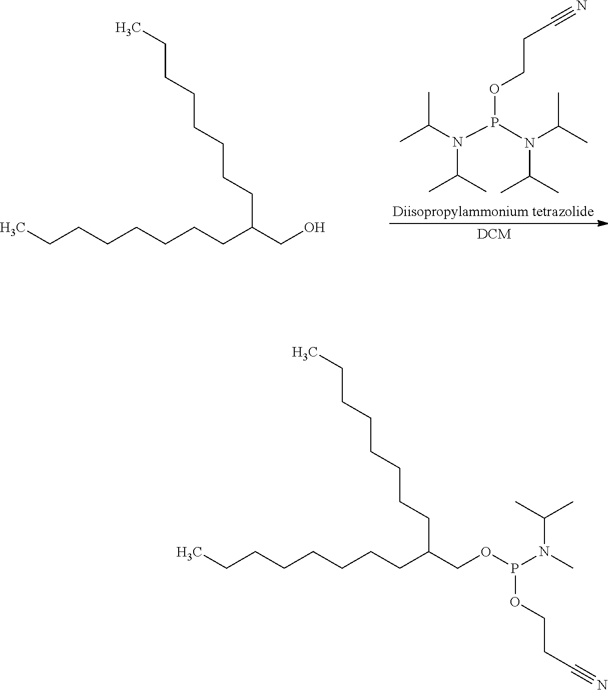 [SMILES: CCCCCCCCC(CO)CCCCCCCC ; CC(C)N(C(C)C)P(OCCC#N)N(C(C)C)C(C)C ; CCCCCCCCC(CCCCCCCC)COP(OCCC#N)N(C)C(C)C]

2-hexyl-1-decanol (1.00 g, 4.13 mmol) and diisopropylamonium tetrazolide (0.353 g, 2.06 mmol) were placed in a flask and the flask was purged with nitrogen. DCM (50 mL) was added to the mixture and 2-Cyanoethyl N,N,N′,N′-tetraisopropylphosphorodiamidite (3.27 mL, 10.3 mmol) was added dropwise . . . . Upon completion of the reaction, 3 mL of triethylamine was added to the reaction and the reaction was concentrated directly onto celite for purification. The crude product was purified buy silica gel chromatography (0:100 EtOAc:hexanes +2% triethylamine to 100:0 EtOAc:Hexanes +2% triethylamine) The product eluted with 100% Hexanes. Fractions containing product were concentrated to 1.32 g (72% yield) of a clear liquid.

### Synthesis of HO-C16 phosphoramidite

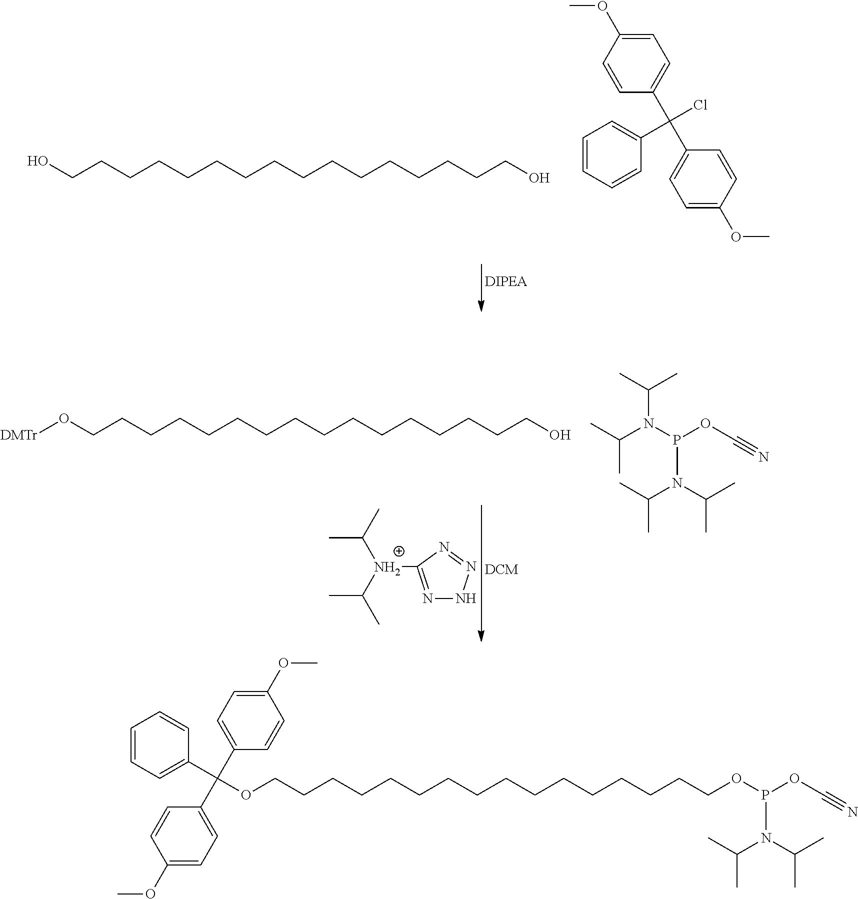

1,16-hexadecanediol, N,N-diisopropylethylamine (0.100 g) was dissolved in 2 mL THF. 4,4′-Dimethoxytrityl chloride (2.2 g, 6.6 mmol) was added slowly as a solid. After 2 h, the reaction was concentrated by rotary evaporation, and the product was purified by column chromatography (25% ethyl acetate/75% hexane).

DMT-O-C₁₆-OH (0.200 g), Bis(diisopropylamino)(2-cyanoethoxy)phosphine (0.227 mL) and BisDiisopropylammonium tetrazolide (0.0611 g) were dissolved in anhydrous DCM at room temperature. The reaction was capped and stirred overnight. Conversion was determined via LC-MS (0.25M NH₄ HCO₃:H₂O buffer system). Celite® was added to the reaction mixture and it was concentrated under vacuum until a white powder remained. The mixture was loaded dry onto a silica column (12 gram) using a EtOAc/Hexanes (1% Triethylamine) solvent system to prevent hydrolysis from the silica gel.^{l} The product was characterized by ³¹PNMR, ¹HNMR, and LC-MS.

### Synthesis of C16 phosporamidite

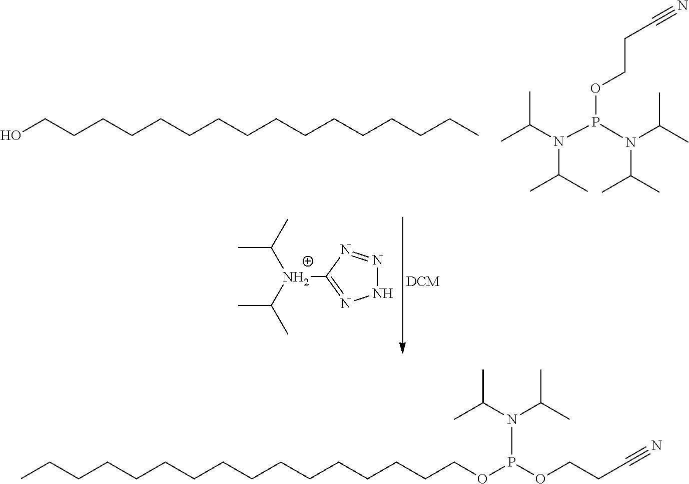

Cetyl alcohol (1.10 g), Bis(diisopropylamino)(2-cyanoethoxy)phosphine (2.88 mL) and BisDiisopropylammonium tetrazolide (0.778 g) were dissolved in a solution of DCM at room temperature. The reaction was capped and stirred overnight. Conversion was determined via LC-MS (0.25M NH₄ HCO₃:H₂O buffer system). Celite® was added to the reaction mixture and it was concentrated under vacuum until a white powder remained. The mixture was loaded dry onto a silica column (12 gram) using a s EtOAc/Hexanes (1% Triethylamine) solvent system to prevent hydrolysis from the silica gel. The desired product was not retained on the column and came out shortly after being loaded. The isolated product was then characterized by LC-MS, ¹HNMR and ³¹PNMR. Final yield: 856.5 mg (93.8%).

### Synthesis of C22 phosporamidite

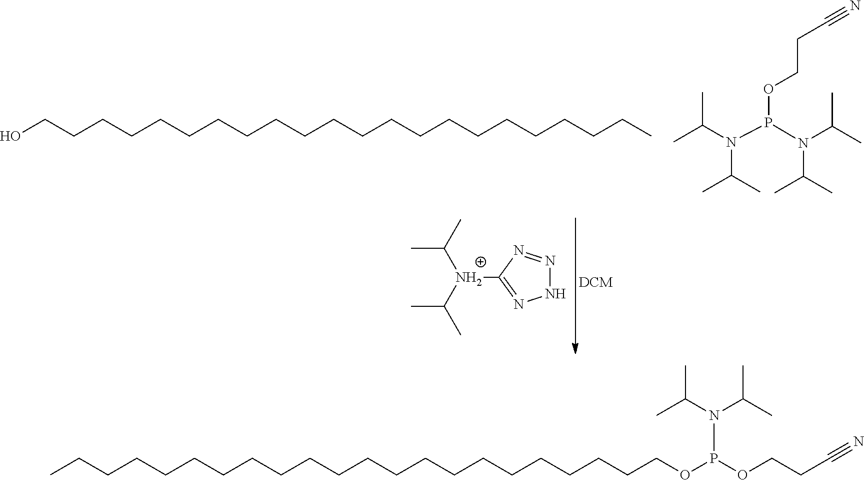

Docosanol (1.10 g), Bis(diisopropylamino)(2-cyanoethoxy)phosphine (2.1 mL) and BisDiisopropylammonium tetrazolide (0.577 g) were dissolved in a solution of DCM at room temperature. The reaction was capped and stirred overnight. Conversion was determined via LC-MS (0.25M NH₄ HCO₃:H₂O buffer system). Celite® was added to the reaction mixture and it was concentrated under vacuum until a white powder remained. The mixture was loaded dry onto a silica column (12 gram) pretreated with 3 mL of triethylamine using a EtOAc/Hexanes (1% Triethylamine) solvent system to prevent hydrolysis from the silica gel. The isolated product was then characterized by LC-MS, ¹HNMR and ³¹PNMR. Final yield: 2.1085 g (118.8%).

### Example 3. Conjugation of Lipid PK/PD Modulator Precursors

Either prior to or after annealing and prior to or after conjugation of one or more targeting ligands, one or more lipid PK/PD modulator precursors can be linked to the RNAi agents disclosed herein. The following describes the general conjugation process used to link lipid PK/PD modulator precursors to the constructs set forth in the Examples depicted herein.

### A. Conjugation of Activated Ester PK/PD Modulators

The following procedure was used to conjugate PK/PD modulators having an activated ester moiety such as TFP (tetrafluorophenoxy) or PNP (para-nitrophenol) to an RNAi agent with an amine-functionalized sense strand, such as C6-NH2, NH2-C6, or (NH2-C6). An annealed RNAi Agent dried by lyophilization was dissolved in DMSO and 10% water (v/v %) at 25 mg/mL. Then 50-100 equivalents of TEA and 3 equivalents of activated ester PK/PD modulator were added to the solution. The solution was allowed to react for 1-2 hours, while monitored by RP-HPLC-MS (mobile phase A 100 mM HFIP, 14 mM TEA; mobile phase B: acetonitrile on an Waters™ XBridge C18 column, Waters Corp.)

The product was then precipitated by adding 12 mL acetonitrile and 0.4 mL PBS and centrifuging the solid to a pellet. The pellet was then re-dissolved in 0.4 mL of 1XPBS and 12 mL of acetonitrile. The resulting pellet was dried on high vacuum for one hour.

### B. Conjugation of Phosphoramidite PK/PD Modulators

PK/PD modulators having a phosphoramidite moiety may be attached on resin using typical oligonucleotide manufacturing conditions.

### C. Hydrolysis of PK/PD Modulators

Certain PK/PD modulators are hydrolyzed in the cleavage and deprotection conditions described in Example 1, above. For example LP-429p, LP-456p, LP-462p, LP-463p, LP-464p, LP-466p, LP-493p, and HO-C16 phosphoramidite all include moieties that are hydrolyzed under the cleavage and deprotection conditions.

LP-465p is hydrolyzed following conjugation to the oligonucleotide strand in a solution of 0.5-1 M potassium carbonate in 1:1 methanol to water and heated to 50-60° C. for about 4 hours.

TABLE A
Conjugate ID Numbers With Chemically Modified Antisense
and Sense Strands (including Linkers and Conjugates) used
in the following examples

| | Sense Strand (Fully | | | |
|---|---|---|---|---|
| AC/ | Modified with | SEQ | | SEQ |
| AD ID | Conjugated Targeting | ID | | ID |
| Number | Ligand) (5′ → 3′) | NO: | Antisense Strand (5′ → 3′) | NO: |
| AC001249 | LP-183a-(NH-C6)s(invAb)sc | 68 | cPrpusUfsusAfgagugagGfaUf | 1 |
| | auuuuaaUfCfCfucacucuaaas( | | uAfaAfaUfsg | |
| | invAb) | | | |
| AC001374 | LP-128a-(NH-C6)s(invAb)sc | 69 | cPrpusUfsusAfgagugagGfaUf | 2 |
| | auuuuaaUfCfCfucacucuaaas( | | uAfaAfaUfsg | |
| | invAb) | | | |
| AC001375 | LP-200a-(NH-C6)s(invAb)sc | 70 | cPrpusUfsusAfgagugagGfaUf | 3 |
| | auuuuaaUfCfCfucacucuaaas( | | uAfaAfaUfsg | |
| | invAb) | | | |
| AC001376 | LP-245a-(NH-C6)s(invAb)sc | 71 | cPrpusUfsusAfgagugagGfaUf | 4 |
| | auuuuaaUfCfCfucacucuaaas( | | uAfaAfaUfsg | |
| | invAb) | | | |
| AC001377 | LP-257a-(NH-C6)s(invAb)sc | 72 | cPrpusUfsusAfgagugagGfaUf | 5 |
| | auuuuaaUfCfCfucacucuaaas( | | uAfaAfaUfsg | |
| | invAb) | | | |
| AC001378 | LP-262a-(NH-C6)s(invAb)sc | 73 | cPrpusUfsusAfgagugagGfaUf | 6 |
| | auuuuaaUfCfCfucacucuaaas( | | uAfaAfaUfsg | |
| | invAb) | | | |
| AC001379 | LP-269a-(NH-C6)s(invAb)sc | 74 | cPrpusUfsusAfgagugagGfaUf | 7 |
| | auuuuaaUfCfCfucacucuaaas( | | uAfaAfaUfsg | |
| | invAb) | | | |
| AC001380 | LP-273a-(NH-C6)s(invAb)sc | 75 | cPrpusUfsusAfgagugagGfaUf | 8 |
| | auuuuaaUfCfCfucacucuaaas( | | uAfaAfaUfsg | |
| | invAb) | | | |
| AC001381 | LP-274a-(NH-C6)s(invAb)sc | 76 | cPrpusUfsusAfgagugagGfaUf | 9 |
| | auuuuaaUfCfCfucacucuaaas( | | uAfaAfaUfsg | |
| | invAb) | | | |
| AC001382 | LP-276a-(NH-C6)s(invAb)sc | 77 | cPrpusUfsusAfgagugagGfaUf | 10 |
| | auuuuaaUfCfCfucacucuaaas( | | uAfaAfaUfsg | |
| | invAb) | | | |
| AC001383 | LP-287a-(NH-C6)s(invAb)sc | 78 | cPrpusUfsusAfgagugagGfaUf | 11 |
| | auuuuaaUfCfCfucacucuaaas( | | uAfaAfaUfsg | |
| | invAb) | | | |
| AC001384 | LP-293a-(NH-C6)s(invAb)sc | 79 | cPrpusUfsusAfgagugagGfaUf | 12 |
| | auuuuaaUfCfCfucacucuaaas( | | uAfaAfaUfsg | |
| | invAb) | | | |
| AC001385 | LP-296a-(NH-C6)s(invAb)sc | 80 | cPrpusUfsusAfgagugagGfaUf | 13 |
| | auuuuaaUfCfCfucacucuaaas( | | uAfaAfaUfsg | |
| | invAb) | | | |
| AC001386 | LP-300a-(NH-C6)s(invAb)sc | 81 | cPrpusUfsusAfgagugagGfaUf | 14 |
| | auuuuaaUfCfCfucacucuaaas( | | uAfaAfaUfsg | |
| | invAb) | | | |
| AC001388 | LP-232a-(NH-C6)s(invAb)sc | 82 | cPrpusUfsusAfgagugagGfaUf | 15 |
| | auuuuaaUfCfCfucacucuaaas( | | uAfaAfaUfsg | |
| | invAb) | | | |
| AC001389 | LP-233a-(NH-C6)s(invAb)sc | 83 | cPrpusUfsusAfgagugagGfaUf | 16 |
| | auuuuaaUfCfCfucacucuaaas( | | uAfaAfaUfsg | |
| | invAb) | | | |
| AC001390 | LP-242a-(NH-C6)s(invAb)sc | 84 | cPrpusUfsusAfgagugagGfaUf | 17 |
| | auuuuaaUfCfCfucacucuaaas( | | uAfaAfaUfsg | |
| | invAb) | | | |
| AC001391 | LP-243a-(NH-C6)s(invAb)sc | 85 | cPrpusUfsusAfgagugagGfaUf | 18 |
| | auuuuaaUfCfCfucacucuaaas( | | uAfaAfaUfsg | |
| | invAb) | | | |
| AC001392 | LP-249a-(NH-C6)s(invAb)sc | 86 | cPrpusUfsusAfgagugagGfaUf | 19 |
| | auuuuaaUfCfCfucacucuaaas( | | uAfaAfaUfsg | |
| | invAb) | | | |
| AC001393 | LP-259a-(NH-C6)s(invAb)sc | 87 | cPrpusUfsusAfgagugagGfaUf | 20 |
| | auuuuaaUfCfCfucacucuaaas( | | uAfaAfaUfsg | |
| | invAb) | | | |
| AC001394 | LP-260a-(NH-C6)s(invAb)sc | 88 | cPrpusUfsusAfgagugagGfaUf | 21 |
| | auuuuaaUfCfCfucacucuaaas( | | uAfaAfaUfsg | |
| | invAb) | | | |
| AC001395 | LP-286a-(NH-C6)s(invAb)sc | 89 | cPrpusUfsusAfgagugagGfaUf | 22 |
| | auuuuaaUfCfCfucacucuaaas( | | uAfaAfaUfsg | |
| | invAb) | | | |
| AC001396 | LP-289a-(NH-C6)s(invAb)sc | 90 | cPrpusUfsusAfgagugagGfaUf | 23 |
| | auuuuaaUfCfCfucacucuaaas( | | uAfaAfaUfsg | |
| | invAb) | | | |
| AC001397 | LP-290a-(NH-C6)s(invAb)sc | 91 | cPrpusUfsusAfgagugagGfaUf | 24 |
| | auuuuaaUfCfCfucacucuaaas( | | uAfaAfaUfsg | |
| | invAb) | | | |
| AC001442 | LP-304a-(NH-C6)s(invAb)sc | 92 | cPrpusUfsusAfgagugagGfaUf | 25 |
| | auuuuaaUfCfCfucacucuaaas( | | uAfaAfaUfsg | |
| | invAb) | | | |
| AC001445 | LP-310a-(NH-C6)s(invAb)sc | 93 | cPrpusUfsusAfgagugagGfaUf | 26 |
| | auuuuaaUfCfCfucacucuaaas( | | uAfaAfaUfsg | |
| | invAb) | | | |
| AC001446 | LP-303a-(NH-C6)s(invAb)sc | 94 | cPrpusUfsusAfgagugagGfaUf | 27 |
| | auuuuaaUfCfCfucacucuaaas( | | uAfaAfaUfsg | |
| | invAb) | | | |
| AC002478 | LP-183a-(NH-C6)s(invAb)sc | 95 | cPrpusGfsasuagAUNAggAfuUf | 28 |
| | cucacuuUfAfAfuccucuaucas( | | aAfaGfugagsg | |
| | invAb) | | | |
| AC002548 | LP-283a-(NH-C6)s(invAb)sc | 96 | cPrpusGfsasuagAUNAggAfuUf | 29 |
| | cucacuuUfAfAfuccucuaucas( | | aAfaGfugagsg | |
| | invAb) | | | |
| AC002549 | LP-383a-(NH-C6)s(invAb)sc | 97 | cPrpusGfsasuagAUNAggAfuUf | 30 |
| | cucacuuUfAfAfuccucuaucas( | | aAfaGfugagsg | |
| | invAb) | | | |
| AC002550 | LP-396a-(NH-C6)s(invAb)sc | 98 | cPrpusGfsasuagAUNAggAfuUf | 31 |
| | cucacuuUfAfAfuccucuaucas( | | aAfaGfugagsg | |
| | invAb) | | | |
| AC002551 | LP-395a-(NH-C6)s(invAb)sc | 99 | cPrpusGfsasuagAUNAggAfuUf | 32 |
| | cucacuuUfAfAfuccucuaucas( | | aAfaGfugagsg | |
| | invAb) | | | |
| AC910361 | (invAb)scauuuuC16AfaUfCfC | 100 | cPrpusUfsusagAfgUfGfaggaU | 33 |
| | fucacucuaaas(invAb) | | fuAfaaausg | |
| AC910860 | LP-293a-(NH-C6)-s(invAb)s | 101 | cPrpusUfsusagAfgUfGfaggaU | 34 |
| | cauuuuAfaUfCfCfucacucuaaa | | fuAfaaausg | |
| | s(invAb) | | | |
| AC912620 | (invAb)scauuuuLP493AfaUfC | 102 | cPrpusUfsusagAfgUfGfaggaU | 35 |
| | fCfucacucuaaas(invAb) | | fuAfaaausg | |
| AD08908 | (NAG37)s(invAb)scauuuuaaU | 103 | cPrpusUfsusAfgagugagGfaUf | 36 |
| | fCfCfucacucuaaas(invAb) | | uAfaAfaUfsg | |
| AD08942 | (NH2-C6)s(invAb)scauuuuaa | 104 | cPrpusUfsusAfgagugagGfaUf | 37 |
| | UfCfCfucacucuaaas(invAb) | | uAfaAfaUfsg | |
| AD11276 | (invAb)sccucacuC16uUfAfAf | 105 | cPrpusGfsasuagAfggAfuUfaA | 38 |
| | uccucuaucas(invAb) | | faGfugagsg | |
| AD11278 | (invAb)sccucacuuUfAfAfucc | 106 | cPrpusGfsasuagAfggAfuUfaA | 39 |
| | uC16cuaucas(invAb) | | faGfugagsg | |
| AD11556 | (NH2-C6)s(invAb)sccucacuu | 107 | cPrpusGfsasuagAUNAggAfuUf | 40 |
| | UfAfAfuccucuaucas(invAb) | | aAfaGfugagsg | |
| AD11691 | LP-183ra-(NH-C6)s(invAb)s | 108 | cPrpuAfgGfauaacagAfuGfaGf | 41 |
| | cuuaacucAfUfCfuguuauccuas | | uuaassg | |
| | (invAb) | | | |
| AD11692 | LP-183ra-(NH-C6)s(invAb)s | 109 | cPrpusGfsasuagAUNAggAfuUf | 42 |
| | ccucacuuUfAfAfuccucuaucas | | aAfaGfugagsg | |
| | (invAb) | | | |
| AD11728 | LP-409a-(NH-C6)s(invAb)sc | 110 | cPrpusGfsasuagAUNAggAfuUf | 43 |
| | cucacuuUfAfAfuccucuaucas( | | aAfaGfugagsg | |
| | invAb) | | | |
| AD11731 | (invAb)sccucacuC16uUfAfAf | 111 | cPrpusGfsasuagAUNAggAfuUf | 44 |
| | uccucuaucas(invAb) | | aAfaGfugagsg | |
| AD11732 | (invAb)sccucacuuUfAfAfucc | 112 | cPrpusGfsasuagAUNAggAfuUf | 45 |
| | uC16cuaucas(invAb) | | aAfaGfugagsg | |
| AD11739 | LP-395a-(NH-C6)s(invAb)sc | 113 | cPrpusGfsasuagAUNAggAfuUf | 46 |
| | cucacuuUfAfAfuccucuaucas( | | aAfaGfugagsg | |
| | invAb) | | | |
| AD11758 | C16a-s(invAb)sccucacuuUfA | 114 | cPrpusGfsasuagAUNAggAfuUf | 47 |
| | fAfuccucuaucas(invAb) | | aAfaGfugagsg | |
| AD11759 | C22a-s(invAb)sccucacuuUfA | 115 | cPrpusGfsasuagAUNAggAfuUf | 48 |
| | fAfuccucuaucas(invAb) | | aAfaGfugagsg | |
| AD11821 | HO-C16a-s(invAb)sccucacuu | 116 | cPrpusGfsasuagAUNAggAfuUf | 49 |
| | UfAfAfuccucuaucas(invAb) | | aAfaGfugagsg | |
| AD11841 | (2C8C12)a-s(invAb)sccucac | 117 | cPrpusGfsasuagAUNAggAfuUf | 50 |
| | uuUfAfAfuccucuaucas(invAb | | aAfaGfugagsg | |
| | ) | | | |
| AD11842 | (2C6C10)a-s(invAb)sccucac | 118 | cPrpusGfsasuagAUNAggAfuUf | 51 |
| | uuUfAfAfuccucuaucas(invAb | | aAfaGfugagsg | |
| | ) | | | |
| AD12261 | LP-293a-(NH-C6)s(invAb)sg | 119 | cPrpusGfsaGfaucacagAfaUfc | 52 |
| | uugaagaUfuCfuGfugaucucas( | | Ufucasasc | |
| | invAb) | | | |
| AD13302 | LP-409a-(NH-C6)s(invAb)sc | 120 | cPrpusUfsusAfgagugagGfaUf | 53 |
| | auuuuaaUfCfCfucacucuaaas( | | uAfaAfaUfsg | |
| | invAb) | | | |
| AD13303 | LP-430a-(C6)s(invAb)scauu | 121 | cPrpusUfsusAfgagugagGfaUf | 54 |
| | uuaaUfCfCfucacucuaaas(inv | | uAfaAfaUfsg | |
| | Ab) | | | |
| AD13304 | LP-431a-(NH-C6)s(invAb)sc | 122 | cPrpusUfsusAfgagugagGfaUf | 55 |
| | auuuuaaUfCfCfucacucuaaas( | | uAfaAfaUfsg | |
| | invAb) | | | |
| AD13306 | LP-435a-(C12)s(invAb)scau | 123 | cPrpusUfsusAfgagugagGfaUf | 56 |
| | uuuaaUfCfCfucacucuaaas(in | | uAfaAfaUfsg | |
| | vAb) | | | |
| AD13308 | LP-439a-s(invAb)scauuuuaa | 124 | cPrpusUfsusAfgagugagGfaUf | 57 |
| | UfCfCfucacucuaaas(invAb) | | uAfaAfaUfsg | |
| AD13309 | LP-429a-(NH-C6)s(invAb)sc | 125 | cPrpusUfsusAfgagugagGfaUf | 58 |
| | auuuuaaUfCfCfucacucuaaas( | | uAfaAfaUfsg | |
| | invAb) | | | |
| AD13310 | LP-44la-(C6)s(invAb)scauu | 126 | cPrpusUfsusAfgagugagGfaUf | 59 |
| | uuaaUfCfCfucacucuaaas(inv | | uAfaAfaUfsg | |
| | Ab) | | | |
| AD13681 | LP-440a-(NH-C6)s(invAb)sc | 127 | cPrpusUfsusAfgagugagGfaUf | 60 |
| | auuuuaaUfCfCfucacucuaaas( | | uAfaAfaUfsg | |
| | invAb) | | | |
| AD13682 | LP-132a-(NH-C6)s(invAb)sc | 128 | cPrpusUfsusAfgagugagGfaUf | 61 |
| | auuuuaaUfCfCfucacucuaaas( | | uAfaAfaUfsg | |
| | invAb) | | | |
| AD13683 | LP-465a-(NH-C6)s(invAb)sc | 129 | cPrpusUfsusAfgagugagGfaUf | 62 |
| | auuuuaaUfCfCfucacucuaaas( | | uAfaAfaUfsg | |
| | invAb) | | | |
| AD13684 | LP-456a-(NH-C6)s(invAb)sc | 130 | cPrpusUfsusAfgagugagGfaUf | 63 |
| | auuuuaaUfCfCfucacucuaaas( | | uAfaAfaUfsg | |
| | invAb) | | | |
| AD13685 | LP-462a-(NH-C6)s(invAb)sc | 131 | cPrpusUfsusAfgagugagGfaUf | 64 |
| | auuuuaaUfCfCfucacucuaaas( | | uAfaAfaUfsg | |
| | invAb) | | | |
| AD13686 | LP-464a-(NH-C6)s(invAb)sc | 132 | cPrpusUfsusAfgagugagGfaUf | 65 |
| | auuuuaaUfCfCfucacucuaaas( | | uAfaAfaUfsg | |
| | invAb) | | | |
| AD13687 | LP-463a-(NH-C6)s(invAb)sc | 133 | cPrpusUfsusAfgagugagGfaUf | 66 |
| | auuuuaaUfCfCfucacucuaaas( | | uAfaAfaUfsg | |
| | invAb) | | | |
| AD13688 | LP-466a-(NH-C6)s(invAb)sc | 134 | cPrpusUfsusAfgagugagGfaUf | 67 |
| | auuuuaaUfCfCfucacucuaaas( | | uAfaAfaUfsg | |
| | invAb) | | | |

As used in Table A, the following notations are used to indicate modified nucleotides, targeting groups, and linking groups:
A=adenosine-3′-phosphate
C=cytidine-3′-phosphate
G=guanosine-3′-phosphate
U=uridine-3′-phosphate
I=inosine-3′-phosphate
a=2′-O-methyladenosine-3′-phosphate
as=2′-O-methyladenosine-3′-phosphorothioate
c=2′-O-methylcytidine-3′-phosphate
cs=2′-O-methylcytidine-3′-phosphorothioate
g=2′-O-methylguanosine-3′-phosphate
gs=2′-O-methylguanosine-3′-phosphorothioate
i=2′-O-methylinosine-3′-phosphate
is=2′-O-methylinosine-3′-phosphorothioate
t=2′-O-methyl-5-methyluridine-3′-phosphate
ts=2′-O-methyl-5-methyluridine-3′-phosphorothioate
u=2′-O-methyluridine-3′-phosphate
us=2′-O-methyluridine-3′-phosphorothioate
Af=2′-fluoroadenosine-3′-phosphate
Afs=2′-fluoroadenosine-3′-phosporothioate
Cf=2′-fluorocytidine-3′-phosphate
Cfs=2′-fluorocytidine-3′-phosphorothioate
Gf=2′-fluoroguanosine-3′-phosphate
Gfs=2′-fluoroguanosine-3′-phosphorothioate
Tf=2′-fluoro-5′-methyluridine-3′-phosphate
Tfs=2′-fluoro-5′-methyluridine-3′-phosphorothioate
Uf=2′-fluorouridine-3′-phosphate
Ufs=2′-fluorouridine-3′-phosphorothioate
dT=2′-deoxythymidine-3′-phosphate
AUNA=2′,3′-seco-adenosine-3′-phosphate
AUNAs=2′,3′-seco-adenosine-3′-phosphorothioate
CUNA=2′,3′-seco-cytidine-3′-phosphate
CUNAs=2′,3′-seco-cytidine-3′-phosphorothioate
GUNA=2′,3′-seco-guanosine-3′-phosphate
GUNAs=2′,3′-seco-guanosine-3′-phosphorothioate
UUNA=2′,3′-seco-uridine-3′-phosphate
UUNAs=2′,3′-seco-uridine-3′-phosphorothioate
a_2N=see Table 4
a_2Ns=see Table 4
(invAb)=inverted abasic deoxyribonucleotide-5′-phosphate, see Table 4
(invAb)s=inverted abasic deoxyribonucleotide-5′-phosphorothioate, see Table 4
s=phosphorothioate linkage
p=terminal phosphate (as synthesized)
vpdN=vinyl phosphonate deoxyribonucleotide
cPrpa=5′-cyclopropyl phosphonate-2′-O-methyladenosine-3′-phosphate (see Table 4)
cPrpas=5′-cyclopropyl phosphonate-2′-O-methyladenosine-3′-phosphorothioate (see Table 4)
cPrpu=5′-cyclopropyl phosphonate-2′-O-methyluridine-3′-phosphate (see Table 4)
cPrpus=5′-cyclopropyl phosphonate-2′-O-methyluridine-3′-phosphorothioate (see Table 4)
(Alk-SS-C6)=see Table 4
(C6-SS-Alk)=see Table 4
(C6-SS-C6)=see Table 4
(6-SS-6)=see Table 4
(C6-SS-Alk-Me)=see Table 4
(NH₂-C6)=see Table 4
(NH-C6)=see Table 4
(NH-C6)s=see Table 4
L6-C6=see Table 4
L6-C6s=see Table 4
cC16=see Table 4
aC16=see Table 4
gC16=see Table 4
uC16=see Table 4
ALNA=see Table 4

### Example 4. In Vivo Administration of Lipid-Linked Rnai Agents in Mice

On study day 1, female C57bl/6 mice were injected with either phosphate buffered saline (PBS) or RNAi agent formulated at 10 μL in PBS. Three (n=3) animals were dosed in each group with 10 μL of PBS or RNAi agent solution. Animals were injected intracerebroventicularly according to the dosing regimen of Table 5.

TABLE 5
Dosing regimen for mice of Example 4.

| Group | Dosing Material | Dosing Regimen |
|---|---|---|
| 1 | PBS | Single Injection on Day 1 |
| 2 | 100 ug AD08942 (No PK/PD | Single Injection on Day 1 |
| | modulator) | |
| 3 | 100 ug AC001249 (LP-183a) | Single Injection on Day 1 |
| 4 | 100 ug AC001374 (LP-128a) | Single Injection on Day 1 |
| 5 | 100 ug AC001375 (LP-200a) | Single Injection on Day 1 |
| 6 | 100 ug AC001380 (LP-273a) | Single Injection on Day 1 |
| 7 | 100 ug AC001381 (LP-274a) | Single Injection on Day 1 |
| 8 | 100 ug AC001382 (LP-276a) | Single Injection on Day 1 |
| 9 | 100 ug AC001384 (LP-293a) | Single Injection on Day 1 |
| 10 | 100 ug AC001389 (LP-233a) | Single Injection on Day 1 |
| 11 | 100 ug AC001390 (LP-242a) | Single Injection on Day 1 |
| 12 | 100 ug AC001391 (LP-243a) | Single Injection on Day 1 |
| 13 | 100 ug AC001392 (LP-249a) | Single Injection on Day 1 |
| 14 | 100 ug AC001393 (LP-259a) | Single Injection on Day 1 |
| 15 | 100 ug AC001394 (LP-260a) | Single Injection on Day 1 |
| 16 | 100 ug AC001377 (LP-257a) | Single Injection on Day 1 |

On study day 15, animals were sacrificed and the thoracic spinal cord, temporal and frontal cortex, and cerebellum were collected. Expression of Superoxidase Dismutase 1 (SOD1) in each tissue was determined using qPCR. Average SOD1 expression for each animal in each tissue was normalized relative to group 1 (PBS). Results are shown in Table 6a-6d, below.

TABLE 6a
Average Relative SOD1 expression in mice
of Example 4, in temporal cortex.

| | | Relative | | |
|---|---|---|---|---|
| | | SOD1 Ex- | Error | Error |
| Group | Description | pression | (High) | (Low) |
| 1 | PBS | 1.000 | 0.050 | 0.053 |
| 2 | 100 ug AD08942 (No PK/PD | 0.512 | 0.033 | 0.035 |
| | modulator) | | | |
| 3 | 100 ug AC001249 (LP-183a) | 0.406 | 0.095 | 0.124 |
| 4 | 100 ug AC001374 (LP-128a) | 0.611 | 0.115 | 0.141 |
| 5 | 100 ug AC001375 (LP-200a) | 0.525 | 0.095 | 0.117 |
| 6 | 100 ug AC001380 (LP-273a) | 0.376 | 0.080 | 0.102 |
| 7 | 100 ug AC001381 (LP-274a) | 0.418 | 0.101 | 0.133 |
| 8 | 100 ug AC001382 (LP-276a) | 0.497 | 0.083 | 0.100 |
| 9 | 100 ug AC001384 (LP-293a) | 0.254 | 0.127 | 0.255 |
| 10 | 100 ug AC001389 (LP-233a) | 0.466 | 0.139 | 0.199 |
| 11 | 100 ug AC001390 (LP-242a) | 0.322 | 0.086 | 0.118 |
| 12 | 100 ug AC001391 (LP-243a) | 0.532 | 0.106 | 0.132 |
| 13 | 100 ug AC001392 (LP-249a) | 0.459 | 0.096 | 0.121 |
| 14 | 100 ug AC001393 (LP-259a) | 0.381 | 0.086 | 0.110 |
| 15 | 100 ug AC001394 (LP-260a) | 0.293 | 0.075 | 0.101 |
| 16 | 100 ug AC001377 (LP-257a) | 0.358 | 0.125 | 0.191 |

TABLE 6b
Average Relative SOD1 expression in
mice of Example 4, in frontal cortex.

| | | Relative | | |
|---|---|---|---|---|
| | | SOD1 Ex- | Error | Error |
| Group | Description | pression | (High) | (Low) |
| 1 | PBS | 1.000 | 0.090 | 0.098 |
| 2 | 100 ug AD08942 (No PK/PD | 0.479 | 0.024 | 0.025 |
| | modulator) | | | |
| 3 | 100 ug AC001249 (LP-183a) | 0.377 | 0.041 | 0.046 |
| 4 | 100 ug AC001374 (LP-128a) | 0.665 | 0.070 | 0.079 |
| 5 | 100 ug AC001375 (LP-200a) | 0.748 | 0.137 | 0.167 |
| 6 | 100 ug AC001380 (LP-273a) | 0.416 | 0.066 | 0.078 |
| 7 | 100 ug AC001381 (LP-274a) | 0.410 | 0.095 | 0.124 |
| 8 | 100 ug AC001382 (LP-276a) | 0.445 | 0.024 | 0.025 |
| 9 | 100 ug AC001384 (LP-293a) | 0.478 | 0.116 | 0.152 |
| 10 | 100 ug AC001389 (LP-233a) | 0.620 | 0.082 | 0.095 |
| 11 | 100 ug AC001390 (LP-242a) | 0.416 | 0.043 | 0.048 |
| 12 | 100 ug AC001391 (LP-243a) | 0.433 | 0.031 | 0.034 |
| 13 | 100 ug AC001392 (LP-249a) | 0.570 | 0.137 | 0.181 |
| 14 | 100 ug AC001393 (LP-259a) | 0.432 | 0.119 | 0.164 |
| 15 | 100 ug AC001394 (LP-260a) | 0.472 | 0.069 | 0.080 |
| 16 | 100 ug AC001377 (LP-257a) | 0.670 | 0.055 | 0.060 |

TABLE 6c
Average Relative SOD1 expression in mice
of Example 4, in thoracic spinal cord.

| | | Relative | | |
|---|---|---|---|---|
| | | SOD1 Ex- | Error | Error |
| Group | Description | pression | (High) | (Low) |
| 1 | PBS | 1.000 | 0.063 | 0.068 |
| 2 | 100 ug AD08942 (No PK/PD | 0.458 | 0.070 | 0.082 |
| | modulator) | | | |
| 3 | 100 ug AC001249 (LP-183a) | 0.150 | 0.011 | 0.012 |
| 4 | 100 ug AC001374 (LP-128a) | 0.119 | 0.014 | 0.016 |
| 5 | 100 ug AC001375 (LP-200a) | 0.145 | 0.038 | 0.051 |
| 6 | 100 ug AC001380 (LP-273a) | 0.225 | 0.054 | 0.070 |
| 7 | 100 ug AC001381 (LP-274a) | 0.229 | 0.024 | 0.027 |
| 8 | 100 ug AC001382 (LP-276a) | 0.234 | 0.019 | 0.021 |
| 9 | 100 ug AC001384 (LP-293a) | 0.234 | 0.030 | 0.035 |
| 10 | 100 ug AC001389 (LP-233a) | 0.168 | 0.024 | 0.027 |
| 11 | 100 ug AC001390 (LP-242a) | 0.301 | 0.100 | 0.149 |
| 12 | 100 ug AC001391 (LP-243a) | 0.168 | 0.029 | 0.035 |
| 13 | 100 ug AC001392 (LP-249a) | 0.168 | 0.018 | 0.020 |
| 14 | 100 ug AC001393 (LP-259a) | ND* | ND* | ND* |
| 15 | 100 ug AC001394 (LP-260a) | 0.209 | 0.074 | 0.115 |
| 16 | 100 ug AC001377 (LP-257a) | 0.148 | 0.018 | 0.021 |

*No data

TABLE 6d
Average Relative SOD1 expression in
mice of Example 4, in cerebellum.

| | | Relative | | |
|---|---|---|---|---|
| | | SOD1 Ex- | Error | Error |
| Group | Description | pression | (High) | (Low) |
| 1 | PBS | 1.000 | 0.039 | 0.040 |
| 2 | 100 ug AD08942 (No PK/PD | 0.483 | 0.022 | 0.023 |
| | modulator) | | | |
| 3 | 100 ug AC001249 (LP-183a) | 0.321 | 0.028 | 0.030 |
| 4 | 100 ug AC001374 (LP-128a) | 0.332 | 0.031 | 0.034 |
| 5 | 100 ug AC001375 (LP-200a) | 0.357 | 0.049 | 0.056 |
| 6 | 100 ug AC001380 (LP-273a) | 0.343 | 0.055 | 0.065 |
| 7 | 100 ug AC001381 (LP-274a) | 0.283 | 0.031 | 0.035 |
| 8 | 100 ug AC001382 (LP-276a) | 0.332 | 0.033 | 0.037 |
| 9 | 100 ug AC001384 (LP-293a) | 0.287 | 0.018 | 0.020 |
| 10 | 100 ug AC001389 (LP-233a) | 0.243 | 0.023 | 0.026 |
| 11 | 100 ug AC001390 (LP-242a) | 0.304 | 0.080 | 0.108 |
| 12 | 100 ug AC001391 (LP-243a) | 0.256 | 0.011 | 0.012 |
| 13 | 100 ug AC001392 (LP-249a) | 0.284 | 0.027 | 0.030 |
| 14 | 100 ug AC001393 (LP-259a) | 0.247 | 0.043 | 0.052 |
| 15 | 100 ug AC001394 (LP-260a) | 0.289 | 0.093 | 0.138 |
| 16 | 100 ug AC001377 (LP-257a) | 0.303 | 0.024 | 0.026 |

### Example 5. In Vivo Administration of Lipid-Linked RNAi Agents in Mice

On study day 1, female C57bl/6 mice were injected with either phosphate buffered saline (PBS) or RNAi agent formulated at 10 μg/L in PBS. Three (n=3) animals were dosed in each group with 10 μL of PBS or RNAi agent solution. Animals were injected intracerebroventicularly according to the dosing regimen of Table 7.

TABLE 7
Dosing regimen for mice of Example 5.

| Group | Dosing Material | Dosing Regimen |
|---|---|---|
| 1 | PBS | Single Injection on Day 1 |
| 2 | 100 ug AD08942 (No PK/PD | Single Injection on Day 1 |
| | modulator) | |
| 3 | 100 ug AC001376 (LP-245a) | Single Injection on Day 1 |
| 4 | 100 ug AC001378 (LP-262a) | Single Injection on Day 1 |
| 5 | 100 ug AC001379 (LP-269a) | Single Injection on Day 1 |
| 6 | 100 ug AC001395 (LP-286a) | Single Injection on Day 1 |
| 7 | 100 ug AC001383 (LP-287a) | Single Injection on Day 1 |
| 8 | 100 ug AC001396 (LP-289a) | Single Injection on Day 1 |
| 9 | 100 ug AC001385 (LP-296a) | Single Injection on Day 1 |
| 10 | 100 ug AD08908 (No PK/PD | Single Injection on Day 1 |
| | modulator) | |
| 11 | 100 ug AC001385 (LP-296a) | Single Injection on Day 1 |
| 12 | 100 ug AC001386 (LP-300a) | Single Injection on Day 1 |
| 13 | 100 ug AC001388 (LP-232a) | Single Injection on Day 1 |
| 14 | 100 ug AC001397 (LP-290a) | Single Injection on Day 1 |
| 15 | 100 ug AC001442 (LP-304a) | Single Injection on Day 1 |
| 16 | 100 ug AC001445 (LP-310a) | Single Injection on Day 1 |
| 17 | 100 ug AC001446 (LP-303a) | Single Injection on Day 1 |
| 18 | 100 ug AC001249 (LP-183a) | Single Injection on Day 1 |

On study day 15, animals were sacrificed and the thoracic spinal cord, temporal and frontal cortex, and cerebellum were collected. Expression of Superoxidase Dismutase 1 (SOD1) in each tissue was determined using qPCR. Average SOD1 expression for each animal in each tissue was normalized relative to group 1 (PBS). Results are shown in Table 8a-8d, below.

TABLE 8a
Average Relative SOD1 expression in mice
of Example 5, in temporal cortex.

| | | Relative | | |
|---|---|---|---|---|
| | | SOD1 Ex- | Error | Error |
| Group | Description | pression | (High) | (Low) |
| 1 | PBS | 1.000 | 0.066 | 0.070 |
| 2 | 100 ug AD08942 (No PK/PD | 0.846 | 0.017 | 0.017 |
| | modulator) | | | |
| 3 | 100 ug AC001376 (LP-245a) | 0.720 | 0.023 | 0.023 |
| 4 | 100 ug AC001378 (LP-262a) | 0.682 | 0.126 | 0.154 |
| 5 | 100 ug AC001379 (LP-269a) | 0.682 | 0.108 | 0.129 |
| 6 | 100 ug AC001395 (LP-286a) | 0.999 | 0.085 | 0.093 |
| 7 | 100 ug AC001383 (LP-287a) | 0.537 | 0.083 | 0.098 |
| 8 | 100 ug AC001396 (LP-289a) | 0.554 | 0.108 | 0.135 |
| 9 | 100 ug AC001385 (LP-296a) | 0.550 | 0.049 | 0.054 |
| 10 | 100 ug AD08908 (No PK/PD | 0.649 | 0.088 | 0.102 |
| | modulator) | | | |
| 11 | 100 ug AC001385 (LP-296a) | 0.588 | 0.052 | 0.057 |
| 12 | 100 ug AC001386 (LP-300a) | 0.472 | 0.039 | 0.042 |
| 13 | 100 ug AC001388 (LP-232a) | 0.318 | 0.048 | 0.057 |
| 14 | 100 ug AC001397 (LP-290a) | 0.396 | 0.020 | 0.022 |
| 15 | 100 ug AC001442 (LP-304a) | 0.416 | 0.011 | 0.011 |
| 16 | 100 ug AC001445 (LP-310a) | 0.349 | 0.072 | 0.091 |
| 17 | 100 ug AC001446 (LP-303a) | 0.384 | 0.042 | 0.047 |
| 18 | 100 ug AC001249 (LP-183a) | 0.443 | 0.041 | 0.045 |

TABLE 8b
Average Relative SOD1 expression in
mice of Example 5, in frontal cortex.

| | | Relative | | |
|---|---|---|---|---|
| | | SOD1 Ex- | Error | Error |
| Group | Description | pression | (High) | (Low) |
| 1 | PBS | 1.000 | 0.048 | 0.051 |
| 2 | 100 ug AD08942 (No PK/PD | 0.899 | 0.066 | 0.072 |
| | modulator) | | | |
| 3 | 100 ug AC001376 (LP-245a) | 0.761 | 0.036 | 0.038 |
| 4 | 100 ug AC001378 (LP-262a) | 0.780 | 0.036 | 0.038 |
| 5 | 100 ug AC001379 (LP-269a) | 0.855 | 0.049 | 0.052 |
| 6 | 100 ug AC001395 (LP-286a) | 0.965 | 0.045 | 0.048 |
| 7 | 100 ug AC001383 (LP-287a) | 0.682 | 0.034 | 0.036 |
| 8 | 100 ug AC001396 (LP-289a) | 0.619 | 0.116 | 0.142 |
| 9 | 100 ug AC001385 (LP-296a) | 0.735 | 0.070 | 0.077 |
| 10 | 100 ug AD08908 (No PK/PD | 0.700 | 0.028 | 0.029 |
| | modulator) | | | |
| 11 | 100 ug AC001385 (LP-296a) | 0.623 | 0.083 | 0.096 |
| 12 | 100 ug AC001386 (LP-300a) | 0.660 | 0.094 | 0.110 |
| 13 | 100 ug AC001388 (LP-232a) | 0.493 | 0.050 | 0.056 |
| 14 | 100 ug AC001397 (LP-290a) | 0.518 | 0.084 | 0.100 |
| 15 | 100 ug AC001442 (LP-304a) | 0.676 | 0.091 | 0.106 |
| 16 | 100 ug AC001445 (LP-310a) | 0.520 | 0.120 | 0.156 |
| 17 | 100 ug AC001446 (LP-303a) | 0.674 | 0.121 | 0.148 |
| 18 | 100 ug AC001249 (LP-183a) | 0.795 | 0.082 | 0.092 |

TABLE 8c
Average Relative SOD1 expression in mice
of Example 5, in thoracic spinal cord.

| | | Relative | | |
|---|---|---|---|---|
| | | SOD1 Ex- | Error | Error |
| Group | Description | pression | (High) | (Low) |
| 1 | PBS | 1.000 | 0.067 | 0.072 |
| 2 | 100 ug AD08942 (No PK/PD | 0.409 | 0.036 | 0.040 |
| | modulator) | | | |
| 3 | 100 ug AC001376 (LP-245a) | 0.398 | 0.060 | 0.071 |
| 4 | 100 ug AC001378 (LP-262a) | 0.282 | 0.068 | 0.090 |
| 5 | 100 ug AC001379 (LP-269a) | 0.344 | 0.054 | 0.064 |
| 6 | 100 ug AC001395 (LP-286a) | 0.646 | 0.075 | 0.085 |
| 7 | 100 ug AC001383 (LP-287a) | 0.235 | 0.026 | 0.029 |
| 8 | 100 ug AC001396 (LP-289a) | 0.331 | 0.064 | 0.080 |
| 9 | 100 ug AC001385 (LP-296a) | 0.451 | 0.164 | 0.258 |
| 10 | 100 ug AD08908 (No PK/PD | 0.522 | 0.162 | 0.234 |
| | modulator) | | | |
| 11 | 100 ug AC001385 (LP-296a) | 0.537 | 0.065 | 0.074 |
| 12 | 100 ug AC001386 (LP-300a) | 0.367 | 0.068 | 0.084 |
| 13 | 100 ug AC001388 (LP-232a) | 0.252 | 0.059 | 0.078 |
| 14 | 100 ug AC001397 (LP-290a) | 0.272 | 0.071 | 0.097 |
| 15 | 100 ug AC001442 (LP-304a) | 0.194 | 0.022 | 0.024 |
| 16 | 100 ug AC001445 (LP-310a) | 0.259 | 0.064 | 0.086 |
| 17 | 100 ug AC001446 (LP-303a) | 0.144 | 0.030 | 0.038 |
| 18 | 100 ug AC001249 (LP-183a) | 0.118 | 0.014 | 0.016 |

TABLE 8d
Average Relative SOD1 expression in
mice of Example 5, in cerebellum.

| | | Relative | | |
|---|---|---|---|---|
| | | SOD1 Ex- | Error | Error |
| Group | Description | pression | (High) | (Low) |
| 1 | PBS | 1.000 | 0.062 | 0.066 |
| 2 | 100 ug AD08942 (No PK/PD | 0.623 | 0.028 | 0.029 |
| | modulator) | | | |
| 3 | 100 ug AC001376 (LP-245a) | 0.454 | 0.024 | 0.025 |
| 4 | 100 ug AC001378 (LP-262a) | 0.438 | 0.040 | 0.044 |
| 5 | 100 ug AC001379 (LP-269a) | 0.373 | 0.016 | 0.017 |
| 6 | 100 ug AC001395 (LP-286a) | 0.767 | 0.138 | 0.168 |
| 7 | 100 ug AC001383 (LP-287a) | 0.398 | 0.026 | 0.028 |
| 8 | 100 ug AC001396 (LP-289a) | 0.493 | 0.019 | 0.020 |
| 9 | 100 ug AC001385 (LP-296a) | 0.525 | 0.066 | 0.075 |
| 10 | 100 ug AD08908 (No PK/PD | 0.573 | 0.034 | 0.036 |
| | modulator) | | | |
| 11 | 100 ug AC001385 (LP-296a) | 0.512 | 0.049 | 0.054 |
| 12 | 100 ug AC001386 (LP-300a) | 0.341 | 0.053 | 0.063 |
| 13 | 100 ug AC001388 (LP-232a) | 0.270 | 0.014 | 0.015 |
| 14 | 100 ug AC001397 (LP-290a) | 0.378 | 0.031 | 0.033 |
| 15 | 100 ug AC001442 (LP-304a) | 0.414 | 0.032 | 0.035 |
| 16 | 100 ug AC001445 (LP-310a) | 0.309 | 0.030 | 0.034 |
| 17 | 100 ug AC001446 (LP-303a) | 0.441 | 0.016 | 0.017 |
| 18 | 100 ug AC001249 (LP-183a) | 0.403 | 0.031 | 0.034 |

### Example 6. In Vivo Administration of Lipid-Linked RNAi Agents in Transgenic Mice

On study day 1, female TgSOD1G93A mice modified to express human SOD were injected with either artificial cerebrospinal fluid (aCSF, obtained from a commercial supplier) or RNAi agent formulated at 3 (g/L in aCSF. Three (n=3) animals were dosed in each group with 10 μL of aCSF or RNAi agent solution in aCSF. Animals were injected intracerebroventicularly according to the dosing regimen of Table 9.

TABLE 9
Dosing regimen for mice of Example 6.

| Group | Dosing Material | Dosing Regimen |
|---|---|---|
| 1 | aCSF | Single Injection on Day 1 |
| 2 | 30 ug AD11556 (No PK/PD | Single Injection on Day 1 |
| | modulator) | |
| 3 | 30 ug AC002478 (LP183a) | Single Injection on Day 1 |
| 4 | 30 ug AD11758 (C16a) | Single Injection on Day 1 |
| 5 | 30 ug AD11759 (C22a) | Single Injection on Day 1 |
| 6 | 30 ug AD11821 (HO-C16a) | Single Injection on Day 1 |
| 7 | 30 ug AD11841 ((2C8C12)a) | Single Injection on Day 1 |
| 8 | 30 ug AD11842 ((2C6C10)a) | Single Injection on Day 1 |
| 9 | 30 ug AD11276 (internal uC16) | Single Injection on Day 1 |
| 10 | 30 ug AD11278 (internal uC16) | Single Injection on Day 1 |
| 11 | 30 ug AD11731 (internal uC16) | Single Injection on Day 1 |
| 12 | 30 ug AD11732 (internal uC16) | Single Injection on Day 1 |

On study day 8, animals were sacrificed and the thoracic spinal cord, cortex, cerebellum and brainstem were collected. Expression of Superoxidase Dismutase 1 (SOD1) in each tissue was determined using qPCR. Average SOD1 expression for each animal in each tissue was normalized relative to group 1 (PBS). Results are shown in Table 10a-10d, below.

TABLE 10a
Average Relative SOD1 expression in mice of Example 6, in cortex.

| | | Relative | | |
|---|---|---|---|---|
| | | SOD1 | Error | Error |
| Group | Description | Expression | (High) | (Low) |
| 1 | aCSF | 1.000 | 0.151 | 0.178 |
| 2 | 30 ug AD11556 (No PK/PD | 0.812 | 0.126 | 0.150 |
| | modulator) | | | |
| 3 | 30 ug AC002478 (LP-183a) | 0.591 | 0.109 | 0.133 |
| 4 | 30 ug AD11758 (C16a) | 0.689 | 0.095 | 0.110 |
| 5 | 30 ug AD11759 (C22a) | 0.809 | 0.130 | 0.155 |
| 6 | 30 ug AD11821 (HO-C16a) | 0.632 | 0.105 | 0.125 |
| 7 | 30 ug AD11841 ((2C8C12)a) | 0.922 | 0.117 | 0.134 |
| 8 | 30 ug AD11842 ((2C6C10)a) | 0.724 | 0.129 | 0.157 |
| 9 | 30 ug AD11276 (internal uC16) | 0.661 | 0.150 | 0.195 |
| 10 | 30 ug AD11278 (internal uC16) | 0.617 | 0.093 | 0.109 |
| 11 | 30 ug AD11731 (internal uC16) | 0.648 | 0.117 | 0.143 |
| 12 | 30 ug AD11732 (internal uC16) | 0.698 | 0.167 | 0.219 |

TABLE 10b
Average Relative SOD1 expression in mice of Example 6, in thoracic
spinal cord.

| | | Relative | | |
|---|---|---|---|---|
| | | SOD1 | Error | Error |
| Group | Description | Expression | (High) | (Low) |
| 1 | aCSF | 1.000 | 0.134 | 0.155 |
| 2 | 30 ug AD11556 (No PK/PD | 0.544 | 0.086 | 0.101 |
| | modulator) | | | |
| 3 | 30 ug AC002478 (LP-183a) | 0.180 | 0.054 | 0.076 |
| 4 | 30 ug AD11758 (C16a) | 0.165 | 0.018 | 0.020 |
| 5 | 30 ug AD11759 (C22a) | 0.469 | 0.060 | 0.069 |
| 6 | 30 ug AD11821 (HO-C16a) | 0.394 | 0.066 | 0.079 |
| 7 | 30 ug AD11841 ((2C8C12)a) | 0.281 | 0.055 | 0.069 |
| 8 | 30 ug AD11842 ((2C6C10)a) | 0.346 | 0.066 | 0.082 |
| 9 | 30 ug AD11276 (internal uC16) | 0.293 | 0.080 | 0.111 |
| 10 | 30 ug AD11278 (internal uC16) | 0.311 | 0.034 | 0.039 |
| 11 | 30 ug AD11731 (internal uC16) | 0.276 | 0.085 | 0.123 |
| 12 | 30 ug AD11732 (internal uC16) | 0.519 | 0.080 | 0.094 |

TABLE 10c
Average Relative SOD1 expression in mice of Example 6, in
cerebellum.

| | | Relative | | |
|---|---|---|---|---|
| | | SOD1 | Error | Error |
| Group | Description | Expression | (High) | (Low) |
| 1 | aCSF | 1.000 | 0.125 | 0.143 |
| 2 | 30 ug AD11556 (No PK/PD | 0.679 | 0.099 | 0.116 |
| | modulator) | | | |
| 3 | 30 ug AC002478 (LP-183a) | 0.473 | 0.069 | 0.080 |
| 4 | 30 ug AD11758 (C16a) | 0.690 | 0.063 | 0.070 |
| 5 | 30 ug AD11759 (C22a) | 0.532 | 0.173 | 0.256 |
| 6 | 30 ug AD11821 (HO-C16a) | 0.474 | 0.100 | 0.127 |
| 7 | 30 ug AD11841 ((2C8C12)a) | 0.802 | 0.096 | 0.109 |
| 8 | 30 ug AD11842 ((2C6C10)a) | 0.473 | 0.062 | 0.071 |
| 9 | 30 ug AD11276 (internal uC16) | 0.359 | 0.058 | 0.069 |
| 10 | 30 ug AD11278 (internal uC16) | 0.559 | 0.075 | 0.087 |
| 11 | 30 ug AD11731 (internal uC16) | 0.506 | 0.122 | 0.162 |
| 12 | 30 ug AD11732 (internal uC16) | 0.524 | 0.045 | 0.049 |

TABLE 10d
Average Relative SOD1 expression in mice of Example 6, in
brainstem.

| | | Relative | | |
|---|---|---|---|---|
| | | SOD1 | Error | Error |
| Group | Description | Expression | (High) | (Low) |
| 1 | aCSF | 1.000 | 0.162 | 0.193 |
| 2 | 30 ug AD11556 (No PK/PD | 0.655 | 0.067 | 0.075 |
| | modulator) | | | |
| 3 | 30 ug AC002478 (LP-183a) | 0.294 | 0.039 | 0.045 |
| 4 | 30 ug AD11758 (C16a) | 0.453 | 0.047 | 0.053 |
| 5 | 30 ug AD11759 (C22a) | 0.399 | 0.058 | 0.068 |
| 6 | 30 ug AD11821 (HO-C16a) | 0.426 | 0.061 | 0.071 |
| 7 | 30 ug AD11841 ((2C8C12)a) | 0.444 | 0.041 | 0.045 |
| 8 | 30 ug AD11842 ((2C6C10)a) | 0.509 | 0.094 | 0.115 |
| 9 | 30 ug AD11276 (internal uC16) | 0.448 | 0.081 | 0.099 |
| 10 | 30 ug AD11278 (internal uC16) | 0.436 | 0.056 | 0.064 |
| 11 | 30 ug AD11731 (internal uC16) | 0.373 | 0.036 | 0.040 |
| 12 | 30 ug AD11732 (internal uC16) | 0.542 | 0.070 | 0.080 |

### Example 7. In Vivo Administration of Lipid-Linked RNAi Agents in Transgenic Mice

On study day 1, female TgSODIG93A mice modified to express human SOD1 were injected with either artificial cerebrospinal fluid (aCSF, obtained from a commercial supplier) or RNAi agent formulated at 3 μg/L in aCSF. Three (n=3) animals were dosed in each group with 10 μL of aCSF or RNAi agent solution in aCSF. Animals were injected intracerebroventicularly according to the dosing regimen of Table 11.

TABLE 11
Dosing regimen for mice of Example 7.

| Group | Dosing Material | Dosing Regimen |
|---|---|---|
| 1 | aCSF | Single Injection on Day 1 |
| 2 | 30 ug AD11556 (No PK/PD | Single Injection on Day 1 |
| | modulator) | |
| 3 | 30 ug AC002478 (LP-183a) | Single Injection on Day 1 |
| 4 | 30 ug AC002548 (LP-283a) | Single Injection on Day 1 |
| 5 | 30 ug AC002549 (LP-383a) | Single Injection on Day 1 |
| 6 | 30 ug AC002550 (LP-396a) | Single Injection on Day 1 |
| 7 | 30 ug AD11739 (LP-395c) | Single Injection on Day 1 |
| 8 | 30 ug AD11692 (LP-183c) | Single Injection on Day 1 |
| 9 | 30 ug AD11728 (LP-409c) | Single Injection on Day 1 |

On study day 8, animals were sacrificed and the thoracic spinal cord, cortex, cerebellum and brainstem were collected. Expression of Superoxidase Dismutase 1 (SOD1) in each tissue was determined using qPCR. Average SOD1 expression for each animal in each tissue was normalized relative to group 1 (PBS). Results are shown in Table 12a-12d, below.

TABLE 12a
Average Relative SOD1 expression in mice of Example 7, in cortex.

| | | Relative | | |
|---|---|---|---|---|
| | | SOD1 | Error | Error |
| Group | Description | Expression | (High) | (Low) |
| 1 | aCSF | 1.000 | 0.037 | 0.039 |
| 2 | 30 ug AD11556 (No PK/PD | 0.920 | 0.078 | 0.086 |
| | modulator) | | | |
| 3 | 30 ug AC002478 (LP-183a) | 0.763 | 0.088 | 0.100 |
| 4 | 30 ug AC002548 (LP-283a) | 0.906 | 0.079 | 0.087 |
| 5 | 30 ug AC002549 (LP-383a) | 0.840 | 0.050 | 0.053 |
| 6 | 30 ug AC002550 (LP-396a) | 0.705 | 0.089 | 0.102 |
| 7 | 30 ug AD11739 (LP-395a) | 0.815 | 0.117 | 0.136 |
| 8 | 30 ug AD11692 (LP-183a) | 0.707 | 0.163 | 0.211 |
| 9 | 30 ug AD11728 (LP-409a) | 0.472 | 0.124 | 0.168 |

TABLE 12b
Average Relative SOD1 expression in mice of Example 7, in thoracic
spinal cord.

| | | Relative | | |
|---|---|---|---|---|
| | | SOD1 | Error | Error |
| Group | Description | Expression | (High) | (Low) |
| 1 | aCSF | 1.000 | 0.051 | 0.054 |
| 2 | 30 ug AD11556 (No PK/PD | 0.770 | 0.155 | 0.194 |
| | modulator) | | | |
| 3 | 30 ug AC002478 (LP-183a) | 0.348 | 0.099 | 0.138 |
| 4 | 30 ug AC002548 (LP-283a) | 0.765 | 0.186 | 0.246 |
| 5 | 30 ug AC002549 (LP-383a) | 0.537 | 0.259 | 0.499 |
| 6 | 30 ug AC002550 (LP-396a) | 0.616 | 0.035 | 0.037 |
| 7 | 30 ug AD11739 (LP-395a) | 0.550 | 0.021 | 0.022 |
| 8 | 30 ug AD11692 (LP-183a) | 0.368 | 0.059 | 0.070 |
| 9 | 30 ug AD11728 (LP-409a) | 0.239 | 0.022 | 0.024 |

TABLE 12c
Average Relative SOD1 expression in mice of Example 7, in
cerebellum.

| | | Relative | | |
|---|---|---|---|---|
| | | SOD1 | Error | Error |
| Group | Description | Expression | (High) | (Low) |
| 1 | aCSF | 1.000 | 0.121 | 0.138 |
| 2 | 30 ug AD11556 (No PK/PD | 0.550 | 0.057 | 0.064 |
| | modulator) | | | |
| 3 | 30 ug AC002478 (LP-183a) | 0.380 | 0.103 | 0.141 |
| 4 | 30 ug AC002548 (LP-283a) | 0.509 | 0.121 | 0.158 |
| 5 | 30 ug AC002549 (LP-383a) | 0.576 | 0.183 | 0.269 |
| 6 | 30 ug AC002550 (LP-396a) | 0.490 | 0.068 | 0.079 |
| 7 | 30 ug AD11739 (LP-395a) | 0.606 | 0.188 | 0.272 |
| 8 | 30 ug AD11692 (LP-183a) | 0.520 | 0.105 | 0.132 |
| 9 | 30 ug AD11728 (LP-409a) | 0.442 | 0.055 | 0.062 |

TABLE 12d
Average Relative SOD1 expression in mice of Example 7, in
brainstem.

| | | Relative | | |
|---|---|---|---|---|
| | | SOD1 | Error | Error |
| Group | Description | Expression | (High) | (Low) |
| 1 | aCSF | 1.000 | 0.134 | 0.155 |
| 2 | 30 ug AD11556 (No PK/PD | 0.815 | 0.183 | 0.237 |
| | modulator) | | | |
| 3 | 30 ug AC002478 (LP-183a) | 0.333 | 0.049 | 0.057 |
| 4 | 30 ug AC002548 (LP-283a) | 0.486 | 0.076 | 0.091 |
| 5 | 30 ug AC002549 (LP-383a) | 0.518 | 0.179 | 0.274 |
| 6 | 30 ug AC002550 (LP-396a) | 0.599 | 0.095 | 0.113 |
| 7 | 30 ug AD11739 (LP-395a) | 0.463 | 0.149 | 0.219 |
| 8 | 30 ug AD11692 (LP-183a) | 0.505 | 0.057 | 0.064 |
| 9 | 30 ug AD11728 (LP-409a) | 0.529 | 0.182 | 0.279 |

### Example 8. In Vivo Knockdown of SOD1 in Transgenic Tg SOD1 G93A Rats

On Study day 1, Tg SOD1 G93A rats were injected with either 30 μL artificial cerebrospinal fluid (aCSF, obtained from a commercial supplier) or 30 μL of compound formulation at a concentration of 0.33, 1.0, 3.33, 10, and 30 mg/mL for groups 2-6, respectively, in aCSF according to Table 13 below:

TABLE 13
Dosing groups for the rats of Example 8.

| Group ID | Animals dosed |
|---|---|
| Group 1 (aCSF) | n = 4 |
| Group 2 (10 ug AD12261) (LP-293a) | n = 4 |
| Group 3 (30 ug AD12261) (LP-293a) | n = 4 |
| Group 4 (100 ug AD12261) (LP-293a) | n = 4 |
| Group 5 (300 ug AD12261) (LP-293a) | n = 4 |
| Group 6 (900 ug AD12261) (LP-293a) | n = 4 |

Four (n=4) rats were dosed in each group. Rats were injected intrathecally on day 1. On day 85, CSF was collected from each animal, then rats were euthanized and the left half of the brain and thoracic spinal cord were collected and stored in 1000 NBF. Tissue samples were taken from the right half of the brain of thoracic spinal cord, cortex, cerebellum and brain stem. Samples were analyzed by qPCR for SOD1 mRNA knockdown. Average results for each group are shown in Table 14 below:

TABLE 14
Relative expression of SOD1 mRNA in various tissues analyzed
by qPCR for each of the dosing groups of Example 8.

| | | Cortex | | | Cerebellum | | |
|---|---|---|---|---|---|---|---|
| | | Group Average | | | Group Average | | |
| | | (n = 4) | | | (n = 4) | | |
| Group | | Rel. | Error | Error | Rel. | Error | Error |
| # | Description | Exp. | (Low) | (High) | Exp. | (Low) | (High) |
| 1 | aCSF | 1.000 | 0.127 | 0.145 | 1.000 | 0.100 | 0.111 |
| 2 | 10 μg AD12261 | 1.148 | 0.085 | 0.092 | 0.963 | 0.106 | 0.119 |
| | (LP-293a) | | | | | | |
| 3 | 30 μg AD12261 | 0.966 | 0.096 | 0.107 | 0.742 | 0.107 | 0.125 |
| | (LP-293a) | | | | | | |
| 4 | 100 μg AD12261 | 0.843 | 0.267 | 0.391 | 0.572 | 0.213 | 0.339 |
| | (LP-293a) | | | | | | |
| 5 | 300 μg AD12261 | 0.870 | 0.279 | 0.410 | 0.501 | 0.153 | 0.221 |
| | (LP-293a) | | | | | | |
| 6 | 900 μg AD12261 | 0.733 | 0.171 | 0.223 | 0.316 | 0.097 | 0.139 |
| | (LP-293a) | | | | | | |
| | | Thoracic Spinal Cord | | | Brainstem | | |
| | | Group Average (n = 4) | | | Group Average (n = 4) | | |
| Group | | Rel. | Error | Error | Rel. | Error | Error |
| # | Description | Exp. | (Low) | (High) | Exp. | (Low) | (High) |
| 1 | aCSF | 1.000 | 0.185 | 0.228 | 1.000 | 0.257 | 0.345 |
| 2 | 10 μg AD12261 | 1.140 | 0.115 | 0.129 | 1.056 | 0.146 | 0.170 |
| | (LP-293a) | | | | | | |
| 3 | 30 μg AD12261 | 0.845 | 0.153 | 0.186 | 0.988 | 0.213 | 0.272 |
| | (LP-293a) | | | | | | |
| 4 | 100 μg AD12261 | 0.595 | 0.250 | 0.430 | 0.843 | 0.300 | 0.465 |
| | (LP-293a) | | | | | | |
| 5 | 300 μg AD12261 | 0.507 | 0.130 | 0.175 | 0.865 | 0.124 | 0.145 |
| | (LP-293a) | | | | | | |
| 6 | 900 μg AD12261 | 0.217 | 0.066 | 0.094 | 0.605 | 0.108 | 0.132 |
| | (LP-293a) | | | | | | |

As shown in Table 14, above, a dose-dependent decrease in SOD1 mRNA expression was observed for transgenic rats treated with AD12261.

### Example 9. In Vivo Knockdown of SOD1 in Cynomolgus Monkeys

On Study day 1, cynomolgus monkeys were injected with either artificial cerebrospinal fluid (aCSF, obtained from a commercial supplier) or a compound formulation containing 45 mg of AD12261 in aCSF according to Table 15 below:

TABLE 15
Dosing groups for the non-human primates of Example 9.

| Group ID | Animals dosed |
|---|---|
| Group 1 (aCSF) | n = 4 |
| Group 2 (45 mg AD12261) (LP-293a)-Day 29 | n = 5 |
| Group 3 (45 mg AD12261) (LP-293a)-Day 85 | n = 5 |
| Group 4 (45 mg AD12261) (LP-293a)-Day 168 | n = 5 |

Four (n=4) monkeys were dosed in group 1 (control) and five (n=5) monkeys were dosed in groups 2, 3 and 4 (trigger treated). Monkeys were injected intrathecally on day 1. On study day 29, animals from Groups 1 and 2 were euthanized and brain and spinal cord tissue was collected from each animal. On study day 85, animals from Group 3 were euthanized and brain and spinal cord tissue was collected from each animal. On study day 168, animals from Group 4 were euthanized and brain and spinal cord tissue was collected from each animal. Samples were analyzed by qPCR for SOD1 mRNA knockdown. Average results for each group, relative to Group 1, are shown in Table 16 below:

TABLE 16
Relative expression of SOD1 mRNA in various tissues analyzed
by qPCR for each of the dosing groups of Example 9.

| | | Frontal Cortex | | | Temporal Cortex | | |
|---|---|---|---|---|---|---|---|
| | | Group Average | | | Group Average | | |
| Group | | Rel. | Error | Error | Rel. | Error | Error |
| # | Description | Exp. | (Low) | (High) | Exp. | (Low) | (High) |
| 1 | aCSF | 1.000 | 0.122 | 0.139 | 1.000 | 0.164 | 0.197 |
| 2-Day 29 | AD12261 | 0.267 | 0.191 | 0.666 | 0.184 | 0.129 | 0.437 |
| | (45 mg) | | | | | | |
| 3-Day 85 | AD12261 | 0.471 | 0.289 | 0.749 | 0.204 | 0.115 | 0.265 |
| | (45 mg) | | | | | | |
| 4-Day 168 | AD12261 | 0.463 | 0.191 | 0.326 | 0.273 | 0.101 | 0.160 |
| | (45 mg) | | | | | | |
| | | Cerebellum (Cortex) | | | Lumbar Spinal Cord | | |
| | | Group Average | | | Group Average | | |
| Group | | Rel. | Error | Error | Rel. | Error | Error |
| # | Description | Exp. | (Low) | (High) | Exp. | (Low) | (High) |
| 1 | aCSF | 1.000 | 0.207 | 0.262 | 1.000 | 0.535 | 1.152 |
| 2-Day 29 | AD12261 | 0.368 | 0.212 | 0.503 | 0.040 | 0.020 | 0.039 |
| | (45 mg) | | | | | | |
| 3-Day 85 | AD12261 | 0.726 | 0.220 | 0.316 | 0.025 | 0.012 | 0.024 |
| | (45 mg) | | | | | | |
| 4-Day 168 | AD12261 | 0.984 | 0.264 | 0.361 | 0.115 | 0.057 | 0.113 |
| | (45 mg) | | | | | | |
| | | Cervical Spinal Cord | | | Motor Cortex | | |
| | | Group Average | | | Group Average | | |
| Group | | Rel. | Error | Error | Rel. | Error | Error |
| # | Description | Exp. | (Low) | (High) | Exp. | (Low) | (High) |
| 1 | aCSF | 1.000 | 0.297 | 0.422 | 1.000 | 0.183 | 0.224 |
| 2-Day 29 | AD12261 | 0.119 | 0.080 | 0.238 | 0.281 | 0.178 | 0.490 |
| | (45 mg) | | | | | | |
| 3-Day 85 | AD12261 | 0.372 | 0.176 | 0.335 | 0.188 | 0.091 | 0.176 |
| | (45 mg) | | | | | | |
| 4-Day 168 | AD12261 | 0.906 | 0.206 | 0.266 | 0.676 | 0.364 | 0.790 |
| | (45 mg) | | | | | | |
| | | Hippocampus | | | Pons | | |
| | | Group Average | | | Group Average | | |
| Group | | Rel. | Error | Error | Rel. | Error | Error |
| # | Description | Exp. | (Low) | (High) | Exp. | (Low) | (High) |
| 1 | aCSF | 1.000 | 0.180 | 0.220 | 1.000 | 0.370 | 0.586 |
| 2-Day 29 | AD12261 | 0.175 | 0.131 | 0.520 | 0.306 | 0.176 | 0.412 |
| | (45 mg) | | | | | | |
| 3-Day 85 | AD12261 | 0.373 | 0.073 | 0.090 | 0.925 | 0.320 | 0.489 |
| | (45 mg) | | | | | | |
| 4-Day 168 | AD12261 | 0.481 | 0.155 | 0.229 | 0.981 | 0.296 | 0.425 |
| | (45 mg) | | | | | | |

| | | Thoracic Spinal Cord | | |
|---|---|---|---|---|
| | | Group Average | | |
| Group | | Rel. | Error | Error |
| # | Description | Exp. | (Low) | (High) |
| 1 | aCSF | 1.000 | 0.185 | 0.227 |
| 2-Day 29 | AD12261 | 0.122 | 0.074 | 0.188 |
| | (45 mg | | | |
| 3-Day 85 | AD12261 | 0.130 | 0.085 | 0.248 |
| | (45 mg) | | | |
| 4-Day 168 | AD12261 | 0.628 | 0.255 | 0.430 |
| | (45 mg) | | | |

As shown in Table 16, above, durable (up to 168 days) reduction of SOD1 mRNA expression was observed in multiple tissues for non-human primates treated with AD12261.

### Example 10. In Vivo Knockdown of SOD1 in Transgenic Tg SOD1 G93A Rats

On study day 1, male TgSOD1G93A rats (Sprague Dawley) modified to express human SOD1 were injected with either artificial cerebrospinal fluid (aCSF, obtained from a commercial supplier) or RNAi agent formulated at 10 mg/mL in aCSF. Four (n=4) animals were dosed in each group with 30 μL of aCSF or RNAi agent solution in aCSF. Animals were injected intrathecal (IT) according to the dosing regimen of Table 17.

TABLE 17
Dosing regimen for mice of Example 10.

| Group | Dosing Material | Dosing Regimen |
|---|---|---|
| 1 | aCSF | Single IT Injection on Day 1 |
| 2 | 300 ug AD13302 (10 mg/mL) | Single IT Injection on Day 1 |
| | (LP-409a) | |
| 3 | 300 ug AD13303 (10 mg/mL) | Single IT Injection on Day 1 |
| | (LP-430a) | |
| 4 | 300 ug AD13304 (10 mg/mL) | Single IT Injection on Day 1 |
| | (LP-431a) | |
| 5 | 300 ug AD13306 (10 mg/mL) | Single IT Injection on Day 1 |
| | (LP-435a) | |
| 6 | 300 ug AD13308 (10 mg/mL) | Single IT Injection on Day 1 |
| | (LP-439a) | |
| 7 | 300 ug AD13309 (10 mg/mL) | Single IT Injection on Day 1 |
| | (LP-429a) | |
| 8 | 300 ug AD13310 (10 mg/mL) | Single IT Injection on Day 1 |
| | (LP-441a) | |

On study day 8, animals were sacrificed and the thoracic spinal cord, cortex (temporal), cerebellum, brainstem, and dorsal root ganglion (lumbar) were collected. Expression of Superoxidase Dismutase 1 (SOD1) in each tissue was determined using qPCR, with PPIA control gene. Average SOD1 expression for each animal in each tissue was normalized relative to group 1 (aCSF). Results are shown in Table 18 below.

TABLE 18
Average Relative SOD1 expression in rats of Example 10

| | | Cortex | | | Thoracic Spinal Cord | | |
|---|---|---|---|---|---|---|---|
| Group | | Rel. | Error | Error | Rel. | Error | Error |
| # | Description | Exp. | (Low) | (High) | Exp. | (Low) | (High) |
| 1 | aCSF | 1.000 | 0.088 | 0.097 | 1.000 | 0.115 | 0.131 |
| 2 | AD13302 (LP-409a) | 1.069 | 0.196 | 0.240 | 0.133 | 0.061 | 0.112 |
| 3 | AD13303 (LP-430a) | 0.815 | 0.149 | 0.182 | 0.189 | 0.019 | 0.021 |
| 4 | AD13304 (LP-431a) | 1.019 | 0.163 | 0.193 | 1.033 | 0.134 | 0.154 |
| 5 | AD13306 (LP-435a) | 0.591 | 0.159 | 0.217 | 0.554 | 0.059 | 0.066 |
| 6 | AD13308 (LP-439a) | 1.110 | 0.115 | 0.128 | 0.466 | 0.167 | 0.259 |
| 7 | AD13309 (LP-429a) | 0.841 | 0.105 | 0.119 | 0.351 | 0.107 | 0.155 |
| 8 | AD13310 (LP-441a) | 0.958 | 0.129 | 0.149 | 0.464 | 0.065 | 0.076 |
| | | Cerebellum | | | Brainstem | | |
| Group | | Rel. | Error | Error | Rel. | Error | Error |
| # | Description | Exp. | (Low) | (High) | Exp. | (Low) | (High) |
| 1 | aCSF | 1.000 | 0.118 | 0.133 | 1.000 | 0.135 | 0.155 |
| 2 | AD13302 (LP-409a) | 0.515 | 0.141 | 0.194 | 0.499 | 0.217 | 0.383 |
| 3 | AD13303 (LP-430a) | 0.678 | 0.260 | 0.423 | 0.521 | 0.242 | 0.453 |
| 4 | AD13304 (LP-431a) | 0.468 | 0.124 | 0.169 | 0.659 | 0.257 | 0.421 |
| 5 | AD13306 (LP-435a) | 0.592 | 0.147 | 0.195 | 0.362 | 0.112 | 0.161 |
| 6 | AD13308 (LP-439a) | 0.707 | 0.134 | 0.166 | 0.573 | 0.182 | 0.267 |
| 7 | AD13309 (LP-429a) | 0.506 | 0.147 | 0.207 | 0.281 | 0.035 | 0.040 |
| 8 | AD13310 (LP-441a) | 0.684 | 0.097 | 0.112 | 0.436 | 0.068 | 0.081 |

| | | Dorsal Root Ganglion | | |
|---|---|---|---|---|
| Group | | Rel. | Error | Error |
| # | Description | Exp. | (Low) | (High) |
| 1 | aCSF | 1.000 | 0.232 | 0.302 |
| 2 | AD13302 (LP-409a) | 0.376 | 0.077 | 0.097 |
| 3 | AD13303 (LP-430a) | 0.433 | 0.142 | 0.212 |
| 4 | AD13304 (LP-431a) | 0.350 | 0.100 | 0.141 |
| 5 | AD13306 (LP-435a) | 0.452 | 0.101 | 0.130 |
| 6 | AD13308 (LP-439a) | 0.288 | 0.047 | 0.056 |
| 7 | AD13309 (LP-429a) | 0.434 | 0.080 | 0.097 |
| 8 | AD13310 (LP-441a) | 0.252 | 0.053 | 0.066 |

### Example 11. In Vivo Knockdown of SOD1 in Transgenic TgSOD1 G93A Rats

On study day 1, male TgSOD1G93A rats (Sprague Dawley) modified to express human SOD1 were injected with either artificial cerebrospinal fluid (aCSF, obtained from a commercial supplier) or RNAi agent formulated at 10 mg/mL in aCSF. Four (n=4) animals were dosed in each group with 30 μL of aCSF or RNAi agent solution in aCSF. Animals were injected intrathecal (IT) according to the dosing regimen of Table 19.

TABLE 19
Dosing regimen for mice of Example 11.

| Group | Dosing Material | Dosing Regimen |
|---|---|---|
| 1 | aCSF | Single IT Injection on Day 1 |
| 2 | 300 ug AC001384 (10 mg/mL) | Single IT Injection on Day 1 |
| | (LP-293a) | |
| 3 | 300 ug AD13681 (10 mg/mL) | Single IT Injection on Day 1 |
| | (LP-440a) | |
| 4 | 300 ug AD13682 (10 mg/mL) | Single IT Injection on Day 1 |
| | (LP-132a) | |
| 5 | 300 ug AD13683 (10 mg/mL) | Single IT Injection on Day 1 |
| | (LP-465a) | |
| 6 | 300 ug AD13684 (10 mg/mL) | Single IT Injection on Day 1 |
| | (LP-456a) | |
| 7 | 300 ug AD13685 (10 mg/mL) | Single IT Injection on Day 1 |
| | (LP-462a) | |
| 8 | 300 ug AD13686 (10 mg/mL) | Single IT Injection on Day 1 |
| | (LP-464a) | |
| 9 | 300 ug AD13687 (10 mg/mL) | Single IT Injection on Day 1 |
| | (LP-463a) | |
| 10 | 300 ug AD13688 (10 mg/mL) | Single IT Injection on Day 1 |
| | (LP-466a) | |

On study day 8, animals were sacrificed and the cortex (temporal), thoracic spinal cord, cerebellum, and brainstem were collected. Expression of Superoxidase Dismutase 1 (SOD1) in each tissue was determined using qPCR, with PPIA control gene. Average SOD1 expression for each animal in each tissue was normalized relative to group 1 (aCSF). Results are shown in Table 20 below.

TABLE 20
Average Relative SOD1 expression in rats of Example 11

| | | Cortex | | | Thoracic Spinal Cord | | |
|---|---|---|---|---|---|---|---|
| Group | | Rel. | Error | Error | Rel. | Error | Error |
| # | Description | Exp. | (Low) | (High) | Exp. | (Low) | (High) |
| 1 | aCSF | 1.000 | 0.119 | 0.135 | 1.000 | 0.033 | 0.035 |
| 2 | AC001384 (LP-293a) | 0.725 | 0.162 | 0.209 | 0.183 | 0.094 | 0.193 |
| 3 | AD13681 (LP-440a) | 0.629 | 0.145 | 0.188 | 0.150 | 0.048 | 0.071 |
| 4 | AD13682 (LP-132a) | 0.845 | 0.116 | 0.134 | 0.141 | 0.041 | 0.058 |
| 5 | AD13683 (LP-465a) | 0.596 | 0.047 | 0.051 | 0.118 | 0.038 | 0.056 |
| 6 | AD13684 (LP-456a) | 0.857 | 0.143 | 0.172 | 0.208 | 0.068 | 0.101 |
| 7 | AD13685 (LP-462a) | 0.465 | 0.047 | 0.053 | 0.083 | 0.007 | 0.008 |
| 8 | AD13686 (LP-464a) | 0.749 | 0.125 | 0.150 | 0.115 | 0.036 | 0.052 |
| 9 | AD13687 (LP-463a) | 0.667 | 0.141 | 0.179 | 0.185 | 0.104 | 0.237 |
| 10 | AD13688 (LP-466a) | 0.689 | 0.188 | 0.259 | 0.210 | 0.130 | 0.337 |
| | | Cerebellum | | | Brainstem | | |
| Group | | Rel. | Error | Error | Rel. | Error | Error |
| # | Description | Exp. | (Low) | (High) | Exp. | (Low) | (High) |
| 1 | aCSF | 1.000 | 0.128 | 0.147 | 1.000 | 0.049 | 0.051 |
| 2 | AC001384 (LP-293a) | 0.454 | 0.159 | 0.244 | 0.434 | 0.143 | 0.213 |
| 3 | AD13681 (LP-440a) | 0.525 | 0.091 | 0.110 | 0.439 | 0.121 | 0.167 |
| 4 | AD13682 (LP-132a) | 0.511 | 0.145 | 0.203 | 0.475 | 0.140 | 0.198 |
| 5 | AD13683 (LP-465a) | 0.409 | 0.082 | 0.102 | 0.405 | 0.073 | 0.090 |
| 6 | AD13684 (LP-456a) | 0.688 | 0.090 | 0.104 | 0.621 | 0.164 | 0.223 |
| 7 | AD13685 (LP-462a) | 0.404 | 0.063 | 0.075 | 0.328 | 0.082 | 0.109 |
| 8 | AD13686 (LP-464a) | 0.508 | 0.051 | 0.057 | 0.496 | 0.118 | 0.155 |
| 9 | AD13687 (LP-463a) | 0.494 | 0.126 | 0.169 | 0.744 | 0.127 | 0.153 |
| 10 | AD13688 (LP-466a) | 0.573 | 0.164 | 0.231 | 0.653 | 0.228 | 0.351 |

### Example 12. In Vivo Knockdown of SOD1 in Transgenic Tg SOD1 G93A Rats

On study day 1, male TgSOD1G93A rats (Sprague Dawley) modified to express human SOD1 were injected with either artificial cerebrospinal fluid (aCSF, obtained from a commercial supplier) or RNAi agent formulated at 10 mg/mL in aCSF. Four (n=4) animals were dosed in each group with 30 μL of aCSF or RNAi agent solution in aCSF. Animals were injected intrathecal (IT) according to the dosing regimen of Table 21.

TABLE 21
Dosing regimen for mice of Example 12.

| Group | Dosing Material | Dosing Regimen |
|---|---|---|
| 1 | aCSF | Single IT Injection on Day 1 |
| 2 | 300 ug AC910361 (10 mg/mL) | Single IT Injection on Day 1 |
| | (internal uC16) | |
| 3 | 300 ug AC910860 (10 mg/mL) | Single IT Injection on Day 1 |
| | (LP-293a) | |
| 4 | 300 ug AC912620 (10 mg/mL) | Single IT Injection on Day 1 |
| | (internal uLP-493a) | |

On study day 8, animals were sacrificed and the cortex, thoracic spinal cord, cerebellum, brainstem, heart, midbrain, and hippocampus were collected. Expression of Superoxidase Dismutase 1 (SOD1) in each tissue was determined using qPCR, with PPIA control gene. Average SOD1 expression for each animal in each tissue was normalized relative to group 1 (aCSF). Results are shown in Table 22 below.

TABLE 22
Average Relative SOD1 expression in rats of Example 12

| | | Cortex | | | Thoracic Spinal Cord | | |
|---|---|---|---|---|---|---|---|
| Group | | Rel. | Error | Error | Rel. | Error | Error |
| # | Description | Exp. | (Low) | (High) | Exp. | (Low) | (High) |
| 1 | aCSF | 1.000 | 0.047 | 0.049 | 1.000 | 0.058 | 0.061 |
| 2 | AC910361 (uC16) | 0.367 | 0.049 | 0.057 | 0.091 | 0.023 | 0.031 |
| 3 | AC910860 (LP-293a) | 0.572 | 0.214 | 0.342 | 0.253 | 0.114 | 0.209 |
| 4 | AC912620 (uLP-493a) | 0.764 | 0.266 | 0.408 | 0.386 | 0.164 | 0.284 |
| | | Cerebellum | | | Brainstem | | |
| Group | | Rel. | Error | Error | Rel. | Error | Error |
| # | Description | Exp. | (Low) | (High) | Exp. | (Low) | (High) |
| 1 | aCSF | 1.000 | 0.184 | 0.226 | 1.000 | 0.092 | 0.101 |
| 2 | AC910361 (uC16) | 0.230 | 0.032 | 0.038 | 0.316 | 0.046 | 0.053 |
| 3 | AC910860 (LP-293a) | 0.338 | 0.150 | 0.268 | 0.508 | 0.151 | 0.214 |
| 4 | AC912620 (uLP-493a) | 0.456 | 0.062 | 0.072 | 0.643 | 0.160 | 0.214 |
| | | Heart | | | Midbrain | | |
| Group | | Rel. | Error | Error | Rel. | Error | Error |
| # | Description | Exp. | (Low) | (High) | Exp. | (Low) | (High) |
| 1 | aCSF | 1.000 | 0.094 | 0.104 | 1.000 | 0.089 | 0.097 |
| 2 | AC910361 (uC16) | 0.775 | 0.114 | 0.133 | 0.421 | 0.106 | 0.142 |
| 3 | AC910860 (LP-293a) | 0.995 | 0.054 | 0.057 | 0.531 | 0.140 | 0.189 |
| 4 | AC912620 (uLP-493a) | 0.966 | 0.153 | 0.182 | 0.702 | 0.147 | 0.185 |

| | | Hippocampus | | |
|---|---|---|---|---|
| Group | | Rel. | Error | Error |
| # | Description | Exp. | (Low) | (High) |
| 1 | aCSF | 1.000 | 0.168 | 0.202 |
| 2 | AC910361 (uC16) | 0.305 | 0.059 | 0.074 |
| 3 | AC910860 (LP-293a) | 0.439 | 0.153 | 0.235 |
| 4 | AC912620 (uLP-493a) | 0.725 | 0.179 | 0.238 |

### EQUIVALENTS AND SCOPE

In the claims articles such as “a,” “an,” and “the” may mean one or more than one unless indicated to the contrary or otherwise evident from the context. Claims or descriptions that include “or” between one or more members of a group are considered satisfied if one, more than one, or all of the group members are present in, employed in, or otherwise relevant to a given product or process unless indicated to the contrary or otherwise evident from the context. The invention includes embodiments in which exactly one member of the group is present in, employed in, or otherwise relevant to a given product or process. The invention includes embodiments in which more than one, or all of the group members are present in, employed in, or otherwise relevant to a given product or process.

Furthermore, the invention encompasses all variations, combinations, and permutations in which one or more limitations, elements, clauses, and descriptive terms from one or more of the listed claims is introduced into another claim. For example, any claim that is dependent on another claim can be modified to include one or more limitations found in any other claim that is dependent on the same base claim. Where elements are presented as lists, e.g., in Markush group format, each subgroup of the elements is also disclosed, and any element(s) can be removed from the group. It should it be understood that, in general, where the invention, or aspects of the invention, is/are referred to as comprising particular elements and/or features, certain embodiments of the invention or aspects of the invention consist, or consist essentially of, such elements and/or features. For purposes of simplicity, those embodiments have not been specifically set forth in haec verba herein. It is also noted that the terms “comprising” and “containing” are intended to be open and permits the inclusion of additional elements or steps. Where ranges are given, endpoints are included. Furthermore, unless otherwise indicated or otherwise evident from the context and understanding of one of ordinary skill in the art, values that are expressed as ranges can assume any specific value or sub-range within the stated ranges in different embodiments of the invention, to the tenth of the unit of the lower limit of the range, unless the context clearly dictates otherwise.

This application refers to various issued patents, published patent applications, journal articles, and other publications, all of which are incorporated herein by reference. If there is a conflict between any of the incorporated references and the instant specification, the specification shall control. In addition, any particular embodiment of the present invention that falls within the prior art may be explicitly excluded from any one or more of the claims. Because such embodiments are deemed to be known to one of ordinary skill in the art, they may be excluded even if the exclusion is not set forth explicitly herein. Any particular embodiment of the invention can be excluded from any claim, for any reason, whether or not related to the existence of prior art.

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1. A compound comprising:
a) an oligonucleotide; and
b) a lipid conjugated to the 5′ position or the 3′ position of one of the terminal nucleotides of the oligonucleotide;
wherein the oligonucleotide comprises at least 15 nucleotides that are complementary to a gene expressed in CNS tissue.

2-4. (canceled)

5. The compound of claim 1, wherein the oligonucleotide comprises a sense strand and an antisense strand, and the lipid is conjugated to the 5′ position of the sense strand.

6-7. (canceled)

8. The compound of claim 1, wherein the lipid is saturated or unsaturated and comprises 10 to 30 carbon atoms.

9. (canceled)

10. The compound of claim 1, wherein the lipid is selected from the group consisting of: 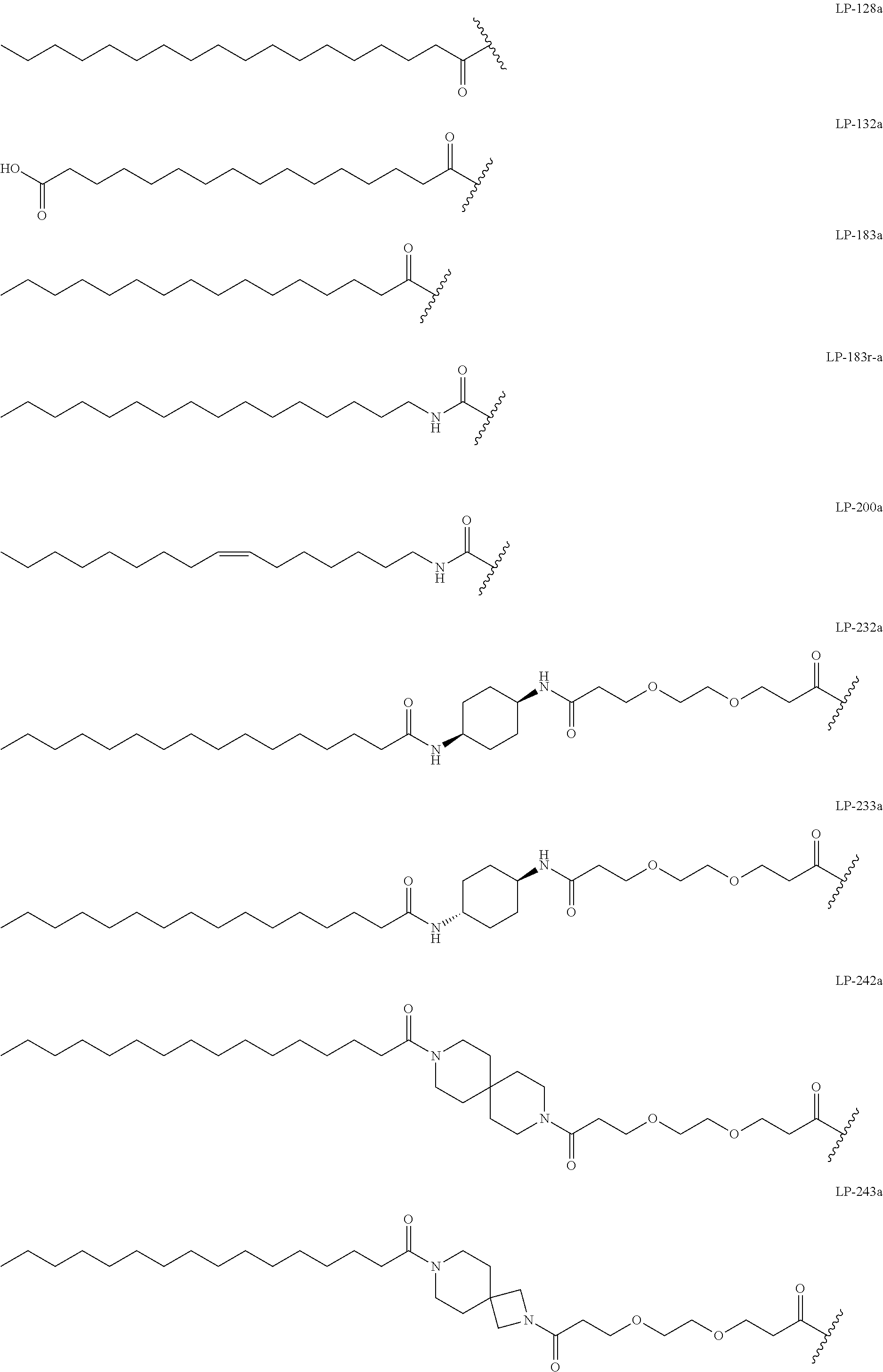 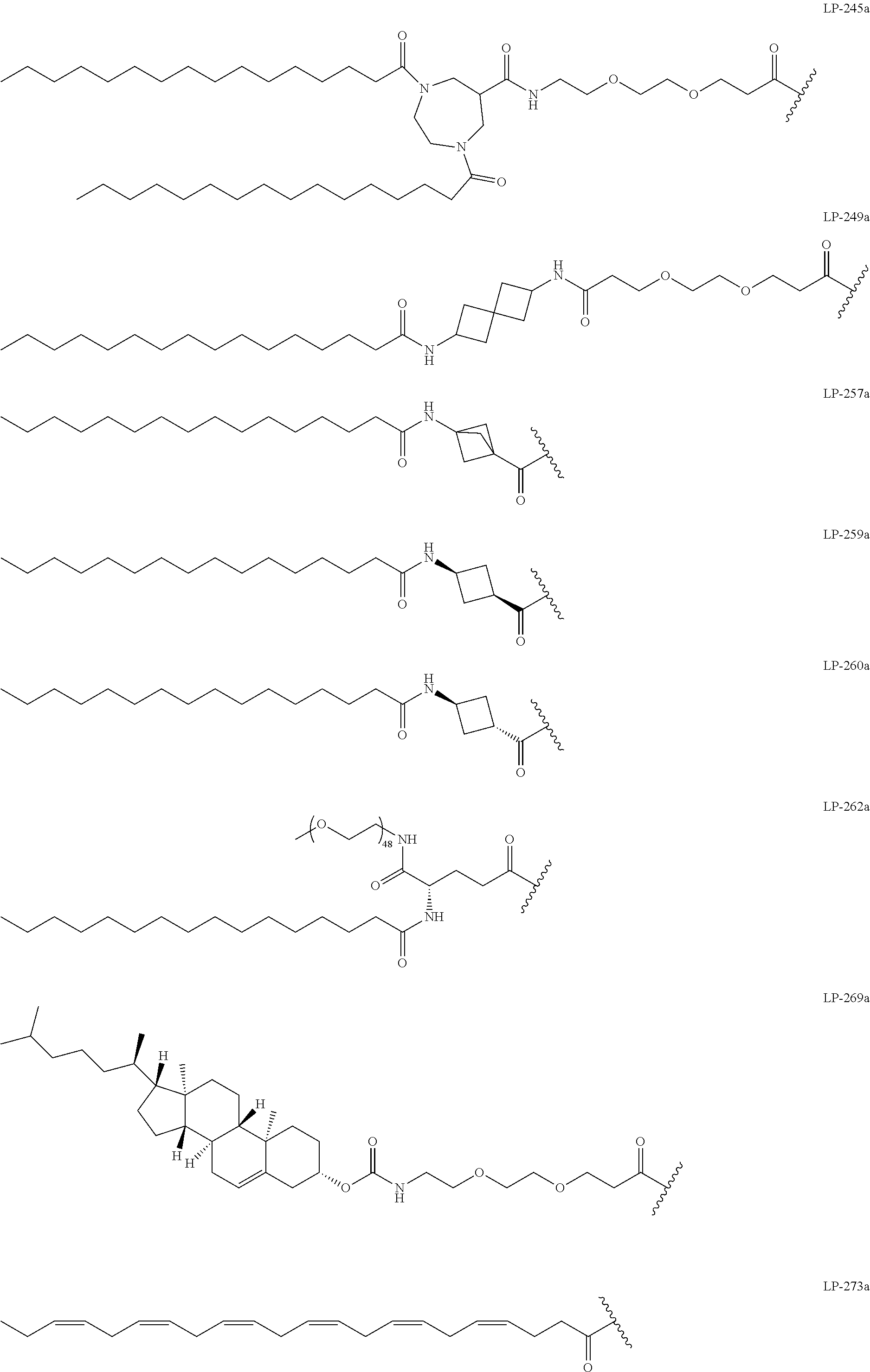 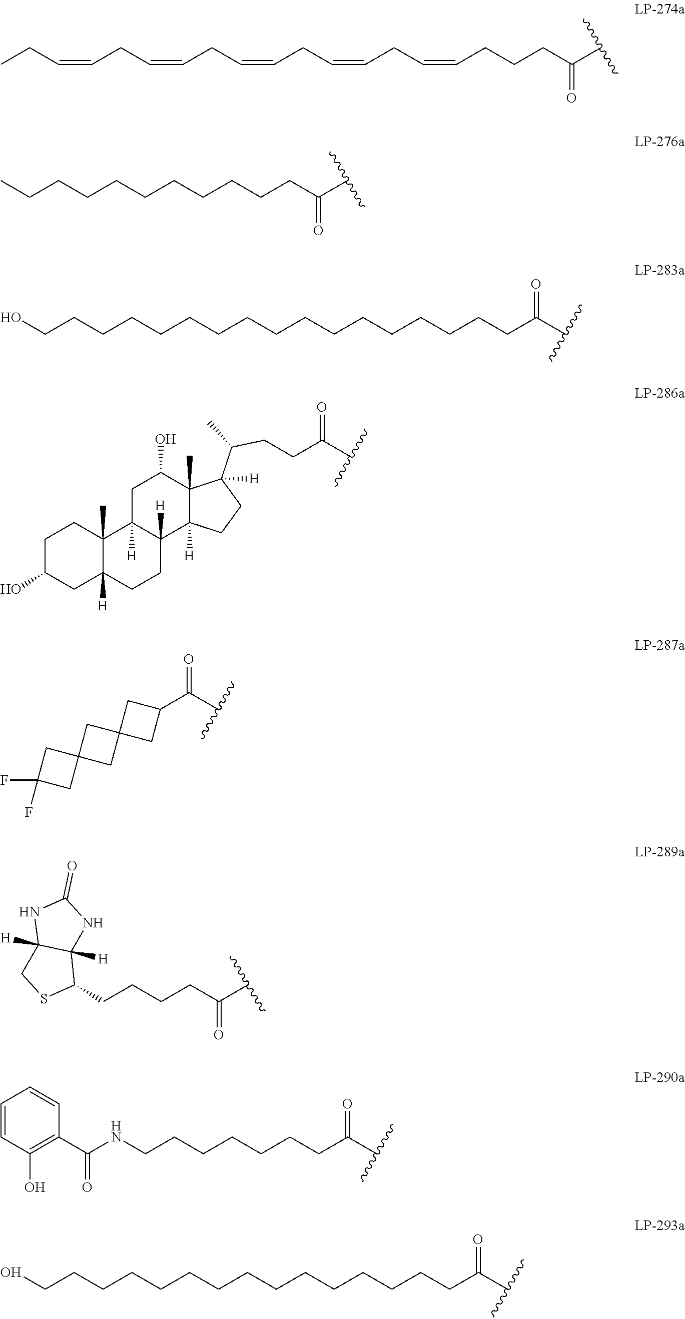 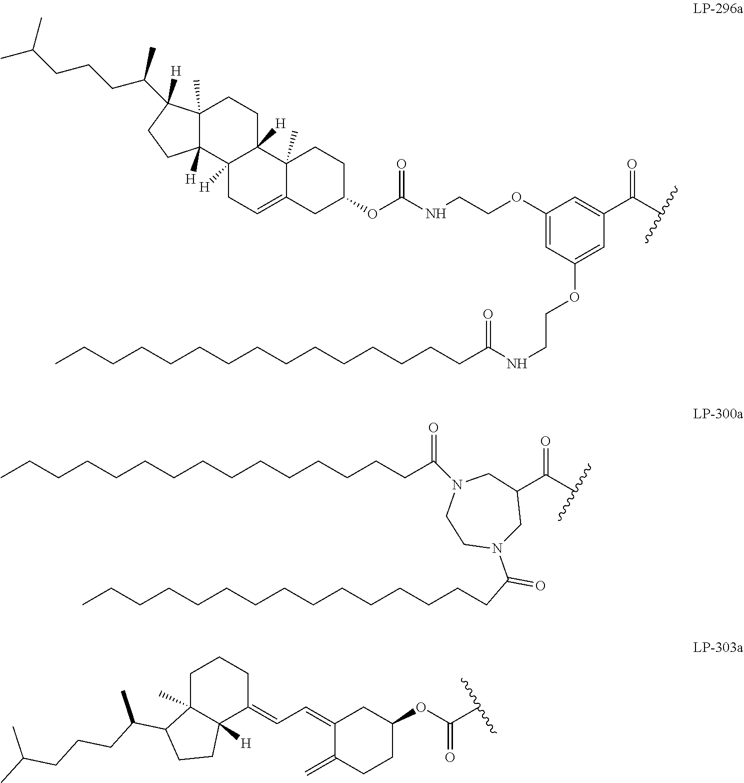 [SMILES: *C(=O)c1cc(OCCNC(=O)CCCCCCCCCCCCCCC)cc(OCCNC(=O)O[C@H]2CC[C@@]3(C)C(=CC[C@H]4[C@@H]5CC[C@H]([C@H](C)CCCC(C)C)[C@@]5(C)CC[C@@H]43)C2)c1 ; *C(=O)C1CN(C(=O)CCCCCCCCCCCCCCC)CCN(C(=O)CCCCCCCCCCCCCCC)C1 ; *C(=O)O[C@H]1CCC(=C)/C(=C\C=C2/CCC[C@]3(C)C([C@H](C)CCCC(C)C)CC[C@@H]23)C1] 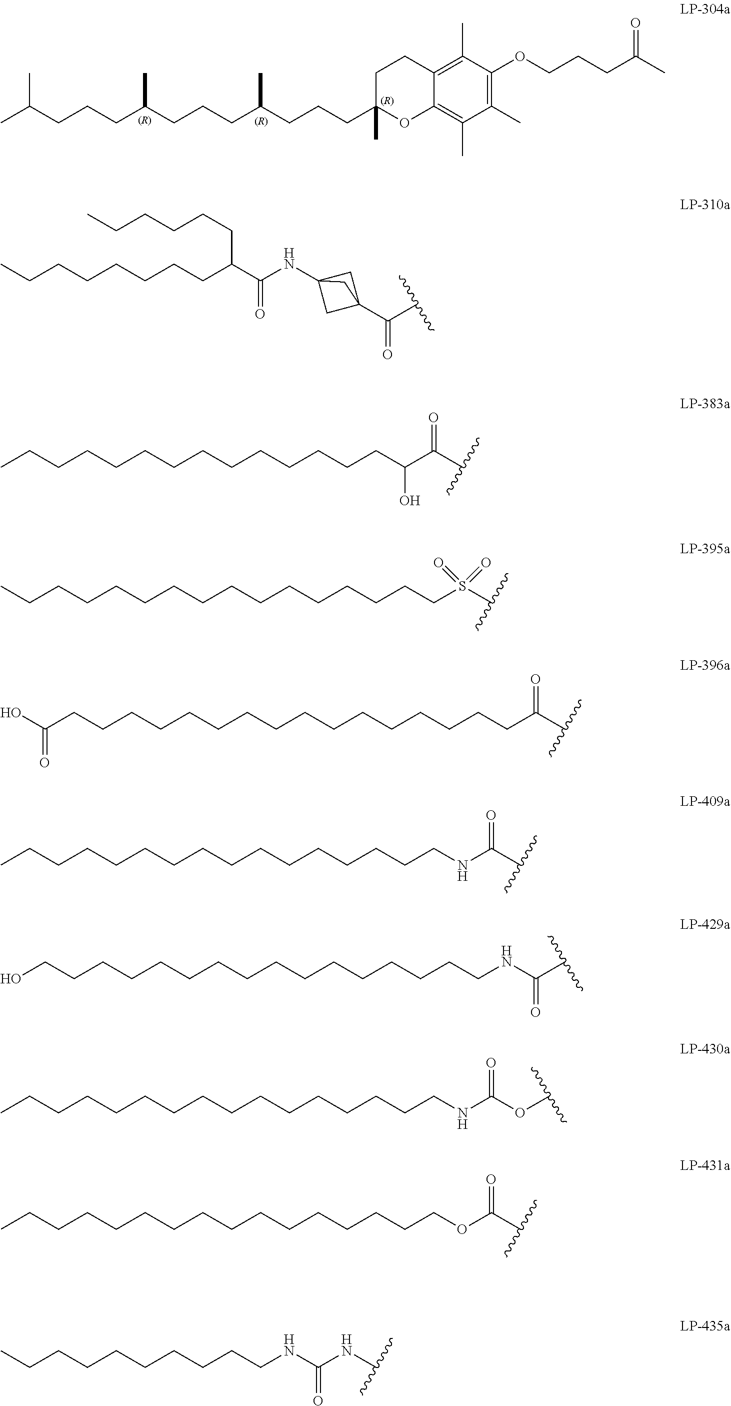 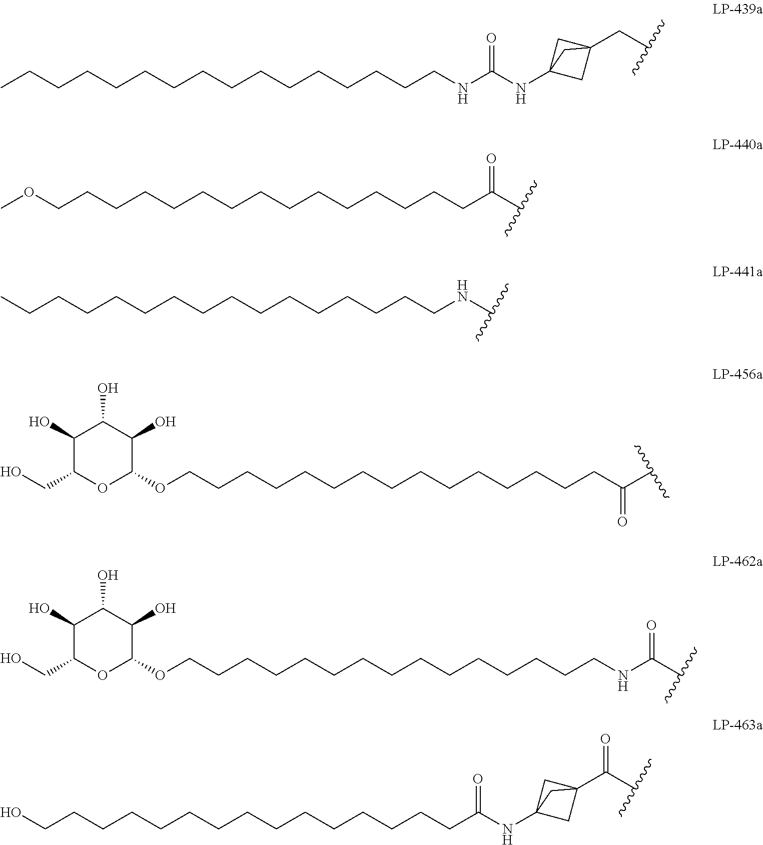 [SMILES: *CC12CC(NC(=O)NCCCCCCCCCCCCCCCC)(C1)C2 ; *C(=O)CCCCCCCCCCCCCCCOC ; *NCCCCCCCCCCCCCCCC ; *C(=O)CCCCCCCCCCCCCCCO[C@@H]1O[C@H](CO)[C@@H](O)[C@H](O)[C@H]1O ; *C(=O)NCCCCCCCCCCCCCCCO[C@@H]1O[C@H](CO)[C@@H](O)[C@H](O)[C@H]1O ; *C(=O)C12CC(NC(=O)CCCCCCCCCCCCCCCO)(C1)C2] 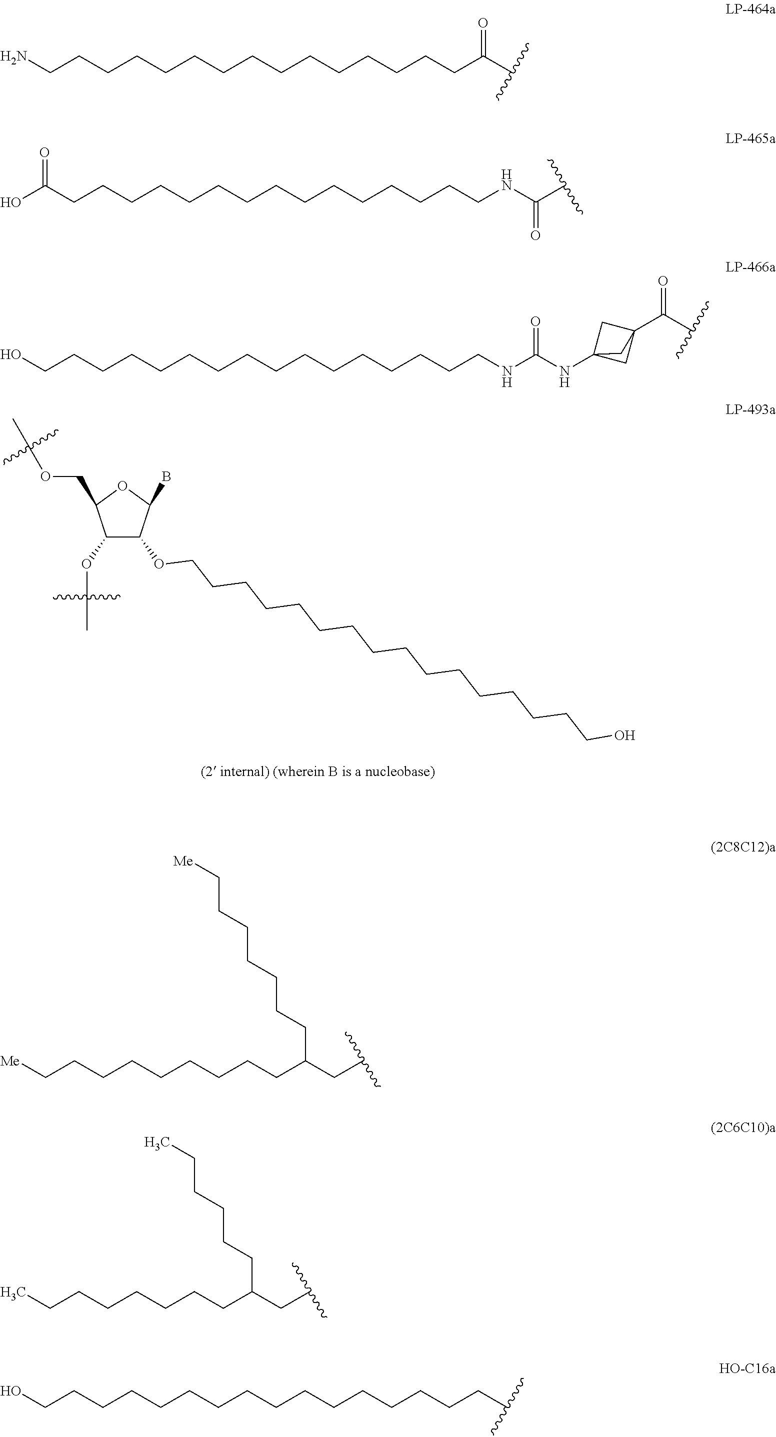 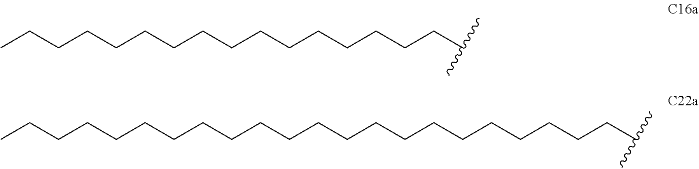 [SMILES: *CCCCCCCCCCCCCCCC ; *CCCCCCCCCCCCCCCCCCCCCC]
wherein [symbol] indicates the point of connection to the oligonucleotide.

11. The compound of claim 1, wherein the oligonucleotide is an RNAi agent.

12. The compound of claim 5, wherein the antisense strand comprises a cyclopropylene (cPrp)-modified nucleotide, which is the 5′-terminal nucleotide of the antisense strand.

13. (canceled)

14. A compound selected from the group consisting of: 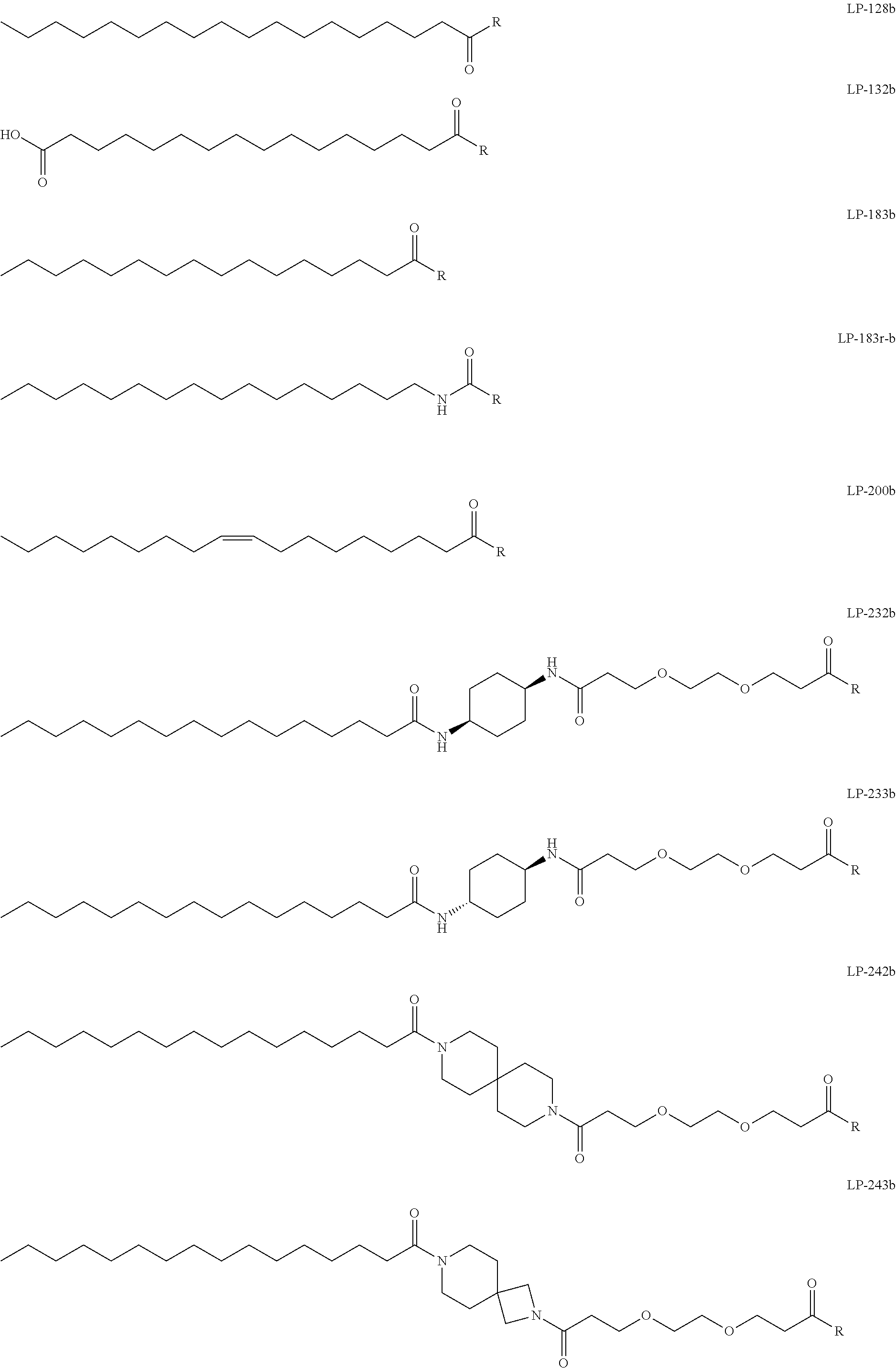 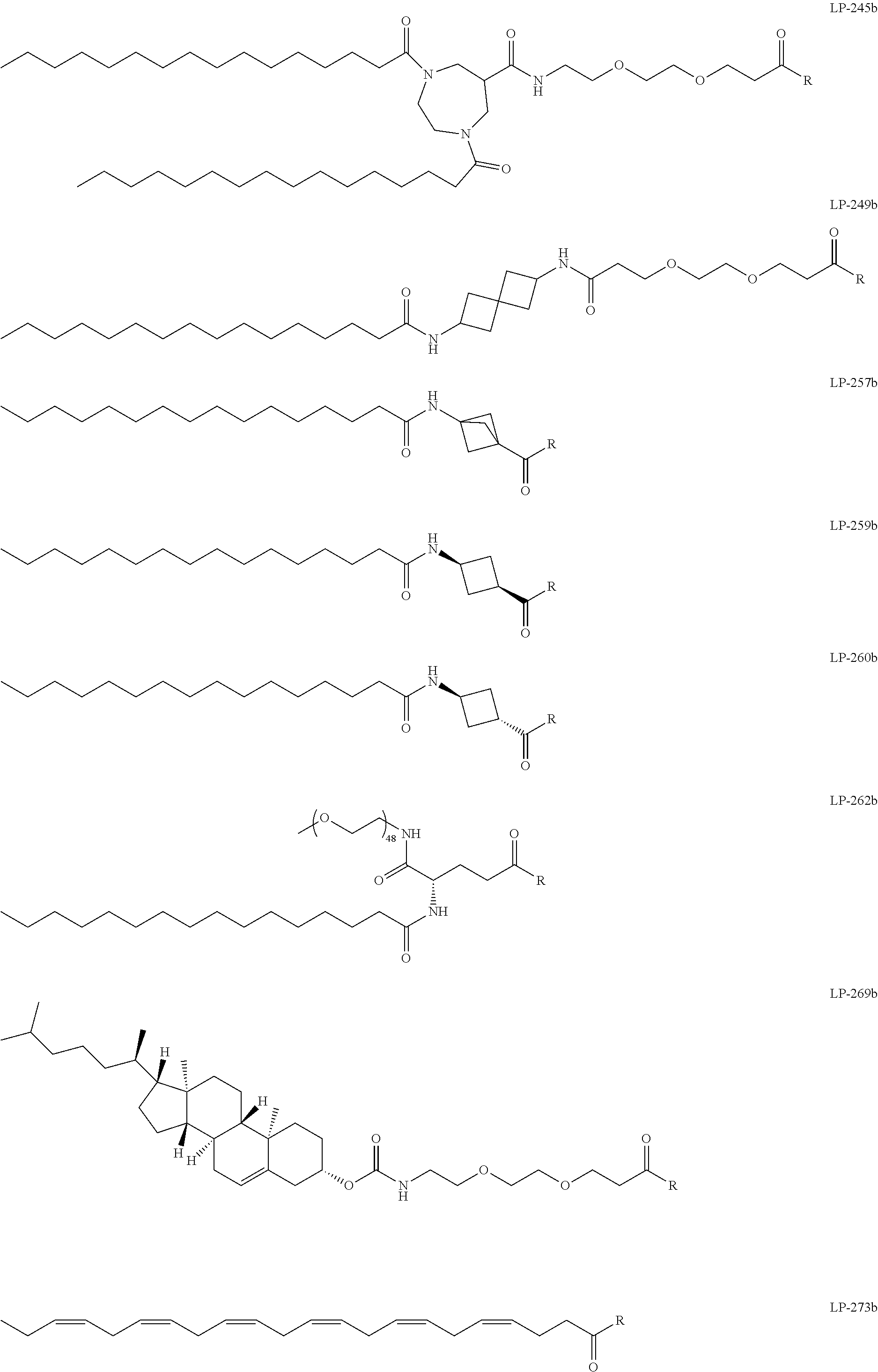 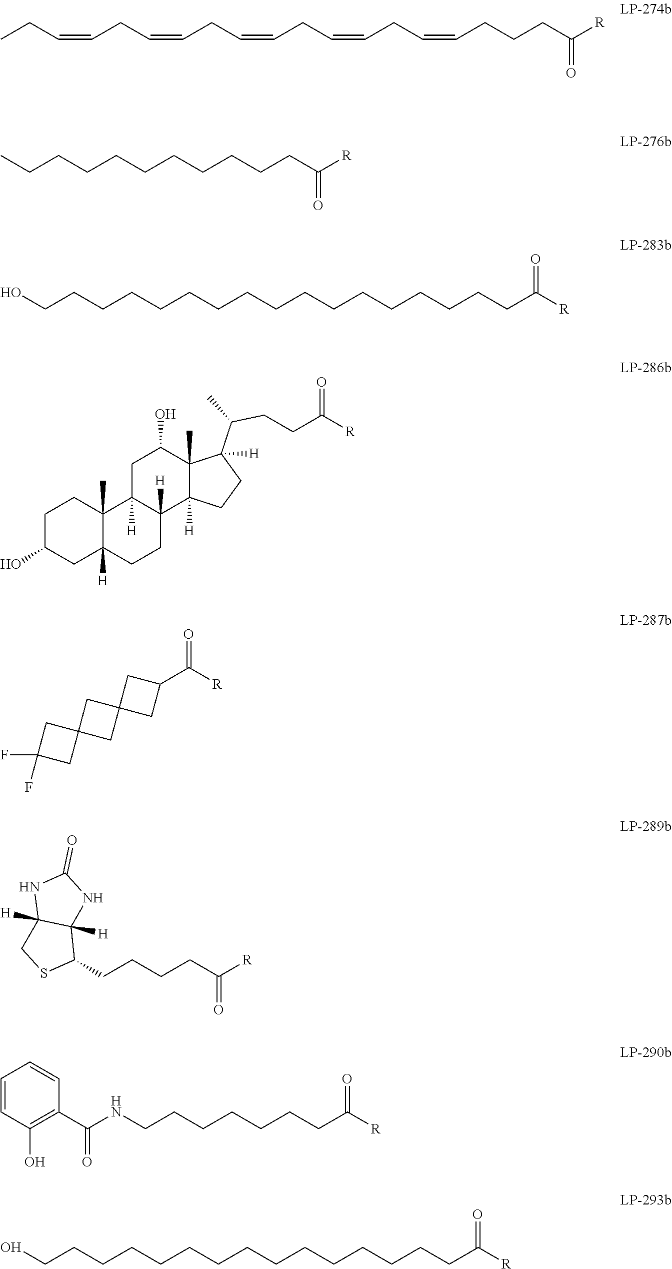 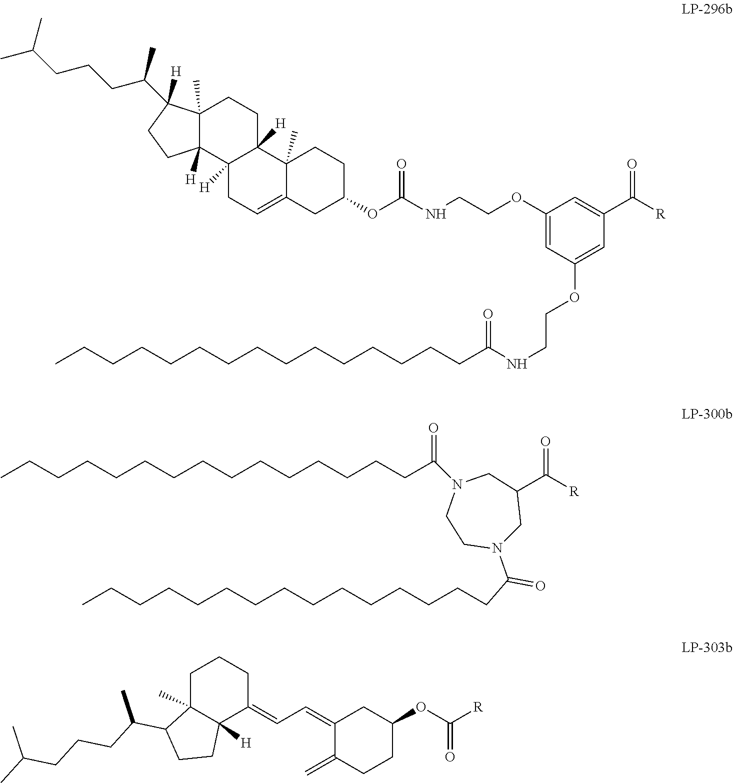 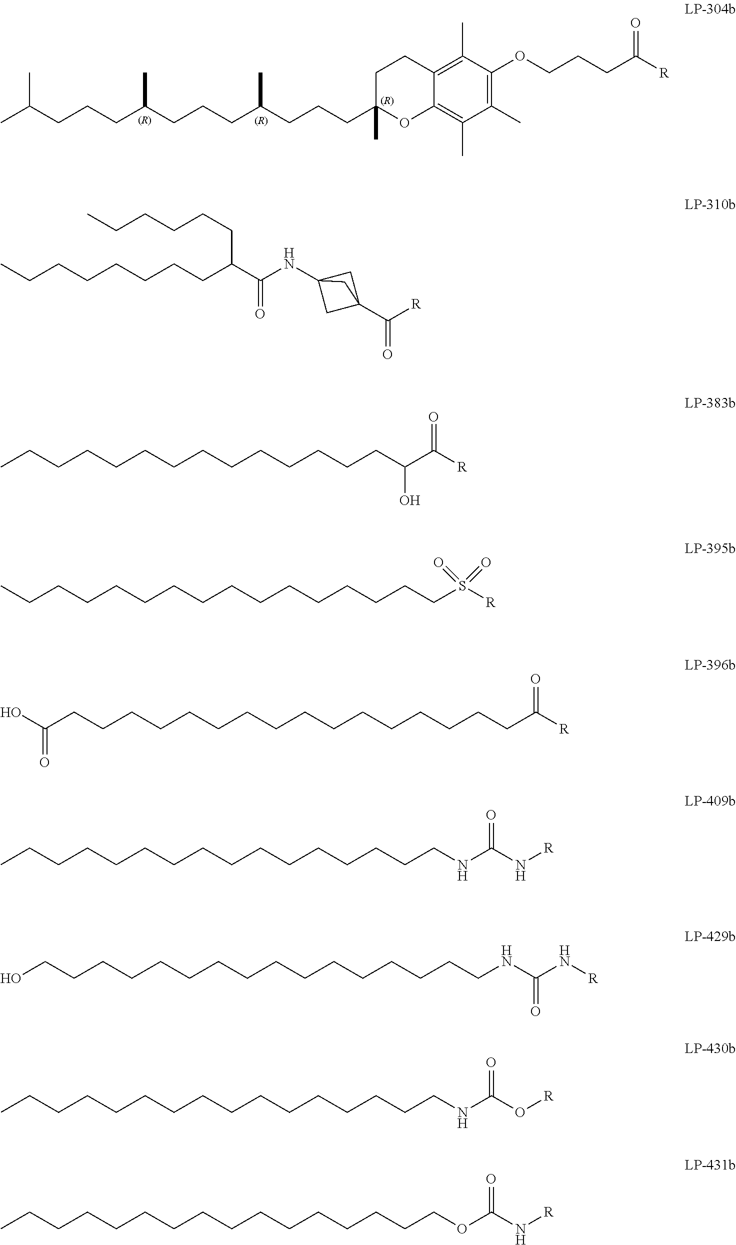 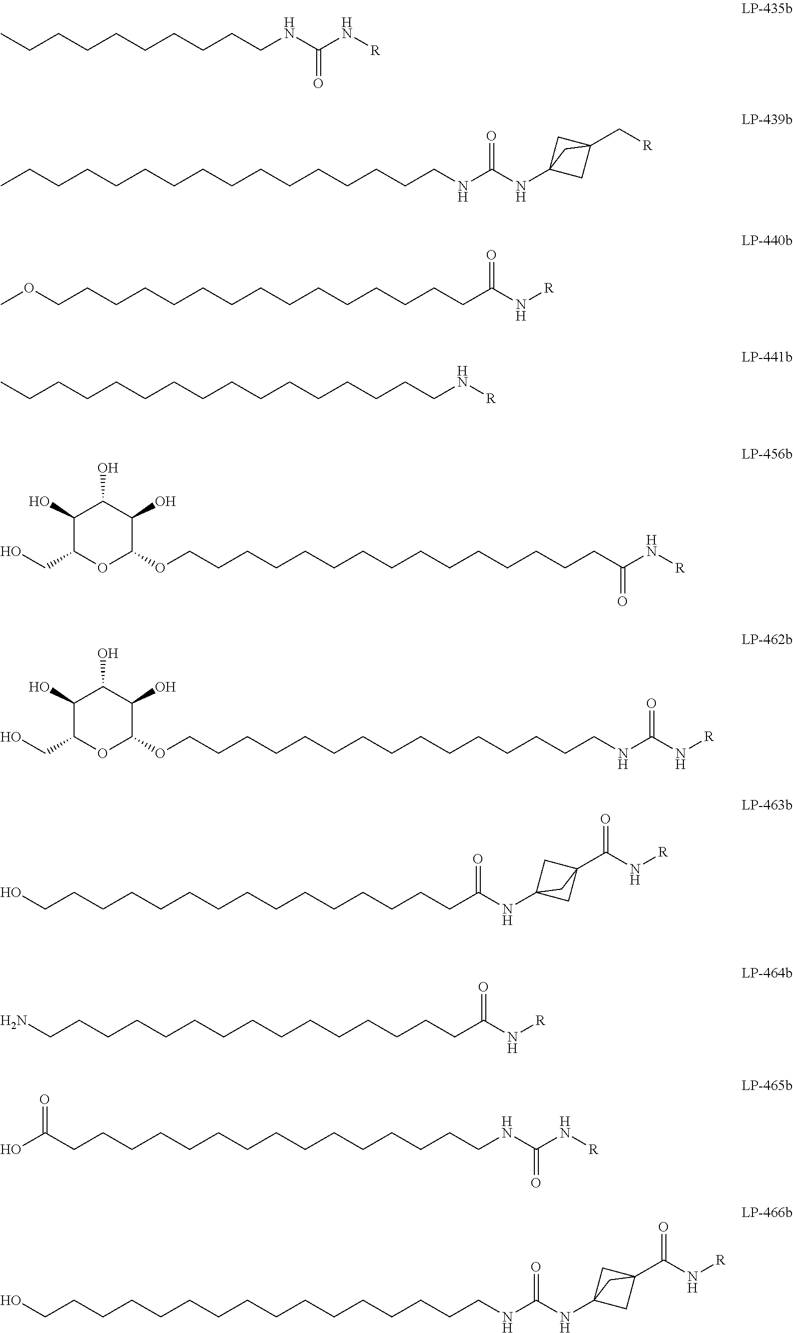 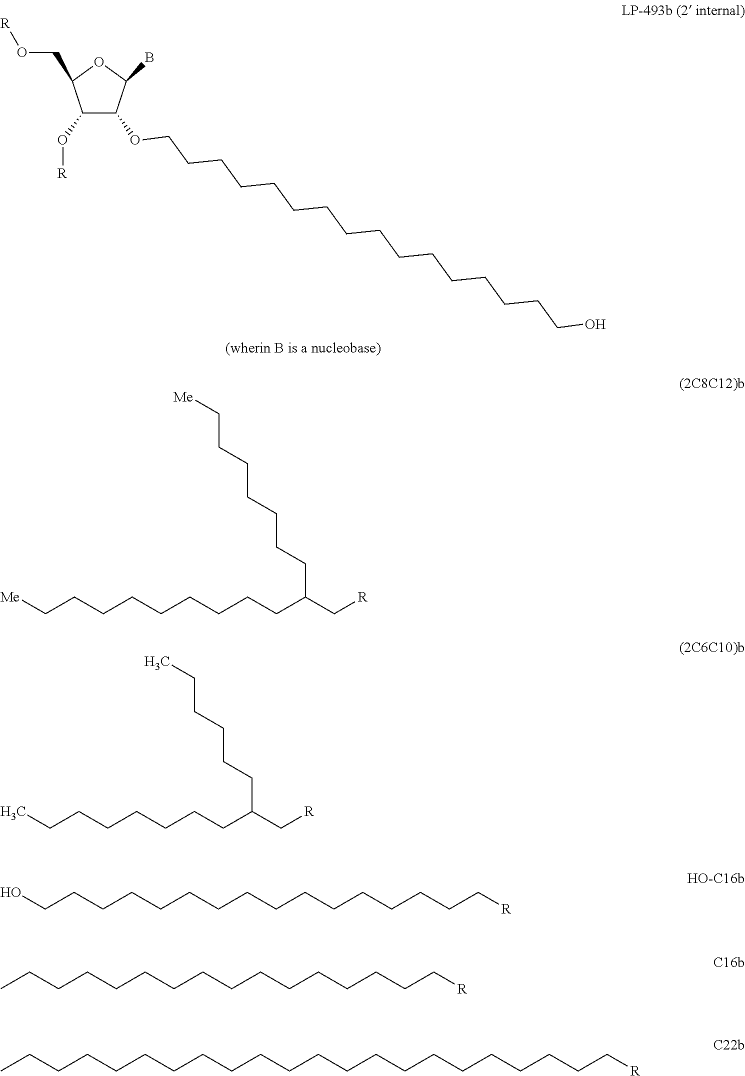
wherein R comprises an oligonucleotide.

15. The compound of claim 14, wherein the oligonucleotide is double-stranded and comprises a sense strand and an antisense strand.

16-17. (canceled)

18. The compound of claim 15, wherein the point of connection to R is on the 5′ terminal nucleotide of the sense strand.

19. (canceled)

20. A compound selected from the group consisting of: 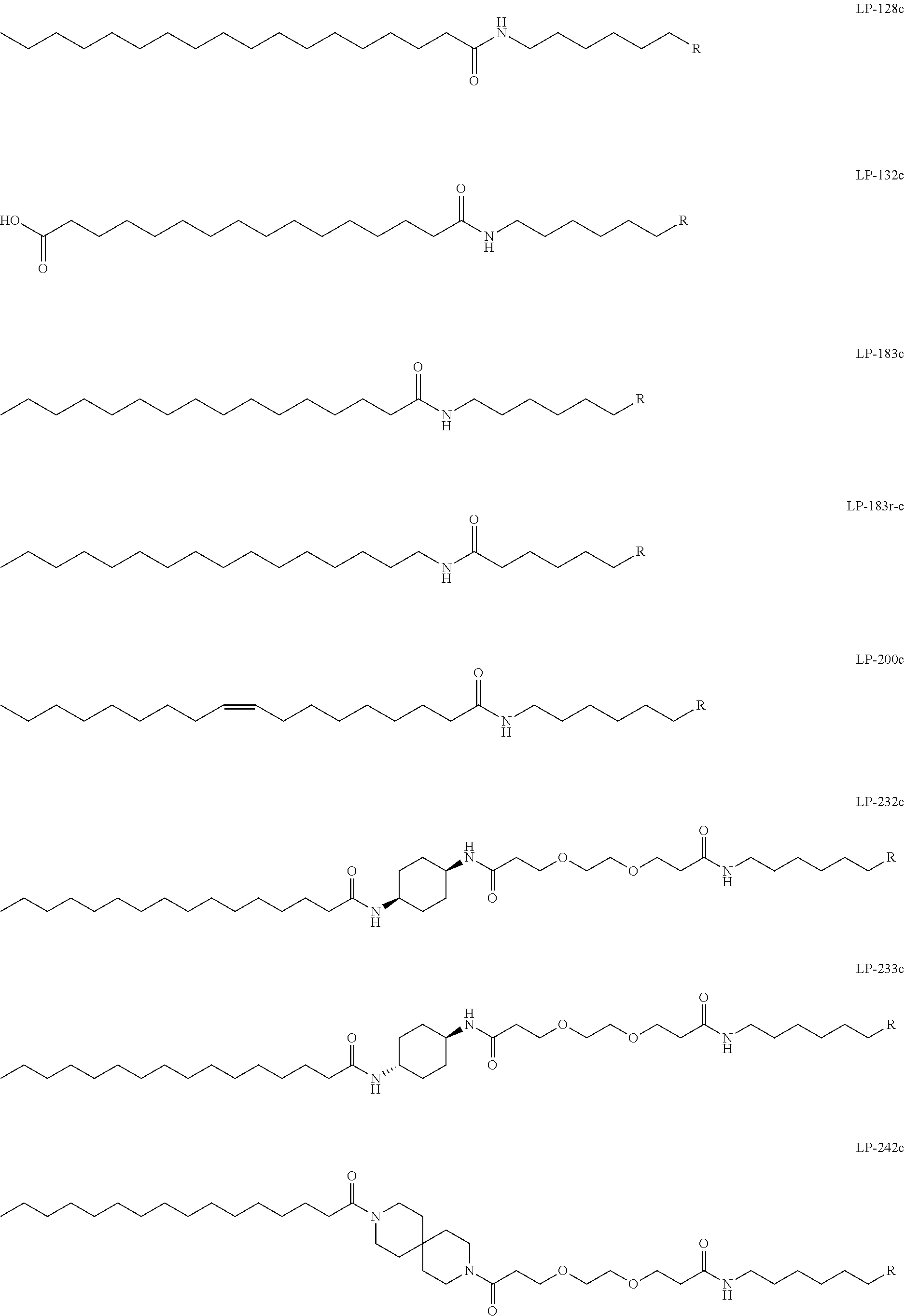 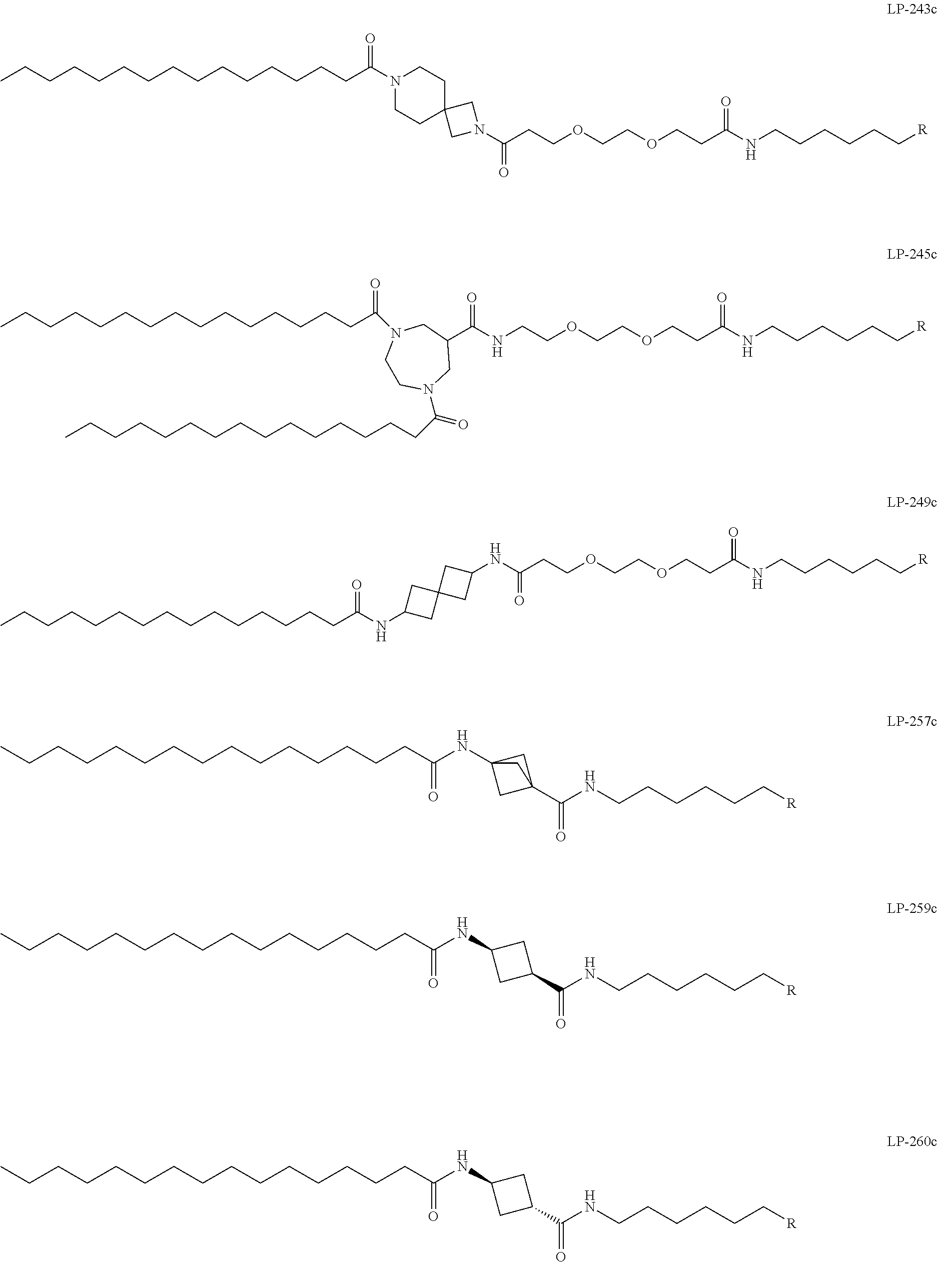 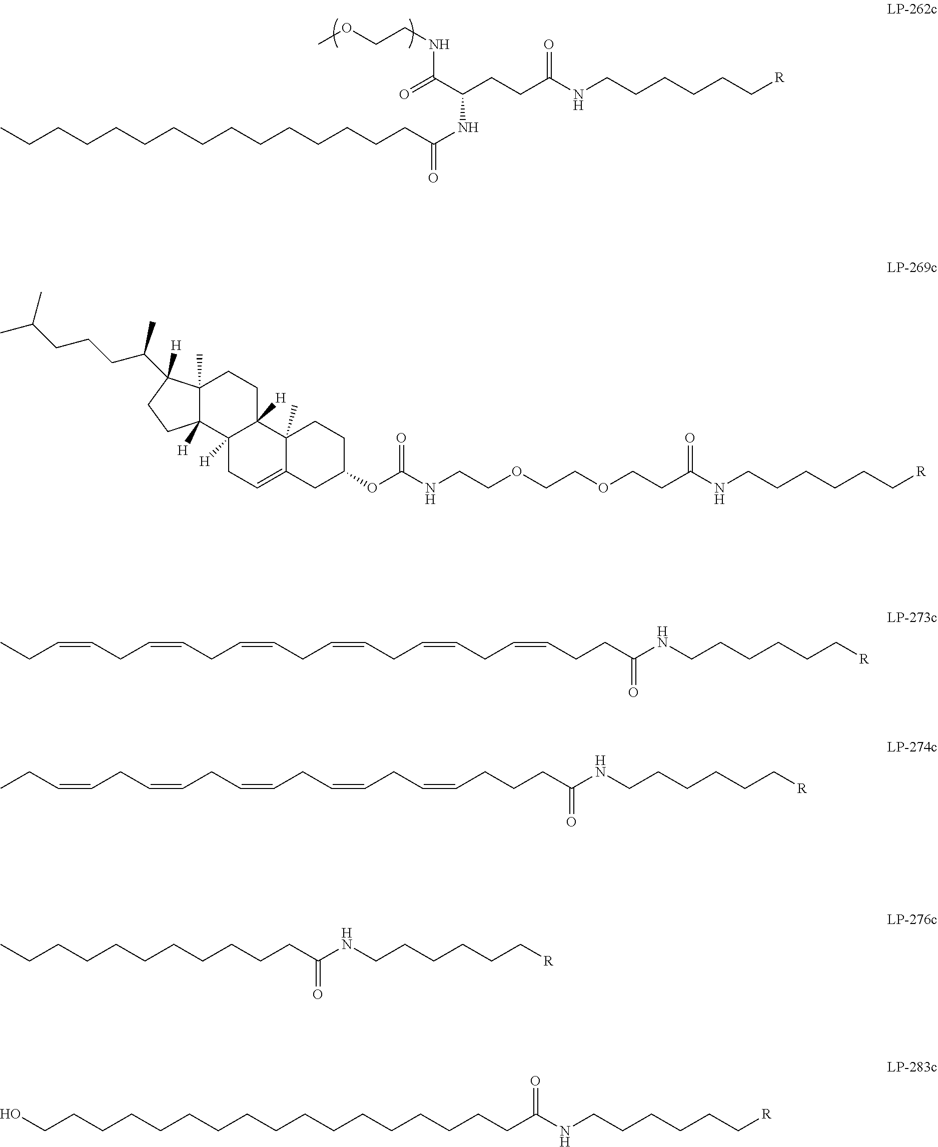 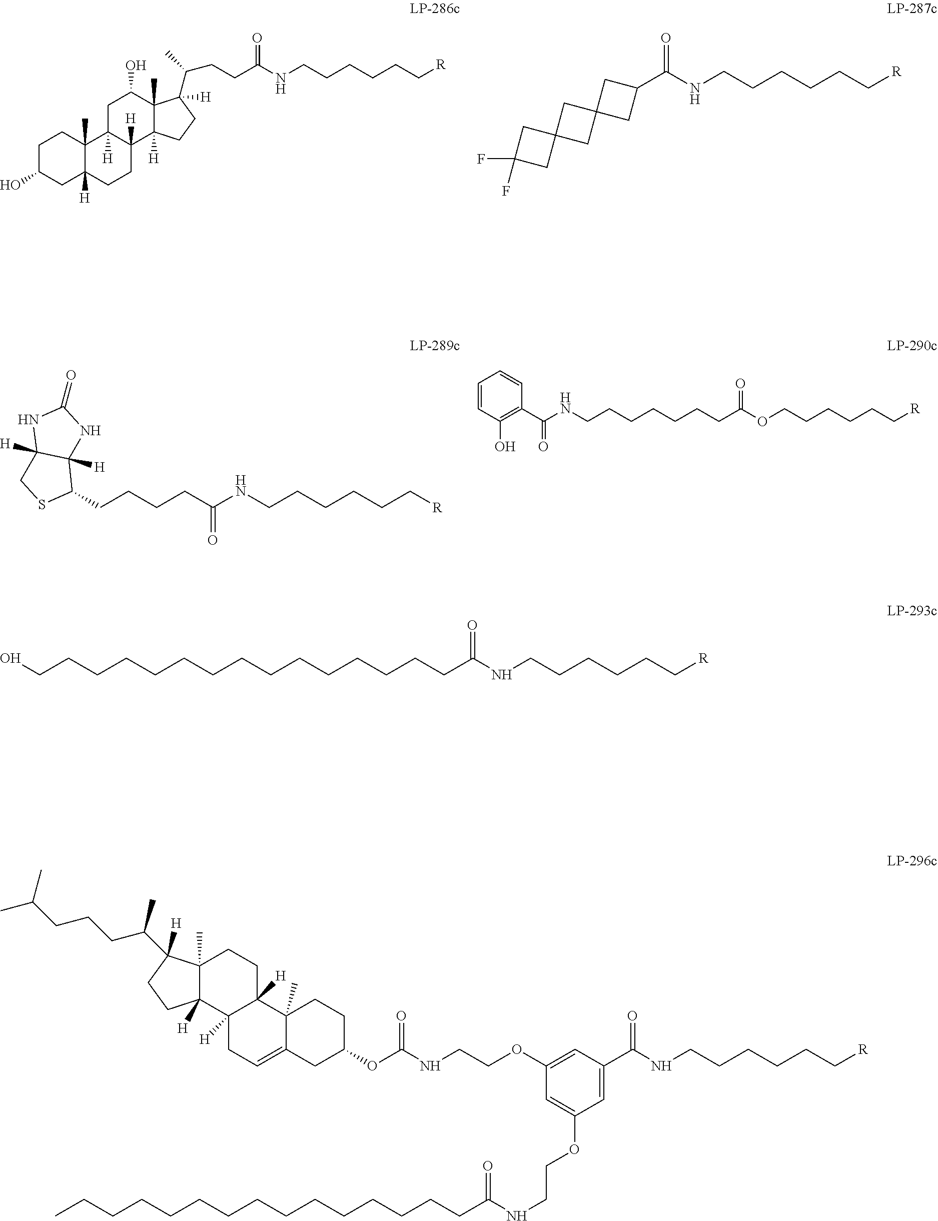 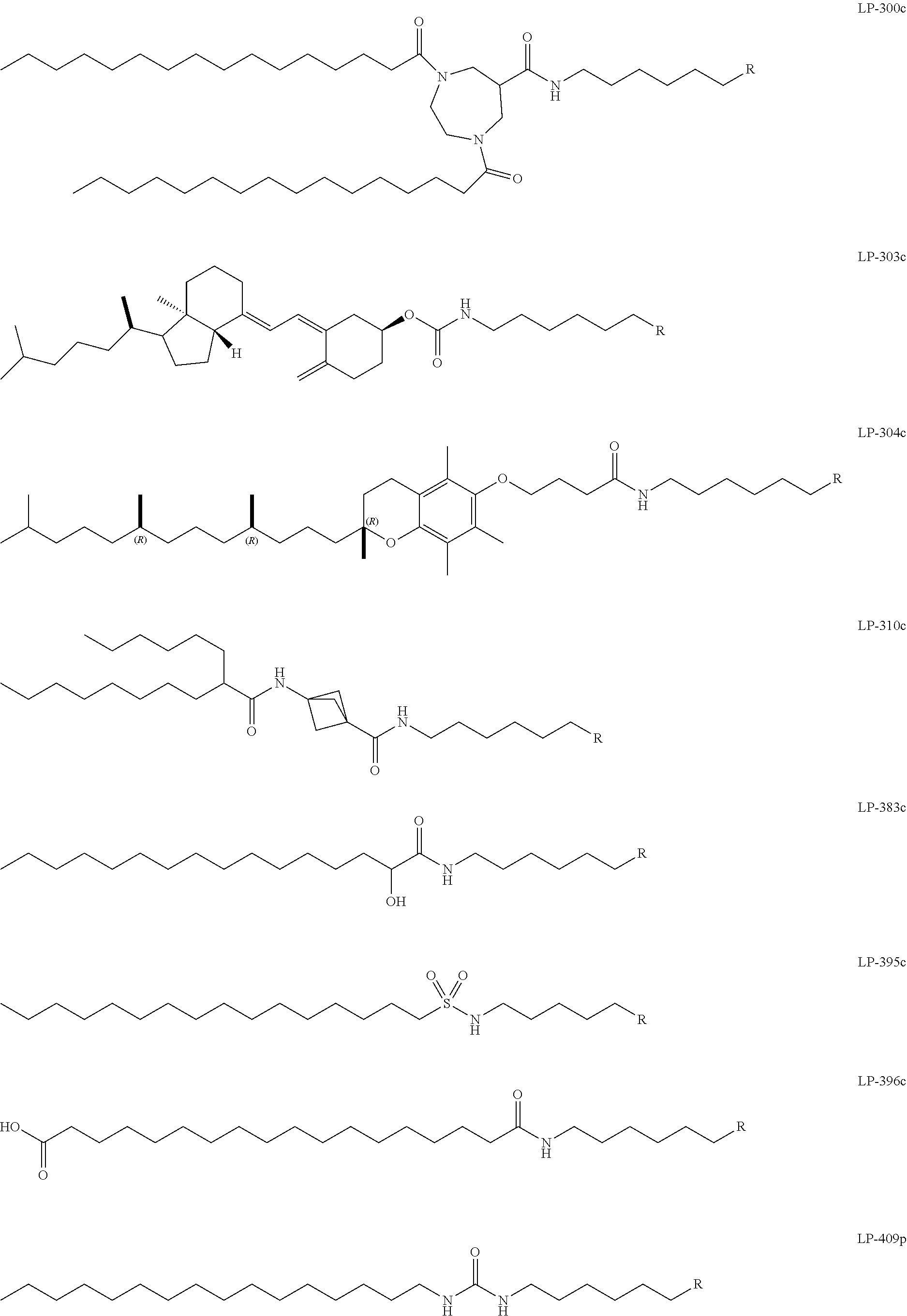 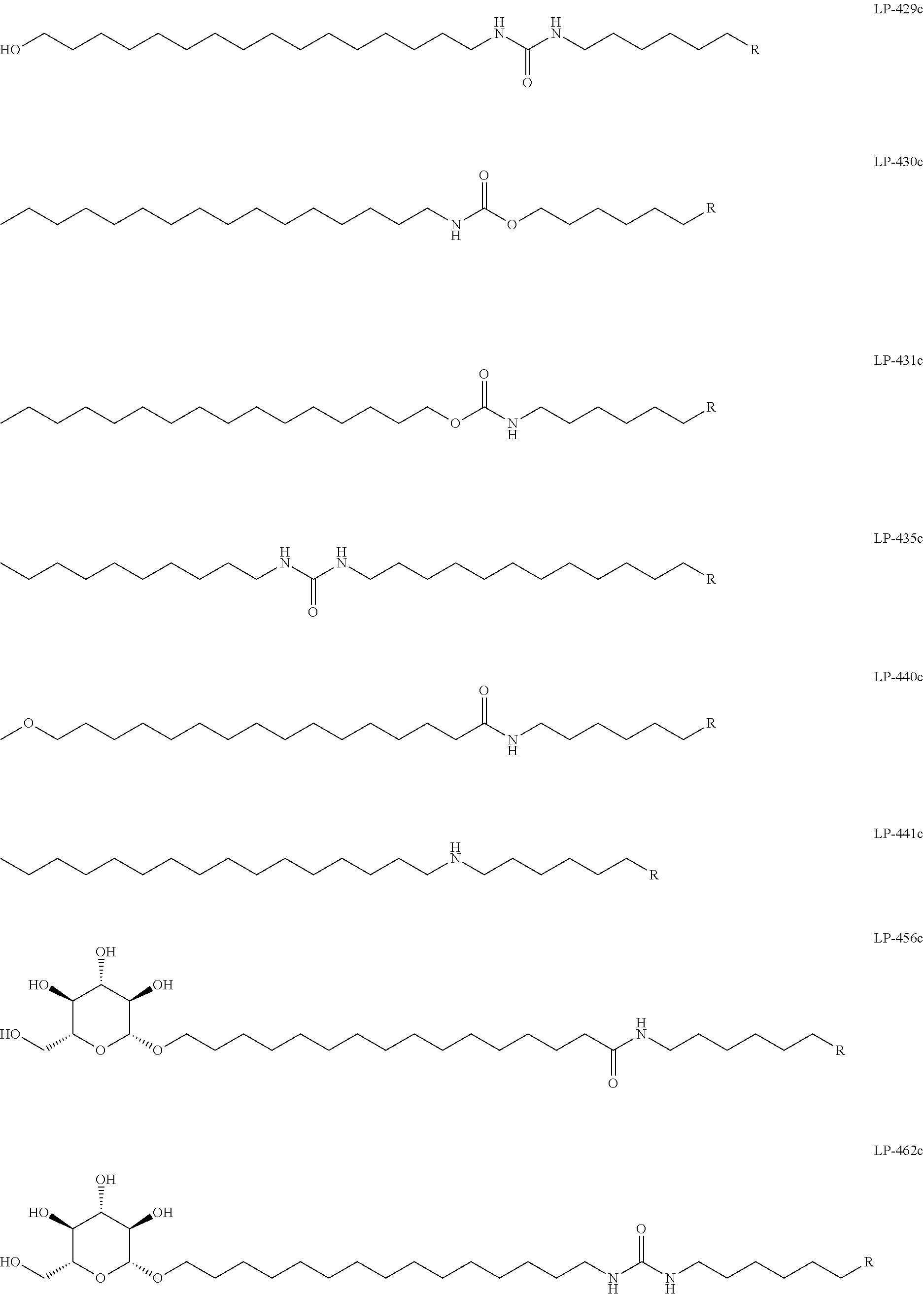 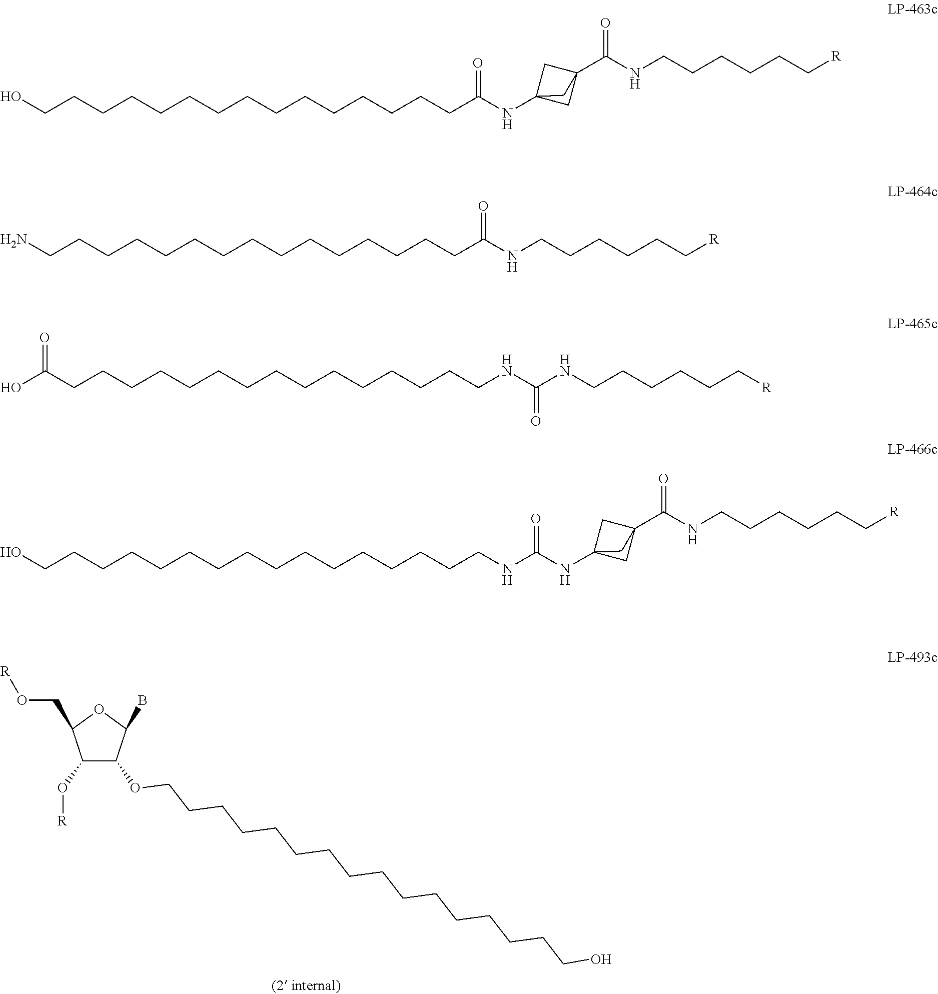
wherein R comprises an oligonucleotide.

21-23. (canceled)

24. The compound of claim 20, wherein the oligonucleotide comprises a sense strand and an antisense strand, and the point of connection to R is on the 5′ terminal nucleotide of the sense strand.

25. (canceled)

26. A compound comprising:
a) an oligonucleotide; and
b) a hydroxy lipid conjugated to an internal nucleotide of the oligonucleotide; wherein the hydroxy lipid comprises a hydroxyl group, and
wherein the oligonucleotide comprises at least 15 nucleotides that are complementary to a gene expressed in CNS tissue.

27. (canceled)

28. The compound of claim 26, wherein the hydroxy lipid is conjugated to the 2′ carbon of the internal nucleotide.

29-31. (canceled)

32. The compound of claim 26, wherein the hydroxy lipid is 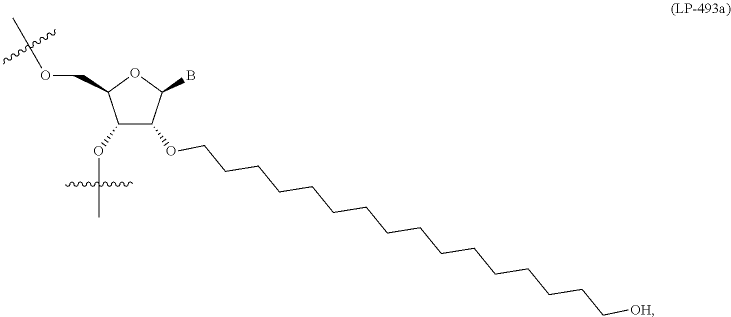
wherein [symbol] indicates the point of connection to the remainder of the oligonucleotide.

33. (canceled)

34. A method for delivering an oligonucleotide to a cell, comprising administering to a subject a compound of claim 1, wherein the cell is part of the central nervous system.

35. (canceled)

36. A method of treating a disease or disorder of the central nervous system, comprising administering to a subject in need thereof a compound of claim 1.

37. The method of claim 36, wherein the disease or disorder is selected from the group consisting of: Alzheimer's Disease, Parkinson's Disease, Huntington's Disease, Amyotrophic lateral sclerosis (ALS), Spinal muscular atrophy (SMA), and Lewy body disease.

38. The method of claim 36, wherein the compound comprises an oligonucleotide comprising a nucleotide sequence that is complementary to a gene expressed in a CNS cell, wherein the gene is selected from the group consisting of Superxoide Dismutase type 1 (SOD1), Amyloid Precursor Protein (APP), Ataxin 2 (ATXN2), Ataxin 3 (ATXN3), Sodium Voltage-Gated Channel Alpha Subunit 9 (SCN9A), Huntingtin (HTT), Alpha-Synuclein (SNCA), chromosome 9 open reading frame 72 (C9orf72), Leucine Rich Repeat Kinase 2 (LRRK2), Adrenoreceptor Alpha 2 A (ADRA2A), and androgen receptor (AR.).

39. (canceled)

40. A compound having the structure: 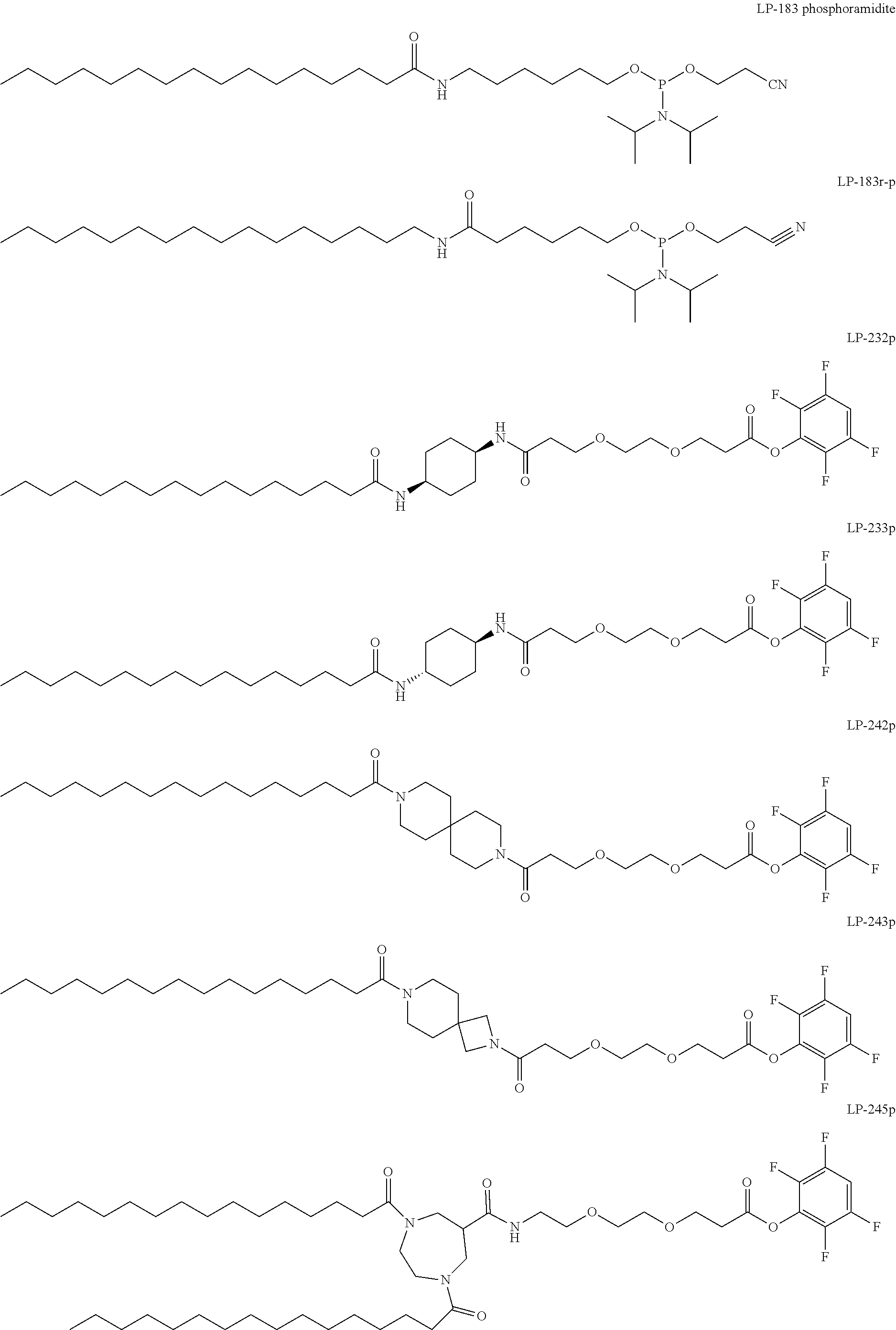 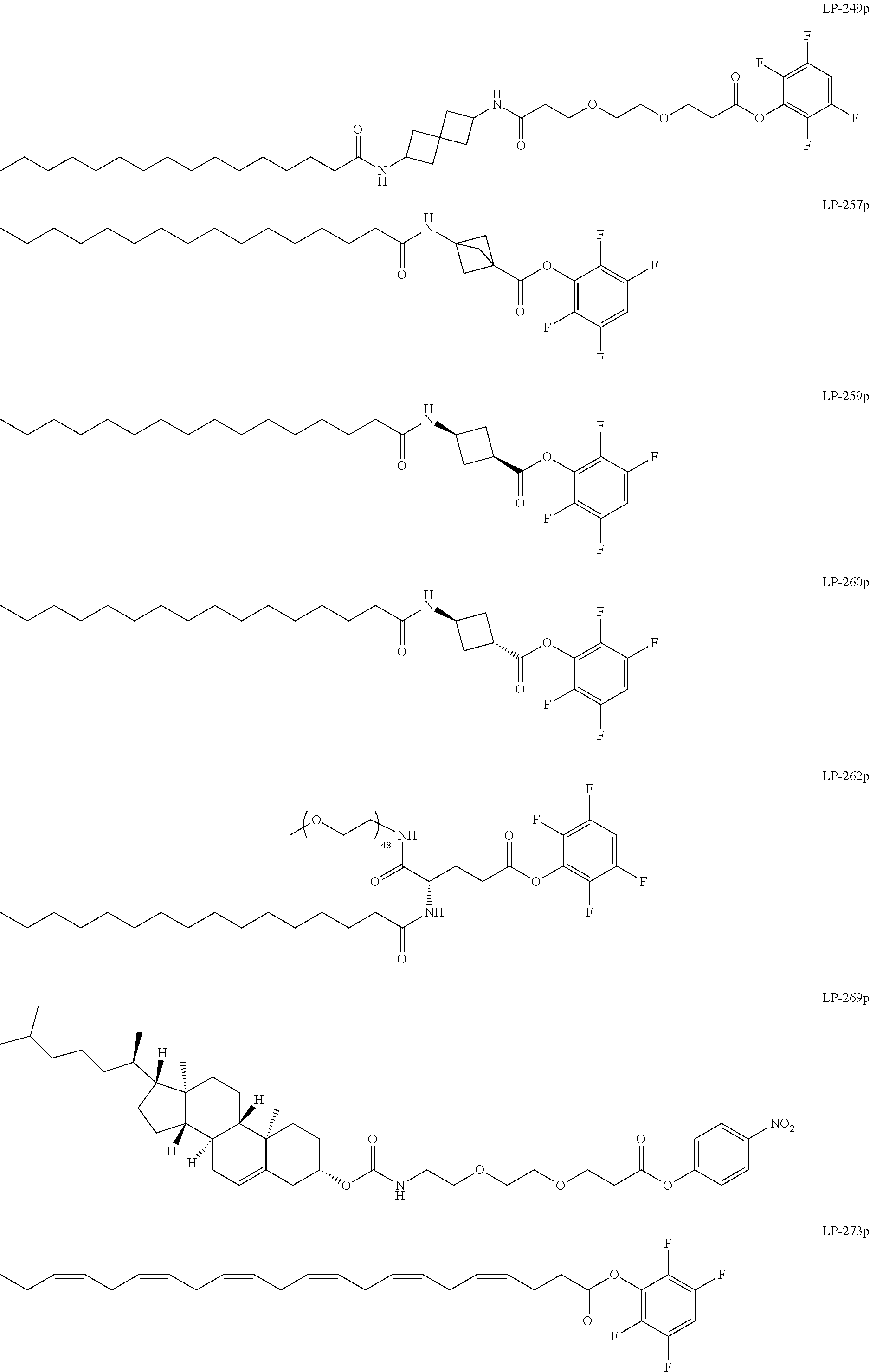 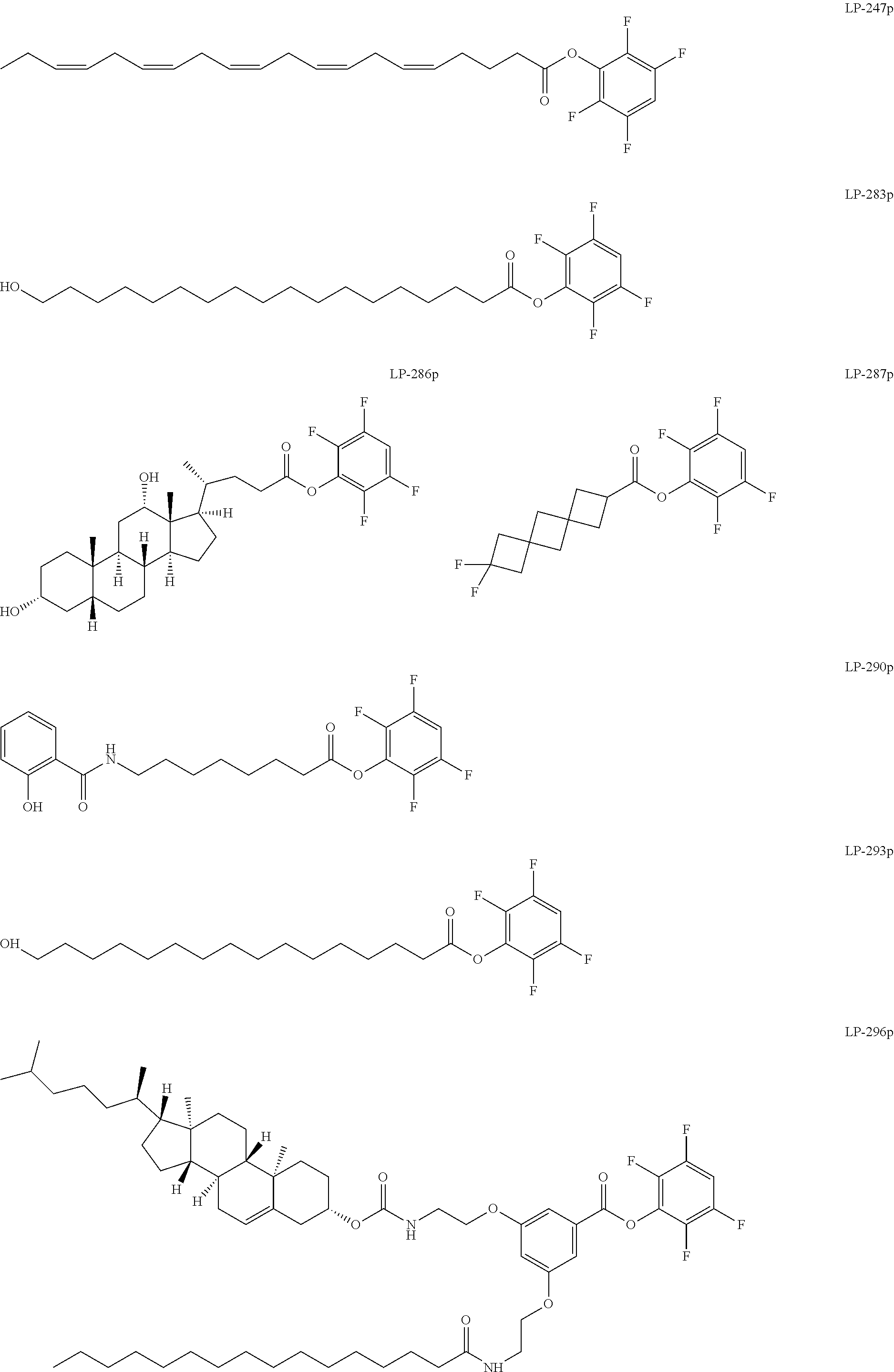 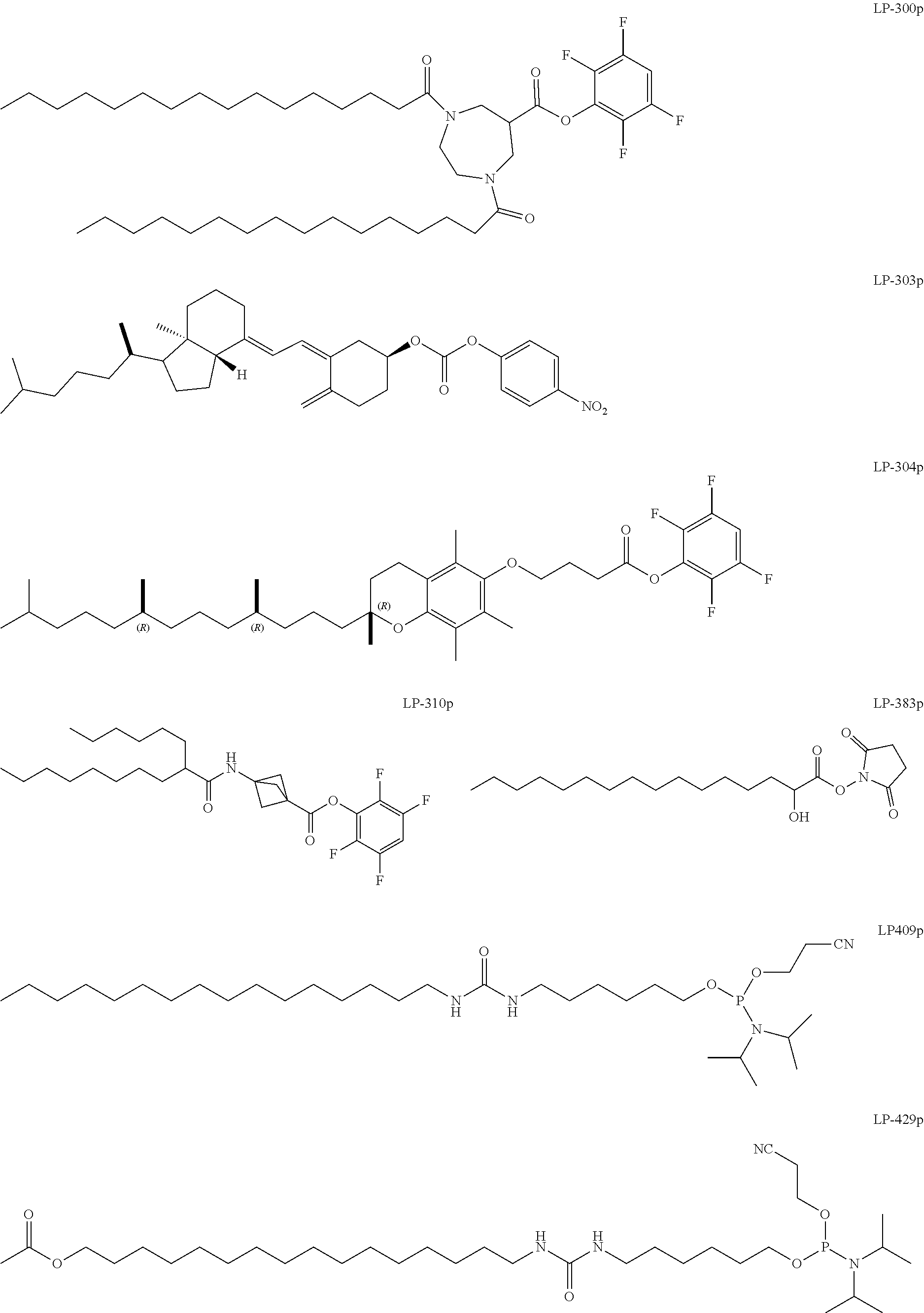 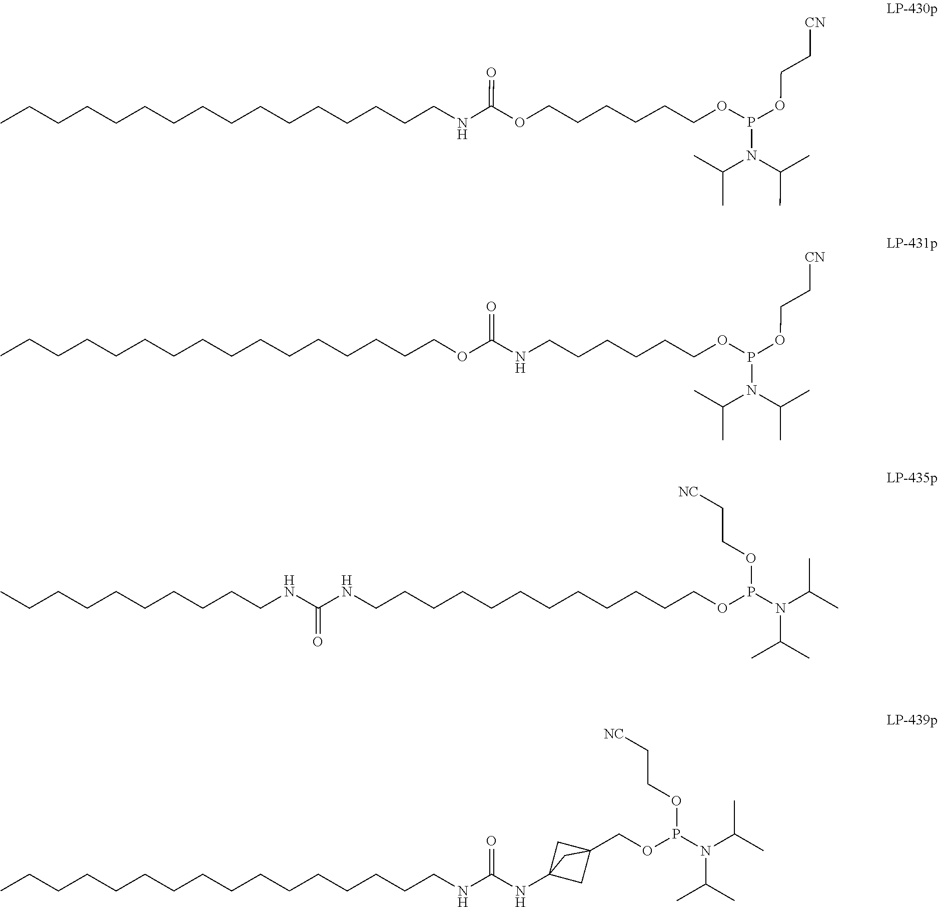 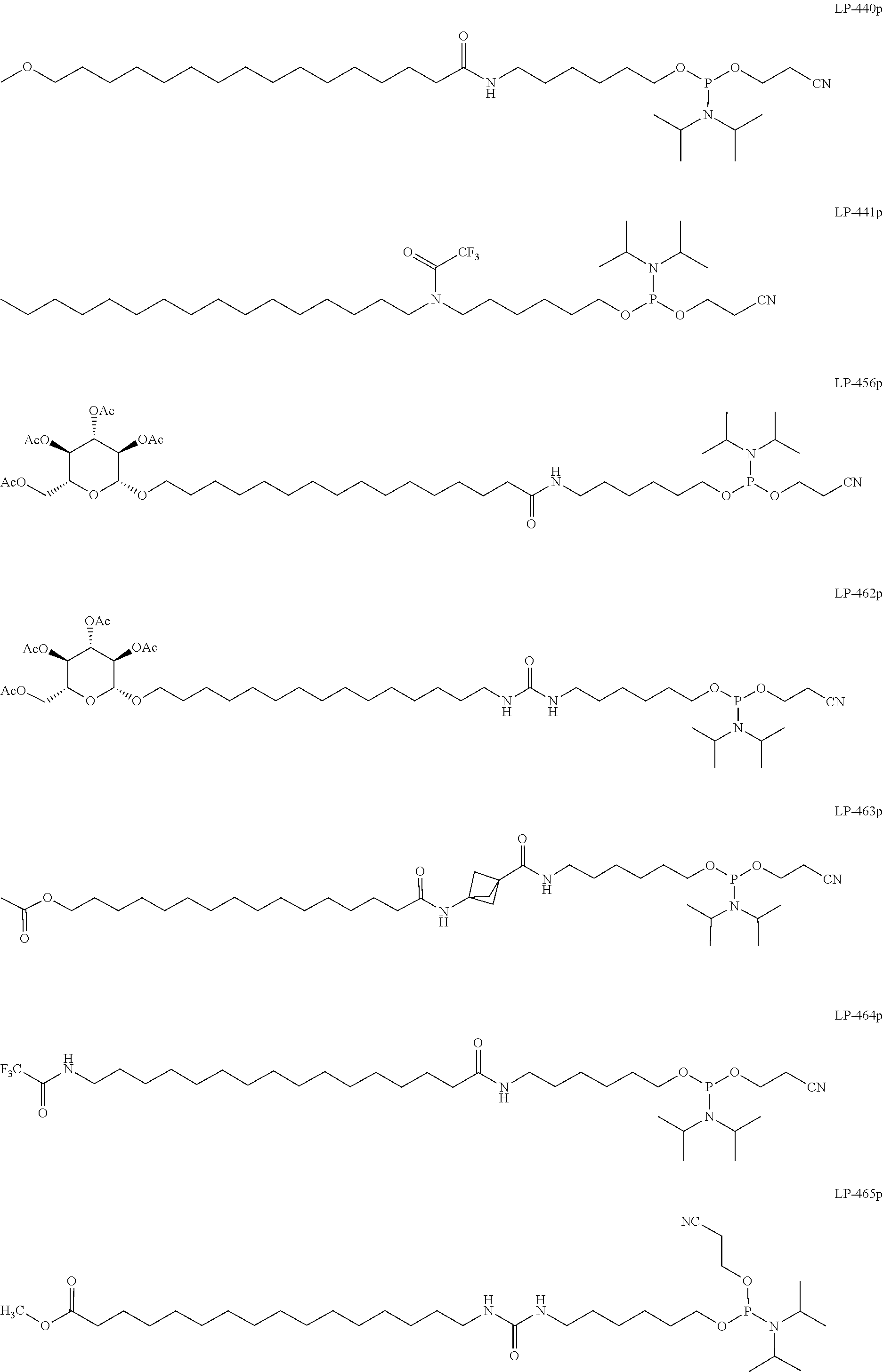 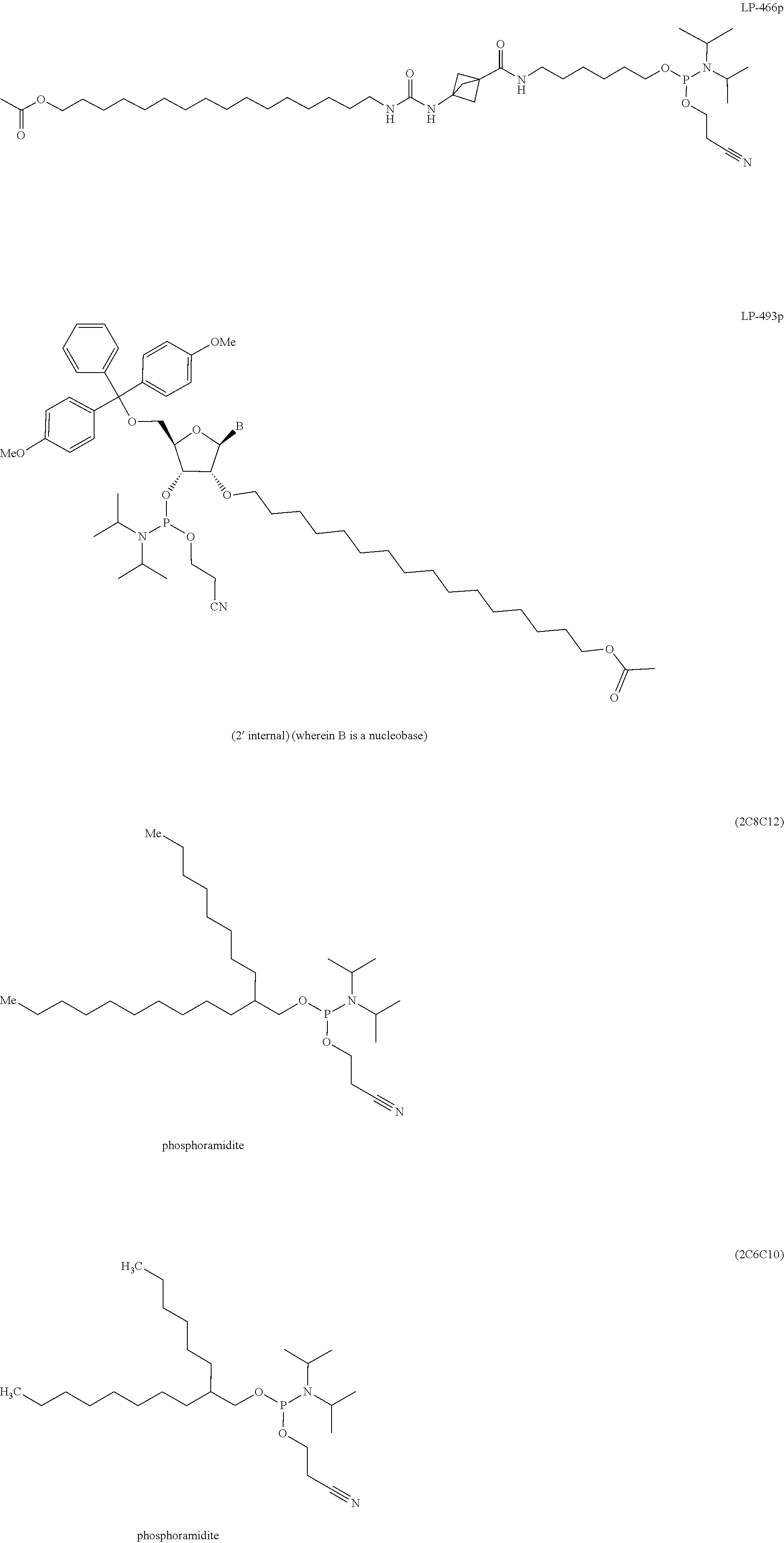 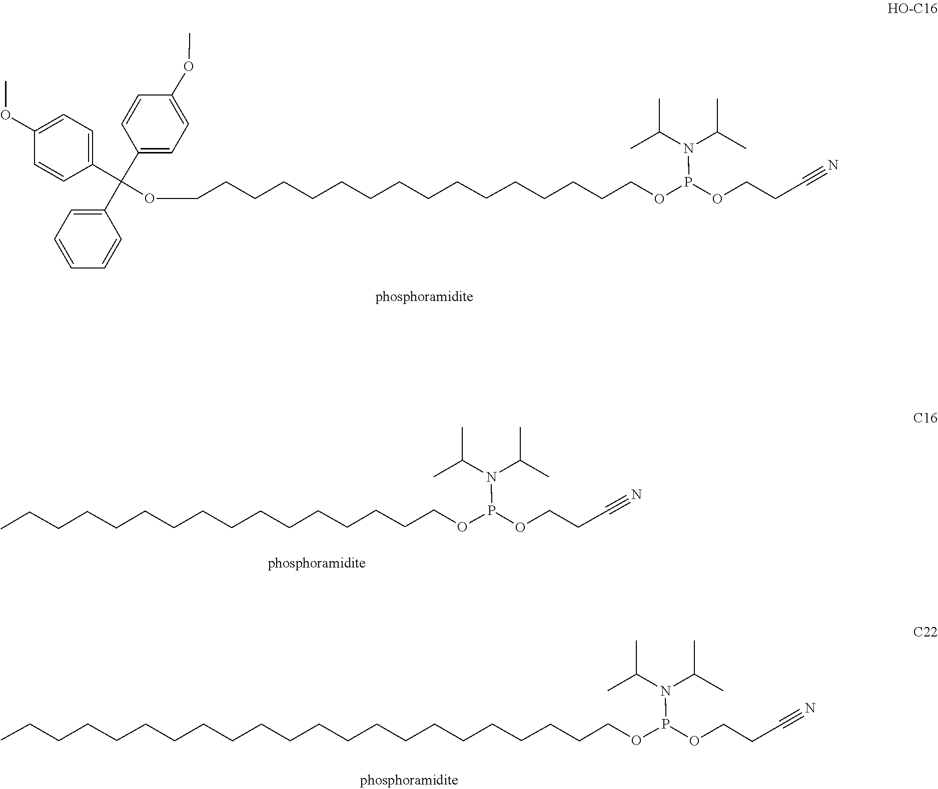 [SMILES: COc1ccc(C(OCCCCCCCCCCCCCCCCOP(OCCC#N)N(C(C)C)C(C)C)(c2ccccc2)c2ccc(OC)cc2)cc1 ; CCCCCCCCCCCCCCCCOP(OCCC#N)N(C(C)C)C(C)C ; CCCCCCCCCCCCCCCCCCCCCCOP(OCCC#N)N(C(C)C)C(C)C]
or a pharmaceutically acceptable salt thereof.

41. A method of synthesizing a compound of claim 1, comprising reacting a compound of claim 40 with an oligonucleotide-containing compound.
